SEQUENCE LISTING (145 sequences)

SEQ ID NO: 1 - protein, 7 aa, synthetic construct
  REGION 1..7: note = RT canonical motif
  REGION 3..5: note = misc_feature - Xaa can be any naturally occurring amino acid
QGXXXSP

SEQ ID NO: 2 - skipped

SEQ ID NO: 3 - DNA, 294 bp, Bordetella phage BP-1
  source 1..294: mol_type = genomic DNA
aagggcaggctgggaaataacgctgctgcgctattcggcggcaactggaacaacacgtcgaactcgggttctcgcgctgcgaactggaacaacgggccgtcgaactcgaacgcgaacatcggggcgcgcggcgtctgtgcccatcaccttcttgcatggctctgccaacgctacggcttggcgggctggcctttcctcaataggtggtcagccggttctgtcctgcttcggcgaacacgttacacggttcggcaaaacgtcgattactgaaaatggaaaggcggggccgacttc

SEQ ID NO: 4 - protein, 328 aa, Bordetella phage BP-1
MGKRHRNLIDQITTWENLLDAYRKTSHGKRRTWGYLEFKEYDLANLLALQAELKAGNYERGPYREFLVYEPKPRLISALEFKDRLVQHALCNIVAPIFEAGLLPYTYACRPDKGTHAGVCHVQAELRRTRATHFLKSDFSKFFPSIDRAALYAMIDKKIHCAATRRLLRVVLPDEGVGIPIGSLTSQLFANVYGGAVDRLLHDELKQRHWARYMDDIVVLGDDPEELRAVFYRLRDFASERLGLKISHWQVAPVSRGINFLGYRIWPTHKLLRKSSVKRAKRKVANFIKHGEDESLQRFLASWSGHAQWADTHNLFTWMEEQYGIACH

SEQ ID NO: 5 - protein, 128 aa, Bordetella phage BP-1
MEPIEEATKCYDQMLIVERYERVISYLYPIAQSIPRKHGVAREMFLKCLLGQVELFIVAGKSNQVSKLYAADAGLAMLRFWLRFLAGIQKPHAMTPHQVETAQVLIAEVGRILGSWIARVNRKGQAGK

SEQ ID NO: 6 - protein, 270 aa, Collinsella stercoris
MNQIVKFTDDSGLAVQVTPDDVRRYICENATEKEVGLFLQLCQTQRLNPFVKDAYLVKYGGAPASMITSYQVFNRRACRDANYDGIKSGVVVLRDGDVVHKRGAACYKKAGEELIGGWAEVRFKDGRETAYAEVALDDYSTGKSNWAKMPGVMIEKCAKAAAWRLAFPDTFQGMYAAEEMDQAQQPEQVRAQAEQPVDLQPIRELFKPYCEHFGITPAEGMTAVCGAVGAEGMHSMTEQQARRARAWMEEEMAAPAVEAEYEVVDEGEVF

SEQ ID NO: 7 - DNA, 987 bp, synthetic construct
  misc_feature 1..987: note = synthetic polynucleotide (recoded bTR)
  source 1..987: mol_type = other DNA
atgggaaagcgccatcgtaaccttattgaccaaatcaccacctgggaaaatttgttagatgcgtaccgtaagactagccacggtaagcgccgcacttgggggtatcttgaatttaaggagtacgatttggccaatttgcttgcacttcaggcggagttgaaggcaggcaattacgagcgcggaccgtatcgcgagtttctggtttacgaaccgaaaccccgcttgattagcgcactggaatttaaagatcgtcttgttcaacacgcgctgtgcaacatcgttgcgccaatctttgaagcaggtttgcttccatatacatacgcgtgtcgtccggataaggggacccatgcaggagtgtgtcatgttcaggcagaattgcgtcgcacgcgcgcgactcattttttgaagagtgactttagcaagttcttcccaagcattgaccgtgccgctctttatgcgatgattgataaaaaaatccactgcgccgctacacgtcgccttttgcgcgttgtcctgccggatgagggagttggaattcccattggcagcttaacctctcagttatttgccaacgtttacgggggcgcggtagatcgtttgttacacgacgagttaaagcagcgccactgggctcgttacatggatgacattgtcgtacttggggatgatccagaagaactgcgcgcggtcttctatcgtttgcgtgattttgcgtccgaacgcttaggtttgaagatttcacattggcaggtagcgcctgtgtctcgtggaatcaattttcttggttaccgcatctggccgacccataaattgctgcgtaagagtagtgtgaagcgcgctaagcgtaaggtggcaaatttcatcaaacacggagaagacgagtcactgcaacgcttccttgcctcgtggtcgggtcacgcccaatgggcggacactcacaatttattcacttggatggaggagcaatatggcatcgcgtgtcattaa

SEQ ID NO: 8 - DNA, 987 bp, synthetic construct
  misc_feature 1..987: note = synthetic polynucleotide (recoded sequence encoding bRT inactive variant with SMAA residues)
  source 1..987: mol_type = other DNA
atgggaaagcgccatcgtaaccttattgaccaaatcaccacctgggaaaatttgttagatgcgtaccgtaagactagccacggtaagcgccgcacttgggggtatcttgaatttaaggagtacgatttggccaatttgcttgcacttcaggcggagttgaaggcaggcaattacgagcgcggaccgtatcgcgagtttctggtttacgaaccgaaaccccgcttgattagcgcactggaatttaaagatcgtcttgttcaacacgcgctgtgcaacatcgttgcgccaatctttgaagcaggtttgcttccatatacatacgcgtgtcgtccggataaggggacccatgcaggagtgtgtcatgttcaggcagaattgcgtcgcacgcgcgcgactcattttttgaagagtgactttagcaagttcttcccaagcattgaccgtgccgctctttatgcgatgattgataaaaaaatccactgcgccgctacacgtcgccttttgcgcgttgtcctgccggatgagggagttggaattcccattggcagcttaacctctcagttatttgccaacgtttacgggggcgcggtagatcgtttgttacacgacgagttaaagcagcgccactgggctcgttctatggcggcgattgtcgtacttggggatgatccagaagaactgcgcgcggtcttctatcgtttgcgtgattttgcgtccgaacgcttaggtttgaagatttcacattggcaggtagcgcctgtgtctcgtggaatcaattttcttggttaccgcatctggccgacccataaattgctgcgtaagagtagtgtgaagcgcgctaagcgtaaggtggcaaatttcatcaaacacggagaagacgagtcactgcaacgcttccttgcctcgtggtcgggtcacgcccaatgggcggacactcacaatttattcacttggatggaggagcaatatggcatcgcgtgtcattaa

SEQ ID NO: 9 - DNA, 987 bp, synthetic construct
  misc_feature 1..987: note = synthetic polynucleotide (recodedsequence encoding bRT variant R74A)
  source 1..987: mol_type = other DNA
atgggaaagcgccatcgtaaccttattgaccaaatcaccacctgggaaaatttgttagatgcgtaccgtaagactagccacggtaagcgccgcacttgggggtatcttgaatttaaggagtacgatttggccaatttgcttgcacttcaggcggagttgaaggcaggcaattacgagcgcggaccgtatcgcgagtttctggtttacgaaccgaaacccgcattgattagcgcactggaatttaaagatcgtcttgttcaacacgcgctgtgcaacatcgttgcgccaatctttgaagcaggtttgcttccatatacatacgcgtgtcgtccggataaggggacccatgcaggagtgtgtcatgttcaggcagaattgcgtcgcacgcgcgcgactcattttttgaagagtgactttagcaagttcttcccaagcattgaccgtgccgctctttatgcgatgattgataaaaaaatccactgcgccgctacacgtcgccttttgcgcgttgtcctgccggatgagggagttggaattcccattggcagcttaacctctcagttatttgccaacgtttacgggggcgcggtagatcgtttgttacacgacgagttaaagcagcgccactgggctcgttacatggatgacattgtcgtacttggggatgatccagaagaactgcgcgcggtcttctatcgtttgcgtgattttgcgtccgaacgcttaggtttgaagatttcacattggcaggtagcgcctgtgtctcgtggaatcaattttcttggttaccgcatctggccgacccataaattgctgcgtaagagtagtgtgaagcgcgctaagcgtaaggtggcaaatttcatcaaacacggagaagacgagtcactgcaacgcttccttgcctcgtggtcgggtcacgcccaatgggcggacactcacaatttattcacttggatggaggagcaatatggcatcgcgtgtcattaa

SEQ ID NO: 10 - DNA, 987 bp, synthetic construct
  misc_feature 1..987: note = synthetic polynucleotide (recoded sequence encoding bRT variant I181N)
  source 1..987: mol_type = other DNA
atgggaaagcgccatcgtaaccttattgaccaaatcaccacctgggaaaatttgttagatgcgtaccgtaagactagccacggtaagcgccgcacttgggggtatcttgaatttaaggagtacgatttggccaatttgcttgcacttcaggcggagttgaaggcaggcaattacgagcgcggaccgtatcgcgagtttctggtttacgaaccgaaaccccgcttgattagcgcactggaatttaaagatcgtcttgttcaacacgcgctgtgcaacatcgttgcgccaatctttgaagcaggtttgcttccatatacatacgcgtgtcgtccggataaggggacccatgcaggagtgtgtcatgttcaggcagaattgcgtcgcacgcgcgcgactcattttttgaagagtgactttagcaagttcttcccaagcattgaccgtgccgctctttatgcgatgattgataaaaaaatccactgcgccgctacacgtcgccttttgcgcgttgtcctgccggatgagggagttggaattcccaacggcagcttaacctctcagttatttgccaacgtttacgggggcgcggtagatcgtttgttacacgacgagttaaagcagcgccactgggctcgttacatggatgacattgtcgtacttggggatgatccagaagaactgcgcgcggtcttctatcgtttgcgtgattttgcgtccgaacgcttaggtttgaagatttcacattggcaggtagcgcctgtgtctcgtggaatcaattttcttggttaccgcatctggccgacccataaattgctgcgtaagagtagtgtgaagcgcgctaagcgtaaggtggcaaatttcatcaaacacggagaagacgagtcactgcaacgcttccttgcctcgtggtcgggtcacgcccaatgggcggacactcacaatttattcacttggatggaggagcaatatggcatcgcgtgtcattaa

SEQ ID NO: 11 - DNA, 387 bp, synthetic construct
  misc_feature 1..387: note = synthetic polynucleotide (recoded bAvd)
  source 1..387: mol_type = other DNA
ttattttccggcttgtcctttacggttcacacgagcaatccaggaccctaagatacggccaacctccgcgataagcacctgagcagtctcgacctggtgcggagtcatggcatgcggcttttgaatgcctgccaaaaagcgaagccagaagcgtaacatcgccaaacccgcgtcagcagcatacagtttcgagacctggtttgatttacctgctacaatgaacagttccacctgtcccaacagacatttcaaaaacatttcgcgtgcgactccatgtttacgagggattgactgagcaatgggatacaagtatgagatgacgcgttcatagcgctctacaatcaacatctgatcgtaacacttggtagcctcttcaataggttccat

SEQ ID NO: 12 - DNA, 966 bp, synthetic construct
  misc_feature 1..966: note = synthetic polynucleotide (recoded XylS modified to remove BsaI restriction site)
  source 1..966: mol_type = other DNA
tcaagccacttcctttttgcattgacgcagggtgtcggaaggcaactcgccgaacgcgctcctatagttttcagcgaagcgtcccaaatgtaagaagccgtagtctagggctatctcagttatactacgcacattggcactgggatcgttcaagcaggcgcggatgctttcgagcttgcggttgcggatgtagttcttcggcgtggtgccggcgtgcttctcgaacaaattgtagagcgagcgtggactcatcatcgccagctccgctaaccgctcaaggctgatattccgtttgagattctcctcaatgaattgaacgactcgctcgaaagacgggttacctttgctgaaaatttcacggctgacattgctgcccagcatttcgagcagcttggaagcgatgatccccgcatagtgctcttggacccgaggcatcgactttgtatgttccgcttcgtcacaaactaacccgagtagattgataaagccatcgagttgctggagattgtgtcgcgcggcgaaacggataccctccctcggcttgtgccaattgttgtcactgcacgcccgatcaaggaccactgagggcaatttaacgataaatttctcgcaatcttctgaataggtcaggtcggcttggtcatccggattgagcagcaatagttcgcccggcgcaaaatagtgctcctggccatggccacgccacaggcaatggcctttgagtattatttgcagatgataacaggtttctaatccaggcgagattaccctcacgctaccgccgtagctgattcgacacagatcgaggcatccgaagattctgtggtgcagcctgcctgccgggcgcccgcccttgggcaggcgaatagagtgcgtaccgacatactggttaacataatcggagactgcatagggctcggcgtggacgaagatctgacttttctcgttcaataagcaaaaatccat

SEQ ID NO: 13 - DNA, 600 bp, synthetic construct
  misc_feature 1..600: note = synthetic polynucleotide (recoded phlF promoter)
  source 1..600: mol_type = other DNA
atggcacgtaccccgtcacgtagtagcattggtagcctgcgtagtccgcatacccataaagcaattctgaccagtaccatcgagatcctgaaagaatgtggttatagcggactgagcattgaaagcgttgcacgtcgtgccggagcaagcaaaccgaccatttatcgttggtggacgaataaagcagcactgattgccgaagtgtatgaaaatgaaagcgaacaggtgcgtaaatttccggatctgggtagctttaaagcagatctggattttttactgcgtaatttatggaaagtttggcgtgaaactatttgcggtgaagcatttcgttgtgttattgcagaagctcagctggatcctgcaaccctgacccagttaaaggatcaatttatggaacgtcgtcgtgagatgccgaaaaaactggttgaaaatgccattagcaatggtgaactgccgaaagataccaatcgtgaacttcttctggatatgatttttggtttttgttggtatcgcctgttaaccgaacagctgaccgttgaacaggatattgaagaatttaccttccttctgattaatggtgtttgtccgggtactcagcgt

SEQ ID NO: 14 - DNA, 813 bp, synthetic construct
  misc_feature 1..813: note = synthetic polynucleotide (recoded CspRecT)
  source 1..813: mol_type = other DNA
atgaaccaaatcgtgaagttcactgacgactctggcctggcggttcaagttactccagacgatgttcgccgttatatctgtgagaacgctactgaaaaagaggtgggcctctttctgcaactctgtcagactcaacgtctgaatccgtttgtgaaagacgcttacctggtgaaatacggcggtgctccagcttctatgattacttcctatcaagtttttaaccgtcgcgcgtgtcgtgatgctaactatgatggtatcaaatctggtgtggttgttctgcgtgacggtgatgttgtgcataaacgtggtgctgcgtgctacaaaaaggcgggtgaggagctcatcggtggttgggcggaagttcgctttaaggatggccgcgagactgcgtatgctgaggtggcgctcgacgactattccaccggcaaatctaattgggcgaaaatgccgggtgttatgatcgaaaaatgcgcgaaggctgctgcttggcgcctcgcgttcccggacacttttcagggcatgtacgctgcggaggaaatggatcaagcgcaacagccagaacaggtgcgcgctcaggcggagcaaccagtggatctccagccaatccgcgaactcttcaagccatattgcgaacacttcggcatcactccggctgagggtatgactgctgtttgtggtgcggtgggcgctgaaggcatgcactctatgaccgagcagcaagctcgccgtgctcgcgcttggatggaggaagaaatggctgcgccagctgtggaagcggagtatgaggttgttgacgagggcgaggtgttttaa

SEQ ID NO: 15 - DNA, 1848 bp, synthetic construct
  misc_feature 1..1848: note = synthetic polynucleotide (recoded mutL (E32K))
  source 1..1848: mol_type = other DNA
atgccaattcaggtcttaccgccacaactggcgaaccagattgccgcaggtgaggtggtcgagcgacctgcgtcggtagtcaaagaactagtgaaaaacagcctcgatgcaggtgcgacgcgtatcgatattgatatcgaacgcggtggggcgaaacttatccgcattcgtgataacggctgcggtatcaaaaaagatgagctggcgctggcgctggctcgtcatgccaccagtaaaatcgcctctctggacgatctcgaagccattatcagcctgggctttcgcggtgaggcgctggcgagtatcagttcggtttcccgcctgacgctcacttcacgcaccgcagaacagcaggaagcctggcaggcctatgccgaagggcgcgatatgaacgtgacggtaaaaccggcggcgcatcctgtggggacgacgctggaggtgctggatctgttctacaacaccccggcgcggcgcaaattcctgcgcaccgagaaaaccgaatttaaccacattgatgagatcatccgccgcattgcgctggcgcgtttcgacgtcacgatcaacctgtcgcataacggtaaaattgtgcgtcagtaccgcgcagtgccggaaggcgggcaaaaagaacggcgcttaggcgcgatttgcggcaccgcttttcttgaacaagcgctggcgattgaatggcaacacggcgatctcacgctacgcggctgggtggccgatccaaatcacaccacgcccgcactggcagaaattcagtattgctacgtgaacggtcgcatgatgcgcgatcgcctgatcaatcacgcgatccgccaggcctgcgaagacaaactgggggccgatcagcaaccggcatttgtgttgtatctggagatcgacccacatcaggtggacgtcaacgtgcaccccgccaaacacgaagtgcgtttccatcagtcgcgtctggtgcatgattttatctatcagggcgtgctgagcgtgctacaacagcaactggaaacgccgctaccgctggacgatgaaccccaacctgcaccgcgttccattccggaaaaccgcgtggcggcggggcgcaatcactttgcagaaccggcagctcgtgagccggtagctccgcgctacactcctgcgccagcatcaggcagtcgtccggctgccccctggccgaatgcgcagccaggctaccagaaacagcaaggtgaagtgtatcgccagcttttgcaaacgcccgcgccgatgcaaaaattaaaagcgccggaaccgcaggaacctgcacttgcggcgaacagtcagagttttggtcgggtactgactatcgtccattccgactgtgcgttgctggagcgcgacggcaacatttcacttttatccttgccagtggcagaacgttggctgcgtcaggcacaattgacgccgggtgaagcgcccgtttgcgcccagccgctgctgattccgttgcggctaaaagtttctgccgaagaaaaatcggcattagaaaaagcgcagtctgccctggcggaattgggtattgatttccagtcagatgcacagcatgtgaccatcagggcagtgcctttacccttacgccaacaaaatttacaaatcttgattcctgaactgataggctacctggcgaagcagtccgtattcgaacctggcaatattgcgcagtggattgcacgaaatctgatgagcgaacatgcgcagtggtcaatggcacaggccataaccctgctggcggacgtggaacggttatgtccgcaacttgtgaaaacgccgccgggtggtctgttacaatctgttgatttacatccggcgataaaagccctgaaagatgagtga

SEQ ID NO: 16 - DNA, 627 bp, synthetic construct
  misc_feature 1..627: note = synthetic polynucleotide (Engineered DGR RNA+ccdB)
  source 1..627: mol_type = other DNA
aagggcaggctgggaaataacgagacctgaattgcgcgcaattaaccctcactaaagggaacaaaagctggagctcttatattccccagaacatcaggttaatggcgtttttgatgtcattttcgcggtggctgagatcagccacttcttccccgataacggacaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatcgtttcactccatccaaaaaaacgggtatggagaaacagtagagagttgcgataaaaagcgtcaggtaggatccgctggtctcatctgtgcccatcaccttcttgcatggctctgccaacgctacggcttggcgggctggcctttcctcaataggtggtcagccggttctgtcctgcttcggcgaacacgttacacggttcggcaaaacgtcgattactgaaaatggaaaggcggggccgacttc

SEQ ID NO: 17 - DNA, 4421 bp, synthetic construct
  misc_feature 1..4421: note = synthetic polynucleotide (plasmid pRL014)
  source 1..4421: mol_type = other DNA
gaagatcatcttattaatcagataaaatatttctagatttcagtgcaatttatctcttcaaatgtagcacgattttacggctagctcagtcctaggtacaatgctagcgaatcattaaagaggagaaaggtactatggcacgtaccccgtcacgtagtagcattggtagcctgcgtagtccgcatacccataaagcaattctgaccagtaccatcgagatcctgaaagaatgtggttatagcggactgagcattgaaagcgttgcacgtcgtgccggagcaagcaaaccgaccatttatcgttggtggacgaataaagcagcactgattgccgaagtgtatgaaaatgaaagcgaacaggtgcgtaaatttccggatctgggtagctttaaagcagatctggattttttactgcgtaatttatggaaagtttggcgtgaaactatttgcggtgaagcatttcgttgtgttattgcagaagctcagctggatcctgcaaccctgacccagttaaaggatcaatttatggaacgtcgtcgtgagatgccgaaaaaactggttgaaaatgccattagcaatggtgaactgccgaaagataccaatcgtgaacttcttctggatatgatttttggtttttgttggtatcgcctgttaaccgaacagctgaccgttgaacaggatattgaagaatttaccttccttctgattaatggtgtttgtccgggtactcagcgttaactaggccataatcgctaccaaattccagaaaacagacgctttcgagcgtcttttttcgttttggtcacgacgtactgaatctgattcgttaccaattgacatgatacgaaacgtaccgtatcgttaaggtggaggcatatcaaaggacgagtgcaggtggcaaaaatgggaaagcgccatcgtaaccttattgaccaaatcaccacctgggaaaatttgttagatgcgtaccgtaagactagccacggtaagcgccgcacttgggggtatcttgaatttaaggagtacgatttggccaatttgcttgcacttcaggcggagttgaaggcaggcaattacgagcgcggaccgtatcgcgagtttctggtttacgaaccgaaaccccgcttgattagcgcactggaatttaaagatcgtcttgttcaacacgcgctgtgcaacatcgttgcgccaatctttgaagcaggtttgcttccatatacatacgcgtgtcgtccggataaggggacccatgcaggagtgtgtcatgttcaggcagaattgcgtcgcacgcgcgcgactcattttttgaagagtgactttagcaagttcttcccaagcattgaccgtgccgctctttatgcgatgattgataaaaaaatccactgcgccgctacacgtcgccttttgcgcgttgtcctgccggatgagggagttggaattcccattggcagcttaacctctcagttatttgccaacgtttacgggggcgcggtagatcgtttgttacacgacgagttaaagcagcgccactgggctcgttacatggatgacattgtcgtacttggggatgatccagaagaactgcgcgcggtcttctatcgtttgcgtgattttgcgtccgaacgcttaggtttgaagatttcacattggcaggtagcgcctgtgtctcgtggaatcaattttcttggttaccgcatctggccgacccataaattgctgcgtaagagtagtgtgaagcgcgctaagcgtaaggtggcaaatttcatcaaacacggagaagacgagtcactgcaacgcttccttgcctcgtggtcgggtcacgcccaatgggcggacactcacaatttattcacttggatggaggagcaatatggcatcgcgtgtcattaataacgttaaagtcagtttcacctgttttacgttaaaacccgcttcggcgggtttttacttttgggtttagccgaacgccccaaaaagcctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtaaagggactagtggatgtttgatctcaaaaaaagcaccttattttccggcttgtcctttacggttcacacgagcaatccaggaccctaagatacggccaacctccgcgataagcacctgagcagtctcgacctggtgcggagtcatggcatgcggcttttgaatgcctgccaaaaagcgaagccagaagcgtaacatcgccaaacccgcgtcagcagcatacagtttcgagacctggtttgatttacctgctacaatgaacagttccacctgtcccaacagacatttcaaaaacatttcgcgtgcgactccatgtttacgagggattgactgagcaatgggatacaagtatgagatgacgcgttcatagcgctctacaatcaacatctgatcgtaacacttggtagcctcttcaataggttccatagaaactttctcctctttaatactagtattatacctaggactgagctagctgtcagtcgggtagcaccagaagtctatagcatgtgcatacctttggtcgaaaaaaaaagcccgcactgtcaggtgcgggcttttttcagtgtttccttgccggattacgccccgccctgccactcatcgcagtattgttgtaattcattaagcattctgccgacatggaagccatcacaaacggcatgatgaacttggatcgccagtggcattaacaccttgtcgccttgcgtataatattttcccatagtgaaaacgggggcgaagaagttgtccatatttgctacgtttaaatcaaaactggtgaaactcacccacggattggcactgacgaaaaacatattttcgataaaccctttagggaaatatgctaagttttcaccgtaacacgccacatcttgactatatatgtgtagaaactgccggaaatcgtcgtggtattctgaccagagcgatgaaaacgtttcagtttgctcatggaaaacggtgtaacaagggtgaacactatcccatatcaccagctcaccgtctttcattgccatacgaaactccggatgtgcattcatcaggcgggcaagaatgtgaataaaggccggataaaacttgtgcttatttttctttacggtttttaaaaaggccgtaatatccagctgaacggtttggttataggtgcactgagcaactgactggaatgcctcaaaatgttctttacgatgccattgacttatatcaactgtagtatatccagtgatttttttctccattttagcttccttagcttgcgaaatctcgataactcaaaaaatagtagtgatcttatttcattatggtgaaagttgtcttacgtgcaacattttcgcaaaaagttggcgctttatcaacactgtccctcctgttcagctaccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctggtacccaattcgccctatagtgagtctcctggaagtgagagggccgcggcaaagccgtttttccataggctccgcccccctgacaagcatcacgaaatctgacgctcaaatcagtggtggcgaaacctgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaa

SEQ ID NO: 18 - DNA, 7924 bp, synthetic construct
  misc_feature 1..7924: note = synthetic polynucleotide (plasmid pRL021-ccdB)
  source 1..7924: mol_type = other DNA
gaagatcctttgatcttttctacggggtctgacgctcagtggaacgaaaactcacgttaagggattttggtcatgactagtgcttggagaaggccatcctgacggatggccttttatgcctttaattaaacgttcgtaatcaagccacttcctttttgcattgacgcagggtgtcggaaggcaactcgccgaacgcgctcctatagttttcagcgaagcgtcccaaatgtaagaagccgtagtctagggctatctcagttatactacgcacattggcactgggatcgttcaagcaggcgcggatgctttcgagcttgcggttgcggatgtagttcttcggcgtggtgccggcgtgcttctcgaacaaattgtagagcgagcgtggactcatcatcgccagctccgctaaccgctcaaggctgatattccgtttgagattctcctcaatgaattgaacgactcgctcgaaagacgggttacctttgctgaaaatttcacggctgacattgctgcccagcatttcgagcagcttggaagcgatgatccccgcatagtgctcttggacccgaggcatcgactttgtatgttccgcttcgtcacaaactaacccgagtagattgataaagccatcgagttgctggagattgtgtcgcgcggcgaaacggataccctccctcggcttgtgccaattgttgtcactgcacgcccgatcaaggaccactgagggcaatttaacgataaatttctcgcaatcttctgaataggtcaggtcggcttggtcatccggattgagcagcaatagttcgcccggcgcaaaatagtgctcctggccatggccacgccacaggcaatggcctttgagtattatttgcagatgataacaggtttctaatccaggcgagattaccctcacgctaccgccgtagctgattcgacacagatcgaggcatccgaagattctgtggtgcagcctgcctgccgggcgcccgcccttgggcaggcgaatagagtgcgtaccgacatactggttaacataatcggagactgcatagggctcggcgtggacgaagatctgacttttctcgttcaataagcaaaaatccatagttcacggttctcttattttaatgtgggctgcttggtgtgatgtagaaaggcgccaagtcgatgaaaatgcatctcgacgtgatgcgtatacgggttacccccattgccacgttgcgccatcctttttgcaatcagtgaccacttttccaagcaaaaataacgccaagcagaacgaagacgttctttttaagaagcgagaacaccagaagttcgtgctgtcggggcatggggcgacgaattggcggataaaggggatctgctggatattacggcctttttaaagaccgtaaagaaaaataagcacaagttttatccggcctttattcacattcttgcccgcctgatgaatgctcatccgtaattacgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgcatgggaattagcttgatctgaccaacgaccggtagcggagctatccaacggcggtataccaggaaaacacacagcaggtacatcagaacagtaccatgactgaagaacaaatagttttttcctgatccataaagcagaacggcctgctccatgacaaatctggctccccaactaatgccccatgcagccagcataaccagcataaagtgcagtgtccggtttgatagggataagtccagccttgcaagaagcggatacaggagtgcaaaaaatggctatctctagtaaggcctaccccttaggctttatgcaacagaaacaataataatggagtcatgaccatgcctaggccgcgggccgcgcgaattcagaaggagaatataccatgaaccaaatcgtgaagttcactgacgactctggcctggcggttcaagttactccagacgatgttcgccgttatatctgtgagaacgctactgaaaaagaggtgggcctctttctgcaactctgtcagactcaacgtctgaatccgtttgtgaaagacgcttacctggtgaaatacggcggtgctccagcttctatgattacttcctatcaagtttttaaccgtcgcgcgtgtcgtgatgctaactatgatggtatcaaatctggtgtggttgttctgcgtgacggtgatgttgtgcataaacgtggtgctgcgtgctacaaaaaggcgggtgaggagctcatcggtggttgggcggaagttcgctttaaggatggccgcgagactgcgtatgctgaggtggcgctcgacgactattccaccggcaaatctaattgggcgaaaatgccgggtgttatgatcgaaaaatgcgcgaaggctgctgcttggcgcctcgcgttcccggacacttttcagggcatgtacgctgcggaggaaatggatcaagcgcaacagccagaacaggtgcgcgctcaggcggagcaaccagtggatctccagccaatccgcgaactcttcaagccatattgcgaacacttcggcatcactccggctgagggtatgactgctgtttgtggtgcggtgggcgctgaaggcatgcactctatgaccgagcagcaagctcgccgtgctcgcgcttggatggaggaagaaatggctgcgccagctgtggaagcggagtatgaggttgttgacgagggcgaggtgttttaaggatccaggagaatacacatgccaattcaggtcttaccgccacaactggcgaaccagattgccgcaggtgaggtggtcgagcgacctgcgtcggtagtcaaagaactagtgaaaaacagcctcgatgcaggtgcgacgcgtatcgatattgatatcgaacgcggtggggcgaaacttatccgcattcgtgataacggctgcggtatcaaaaaagatgagctggcgctggcgctggctcgtcatgccaccagtaaaatcgcctctctggacgatctcgaagccattatcagcctgggctttcgcggtgaggcgctggcgagtatcagttcggtttcccgcctgacgctcacttcacgcaccgcagaacagcaggaagcctggcaggcctatgccgaagggcgcgatatgaacgtgacggtaaaaccggcggcgcatcctgtggggacgacgctggaggtgctggatctgttctacaacaccccggcgcggcgcaaattcctgcgcaccgagaaaaccgaatttaaccacattgatgagatcatccgccgcattgcgctggcgcgtttcgacgtcacgatcaacctgtcgcataacggtaaaattgtgcgtcagtaccgcgcagtgccggaaggcgggcaaaaagaacggcgcttaggcgcgatttgcggcaccgcttttcttgaacaagcgctggcgattgaatggcaacacggcgatctcacgctacgcggctgggtggccgatccaaatcacaccacgcccgcactggcagaaattcagtattgctacgtgaacggtcgcatgatgcgcgatcgcctgatcaatcacgcgatccgccaggcctgcgaagacaaactgggggccgatcagcaaccggcatttgtgttgtatctggagatcgacccacatcaggtggacgtcaacgtgcaccccgccaaacacgaagtgcgtttccatcagtcgcgtctggtgcatgattttatctatcagggcgtgctgagcgtgctacaacagcaactggaaacgccgctaccgctggacgatgaaccccaacctgcaccgcgttccattccggaaaaccgcgtggcggcggggcgcaatcactttgcagaaccggcagctcgtgagccggtagctccgcgctacactcctgcgccagcatcaggcagtcgtccggctgccccctggccgaatgcgcagccaggctaccagaaacagcaaggtgaagtgtatcgccagcttttgcaaacgcccgcgccgatgcaaaaattaaaagcgccggaaccgcaggaacctgcacttgcggcgaacagtcagagttttggtcgggtactgactatcgtccattccgactgtgcgttgctggagcgcgacggcaacatttcacttttatccttgccagtggcagaacgttggctgcgtcaggcacaattgacgccgggtgaagcgcccgtttgcgcccagccgctgctgattccgttgcggctaaaagtttctgccgaagaaaaatcggcattagaaaaagcgcagtctgccctggcggaattgggtattgatttccagtcagatgcacagcatgtgaccatcagggcagtgcctttacccttacgccaacaaaatttacaaatcttgattcctgaactgataggctacctggcgaagcagtccgtattcgaacctggcaatattgcgcagtggattgcacgaaatctgatgagcgaacatgcgcagtggtcaatggcacaggccataaccctgctggcggacgtggaacggttatgtccgcaacttgtgaaaacgccgccgggtggtctgttacaatctgttgatttacatccggcgataaaagccctgaaagatgagtgatctagagtcgacctgcaggcatgcaagcttgcggccgcgtcgtgactgggaaaaccctggcgactagtcttggactcctgttgatagatccagtaatgacctcagaactccatctggatttgttcagaacgctcggttgccgccgggcgttttttattggtgagaatccaggggtccccaataattacgatttaaatttgtgtctcaaaatctctgatgttacattgcacaagataaaaatatatcatcatgaacaataaaactgtctgcttacataaacagtaatacaaggggtgttatgagccatattcagcgtgaaacgagctgtagccgtccgcgtctgaacagcaacatggatgcggatctgtatggctataaatgggcgcgtgataacgtgggtcagagcggcgcgaccatttatcgtctgtatggcaaaccggatgcgccggaactgtttctgaaacatggcaaaggcagcgtggcgaacgatgtgaccgatgaaatggtgcgtctgaactggctgaccgaatttatgccgctgccgaccattaaacattttattcgcaccccggatgatgcgtggctgctgaccaccgcgattccgggcaaaaccgcgtttcaggtgctggaagaatatccggatagcggcgaaaacattgtggatgcgctggccgtgtttctgcgtcgtctgcatagcattccggtgtgcaactgcccgtttaacagcgatcgtgtgtttcgtctggcccaggcgcagagccgtatgaacaacggcctggtggatgcgagcgattttgatgatgaacgtaacggctggccggtggaacaggtgtggaaagaaatgcataaactgctgccgtttagcccggatagcgtggtgacccacggcgattttagcctggataacctgattttcgatgaaggcaaactgattggctgcattgatgtgggccgtgtgggcattgcggatcgttatcaggatctggccattctgtggaactgcctgggcgaatttagcccgagcctgcaaaaacgtctgtttcagaaatatggcattgataatccggatatgaacaaactgcaatttcatctgatgctggatgaatttttctaataattaattggaccgcggtccgcgcgttgtccttttccgctgcataaccctgcttcggggtcattatagcgattttttcggtatatccatcctttttcgcacgatatacaggattttgccaaagggttcgtgtagaccgggcccattaagttctgtgctaggaggtgactgaagtatattttaggaattctaaagatctttgacagctagctcagtcctaggtataatactagtaagggcaggctgggaaataacgagacctgaattgcgcgcaattaaccctcactaaagggaacaaaagctggagctcttatattccccagaacatcaggttaatggcgtttttgatgtcattttcgcggtggctgagatcagccacttcttccccgataacggacaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatcgtttcactccatccaaaaaaacgggtatggagaaacagtagagagttgcgataaaaagcgtcaggtaggatccgctggtctcatctgtgcccatcaccttcttgcatggctctgccaacgctacggcttggcgggctggcctttcctcaataggtggtcagccggttctgtcctgcttcggcgaacacgttacacggttcggcaaaacgtcgattactgaaaatggaaaggcggggccgacttcggtagtgcagcgcgatcgtaatcaggatcccatggtacgcgtgctagaggcatcaaataaaacgaaaggctcagtcgaaagactgggcctttcgttttatctgttgtttgtcggtgaacgctctcctgagtaggacaaatccgccgccctagaccctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgaggctttttggggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaa

SEQ ID NO: 19 - DNA, 70 bp, synthetic construct
  misc_feature 1..70: note = synthetic polynucleotide (TR_AM011)
  source 1..70: mol_type = other DNA
gtttggcggtctgggtgccttcatacggacggccctcgccttcgccttcgatctcgaactcgtgaccgtt

SEQ ID NO: 20 - DNA, 5265 bp, synthetic construct
  misc_feature 1..5265: note = synthetic polynucleotide (pRL038-ccdB)
  source 1..5265: mol_type = other DNA
gaagatcatcttattaatcagataaaatatttctagatttcagtgcaatttatctcttcaaatgtagcacgattttacggctagctcagtcctaggtacaatgctagcgaatcattaaagaggagaaaggtactatggcacgtaccccgtcacgtagtagcattggtagcctgcgtagtccgcatacccataaagcaattctgaccagtaccatcgagatcctgaaagaatgtggttatagcggactgagcattgaaagcgttgcacgtcgtgccggagcaagcaaaccgaccatttatcgttggtggacgaataaagcagcactgattgccgaagtgtatgaaaatgaaagcgaacaggtgcgtaaatttccggatctgggtagctttaaagcagatctggattttttactgcgtaatttatggaaagtttggcgtgaaactatttgcggtgaagcatttcgttgtgttattgcagaagctcagctggatcctgcaaccctgacccagttaaaggatcaatttatggaacgtcgtcgtgagatgccgaaaaaactggttgaaaatgccattagcaatggtgaactgccgaaagataccaatcgtgaacttcttctggatatgatttttggtttttgttggtatcgcctgttaaccgaacagctgaccgttgaacaggatattgaagaatttaccttccttctgattaatggtgtttgtccgggtactcagcgttaactaggccataatcgctaccaaattccagaaaacagacgctttcgagcgtcttttttcgttttggtcacgacgtactgaatctgattcgttaccaattgacatgatacgaaacgtaccgtatcgttaaggtggaggcatatcaaaggacgagtgcaggtggcaaaaatgggaaagcgccatcgtaaccttattgaccaaatcaccacctgggaaaatttgttagatgcgtaccgtaagactagccacggtaagcgccgcacttgggggtatcttgaatttaaggagtacgatttggccaatttgcttgcacttcaggcggagttgaaggcaggcaattacgagcgcggaccgtatcgcgagtttctggtttacgaaccgaaaccccgcttgattagcgcactggaatttaaagatcgtcttgttcaacacgcgctgtgcaacatcgttgcgccaatctttgaagcaggtttgcttccatatacatacgcgtgtcgtccggataaggggacccatgcaggagtgtgtcatgttcaggcagaattgcgtcgcacgcgcgcgactcattttttgaagagtgactttagcaagttcttcccaagcattgaccgtgccgctctttatgcgatgattgataaaaaaatccactgcgccgctacacgtcgccttttgcgcgttgtcctgccggatgagggagttggaattcccattggcagcttaacctctcagttatttgccaacgtttacgggggcgcggtagatcgtttgttacacgacgagttaaagcagcgccactgggctcgttacatggatgacattgtcgtacttggggatgatccagaagaactgcgcgcggtcttctatcgtttgcgtgattttgcgtccgaacgcttaggtttgaagatttcacattggcaggtagcgcctgtgtctcgtggaatcaattttcttggttaccgcatctggccgacccataaattgctgcgtaagagtagtgtgaagcgcgctaagcgtaaggtggcaaatttcatcaaacacggagaagacgagtcactgcaacgcttccttgcctcgtggtcgggtcacgcccaatgggcggacactcacaatttattcacttggatggaggagcaatatggcatcgcgtgtcattaataacgttaaagtcagtttcacctgttttacgttaaaacccgcttcggcgggtttttacttttgggtttagccgaacgccccaaaaagcctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtaaagggactagtggatgtttgatctcaaaaaaagcaccttattttccggcttgtcctttacggttcacacgagcaatccaggaccctaagatacggccaacctccgcgataagcacctgagcagtctcgacctggtgcggagtcatggcatgcggcttttgaatgcctgccaaaaagcgaagccagaagcgtaacatcgccaaacccgcgtcagcagcatacagtttcgagacctggtttgatttacctgctacaatgaacagttccacctgtcccaacagacatttcaaaaacatttcgcgtgcgactccatgtttacgagggattgactgagcaatgggatacaagtatgagatgacgcgttcatagcgctctacaatcaacatctgatcgtaacacttggtagcctcttcaataggttccatagaaactttctcctctttaatactagtattatacctaggactgagctagctgtcagtcgggtagcaccagaagtctatagcatgtgcatacctttggtcgaaaaaaaaagcccgcactgtcaggtgcgggcttttttcagtgtttccttgccggagaagtcggccccgcctttccattttcagtaatcgacgttttgccgaaccgtgtaacgtgttcgccgaagcaggacagaaccggctgaccacctattgaggaaaggccagcccgccaagccgtagcgttggcagagccatgcaagaaggtgatgggcacagatgagaccagcggatcctacctgacgctttttatcgcaactctctactgtttctccatacccgtttttttggatggagtgaaacgatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtgtccgttatcggggaagaagtggctgatctcagccaccgcgaaaatgacatcaaaaacgccattaacctgatgttctggggaatataagagctccagcttttgttccctttagtgagggttaattgcgcgcaattcaggtctcgttatttcccagcctgcccttactatgcaaccattatcaccgccagaggtaaaattgtcaacacgcacggtgttagctcaaaaataaacaaaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctttacgccccgccctgccactcatcgcagtattgttgtaattcattaagcattctgccgacatggaagccatcacaaacggcatgatgaacttggatcgccagtggcattaacaccttgtcgccttgcgtataatattttcccatagtgaaaacgggggcgaagaagttgtccatatttgctacgtttaaatcaaaactggtgaaactcacccacggattggcactgacgaaaaacatattttcgataaaccctttagggaaatatgctaagttttcaccgtaacacgccacatcttgactatatatgtgtagaaactgccggaaatcgtcgtggtattctgaccagagcgatgaaaacgtttcagtttgctcatggaaaacggtgtaacaagggtgaacactatcccatatcaccagctcaccgtctttcattgccatacgaaactccggatgtgcattcatcaggcgggcaagaatgtgaataaaggccggataaaacttgtgcttatttttctttacggtttttaaaaaggccgtaatatccagctgaacggtttggttataggtgcactgagcaactgactggaatgcctcaaaatgttctttacgatgccattgacttatatcaactgtagtatatccagtgatttttttctccattttagcttccttagcttgcgaaatctcgataactcaaaaaatagtagtgatcttatttcattatggtgaaagttgtcttacgtgcaacattttcgcaaaaagttggcgctttatcaacactgtccctcctgttcagctaccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctggtacccaattcgccctatagtgagtctcctggaagtgagagggccgcggcaaagccgtttttccataggctccgcccccctgacaagcatcacgaaatctgacgctcaaatcagtggtggcgaaacctgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaa

SEQ ID NO: 21 - DNA, 113 bp, synthetic construct
  misc_feature 1..113: note = synthetic polynucleotide (TR_AM001)
  source 1..113: mol_type = other DNA
cgctgctgcgctattcggcggcaactggaacaacacgtcgaactcgggttctcgcgctgcgaactggaacaacgggccgtcgaactcgaacgcgaacatcggggcgcgcggcg

SEQ ID NO: 22 - DNA, 40 bp, synthetic construct
  misc_feature 1..40: note = synthetic polynucleotide (TR_AM004)
  source 1..40: mol_type = other DNA
ttatatggcttttggttcgtttctttcgcaaacgcttgag

SEQ ID NO: 23 - DNA, 100 bp, synthetic construct
  misc_feature 1..100: note = synthetic polynucleotide (TR_AM007)
  source 1..100: mol_type = other DNA
tgccgtatgtttccttatatggcttttggttcgtttctttcgcaaacgcttgagttgcgcctcctgccagcagtgcggtagtaaaggttaatactgttgc

SEQ ID NO: 24 - DNA, 70 bp, synthetic construct
  misc_feature 1..70: note = synthetic polynucleotide (TR_AM009)
  source 1..70: mol_type = other DNA
tttgtggcctttatcttctacgtagtgaggatctctcagcgtatggttgtcgcctgagctgtagttgcct

SEQ ID NO: 25 - DNA, 70 bp, synthetic construct
  misc_feature 1..70: note = synthetic polynucleotide (TR_AM010)
  source 1..70: mol_type = other DNA
cgtgatagtttgcgacagtgccgtcagcgttttgtaatggccagctgtcccaaacgtccaggccttttgc

SEQ ID NO: 26 - DNA, 61 bp, synthetic construct
  misc_feature 1..61: note = synthetic oligonucleotide (AM001)
  source 1..61: mol_type = other DNA
ataacgctgctgcgctattcggcggcaactggaacaacacgtcgaactcgggttctcgcgc

SEQ ID NO: 27 - DNA, 61 bp, synthetic construct
  misc_feature 1..61: note = synthetic oligonucleotide (AM002)
  source 1..61: mol_type = other DNA
cgcagcgcgagaacccgagttcgacgtgttgttccagttgccgccgaatagcgcagcagcg

SEQ ID NO: 28 - DNA, 56 bp, synthetic construct
  misc_feature 1..56: note = synthetic oligoucleotide (AM030)
  source 1..56: mol_type = other DNA
tgcgaactggaacaacgggccgtcgaactcgaacgcgaacatcggggcgcgcggcg

SEQ ID NO: 29 - DNA, 56 bp, synthetic construct
  misc_feature 1..56: note = synthetic oligonucleotide (AM031)
  source 1..56: mol_type = other DNA
cagacgccgcgcgccccgatgttcgcgttcgagttcgacggcccgttgttccagtt

SEQ ID NO: 30 - DNA, 44 bp, synthetic construct
  misc_feature 1..44: note = synthetic oligonucleotide (AM007)
  source 1..44: mol_type = other DNA
ataattatatggcttttggttcgtttctttcgcaaacgcttgag

SEQ ID NO: 31 - DNA, 44 bp, synthetic construct
  misc_feature 1..44: note = synthetic oligonucleotide (AM008)
  source 1..44: mol_type = other DNA
cagactcaagcgtttgcgaaagaaacgaaccaaaagccatataa

SEQ ID NO: 32 - DNA, 54 bp, synthetic construct
  misc_feature 1..54: note = synthetic oligonucleotide (AM017)
  source 1..54: mol_type = other DNA
ataatgccgtatgtttccttatatggcttttggttcgtttctttcgcaaacgct

SEQ ID NO: 33 - DNA, 54 bp, synthetic construct
  misc_feature 1..54: note = synthetic oligonucleotide (AM018)
  source 1..54: mol_type = other DNA
ctcaagcgtttgcgaaagaaacgaaccaaaagccatataaggaaacatacggca

SEQ ID NO: 34 - DNA, 50 bp, synthetic construct
  misc_feature 1..50: note = synthetic oligonucleotide (AM019)
  source 1..50: mol_type = other DNA
tgagttgcgcctcctgccagcagtgcggtagtaaaggttaatactgttgc

SEQ ID NO: 35 - DNA, 50 bp, synthetic construct
  misc_feature 1..50: note = synthetic oligonucleotide (AM020)
  source 1..50: mol_type = other DNA
cagagcaacagtattaacctttactaccgcactgctggcaggaggcgcaa

SEQ ID NO: 36 - DNA, 74 bp, synthetic construct
  misc_feature 1..74: note = synthetic oligonucleotide (AM021)
  source 1..74: mol_type = other DNA
ataatttgtggcctttatcttctacgtagtgaggatctctcagcgtatggttgtcgcctgagctgtagttgcct

SEQ ID NO: 37 - DNA, 74 bp, synthetic construct
  misc_feature 1..74: note = synthetic oligonucleotide (AM022)
  source 1..74: mol_type = other DNA
cagaaggcaactacagctcaggcgacaaccatacgctgagagatcctcactacgtagaagataaaggccacaaa

SEQ ID NO: 38 - DNA, 74 bp, synthetic construct
  misc_feature 1..74: note = synthetic oligonucleotide (AM024)
  source 1..74: mol_type = other DNA
ataacgtgatagtttgcgacagtgccgtcagcgttttgtaatggccagctgtcccaaacgtccaggccttttgc

SEQ ID NO: 39 - DNA, 74 bp, synthetic construct
  misc_feature 1..74: note = syntthetic oligonucleotide (AM025)
  source 1..74: mol_type = other DNA
cagagcaaaaggcctggacgtttgggacagctggccattacaaaacgctgacggcactgtcgcaaactatcacg

SEQ ID NO: 40 - DNA, 74 bp, synthetic construct
  misc_feature 1..74: note = synthetic oligonucleotide (AM027)
  source 1..74: mol_type = other DNA
ataagtttggcggtctgggtgccttcatacggacggccctcgccttcgccttcgatctcgaactcgtgaccgtt

SEQ ID NO: 41 - DNA, 74 bp, synthetic construct
  misc_feature 1..74: note = synthetic oligonucleotide (AM028)
  source 1..74: mol_type = other DNA
cagaaacggtcacgagttcgagatcgaaggcgaaggcgagggccgtccgtatgaaggcacccagaccgccaaac

SEQ ID NO: 42 - DNA, 68 bp, synthetic construct
  misc_feature 1..68: note = synthetic oligonucleotide TR_RL016
  source 1..68: mol_type = other DNA
tgccgtatgtttccttatatggcttttggttcgtttctttcgcaaacgcttgagttgcgcctcctgcc

SEQ ID NO: 43 - DNA, 90 bp, synthetic construct
  misc_feature 1..90: note = synthetic polynucleotide
  source 1..90: mol_type = other DNA
atactaagtatttgtggcctttatcttctacgtagtgaggatctctcagcgtatggttgtcgcctgagctgtagttgccttcatcgatga

SEQ ID NO: 44 - DNA, 90 bp, synthetic construct
  misc_feature 1..90: note = synthetic polynucleotide
  source 1..90: mol_type = other DNA
tatgattcataaacaccggaaatagaagatgcatcactcctagagagtcgcataccaacagcggactcgacatcaacggaagtagctact

SEQ ID NO: 45 - protein, 30 aa, synthetic construct
  REGION 1..30: note = synthetic polypeptide
VLYKHGKDEVYHPDRLTHNDGSSYNGEDIF

SEQ ID NO: 46 - DNA, 90 bp, synthetic construct
  misc_feature 1..90: note = synthetic polynucleotide
  source 1..90: mol_type = other DNA
atactaagtatttgtggccttttccttctacgtagtgaggatctctcagcgtatggttgtcgcctgagctgtagttgccttcatcgatga

SEQ ID NO: 47 - DNA, 90 bp, synthetic construct
  misc_feature 1..90: note = synthetic polynucleotide
  source 1..90: mol_type = other DNA
atactaagtatttgtggcctttgtcttctgcgtggtgaggatctctcagcgtatggttgtcgcctgagctgtagttgccttcatcgatga

SEQ ID NO: 48 - DNA, 90 bp, synthetic construct
  misc_feature 1..90: note = synthetic polynucleotide
  source 1..90: mol_type = other DNA
atactaagtatttgtggcctttatcttctacgtagtgaggatctctcggcgtatggttgtcgcctgagctgtagttgccttcatcgatga

SEQ ID NO: 49 - DNA, 90 bp, synthetic construct
  misc_feature 1..90: note = synthetic polynucleotide
  source 1..90: mol_type = other DNA
atactaagtatttgtggccttgatcttctacgtagtgaggttctctcagcgtgtggttgtcgcctgagctgtagttgccttcatcgatga

SEQ ID NO: 50 - DNA, 90 bp, synthetic construct
  misc_feature 1..90: note = synthetic polynucleotide
  source 1..90: mol_type = other DNA
atgtggtagccgtgatagtttgcgacagtgccgtcagcgttttgtaatggccagctgtcccaaacgtccaggccttttgcagaagagata

SEQ ID NO: 51 - DNA, 90 bp, synthetic construct
  misc_feature 1..90: note = synthetic polynucleotide
  source 1..90: mol_type = other DNA
tacaccatcggcactatcaaacgctgtcacggcagtcgcaaaacattaccggtcgacagggtttgcaggtccggaaaacgtcttctctat

SEQ ID NO: 52 - protein, 30 aa, synthetic construct
  REGION 1..30: note = synthetic polypeptide
IHYGHYNAVTGDANQLPWSDWVDLGKASSI

SEQ ID NO: 53 - DNA, 90 bp, synthetic construct
  misc_feature 1..90: note = synthetic polynucleotide
  source 1..90: mol_type = other DNA
atgtggtagccgtgatagtttgcgacagtgccgtcagcgttttgtaatggccagctgtcccagtcgtccaggccttttgcagaagagata

SEQ ID NO: 54 - DNA, 90 bp, synthetic construct
  misc_feature 1..90: note = synthetic polynucleotide
  source 1..90: mol_type = other DNA
atgtggtagccgtgatagtttgcgacagtgccgtcagcgttttgtaatggccggctgtcccaaacgtccaggccttttgcagaagagata

SEQ ID NO: 55 - DNA, 90 bp, synthetic construct
  misc_feature 1..90: note = synthetic polynucleotide
  source 1..90: mol_type = other DNA
atgtggtagccgtgttagtttgcgacagtgccgtcagcgttttgtcatggccagctgtcccaaacgtccaggccttttgcagaagagata

SEQ ID NO: 56 - DNA, 90 bp, synthetic construct
  misc_feature 1..90: note = synthetic polynucleotide
  source 1..90: mol_type = other DNA
atgtggtagccgtgatagtttgcgtcagtgccgtcagcgttttgtactggccagctgtcccaaacgtccaggccttttgcagaagagata

SEQ ID NO: 57 - DNA, 88 bp, synthetic construct
  misc_feature 1..88: note = synthetic polynucleotide
  source 1..88: mol_type = other DNA
atatgggaaatgccgtatgtttccttatatggcttttggttcgtttctttcgcaaacgcttgagttgcgcctcctgccagcagtgcgg

SEQ ID NO: 58 - DNA, 88 bp, synthetic construct
  misc_feature 1..88: note = synthetic polynucleotide
  source 1..88: mol_type = other DNA
tataccctttacggcatacaaaggaatataccgaaaaccaagcaaagaaagcgtttgcgaactcaacgcggaggacggtcgtcacgcc

SEQ ID NO: 59 - protein, 30 aa, synthetic construct
  REGION 1..30: note = synthetic polypeptide
IHSIGYTEKYPKQNTEKAFAQTAGGALLAT

SEQ ID NO: 60 - DNA, 88 bp, synthetic construct
  misc_feature 1..88: note = synthetic polynucleotide
  source 1..88: mol_type = other DNA
atatgggaaatgccgtatgtttccttatatggcttttggttcgtttctttcgcttacgcttgagttgcgcctcctgccagcagtgcgg

SEQ ID NO: 61 - DNA, 88 bp, synthetic construct
  misc_feature 1..88: note = synthetic polynucleotide
  source 1..88: mol_type = other DNA
atatgggaaatgccgtgtgtttccttctatggcttttggttcgtttctttcgcgaacgcttgagttgcgcctcctgccagcagtgcgg

SEQ ID NO: 62 - DNA, 88 bp, synthetic construct
  misc_feature 1..88: note = synthetic polynucleotide
  source 1..88: mol_type = other DNA
atatgggaaatgccgtatgtttccttttatggctcttggttcgtttctttcgcgtacgcttgagttgcgcctcctgccagcagtgcgg

SEQ ID NO: 63 - DNA, 88 bp, synthetic construct
  misc_feature 1..88: note = synthetic polynucleotide
  source 1..88: mol_type = other DNA
atatgggaaatgccgtgtgtttccttctatggcttttggttcgtttctttcgctttcgcttgggttgcgcctcctgccagcagtgcgg

SEQ ID NO: 64 - DNA, 60 bp, synthetic construct
  misc_feature 1..60: note = synthetic polynucleotide
  source 1..60: mol_type = other DNA
gtatgtttccttatatggcttttggttcgtttctttcgcaaacgcttgagttgcgcctcc

SEQ ID NO: 65 - DNA, 60 bp, synthetic construct
  misc_feature 1..60: note = synthetic polynucleotide
  source 1..60: mol_type = other DNA
catacaaaggaatataccgaaaaccaagcaaagaaagcgtttgcgaactcaacgcggagg

SEQ ID NO: 66 - protein, 20 aa, synthetic construct
  REGION 1..20: note = synthetic polynucleotide
YTEKYPKQNTEKAFAQTAGG

SEQ ID NO: 67 - DNA, 60 bp, synthetic construct
  misc_feature 1..60: note = synthetic polynucleotide
  source 1..60: mol_type = other DNA
gtatgtttccttatatggcttttggttcgtttctttcgcctacgcttgagttgcgcctcc

SEQ ID NO: 68 - DNA, 112 bp, synthetic construct
  misc_feature 1..112: note = synthetic polynucleotide
  source 1..112: mol_type = other DNA
gggaaatgccgtatgtttccttatatggcttttggttcgtttctttcgcaaacgcttgagttgcgcctcctgccagcagtgcggtagtaaaggttaatactgttgcttgttt

SEQ ID NO: 69 - DNA, 112 bp, synthetic construct
  misc_feature 1..112: note = synthetic polynucleotide
  source 1..112: mol_type = other DNA
ccctttacggcatacaaaggaatataccgaaaaccaagcaaagaaagcgtttgcgaactcaacgcggaggacggtcgtcacgccatcatttccaattatgacaacgaacaaa

SEQ ID NO: 70 - protein, 37 aa, synthetic construct
  REGION 1..37: note = synthetic polypeptide
SIGYTEKYPKQNTEKAFAQTAGGALLATTFTLVTAQK

SEQ ID NO: 71 - DNA, 112 bp, synthetic construct
  misc_feature 1..112: note = synthetic polynucleotide
  source 1..112: mol_type = other DNA
gggaaatgccgtgtgtttccttatatggcttttggttcgtttctttcgcttacgcttgagttgcgcctcctgccagcagtgcggtagtaaaggttaatactgttgcttgttt

SEQ ID NO: 72 - DNA, 112 bp, synthetic construct
  misc_feature 1..112: note = synthetic polynucleotide
  source 1..112: mol_type = other DNA
gggaaatgccgtatgtttccttatatggcttttggttcgtttctttcggtttcgcttgagttgcgcctcctgccggcagtgcggtagtaaaggttgatactgttgcttgttt

SEQ ID NO: 73 - DNA, 112 bp, synthetic construct
  misc_feature 1..112: note = synthetic polynucleotide
  source 1..112: mol_type = other DNA
gggaaatgccgtatgtttccttttatggcttttggttcgtttctttcgcaaacgcttgggttgcgcctcctgccagcggtgcggtagtaaaggtttatactgttgcttgttt

SEQ ID NO: 74 - DNA, 112 bp, synthetic construct
  misc_feature 1..112: note = synthetic polynucleotide
  source 1..112: mol_type = other DNA
gggaaatgccgtatgtttccttatatggcttttggttcgtttctttcgccttcgcttgagttgcgcctcctgccagcggtgcggtagtaaaggttaatactgttgcttgttt

SEQ ID NO: 75 - DNA, 90 bp, synthetic construct
  misc_feature 1..90: note = synthetic polynucleotide
  source 1..90: mol_type = other DNA
gtcactttcagtttggcggtctgggtgccttcatacggacggccctcgccttcgccttcgatctcgaactcgtgaccgttaacagaaccc

SEQ ID NO: 76 - DNA, 90 bp, synthetic construct
  misc_feature 1..90: note = synthetic polynucleotide
  source 1..90: mol_type = other DNA
cagtgaaagtcaaaccgccagacccacggaagtatgcctgccgggagcggaagcggaagctagagcttgagcactggcaattgtcttggg

SEQ ID NO: 77 - DNA, 90 bp, synthetic construct
  misc_feature 1..90: note = synthetic polynucleotide
  source 1..90: mol_type = other DNA
gtcactttcagtttggcggtctgggtgccttcatacggacggccctcgccttcgccttcgatctcggtctcgtgaccgttaacagaaccc

SEQ ID NO: 78 - DNA, 90 bp, synthetic construct
  misc_feature 1..90: note = synthetic polynucleotide
  source 1..90: mol_type = other DNA
gtcactttcagtttggcggtctgggtgccttcatgcgggcggccctcgccttcgccttcgctctcgaactcgtgaccgttaacagaaccc

SEQ ID NO: 79 - DNA, 90 bp, synthetic construct
  misc_feature 1..90: note = synthetic polynucleotide
  source 1..90: mol_type = other DNA
gtcactttcagtttggcggtctgggtgccttcacacggacggccctcgccttcgccttcgatctcggcctcgtgaccgttaacagaaccc

SEQ ID NO: 80 - DNA, 90 bp, synthetic construct
  misc_feature 1..90: note = synthetic polynucleotide
  source 1..90: mol_type = other DNA
gtcactttcagtttggcggtctgggtgccttcatgcggacggccctcgccttcgccttcggtctcgtcctcgtgaccgttaacagaaccc

SEQ ID NO: 81 - DNA, 90 bp, synthetic construct
  misc_feature 1..90: note = synthetic polynucleotide
  source 1..90: mol_type = other DNA
atactaagtatttgtggcctttctcttctacgtggtgaggatctctcagcgtatggttgtcgcctgagctgtagttgccttcatcgatga

SEQ ID NO: 82 - DNA, 90 bp, synthetic construct
  misc_feature 1..90: note = synthetic polynucleotide
  source 1..90: mol_type = other DNA
atactaagtatttgtggcctttgtcttctacgtagtgaggatctctcggcgtatggttgtcgcctgagctgtagttgccttcatcgatga

SEQ ID NO: 83 - DNA, 90 bp, synthetic construct
  misc_feature 1..90: note = synthetic polynucleotide
  source 1..90: mol_type = other DNA
atactaagtatttgtggccttgttcttctacgtagtgaggatctctcagcgtgtggttgtcgcctgggctgtagttgccttcatcgatga

SEQ ID NO: 84 - DNA, 90 bp, synthetic construct
  misc_feature 1..90: note = synthetic polynucleotide
  source 1..90: mol_type = other DNA
atactaagtatttgtggcctttatcttctacgtagtgaggttctctcagcgtgtggttgtcgcctgagctgtagttgccttcatcgatga

SEQ ID NO: 85 - DNA, 86 bp, synthetic construct
  misc_feature 1..86: note = synthetic polynucleotide
  source 1..86: mol_type = other DNA
cactttcagtttggcggtctgggtgccttcatacggacggccctcgccttcgccttcgatctcgaactcgtgaccgttaacagaac

SEQ ID NO: 86 - DNA, 86 bp, synthetic construct
  misc_feature 1..86: note = synthetic polynucleotide
  source 1..86: mol_type = other DNA
gtgaaagtcaaaccgccagacccacggaagtatgcctgccgggagcggaagcggaagctagagcttgagcactggcaattgtcttg

SEQ ID NO: 87 - DNA, 86 bp, synthetic construct
  misc_feature 1..86: note = synthetic polynucleotide
  source 1..86: mol_type = other DNA
cactttcagtttggcgggctgggtgccttcatacgggcggccctcgccttcgccttcgatctcgaactcgtgaccgttaacagaac

SEQ ID NO: 88 - DNA, 86 bp, synthetic construct
  misc_feature 1..86: note = synthetic polynucleotide
  source 1..86: mol_type = other DNA
cactttcagtttggcggtctgggtgccttcgtgcgggcggccctcgccttcgccttcgatctcgtcctcgtgaccgttaacagaac

SEQ ID NO: 89 - DNA, 88 bp, synthetic construct
  misc_feature 1..88: note = synthetic polynucleotide
  source 1..88: mol_type = other DNA
actaagtatttgtggcctttatcttctacgtagtgaggatctctcagcgtatggttgtcgcctgagctgtagttgccttcatcgatga

SEQ ID NO: 90 - DNA, 88 bp, synthetic construct
  misc_feature 1..88: note = synthetic polynucleotide
  source 1..88: mol_type = other DNA
tgattcataaacaccggaaatagaagatgcatcactcctagagagtcgcataccaacagcggactcgacatcaacggaagtagctact

SEQ ID NO: 91 - DNA, 88 bp, synthetic construct
  misc_feature 1..88: note = synthetic polynucleotide
  source 1..88: mol_type = other DNA
actaagtatttgtggcctttttcttctacgtagtgaggttctctcagcgtttggttgtcgcctgagctgtagttgccttcatcgatga

SEQ ID NO: 92 - DNA, 88 bp, synthetic construct
  misc_feature 1..88: note = synthetic polynucleotide
  source 1..88: mol_type = other DNA
actaagtatttgtggcctttatcttcgacgtggtggggatctctcagcgtgtggttgtcgcctgagctgtagttgccttcatcgatga

SEQ ID NO: 93 - DNA, 150 bp, synthetic construct
  misc_feature 1..150: note = synthetic polynucleotide
  source 1..150: mol_type = other DNA
cgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacg

SEQ ID NO: 94 - DNA, 5103 bp, synthetic construct
  source 1..5103: mol_type = other DNA
agtcacacatagacagcctgaaacaggcgatgctgcttatcgaatcaaagctgccgacaacacgggagccagtgacgcctcccgtggggaaaaaatcatggcaattctggaagaaatagcgctttcagccggcaaacctgaagccggatctgcgattctgataacaaactagcaacaccagaacagcccgtttgcgggcagcaaaacccgtaccctaggtctagggcggcggatttgtcctactcaggagagcgttcaccgacaaacaacagataaaacgaaaggcccagtctttcgactgagcctttcgttttatttgatgcctggtttgtagagttcatccatgccgtgcgtgatacctgctgcagtaacgaactccagcagcaccatgtggtcgcgcttttcgttcgggtctttggacagtttagactgggtggacaggtagtggttatccggcagcagaaccggaccatcaccgatcggagtgttctgctggtagtggtccgccagctgtacgctaccgtcttcaacgttatggcgaattttgaagttagctttgataccgttcttctgtttgtctgcggtgatgtaaacgttatgggagttgaagttatattccagtttgtggcccaggatgttgccgtcctctttgaaatcaatgcctttcagttcaatacggttcaccagagtatcaccttcaaatttaacctctgcacgggttttgtaggtgccatcgtctttgaaagaaatggtgcgctcctgtacataaccttccggcattgcagatttgaagaaatcatgctgcttcatgtgatccgggtaacgagaaaaacactgaacaccataggtcagggtagtcaccagagtcggccatggaaccggcagtttaccggtagtgcagatgaatttcagggtcagtttaccgttggttgcatcaccttcaccttcaccacgaacagagaatttgtggccgttaacatcaccatccagttcaaccaggatcggaacaacaccggtgaacagttcttcacctttactcatttttgcctcctaactaggtcatttgatatgcctccggatatcactctatcaatgatagagagcttattttaattatgctctatcaatgatagagtgtcaatattttttttagtttttcatgaactcgaggggatccaaataaaaaactagtttgacaaataactctatcaatgatataatgtcaacaaaaaggaggaattaatgatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaagacgtcggaattgccagctggggcgccctctggtaaggttgggaagccctgcaaagtaaactggatggctttcttgccgccaaggatctgatggcgcaggggatcaagatctgatcaagagacaggatgaggatcgtttcgctttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaagcgggactctggggttcgagagctcgcttggactcctgttgatagatccagtaatgacctcagaactccatctggatttgttcagaacgctcggttgccgccgggcgttttttattggtgagaatccaagcactagggacagtaagacgggtaagcctgttgatgataccgctgccttactgggtgcattagccagtctgaatgacctgtcacgggataatccgaagtggtcagactggaaaatcagagggcaggaactgctgaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccagatctcccgggaaaaggactagtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacgtaacggactaagaaaaacactacacgatgctttaactgcaaaaattcagctcaccagttttgaggcaaaatttttgagtgacatgcaaagtaagcatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttagatatcaaagggaaaactgtccatatgcacagatgaaaacggtgtaaaaaagatagatacatcagagcttttacgagtttttggtgcatttaaagctgttcaccatgaacagatcgacaatgtaacagatgaacagcatgtaacacctaatagaacaggtgaaaccagtaaaacaaagcaactagaacatgaaattgaacacctgagacaacttgttacagctcaac

SEQ ID NO: 95 - DNA, 70 bp, synthetic construct
  source 1..70: mol_type = other DNA
tttgtggcctttatcttctacgtagtgaggatctcacagcgtatggttgtcgcctgagctgtagttgcct

SEQ ID NO: 96 - DNA, 825 bp, synthetic construct
  source 1..825: mol_type = other DNA
aaaaagactgaggtgcagactggcggtttctctaaggagtccattctgccgaagcgcaactccgacaagctgatcgcgcgtaagaaggactgggatccgaagaagtacggtggcttcgattctccgaccgtggcgtactctgttctggtggtcgccaaggtcgagaagggtaagtctaagaagctgaagtctgtgaaggagcttctgggcatcaccattatggagcgttcttctttcgagaagaacccgattgacttcctcgaggcgaaggggtacaaggaggtgaagaaggatctgattatcaagctgccgaagtactccctgttcgagctggagaatggtcgtaagcgtatgctggcgtctgcgggtgagctgcagaaggggaacgagttggcccttccgtccaagtacgtgaacttcctgtacctggcctcgcactacgagaagctgaagggttctccggaggataatgagcagaagcagctgttcgtggagcagcacaagcactacctggacgagattattgagcagatttctgagttttctaagcgcgtgattctggcggacgcgaatctggataaggtcctgtctgcctacaataagcaccgtgataagccgatccgtgagcaggcggagaacatcattcacctgttcacgctgactaatcttggtgctccggcggccttcaagtacttcgacaccacgatcgatcgtaagcgttacacctccactaaggaggtcctggatgcgactcttattcaccagtctatcactggcctgtacgagactcgtattgatctgagtcagttgggcggtgactaataa

SEQ ID NO: 97 - DNA, 825 bp, synthetic construct
  source 1..825: mol_type = other DNA
aaaaagacggaggtacagacgggcggcttcagcaaggaaagcatcctgccgaaacgcaacagcgacaaactgatcgcgcgcaagaaagactgggacccgaagaaatacggcggcttcgacagcccaaccgtggcatacagcgtgctggtagtcgccaaagtcgaaaagggcaaaagcaaaaaactgaaaagcgtgaaagaactgctgggcatcaccatcatggaacgcagcagcttcgaaaaaaacccgatcgacttcctcgaagcgaaggggtacaaggaagtaaagaaagacctgatcatcaaactgccgaagtacagcctgttcgaactggaaaacggccgcaaacgcatgctggcgagcgcgggcgaactgcaaaaagggaacgaactggccctgccgagcaagtacgtgaacttcctgtacctggccagccactacgagaaactgaaaggcagcccggaagacaacgagcagaaacagctgttcgtggaacagcacaaacactacctggacgagatcatcgaacagatcagcgagttcagcaaacgcgtaatcctggcggacgcgaacctggacaaagtcctgagcgcctacaacaaacaccgcgacaaaccgatccgcgaacaggcagaaaacatcatccacctgttcacgctgacgaacctgggcgccccggcagccttcaaatacttcgacaccacgatcgaccgcaaacgctacaccagcacgaaagaagtcctggacgcaacgctgatccaccagagcatcacgggcctgtacgaaacgcgcatcgacctgagtcagctgggcggcgactaataa

SEQ ID NO: 98 - DNA, 735 bp, synthetic construct
  source 1..735: mol_type = other DNA
aaaaagggcggtagcggtatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggttccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaggacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaataa

SEQ ID NO: 99 - DNA, 825 bp, synthetic construct
  source 1..825: mol_type = other DNA
aaaaagactgaggtgcagactggcggtttctctaaggagtccattctgccgaagcgcaactccgacaagctgatcgcgcgtaagaaggactgggatccgaagaagtacggtggcttcgattctccgaccgtggcgtaatctgttctggtggtcgccaaggtcgagaagggtaagtctaagaagctgaagtctgtgaaggagcttctgggcatcaccattatggagcgttcttctttcgagaagaacccgattgacttcctcgaggcgaaggggtacaaggaggtgaagaaggatctgattatcaagctgccgaagtactccctgttcgagctggagaatggtcgtaagcgtatgctggcgtctgcgggtgagctgcagaaggggaacgagttggcccttccgtccaagtacgtgaacttcctgtacctggcctcgcactacgagaagctgaagggttctccggaggataatgagcagaagcagctgttcgtggagcagcacaagcactacctggacgagattattgagcagatttctgagttttctaagcgcgtgattctggcggacgcgaatctggataaggtcctgtctgcctacaataagcaccgtgataagccgatccgtgagcaggcggagaacatcattcacctgttcacgctgactaatcttggtgctccggcggccttcaagtacttcgacaccacgatcgatcgtaagcgttacacctccactaaggaggtcctggatgcgactcttattcaccagtctatcactggcctgtacgagactcgtattgatctgagtcagttgggcggtgactaataa

SEQ ID NO: 100 - DNA, 825 bp, synthetic construct
  source 1..825: mol_type = other DNA
aaaaagactgaggtgcagactggcggtttctctaaggagtccattctgccgaagcgcaactccgacaagctgatcgcgcgtaagaaggactgggatccgaagaagtacggtggcttcgattctccgaccgtggcgtaatctgtttaggtggtcgccaaggtcgagaagggtaagtctaagaagctgaagtctgtgaaggagcttctgggcatcaccattatggagcgttcttctttcgagaagaacccgattgacttcctcgaggcgaaggggtacaaggaggtgaagaaggatctgattatcaagctgccgaagtactccctgttcgagctggagaatggtcgtaagcgtatgctggcgtctgcgggtgagctgcagaaggggaacgagttggcccttccgtccaagtacgtgaacttcctgtacctggcctcgcactacgagaagctgaagggttctccggaggataatgagcagaagcagctgttcgtggagcagcacaagcactacctggacgagattattgagcagatttctgagttttctaagcgcgtgattctggcggacgcgaatctggataaggtcctgtctgcctacaataagcaccgtgataagccgatccgtgagcaggcggagaacatcattcacctgttcacgctgactaatcttggtgctccggcggccttcaagtacttcgacaccacgatcgatcgtaagcgttacacctccactaaggaggtcctggatgcgactcttattcaccagtctatcactggcctgtacgagactcgtattgatctgagtcagttgggcggtgactaataa

SEQ ID NO: 101 - DNA, 825 bp, synthetic construct
  source 1..825: mol_type = other DNA
aaaaagactgaggtgcagactggcggtttctctaaggagtccattctgccgaagcgcaactccgacaagctgatcgcgcgtaagaaggactgggatccgaagaagtacggtggcttcgattctccgaccgtggcgtactctgttctggtggtcgccaaggtcgagaagggtaagtctaagaagctgaagtctgtgaaggagcttctgggcatcaccattatggagcgttcttctttcgagaagaacccgattgacttcctcgaggcgaaggggtacaaggaggtgaagaaggatctgattatcaagctgccgaagtactccctgttcgagctggagaatggtcgtaagcgtatgctggcgtaggcgggtgagtagcagaaggggaacgagttggcccttccgtccaagtacgtgaacttcctgtacctggcctcgcactacgagaagctgaagggttctccggaggataatgagcagaagcagctgttcgtggagcagcacaagcactacctggacgagattattgagcagatttctgagttttctaagcgcgtgattctggcggacgcgaatctggataaggtcctgtctgcctacaataagcaccgtgataagccgatccgtgagcaggcggagaacatcattcacctgttcacgctgactaatcttggtgctccggcggccttcaagtacttcgacaccacgatcgatcgtaagcgttacacctccactaaggaggtcctggatgcgactcttattcaccagtctatcactggcctgtacgagactcgtattgatctgagtcagttgggcggtgactaataa

SEQ ID NO: 102 - DNA, 825 bp, synthetic construct
  source 1..825: mol_type = other DNA
aaaaagacggaggtacagacgggcggcttcagcaaggaaagcatcctgccgaaacgcaacagcgacaaactgatcgcgcgctagaaagactgggacccgaagaaatacggcggcttcgacagcccaaccgtggcatacagcgtgctggtagtcgccaaagtcgaaaagggcaaaagcaaaaaactgaaaagcgtgaaagaactgctgggcatcaccatcatggaacgcagcagcttcgaaaaaaacccgatcgacttcctcgaagcgaaggggtacaaggaagtaaagaaagacctgatcatcaaactgccgaagtacagcctgttcgaactggaaaacggccgcaaacgcatgctggcgagcgcgggcgaactgcaaaaagggaacgaactggccctgccgagcaagtacgtgaacttcctgtacctggccagccactacgagaaactgaaaggcagcccggaagacaacgagcagaaacagctgttcgtggaacagcacaaacactacctggacgagatcatcgaacagatcagcgagttcagcaaacgcgtaatcctggcggacgcgaacctggacaaagtcctgagcgcctacaacaaacaccgcgacaaaccgatccgcgaacaggcagaaaacatcatccacctgttcacgctgacgaacctgggcgccccggcagccttcaaatacttcgacaccacgatcgaccgcaaacgctacaccagcacgaaagaagtcctggacgcaacgctgatccaccagagcatcacgggcctgtacgaaacgcgcatcgacctgagtcagctgggcggcgactaataa

SEQ ID NO: 103 - DNA, 825 bp, synthetic construct
  source 1..825: mol_type = other DNA
aaaaagacggaggtacagacgggcggcttcagcaaggaaagcatcctgccgaaacgcaacagcgacaaactgatcgcgtgatagaaagactgggacccgaagaaatacggcggcttcgacagcccaaccgtggcatacagcgtgctggtagtcgccaaagtcgaaaagggcaaaagcaaaaaactgaaaagcgtgaaagaactgctgggcatcaccatcatggaacgcagcagcttcgaaaaaaacccgatcgacttcctcgaagcgaaggggtacaaggaagtaaagaaagacctgatcatcaaactgccgaagtacagcctgttcgaactggaaaacggccgcaaacgcatgctggcgagcgcgggcgaactgcaaaaagggaacgaactggccctgccgagcaagtacgtgaacttcctgtacctggccagccactacgagaaactgaaaggcagcccggaagacaacgagcagaaacagctgttcgtggaacagcacaaacactacctggacgagatcatcgaacagatcagcgagttcagcaaacgcgtaatcctggcggacgcgaacctggacaaagtcctgagcgcctacaacaaacaccgcgacaaaccgatccgcgaacaggcagaaaacatcatccacctgttcacgctgacgaacctgggcgccccggcagccttcaaatacttcgacaccacgatcgaccgcaaacgctacaccagcacgaaagaagtcctggacgcaacgctgatccaccagagcatcacgggcctgtacgaaacgcgcatcgacctgagtcagctgggcggcgactaataa

SEQ ID NO: 104 - DNA, 825 bp, synthetic construct
  source 1..825: mol_type = other DNA
aaaaagacggaggtacagacgggcggcttcagcaaggaaagcatcctgccgaaacgcaacagcgacaaactgatcgcgcgcaagaaagactgggacccgaagaaatacggcggcttcgacagcccaaccgtggcatacagcgtgctggtagtcgccaaagtcgaaaagggcaaaagcaaaaaactgaaaagcgtgaaagaactgctgggcatcaccatcatggaacgcagcagcttcgaaaaaaacccgatcgacttcctcgaagcgaaggggtacaaggaagtaaagaaagacctgatcatcaaactgccgaagtacagcctgttcgaactggaaaacggccgcaaacgcatgctggcgagcgcgggcgaactgcaaaaagggaacgaactggccctgccgagcaagtacgtgaacttcctgtacctggccagccactacgagaaactgaaaggcagcccggaagacaacgagcagaaacagctgttcgtggaacagcacaaacactacctggacgagatcatcgaacagatcagcgagttcagcaaacgcgtaatcctggcggacgcgaacctggacaaagtcctgagcgcctacaacaaacaccgcgacaaaccgatccgcgaacaggcagaaaacatcatccacctgttcacgctgacgaacctgggcgccccggcagccttcaaatacttcgacaccacgatcgaccgctagtgatacaccagcacgaaagaagtcctggacgcaacgctgatccaccagagcatcacgggcctgtacgaaacgcgcatcgacctgagtcagctgggcggcgactaataa

SEQ ID NO: 105 - DNA, 7873 bp, synthetic construct
  source 1..7873: mol_type = other DNA
ggatcggcctatgaactgtcgactcgaggccttgacaggtacctcatggatacctataatgccgtgggtgatggtttgatacaaaggagaaatctagatggtttccaagggcgaggaggataacatggctatcattaaagagttcatgcgcttcaaagttcacatggagggttctgttaacggtcacgagttcgagatcgaaggcgaaggcgagggccgtccgtatgaaggcacccagaccgccaaactgaaagtgactaaaggcggcccgctgccttttgcgtgggacatcctgagcccgcaatttatgtacggttctaaagcttatgttaaacacccagcggatatcccggactatctgaagctgtcttttccggaaggtttcaagtgggaacgcgtaatgaattttgaagatggtggtgtcgtgaccgtcactcaggactcctccctgcaggatggcgagttcatctataaagttaaactgcgtggtactaattttccatctgatggcccggtgatgcagaagaagacgatgggttgggaggcgtctagcgaacgcatgtatccggaagatggtgcgctgaaaggcgaaattaaacagcgcctgaaactgaaagatggcggccattatgacgctgaagtgaaaaccacgtacaaagccaagaaacctgtgcagctgcctggcgcgtacaatgtgaatattaaactggacatcacctctcataatgaagattatacgatcgtagagcaatatgagcgcgcggagggtcgtcattctaccggtggcatggatgagctgtacaaataatgcgtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaaggcatgcctcgagatgcatggcgcctaacctaaactgacaggcatcaaattaagcagaaggccatcctgacggatggcctttttgcgtttcgaacaattgaaaaaacctcgcgccttacctgttgagtaatagtcaaaagcctccggtcggaggcttttgactttctgcttactgaatttcggtggtgccgttaattaaccggtgggccctcatgataataatggtttcttagacgtccgaagttcctattctctagaaagtataggaacttccctaggtcagccaaacgtctcttcaggccactgactagcgataactttccccacaacggaacaactctcattgcatgggatcattgggtactgtgggtttagtggttgtaaaaacacctgaccgctatccctgatcagtttcttgaaggtaaactcatcacccccaagtctggctatgcagaaatcacctggctcaacagcctgctcagggtcaacgagaattaacattccgtcaggaaagcttggcttggagcctgttggtgcggtcatggaattaccttcaacctcaagccagaatgcagaatcactggcttttttggttgtgcttacccatctctccgcatcacctttggtaaaggttctaagcttaggtgagaacatccctgcctgaacatgagaaaaaacagggtactcatactcacttctaagtgacggctgcatactaaccgcttcatacatctcgtagatttctctggcgattgaagggctaaattcttcaacgctaactttgagaatttttgtaagcaatgcggcgttataagcatttaatgcattgatgccattaaataaagcaccaacgcctgactgccccatccccatcttgtctgcgacagattcctgggataagccaagttcatttttctttttttcataaattgctttaaggcgacgtgcgtcctcaagctgctcttgtgttaatggtttcttttttgtgctcatacgttaaatctatcaccgcaagggataaatatctaacaccgtgcgtgttgactattttacctctggcggtgataatggttgcatgtactaaggaggttgtatacgcgtcgtcgatgggttcgggaatgcaggatattcccacctccggttaaggatggaagagagtatctgttccacgaatcagcggtaaaggttgacttaaatcgaccagtaacaggtggccttttgaagaggatcagaaatgggaagaaggcgaagtcatgagcgccgggatttaccccctaacctttatataagaaacaatggatattactgctacagggacccaaggacgggtaaagagtttggattaggcagagacaggcgaatcgcaatcactgaagctatacaggccaacattgagttattttcaggacacaaacacaagcctctgacagcgagaatcaacagtgataattccgttacgttacattcatggcttgatcgctacgaaaaaatcctggccagcagaggaatcaagcagaagacactcataaattacatgagcaaaattaaagcaataaggaggggtctgcctgatgctccacttgaagacatcaccacaaaagaaattgcggcaatgctcaatggatacatagacgagggcaaggcggcgtcagccaagttaatcagatcaacactgagcgatgcattccgagaggcaatagctgaaggccatataacaacaaaccatgtcgctgccactcgcgcagcaaaatcagaggtaaggagatcaagacttacggctgacgaatacctgaaaatttatcaagcagcagaatcatcaccatgttggctcagacttgcaatggaactggctgttgttaccgggcaacgagttggtgatttatgcgaaatgaagtggtctgatatcgtagatggatatctttatgtcgagcaaagcaaaacaggcgtaaaaattgccatcccaacagcattgcatattgatgctctcggaatatcaatgaaggaaacacttgataaatgcaaagagattcttggcggagaaaccataattgcatctactcgtcgcgaaccgctttcatccggcacagtatcaaggtattttatgcgcgcacgaaaagcatcaggtctttccttcgaaggggatccgcctacctttcacgagttgcgcagtttgtctgcaagactctatgagaagcagataagcgataagtttgctcaacatcttctcgggcataagtcggacaccatggcatcacagtatcgtgatgacagaggcagggagtgggacaaaattgaaatcaaataatgattttattttgactgatagtgacctgttcgttgcaacaaattgataagcaatgccaagcttggcactggctgatcagctagcccatgggtatggacagttttccctttgatatgtaacgcacgttgtgtctcaaaatctctgatgttacattgcacaagataaaaatatatcatcatgaacaataaaactgtctgcttacataaacagtaatacaaggggtgttatgagccatattcaacgggaaacgtcttgctcccgtccgcgcttaaactccaacatggacgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtccgtctcaactggctgacggagtttatgcctctcccgaccatcaagcattttatccgtactcctgatgatgcgtggttactcaccaccgcgattcctgggaaaacagccttccaggtattagaagaatatcctgattcaggtgaaaatattgttgatgcgctggccgtgttcctgcgccggttacattcgattcctgtttgtaattgtccttttaacagcgatcgtgtatttcgtcttgctcaggcgcaatcacgcatgaataacggtttggttgatgcgagtgattttgatgacgagcgtaatggctggcctgttgaacaagtctggaaagaaatgcacaagctcttgccattctcaccggattcagtcgtcactcatggtgatttctcacttgataaccttatttttgacgaggggaaattaataggttgtattgatgttggacgggtcggaatcgcagaccgttaccaggaccttgccattctttggaactgcctcggtgagttttctccttcattacagaaacggctttttcaaaaatatggtattgataatcctgatatgaataaattgcagtttcatttgatgctcgatgagtttttctaataatactagcagaaatcatccttagcgaaagctaaggattttttttatctgattaccgcctttgagtgagcgtcgacctagtgcggccgcaagatccggccacgatgcgtccggcgtagaggatctgaagatcagcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatgtaacgcactgagaagcccttagagcctctcaaagcaattttgagtgacacaggaacacttaacggctgacatgggaattaggaagttcctattccgaagttcctattctctagaaagtataggaacttccatatgccatggcatcacagtatcgtgatgacagaggcagggagtgggacaaaattgaaatcaaataatgattttattttgactgatagtgacctgttcgttgcaacaaattgataagcaatgcttttttataatgccaacttagtataaaaaagctgaacgagaaacgtaaaatgatataaatatcaatatattaaattagattttgcataaaaaacagactacataatactgtaaaacacaacatatatgcagtcactatgaatcaactacttagatggtattagtgacctgtaacagagcattagcgcaaggtgatttttgtcttcttgcgctaattttttgtcatcaaacctgtcgcactccagagaagcacaaagcctcgcaatccagtgcaaagctagcttcttcgtctgtttctactggtattggcacaaacctgattccaatttgagcaaggctatgtgccatctcgatactcgttcttaactcaacagaagatgctttgtgcatacagcccctcgtttattatttatctcctcagccagccgctgtgctttcagtggatttcggataacagaaaggccgggaaatacccagcctcgctttgtaacggagtagagacgaaagtgattgcgcctacccggatattatcgtgaggatgcgtcatcgccattaattcactgatcagtgatagctgtcaaacatgagaattgatccggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatgctgcacatgacattaacctataaaaataggcgtatcacgaggccctttcgtcttcaagaattaattcccaattccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggaattaattccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggaattaattccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggaattaattccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggaattgg

SEQ ID NO: 106 - DNA, 8493 bp, synthetic construct
  source 1..8493: mol_type = other DNA
gattagcagaaagtcaaaagcctccgaccggaggcttttgactaaaacttcccttggggttatcattggggctcactcaaaggcggtaatcagataaaaaaaatccttagctttcgctaaggatgatttctgctagagctgtcagaccaagtttacgagctcgcttggactcctgttgatagatccagtaatgacctcagaactccatctggatttgttcagaacgctcggttgccgccgggcgctttttattggtgagaatccaagcactagggacagtaagacgggtaagcctgttgatgataccgctgccttactgggtgcattagccagtctgaatgacctgtcacgggataatccgaagtggccagactggaaaatcagagggcaggaactgctgaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggacatgtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacataacggactaagaaaaacactacacgatgctttaactgcaaaaattcagctcaccagttttgaggcaaaatttttgagtgacatgcaaagtaagtatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttacatatcaaagggaaaactgtccatatgcacagatgaaaacggtgtaaaaaagatagatacatcagagcttttacgagtttttggtgtattcaaagctgttcaccatgaacagatcgacaatgtaactactagagctaaatacattcaaatatctatccgctcatgagacaataaccctgataaatgcttcaataatattgaaaaaggaagaatatgagtattcaacatttccgtgtcgcccttattcccttttttgcggcattttgccttcctgtttttgctcacccagaaacgctggtgaaagtaaaagatgccgaagatcagttgggtgcacgtgtgggttacatcgaactggacctcaacagcggtaagattcttgagagttttcgccccgaagaacgtttcccaatgatgagcacttttaaagttctgctctgtggcgcggtattatcccgtattgacgccgggcaagagcaactcggtcgccgcatacactattctcagaatgacttggttgagtactcaccagtcacagaaaagcatcttacggacggcatgacagtacgcgaattatgcagcgctgccataaccatgagtgataacacggcggccaacttacttctgacaacgatcggaggaccgaaggagcttaccgcttttttgcacaacatgggtgatcatgtaactcgccttgatcgttgggaaccggagctgaatgaagccataccaaacgacgagcgtgacaccacgatgcctgtagctatggcaacaacgttgcgcaaactcttaactggcgaacttcttactctcgcttcccggcaacaattaatagactggatggaggcggataaagttgcaggaccacttctgcgctcggcccttccggctggctggtttattgctgataaatctggagccggtgagcgtgggtcccgcggtattattgcagccctggggccagatggtaagccctcccgtatcgtagttatctacacgacggggagccaggcaactatggacgaacgtaatcgccagatcgctgagataggtgcctccctgattaagcattggtaataatactagctccggcaaaaaaacgggcaaggtgtcaccaccctgccctttttctttaaaaccgaaaagattacttcgcgtttgccacctgacgtctaagaaaaggaatattcagcaatttgcccgtgccgaagaaaggcccacccgtgaaggtgagccagtgagttgattgctacgtaattagtttttccataaaactaaagtaagtgtaaacctattcattgttttaaaaatatctcttgccagtcacgttacgttattagttagttaccatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctcctttttgttgacattatatcattgatagagttatttgtcaaactagttttttatttggatcccctcgagttcatgaaaaactaaaaaaaatattgacactctatcattgatagagcataattaaaataagctccctatcagtgatagagaggaggcatatcaaaggacgagtgcaggtggcaaaaatggataagaaatacagcataggcttagctatcggcacaaatagcgtcggatgggcggtgatcactgatgaatataaagttccgtctaaaaagttcaaggtactgggtaatacagatcgccatagtatcaaaaagaacttaatcggtgcgcttctgttcgattccggcgaaaccgcagaagcaacacgtctgaaacgcaccgctcgtcgccgttacacccgtcgtaaaaaccgcatctgctacctgcaagaaatcttctctaacgaaatggctaaagtagatgacagcttttttcaccgtctggaagaatcatttctggtggaagaagataaaaagcacgaacgtcatccaatcttcggcaacattgtggacgaagtagcgtatcacgaaaaatacccgactatctatcacctgcgcaaaaagctggtcgattcgacggataaggccgatctgcgtctgatctatctggccttagcgcatatgattaagttccgtggtcatttcctgatcgaaggcgacctgaatccagacaacagcgatgtagacaaactgttcatccagctggtgcaaacctataaccagctgtttgaagaaaacccaattaatgctagcggtgttgacgcgaaagcgatcttgtccgcacgcctgtccaaatcccgtcgtctggaaaacttaattgcgcaactgccgggtgagaagaaaaacggactgttcggcaatctgatcgctcttagcttgggactgaccccgaacttcaaaagcaacttcgatctggcagaggacgcaaaacttcaacttagcaaagatacgtatgacgatgacttggataacttactggcccagatcggagatcagtacgctgatctgtttctggcggcaaagaacttatcagacgctattctcctgtctgatattcttcgtgtgaataccgaaatcaccaaagcaccgctttctgcatccatgattaaacgctatgacgaacatcaccaagatctgactcttctgaaagcgctggtacggcaacaactgccggagaagtacaaggagatcttctttgaccaatccaaaaacggctacgcgggttatattgacgggggtgcaagccaagaggagttctacaaattcatcaagccaatcttagaaaaaatggatggcacggaagaattacttgttaaactgaatcgtgaggatctgcttcgtaaacagcgtaccttcgacaacggtagcattccgcaccagatccacttaggtgaactgcacgctatcctgcgtcgccaagaggatttttacccgttcctgaaagataatcgtgaaaaaatcgaaaaaatcctgacctttcgtatcccgtattatgtcggcccgctggcgcgtggcaactcccgtttcgcgtggatgactcgcaaatccgaagaaactattaccccgtggaacttcgaggaagtggttgacaaaggcgcaagcgcccaatccttcatcgagcgcatgactaactttgataaaaacctgccgaacgaaaaggtactgccgaaacactcccttctgtacgaatacttcaccgtgtacaacgagctgactaaagtaaagtatgtgactgagggcatgcgtaaacctgcattcctgagcggtgaacagaaaaaagcaattgttgatttactgtttaaaaccaaccgtaaagtaaccgttaaacagctgaaagaggactacttcaagaaaatcgaatgcttcgactccgtcgagattagtggagttgaagatcgttttaatgcaagtttaggcacgtatcacgatttattaaagatcattaaagacaaagatttcttggacaacgaagaaaatgaggacatcttagaggacatcgtcctgaccctgactctgttcgaagatcgtgaaatgattgaagaacgcttgaagacgtatgctcacctgtttgacgataaagtaatgaaacaactgaaacgtcgccgttatactggctggggccgtctgagccgtaaactgattaacggtatccgtgacaaacagtccggtaaaactattctggacttcctgaaatctgacggcttcgcaaaccgtaacttcatgcaactgattcacgacgattccctgaccttcaaagaggacatccagaaagctcaggtttctggtcaaggtgattctctgcacgagcatatcgccaatttagcaggtagtccggcgatcaaaaaaggtatcctgcaaaccgtgaaagtggtggatgagcttgtgaaagttatgggtcgtcacaaaccggaaaacattgttatcgagatggctcgtgaaaaccaaacgacccagaagggacagaaaaactcccgcgaacgcatgaaacgtatcgaggagggtattaaagaacttggctctcagattctgaaagaacaccctgttgaaaatacccaactgcaaaatgaaaaactgtacctgtactacctgcaaaatggtcgtgacatgtatgtagatcaggagctggacatcaaccgcctctccgattacgacgttgacgccattgttccgcagtcttttctgaaagatgattccattgataacaaagtactcacccgtagcgataaaaaccgtgggaagagtgacaacgttccatcggaagaagtagttaagaaaatgaagaactattggcgtcaactgcttaacgcgaaactgattactcaacgtaaatttgataacctgaccaaagctgaacgtggcggtttgtctgagctggataaggcgggttttattaaacgtcaactggtagaaactcgccagattacaaaacatgttgctcagattctggactctcgtatgaacactaaatacgatgaaaatgacaaactgatccgcgaagttaaggttattaccctgaaatctaagctggtttccgacttccgtaaagatttccaattctataaagtgcgcgagattaacaactatcaccacgcgcacgacgcatatctgaatgcagttgttggcacggcactgatcaaaaaatatccgaaactggaaagcgaatttgtgtacggcgattataaagtttacgacgtgcgcaaaatgatcgccaaatctgaacaggaaattggcaaagcaaccgctaaatactttttctactcaaacattatgaatttcttcaaaaccgaaatcaccttagcgaatggcgaaattcgtaaacgccctctgatcgaaaccaacggcgaaacgggtgagatcgtgtgggacaaaggtcgtgatttcgctactgtccgcaaagttctgtccatgcctcaagtaaacatcgttaaaaacgagacctgaattgcgcgcaattaaccctcactaaagggaacaaaagctggagctcttatattccccagaacatcaggttaatggcgtttttgatgtcattttcgcggtggctgagatcagccacttcttccccgataacggacaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatcgtttcactccatccaaaaaaacgggtatggagaaacagtagagagttgcgataaaaagcgtcaggtaggatccgctggtctctaaggcgttaaaccaggcatcaaataaaacgaaaggctcagtcgaaagactgggcctttcgttttatctgttgtttgtcggtgaacgctctctactagagtcacactggctcaccttcgggtgggcctttctgcgtttataatatactcctaaattaacttttaaagcaatgaaaatagtgaacattataactgttgtgtaacagaatgcaattagcatattactgttacacaaattagtacagtttctatgttttgacatacatttgatgaaaattgtacataatttatgtgaaaaaaatcacaacaaacatgctacaattagttgagaccagtctaggtctcggttttagagctagaaatagcaagttaaaataaggctagtccgttatcaacttgaaaaagtggcaccgagtcggtgctttttttggagatctgtccatacccatggattcttcgtctgtttctactccatcagtctgacgaccaagagagccataaacaccaatagccttaac

SEQ ID NO: 107 - DNA, 20 bp, synthetic construct
  source 1..20: mol_type = other DNA
catggatacctataatgccg

SEQ ID NO: 108 - DNA, 70 bp, synthetic construct
  source 1..70: mol_type = other DNA
cggtggcttcgattctccgaccgtggcgtaatctgttctggtggtcgccaaggtcgagaagggtaagtct

SEQ ID NO: 109 - DNA, 80 bp, synthetic construct
  source 1..80: mol_type = other DNA
aagtacggtggcttcgattctccgaccgtggcgaaatctgttctagtggtcgccaaggtcgagaagggtaagtctaagaa

SEQ ID NO: 110 - DNA, 113 bp, synthetic construct; identical to SEQ ID NO: 21
  source 1..113: mol_type = other DNA

SEQ ID NO: 111 - DNA, 70 bp, synthetic construct
  source 1..70: mol_type = other DNA
cggtggcttcgattctccgaccgtggcgtaatctgtttaggtggtcgccaaggtcgagaagggtaagtct

SEQ ID NO: 112 - DNA, 70 bp, synthetic construct
  source 1..70: mol_type = other DNA
agccgccgtatttcttcgggtcccagtctttctagcgcgcgatcagtttgtcgctgttgcgtttcggcag

SEQ ID NO: 113 - DNA, 70 bp, synthetic construct
  source 1..70: mol_type = other DNA
agccgccgtatttcttcgggtcccagtctttctatcacgcgatcagtttgtcgctgttgcgtttcggcag

SEQ ID NO: 114 - DNA, 71 bp, synthetic construct
  source 1..71: mol_type = other DNA
ggtggatcagcgttgcgtccaggacttctttcgtgctggtgtatcactagcggtcgatcgtggtgtcgaag

SEQ ID NO: 115 - DNA, 60 bp, synthetic construct
  source 1..60: mol_type = other DNA
gcttcgattcaccgaccgtggcgaaatctgttctggtggtcgccaaggtcgagaagggta

SEQ ID NO: 116 - DNA, 70 bp, synthetic construct
  source 1..70: mol_type = other DNA
gattctccgaccgtggcgaaatcagttctggtggtcgccaaggtcgagaagggtaagtctaagaagctga

SEQ ID NO: 117 - DNA, 67 bp, synthetic construct
  source 1..67: mol_type = other DNA
tggtcgtaagcgtatgctggcgtaggcgggtgagtagcagaaggggaacgagttggcccttccgtcc

SEQ ID NO: 118 - DNA, 60 bp, synthetic construct
  source 1..60: mol_type = other DNA
tacggtggcttcaaatctccgaccgtggcgaaatctgttaaagtagtcgccaaggtcgag

SEQ ID NO: 119 - DNA, 70 bp, synthetic construct
  source 1..70: mol_type = other DNA
tacggtggcttcaaatctccgaccgtagcgaaatctgttaaagtggtcgccaaggtcgagaagggtaagt

SEQ ID NO: 120 - DNA, 80 bp, synthetic construct
  source 1..80: mol_type = other DNA
gaagtacggtggcttcaaatctccgaccgtggcgtactctgttctggtggtagccaaggtcgagaagggtaagtctaaga

SEQ ID NO: 121 - DNA, 60 bp, synthetic construct
  source 1..60: mol_type = other DNA
aagcgtatgctggcgtctgcaaaaaaactgcagaaggggaacgagttggcccttccgtcc

SEQ ID NO: 122 - DNA, 70 bp, synthetic construct
  source 1..70: mol_type = other DNA
aatggtcgtaagcgtatgctggcatctgcgaaaaaactgcagaaggggaacgagttggcccttccgtcca

SEQ ID NO: 123 - DNA, 80 bp, synthetic construct
  source 1..80: mol_type = other DNA
ctggagaatggtcgtaagcgaatgctggcgtctgcgaatgaactgcagaaggggaacgagttggcccttccgtccaagta

SEQ ID NO: 124 - DNA, 60 bp, synthetic construct
  source 1..60: mol_type = other DNA
tcccagtctttaaaaaacgcgatcagtttgtcgctgttgcgtttaggaaagatgctttcc

SEQ ID NO: 125 - DNA, 70 bp, synthetic construct
  source 1..70: mol_type = other DNA
cgggtcccagtctttaaaaaaagcgatcagtttgtcgctgttgcgtttcggaaagatgctttccttgctg

SEQ ID NO: 126 - DNA, 80 bp, synthetic construct
  source 1..80: mol_type = other DNA
gccgccgtatttcttcgggtcccagtctttaaaaaacgcgatcagtttgtcgctgttgcgtttaggaaagatgctttcct

SEQ ID NO: 127 - DNA, 59 bp, synthetic construct
  source 1..59: mol_type = other DNA
gcgttgcgtccaggacttctttcgtgctaaagtaaaatttacgatcgatcgtggtgtcg

SEQ ID NO: 128 - DNA, 73 bp, synthetic construct
  source 1..73: mol_type = other DNA
gctctggtggatcagcgtagcgtccaggacttctttcgtgctaaagtaaaatttgcgatcgatcgtggtgtcg

SEQ ID NO: 129 - DNA, 80 bp, synthetic construct
  source 1..80: mol_type = other DNA
gcgttgcgtccaggacttctttcgtgctaaagtaaaatttacgatcgatcgtggtgtcgaagtatttgaaggctgccggg

SEQ ID NO: 130 - DNA, 96 bp, synthetic construct
  source 1..96: mol_type = other DNA
gatccgaagaagtacggtggcttcgattctccgaccgtggcgtaatctgttctggtggtcgccaaggtcgagaagggtaagtctaagaagctgaag

SEQ ID NO: 131 - DNA, 96 bp, synthetic construct
  source 1..96: mol_type = other DNA
ctaggcttcttcatgccaccgaagctaagaggctggcaccgcattagacaagaccaccagcggttccagctcttcccattcagattcttcgacttc

SEQ ID NO: 132 - protein, 32 aa, synthetic construct
DPKKYGGFDSPTVASVLVVAKVEKGKSKKKLK

SEQ ID NO: 133 - DNA, 96 bp, synthetic construct
  source 1..96: mol_type = other DNA
gatccgaagaagtacggtggcttcgattctccgaccgtggcgaggtctgttctggtggtcgccaaggtcgagaagggtaagtctaagaagctgaag

SEQ ID NO: 134 - DNA, 96 bp, synthetic construct
  source 1..96: mol_type = other DNA
gatccgaagaagtacggtggcttcgattctccgaccgtggcgttatctgttctggtggtcgccaaggtcgagaagggtaagtctaagaagctgaag

SEQ ID NO: 135 - DNA, 96 bp, synthetic construct
  source 1..96: mol_type = other DNA
gatccgaagaagtacggtggcttcgattctccgaccgtggcgttttctgttctggtggtcgccaaggtcgagaagggtaagtctaagaagctgaag

SEQ ID NO: 136 - DNA, 96 bp, synthetic construct
  source 1..96: mol_type = other DNA
gatccgaagaagtacggtggcttcgattctccgaccgtggcggaatctgttctggtggtcgccaaggtcgagaagggtaagtctaagaagctgaag

SEQ ID NO: 137 - DNA, 96 bp, synthetic construct
  source 1..96: mol_type = other DNA
gatccgaagaagtacggtggcttcgagtctccgaccgtggcgtggtctgttctggtggtcgccaaggtcgagaagggtaagtctaagaagctgaag

SEQ ID NO: 138 - DNA, 96 bp, synthetic construct
  source 1..96: mol_type = other DNA
gatccgaagaagtacggtggcttcgattctccgcccgtggcgttatctgttctggtggtcgccaaggtcgagaagggtaagtctaagaagctgaag

SEQ ID NO: 139 - DNA, 106 bp, synthetic construct
  source 1..106: mol_type = other DNA
actgggatccgaagaagtacggtggcttcgattctccgaccgtggcgtaatctgttctggtggtcgccaaggtcgagaagggtaagtctaagaagctgaagtctgt

SEQ ID NO: 140 - DNA, 106 bp, synthetic construct
  source 1..106: mol_type = other DNA
tgaccctaggcttcttcatgccaccgaagctaagaggctggcaccgcattagacaagaccaccagcggttccagctcttcccattcagattcttcgacttcagaca

SEQ ID NO: 141 - protein, 35 aa, synthetic construct
DWDPKKYGGFDSPTVASVLVVAKVEKGKSKKLKSV

SEQ ID NO: 142 - DNA, 106 bp, synthetic construct
  source 1..106: mol_type = other DNA
actgggatccgaagaagtacggtggcttcgattctccgaccgtggcgaaatctgttctggtggtcgccaaggtcgagaagggtaagtctaagaagctgaagtctgt

SEQ ID NO: 143 - DNA, 106 bp, synthetic construct
  source 1..106: mol_type = other DNA
actgggatccgaagaagtacggtggcttcgattctccggccgtggcggaatctgttctagtggtcgccaaggtcgagaagggtaagtctaagaagctgaagtctgt

SEQ ID NO: 144 - DNA, 106 bp, synthetic construct
  source 1..106: mol_type = other DNA
actgggatccgaagaagtacggtggcttcgattctccgaccgtggcgaaatctgttctggtggtcgccgaggtcgagaagggtaagtctaagaagctgaagtctgt

SEQ ID NO: 145 - DNA, 106 bp, synthetic construct
  source 1..106: mol_type = other DNA
actgggatccgaagaagtacggtggcttcgattctccgactgtggcgtaatctgttctggtggtcgccaaggtcgagaagggtaagtctaagaagctgaagtctgt

(19) United States
(12) Patent Application Publication
(10) Pub. No.: US 2026/0043172 A1
(43) Pub. Date: Feb. 12, 2026

(54) METHODS AND SYSTEMS FOR GENERATING NUCLEIC ACID DIVERSITY IN CRISPR-ASSOCIATED GENES

(71) Applicant: INSTITUT PASTEUR, PARIS 15 (FR)

(72) Inventors: David BIKARD, PARIS (FR); Raphael LAURENCEAU, PARIS (FR); William ROSTAIN, PARIS (FR)

(21) Appl. No.: 19/102,556

(22) PCT Filed: Aug. 14, 2023
(86) PCT No.: PCT/EP2023/072363

§ 371 Date: Feb. 10, 2025

Related U.S. Application Data
(60) Provisional application No. 63/397,915, filed on Aug. 15, 2022.

(51) Int. Cl. C40B 40/08; C12N 9/12; C12N 9/22; C12N 15/10; C12N 15/113; C40B 30/06; C40B 40/10; C40B 50/06

(52) U.S. Cl. CPC C40B 40/08; C12N 9/1276; C12N 9/222; C12N 15/102; C12N 15/113; C12Y 207/07049; C40B 30/06; C40B 40/10; C40B 50/06; C12N 2310/20

(57) ABSTRACT

Provided are methods comprising expressing in a recombinant cell comprising a Cas gene a recombinant error-prone reverse transcriptase (RT) and a recombinant spacer RNA comprising a target sequence for mutagenesis of a DNA sequence in the Cas gene; making a mutagenized cDNA polynucleotide homologous to the DNA sequence in the recombinant cell; expressing a recombinant recombineering system in the recombinant cell; and recombining the mutagenized cDNA with the homologous DNA sequence of the Cas gene in the recombinant cell. Also provided are recombinant cells comprising recombinant coding sequences for a recombinant Cas protein, recombinant error-prone reverse transcriptase (RT), recombinant spacer RNA comprising the target sequence, and recombinant recombineering system.

### FIELD OF THE INVENTION

The invention relates to a method for generating targeted nucleic acid diversity in CRISPR-associated (Cas) genes in vivo in a recombinant cell. The invention further relates to a recombinant cell system for generating targeted nucleic acid diversity in Cas genes and to their uses for the generation and screening of Cas libraries in vivo.

### BACKGROUND

Since its discovery and repurposing as a genetic engineering tool, Clustered Regularly Interspersed Short Palindromic Repeats (CRISPR)/CRISPR-associated protein 9 (Cas9) derived tools have flourished, with applications including genome engineering, base-editing, RNA-guided transcriptional repression and activation or chromatin remodelling. Most of these tools rely on the programmable nature of Cas9 targeting to DNA, which also applies to its catalytically dead variant dCas9. This programmability is governed by two factors: a guide RNA (gRNA), which is homologous and can be modified, and the Protospacer Adjacent Motif (PAM), which is fixed and must be present next to the desired target. For instance, the PAM sequence of the S. pyogenes Cas9 is NGG, so the wild-type enzymes and systems that use it can only be targeted to sequences adjacent to an NGG sequence. There has been interest in engineering variants to relax this requirement to permit the targeting of other sequences. This has included the production and screening of Cas9 libraries using various methods. Aside from PAM-modified Cas9 and dCas9 variants, there is also interest in modifying the ability of variants to bind DNA more or less strongly or more or less specifically, which can also be achieved through the production and screening of DNA libraries of Cas9 variants.

Directed evolution mimics natural selection with the goal to generate useful variants of nucleic acids and/or proteins of interest. Mutations can be introduced in genes either randomly, through mutagenic agents, or in a targeted manner in a gene of interest, optionally followed by selection for a trait of interest. When the goal is to evolve a specific gene or set of genes, targeted diversity generation may be useful to limit the chances that mutations outside of the genes of interest will be selected. Targeted mutagenesis can also ensure that many more sequences of the target gene are being evaluated than what would otherwise be possible through purely random mutagenesis approaches. Careful design of the targeted approach can also ensure an efficient exploration of the sequence space, for instance by exploring sequence variation at specific residues of interest or by avoiding non-sense mutations. This targeted mutagenesis has typically been conducted in vitro through various molecular biology techniques including error-prone PCR, or through the rational design and construction of plasmid libraries. These steps can, however, be cumbersome, especially when many cycles of evolution are performed. The ability to diversify sequences in a targeted manner directly in vivo is a long-standing goal of directed evolution and a step towards continuous evolution setups where both diversification and selection can happen in vivo.

Examples of targeted diversity generation exist in nature. Diversity generation in antibodies is a key feature of human adaptive immune system. In bacteria, diversity generating retroelements (DGRs) are able to introduce controlled sequence diversity in phage proteins and bacterial proteins involved in the interaction with their environment. DGRs, initially characterized in the Bordetella bacteriophage BPP-1 [1], are found in a wide range of phage, bacteria, and archaea [2]. In DGR recombination, a variable region within the genome will be overwritten by a DNA fragment produced from a near repeat template region in a process involving transcription, error-prone reverse transcription of the template and recombination. The error-prone reverse transcription ensures the introduction of genetic diversity at the variable region. In the DGR systems characterized to date, two DGR proteins are necessary for this process, a reverse transcriptase major subunit (RT) and an accessory subunit (Avd) that together form the active reverse transcriptase complex ([1]; [3];[4]; [5]; [6];[7]). An alternative accessory gene consisting of an HRDC (helicase and RNase D C-terminal) domain was also identified in some DGRs by bioinformatic analysis [3]. Most variable regions have been identified within a few kilobase pairs (kb) of the template region and the two DGR proteins ([3]; [2]). The template region that defines the mutagenesis window is embedded within the Avd and RT coding sequences, inside a transcribed RNA segment starting from the end of the AVD gene to the start of the RT gene, named Spacer RNA, the DGR RNA or DGR Spacer RNA. A cDNA copy is unfaithfully generated from the mRNA by the DGR RT complex in a self-priming process [6]. A specific bias in the DGR RT incorporates random nucleotides in place of adenines. The variable region is then overwritten using this cDNA copy, resulting in the acquisition of A to N mutations in the gene. Due to the location of A residues within the sequence, the overall protein structure, typically a C-type lectin fold, is typically preserved while key residues in the binding groove are changed ([1]; [8]). In the case of Bordetella, DGR recombination can introduce a diversity of 10¹³ unique amino acid sequences. However, the positions of the A nucleotides in the codons (i.e. exclusively in the first and second positions of the codons) negate the possibility of non-sense mutations occurring ([1]; [8]). A DGR system has already been harnessed to redirect the mutagenesis towards a target sequence of choice [9], however this was achieved only by using the DGR in its native host, a Bordetella strain, and maintaining the requirement of a recognition sequence to be placed next to the desired mutagenesis window (the IMH sequence), which dramatically limits its possible applications as a genetic tool.

While DGRs have yet to be harnessed in directed evolution setups, a large number of artificial targeted mutagenesis strategies have been proposed, and have multiplied in recent years, demonstrating a pressing need for improvement in this field ([10]; [11]). Indeed, the ability to precisely mutagenize a particular segment of coding DNA is at the cornerstone of applications that extend to all subfields of biotechnology, from enzyme engineering, vaccine development, to diagnostics developments. Recently reviewed by Csörgö et al. [10], targeted mutagenesis technologies can be classified across several parameters including mutagenesis rate and span, and the conditions in which the library of variant sequences are generated.

Only a handful of targeted mutagenesis technologies, out of the dozen that have been developed to date, allow for in vivo mutagenesis.

In the EvolvR system, a D10A Cas9 nickase (Cas9n1) is used to localize a fused error-prone nick-translating DNA polymerase to a desired region of the genome (Halperin et al. 2018). Cas9n1 nicks one strand, generating a 3′ end that can be extended by the fused DNA polymerase followed by repair [13]. Such re-polymerization results in nucleotide misincorporation and can cause a peak 10⁸-fold increase in the DNA mutation rate immediately upstream of the Cas9 nick site, around 1 mutation per 10² nucleotides per generation [13]. By altering the fused polymerase, the EvolvR system can be modulated to alter the mutation rate as well as increase or decrease the size of the window where mutations preferentially occur. A limitation of EvolvR is its propensity to introduce nonsense mutations. The overall E. coli mutation rate is also affected by the presence of the mutagenic polymerase fusion increased between 120-fold to 555-fold, and raising the risk to select mutations outside the region of interest.

The T7-DIVA system relies on a mutagenic T7 RNA polymerase-Base Deaminase fusion (BD-T7RNAP). The mutagenesis window is delineated upstream by the T7 promoter, and downstream by the targeting with dCas9 to serve as a “roadblock” for BD-T7RNAP elongation [14]. The requirement for a T7 promoter means that mutagenesis of the target sequence in its native genomic context is not feasible, and the Base Deaminase mutation profile being restricted to a single possible nucleotide substitution (for example C>T) limits its ability to generate tailored mutagenesis for exploring protein sequence diversity.

A system developed by Simon et al. relies on engineered retrons (another bacterial retroelement, unrelated to DGRs). The mutagenesis activity results from coupling the retron with a mutagenic T7 RNA polymerase [15]. They obtain mutation rates in the targeted region 190-fold higher than background cellular mutation rates (up to 6.3×10⁻⁷ per generation) over a mutagenesis window restricted to 31 bp (thus covering only a maximum of 10 amino acids in a protein-coding sequence). This limits its ability to generate tailored mutagenesis for exploring protein sequence diversity.

Overall, these methods suffer from a low mutagenesis rate. In addition, none of the techniques available to date provide control over the exact position of the bases that are mutated nor offer mechanisms to ensure that the mutations introduced will not generate stop codons. Accordingly, there exists a great need to develop additional methods, systems, compositions, and manufactures for generating sequence diversity in Cas genes and applications of using it. This invention meets these and other needs in certain embodiments.

### SUMMARY

This invention provides an in vivo targeted diversity generation strategy of CRISPR-associated (Cas) genes based on the use of a mutagenic reverse transcriptase, producing mutagenized cDNA oligos homologous to a desired target sequence, which are then recombined within a target region anywhere on the genome or recombinant vector via oligo recombineering (FIG. 1). A functional implementation of the strategy in the model laboratory organism E. coli is demonstrated, enabling various applications in directed evolution of Cas proteins.

The approach relies on two critical achievements disclosed herein for the first time: 1) The expression of a functional plasmid-based mutagenic retroelement platform (or system) in E. coli (inspired from natural DGRs); and 2) The coupling of this system with oligonucleotide recombineering, enabling the incorporation of mutations in a target region anywhere on the genome or recombinant vector (FIG. 1). This system is named DGR Recombineering or DGRec.

These two combined elements represent a major achievement for directed evolution applications, as an unprecedented number of protein sequence variants can be produced in vivo, in a highly targeted manner, from a flexible plasmid-borne system. In certain embodiments, 20 to 500 bp DNA sequence from a host genome or recombinant vector can be densely mutagenized, simply by specifying the mutagenesis target into the DGR Spacer RNA locus. In some embodiments, a plurality of DGR spacer RNAs are used, which increase the target size achievable beyond the size requirements of a single DGR spacer RNA and enables the mutagenesis of stretches of sequences that are not contiguous.

Moreover, the mutagenesis profile may be highly specific and predictable. When using a reverse-transcriptase from a DGR system, adenine positions may in certain embodiments be substituted with roughly 25% chance with an A, T, C or G nucleotide [7]. This predictable mutagenesis provides flexibility in designing both the cDNA template, as well as giving the option to recode the target gene sequence, placing codons that favor some amino acids over others.

Finally, the DGRec system has a great potential for transposability in Eukaryotic cells. Another bacterial retroelement (the Ec86 retron) has recently been successfully expressed for genetic editing applications in different eukaryotic cells including human cells [18]-[20]. Furthermore, despite DNA repair mechanisms are significantly different in eukaryotic and prokaryotic cells, the method of oligonucleotide recombineering originally developed uniquely in bacteria has also been successfully used in eukaryotic cells [21], suggesting that the DGRec method should be easily transposable to eukaryotes.

DGRec system permits the creation of DNA libraries in vivo, which are then screened to select for functional protein variants. The present invention provides adaptation of the DGRec system to generate Cas libraries in vivo, coupled with selection methods which permit the isolation of DGRec-generated Cas variants with modified amino acid sequences and novel properties, such as for example dead Cas variants with improved ability to repress transcription, Cas variants that can recognize non-canonical PAM sequences or other dead Cas or Cas variants.

In a first aspect, the invention provides methods comprising expressing in a recombinant cell comprising a CRISPR-associated (Cas) gene, in particular a recombinant Cas gene, a recombinant error-prone reverse transcriptase (RT) and recombinant spacer RNA comprising a target sequence for mutagenesis of a DNA sequence in the Cas gene; making a mutagenized cDNA polynucleotide homologous to the DNA sequence in the recombinant cell; expressing a recombinant recombineering system in the recombinant cell; and recombining the mutagenized cDNA with the homologous DNA sequence of the Cas gene in the recombinant cell. In some embodiments of the methods, the recombinant error-prone reverse transcriptase (RT) comprises the motif I/LGXXXSQ (SEQ ID NO: 2). In some embodiments, the recombinant error-prone RT is an engineered recombinant error-prone RT derived from a non-mutagenic reverse-transcriptase; preferably the recombinant error-prone RT is a mutant Ec86 retron reverse transcriptase comprising the replacement of the motif QGXXXSP (SEQ ID NO: 1) with the motif I/LGXXXSQ (SEQ ID NO: 2).

In a second aspect, the invention provides methods comprising expressing in a recombinant cell comprising a CRISPR-associated (Cas) gene, in particular a recombinant Cas gene, a recombinant DGR reverse transcriptase major subunit (RT), recombinant DGR accessory subunit (Avd), and recombinant DGR spacer RNA comprising a target sequence for mutagenesis of a DNA sequence in the Cas gene; making a mutagenized cDNA polynucleotide homologous to the DNA sequence in the recombinant cell; expressing a recombinant recombineering system in the recombinant cell; and recombining the mutagenized cDNA with the homologous DNA sequence of the Cas gene in the recombinant cell. In some embodiments the recombinant DGR RT, recombinant DGR Avd, recombinant DGR spacer RNA and recombinant recombineering system are all expressed from one or a plurality of recombinant plasmids together comprising coding sequences for the recombinant Cas protein, recombinant DGR RT, recombinant DGR Avd, recombinant DGR spacer RNA, and recombinant recombineering system; preferably together further comprising coding sequence(s) for at least one recombinant CRISPR guide RNA. In some embodiments the coding sequences for the recombinant DGR RT and recombinant DGR Avd are present on the same plasmid. In some embodiments the coding sequence for the DGR RT is operatively linked to an inducible promoter. In some embodiments the coding sequences for the recombinant DGR Avd and recombinant DGR spacer RNA are operatively linked to constitutive promoter(s). In some embodiments the recombinant DGR RT, the recombinant DGR Avd, and recombinant DGR spacer RNA are from the Bordetella bacteriophage BPP-1. In some embodiments, the coding sequence for the recombinant Cas protein is on a different plasmid, preferably together with the coding sequences for the at least one recombinant CRISPR guide RNA, optionally wherein the coding sequences for the recombinant Cas protein and at least one recombinant CRISPR guide RNA are operatively linked to inducible promoter(s). In some embodiments, the CRISPR guide RNA is targeted to a sequence with a non-canonical PAM sequence.

In some embodiments, the recombinant error-prone RT has adenine mutagenesis activity; preferably wherein the recombinant error-prone RT is a DGR RT comprising a mutation that decreases its error rate at adenine position selected from the group consisting of: R74A and 1181N, the positions being indicated by alignment with SEQ ID NO: 4.

In some embodiments, the Cas gene is Cas9 gene, Cas12 or Cas13 gene; preferably the Cas9 gene is chosen from Streptococcus pyogenes, Staphylococcus aureus, Streptococcus thermophilus or Streptococcus canis Cas9 genes, and homologs, orthologs thereof, or modified versions thereof. In some embodiments, the Cas gene encodes an enzymatically active endonuclease. In some embodiments, the Cas gene encodes an enzymatically inactive endonuclease. In some embodiments, the homologous sequence of the Cas gene that is targeted for mutagenesis by the DGRec system (mutagenesis target) is in the PAM interacting domain (PID). In some embodiments, the Cas gene comprises at least one nonsense mutation (stop codon) in the mutagenesis target. In some particular embodiments, the nonsense mutation(s) are in the PAM interacting domain (PID), preferably at or in close proximity to one or more of positions L1111, R1122, K1123, D1135, Y1141, L1144, S1216, G1218, E1219, L1220, A1322, K1334, R1335, and T1337 said positions being indicated by alignment with SpCas9 reference sequence. In some particular embodiments, the Cas gene encodes an enzymatically inactive endonuclease (dead Cas or dCas) and further comprises at least one nonsense mutation (stop codon) in the mutagenesis target, in particular the PAM interacting domain (PID), preferably at one or more of the above disclosed positions, or close to one of the disclosed positions.

In some embodiments of the methods the mutagenized target sequence comprises 70 base pairs. In some embodiments of the methods the mutagenized target sequence is from 50 to 120 base pairs long. In some embodiments of the methods the mutagenized target sequence is from 70 to 100 base pairs long. In some embodiments of the method the mutagenized target sequence is from 40 to 200 (40, 50, 70, 100, 120, 150, 175, 200) base pairs long or more, in particular 40 to 300 (40, 50, 70, 100, 120, 150, 175, 200, 225, 250, 275 or 300) base pairs long or more. In some embodiments of the methods, the mutagenized target sequence comprises less than 40 base pairs, in particular 30, 20 base pairs or less.

In some embodiments of the methods the recombinant recombineering system is different from DGR retrohoming. In some embodiments of the methods the recombinant recombineering system is single-stranded annealing protein mediating oligo recombineering, preferably selected from the group consisting of: the phage lambda's Red Beta protein, the functional homolog RecT and variants thereof such as PapRecT and CspRecT, in particular CspRecT. In some embodiments of the methods the recombination frequency is at least 0.01%.

In some embodiments, the adenine content and/or position(s) in the target sequence and/or homologous DNA sequence in the recombinant cell is modified to modulate recombination frequency or control sequence diversity.

In some embodiments of the methods the recombination frequency is 0.1%. In some embodiments of the methods the recombination frequency is at least 1%; preferably 3% or more; more preferably 10% or more. In some embodiments the methods further comprise expressing the mutagenized sequence.

In some embodiments of the methods the recombinant cell is a eukaryotic cell. In some embodiments of the methods the recombinant cell is a prokaryotic cell. In some embodiments of the methods the prokaryotic cell is a bacterial cell. In some embodiments of the methods the bacterial cell expresses mutL* (dominant negative mutL). In some embodiments of the methods the bacterial cell is an E. coli cell. In some embodiments of the methods the E. coli is deleted for the two exonucleases SbcB and RecJ to increase recombineering efficiency.

In some embodiments of the methods, the recombinant cell comprises at least two spacer RNAs comprising a target sequence; in particular at least two DGR spacer RNAs comprising a target sequence; preferably wherein the multiple spacer RNAs target the same gene in the recombinant cell.

Another aspect of the invention relates to a method of generating a library of Cas protein variants comprising:
expressing in a recombinant cell comprising a recombinant Cas gene, a recombinant error-prone reverse transcriptase (RT) and recombinant spacer RNA comprising a target sequence for mutagenesis of a DNA sequence in the recombinant Cas gene;
making a mutagenized cDNA polynucleotide homologous to the DNA sequence in the recombinant cell;
expressing a recombineering system in the recombinant cell;
recombining the mutagenized cDNA with the homologous DNA sequence of the recombinant Cas gene in the recombinant cell according to the method of generating nucleic acid diversity of the present disclosure; and
expressing the recombinant Cas gene comprising the mutagenized DNA sequence in the recombinant cell to generate a library of expressed Cas protein variants.

Another aspect of the invention relates to a method of selection and/or screening of a library of Cas protein variants, comprising:
a) generating a library of expressed Cas protein variants in a recombinant cell according to the method of the present disclosure; and
b) selecting and/or screening the activity of the expressed Cas protein variants.

In some embodiments, the selecting and/or screening step is advantageously performed in the recombinant cell according to the present disclosure. In some embodiments, the recombinant cell further comprises at least one marker for the selection and/or screening of the activity of the expressed Cas protein variants; the screening marker is preferably a fluorescent reporter gene, in particular the mCherry gene and/or the selection marker is SacB gene; the at least one selection and/or screening marker is preferably inserted in the genome of the recombinant cell. In some embodiments, the step a) and/or the step b) are repeated at least one time.

Also provided are libraries of Cas gene mutagenized sequences made according to a method of this invention.

Also provided are libraries of recombinant cells comprising the library of Cas gene mutagenized sequences.

Also provided are recombinant cells comprising recombinant coding sequences for a recombinant Cas protein, a recombinant error-prone reverse transcriptase (RT) and at least one recombinant spacer RNA comprising a target sequence. In some embodiments the cell further comprises the recombinant error-prone reverse transcriptase (RT) and recombinant spacer RNA comprising the target sequence. In some embodiments the cell further comprises coding sequences for at least one recombinant CRISPR guide RNA.

Also provided are recombinant cells comprising recombinant coding sequences for a recombinant Cas protein, a recombinant DGR RT, a recombinant DGR Avd, and at least one recombinant DGR spacer RNA comprising the target sequence. In some embodiments the cell further comprises coding sequences for at least one recombinant CRISPR guide RNA. In some embodiments, the recombinant cell comprises one or a plurality of recombinant plasmids that together comprise the coding sequences for the recombinant Cas protein, the recombinant DGR RT, the recombinant DGR Avd, and at least one recombinant DGR spacer RNA comprising a target sequence for mutagenesis of a DNA sequence in the recombinant Cas gene. In some embodiments the recombinant cell further comprises the recombinant DGR RT, recombinant DGR Avd, and recombinant DGR spacer RNA comprising the target sequence. In some embodiments the coding sequences for the recombinant DGR RT and recombinant DGR Avd are present on the same plasmid. In some embodiments the coding sequence for the DGR RT is operatively linked to an inducible promoter. In some embodiments the coding sequences for the recombinant DGR Avd and recombinant DGR spacer RNA are operatively linked to constitutive promoters. In some embodiments the recombinant DGR RT, the recombinant DGR Avd, and recombinant DGR spacerRNA are from the Bordetella bacteriophage BPP-1. In some embodiments, the coding sequence for the recombinant Cas protein is on a different plasmid, preferably together with the coding sequence(s) for the recombinant CRISPR guide RNA(s). Optionally, the coding sequence for the recombinant Cas protein or the coding sequences for the recombinant Cas protein and recombinant CRISPR guide RNA(s) are operatively linked to constitutive promoter(s)

In some embodiments the target sequence comprises 70 base pairs. In some embodiments the target sequence is from 50 to 120 base pairs long. In some embodiments the target sequence is from 70 to 100 base pairs long. In some embodiments the target sequence is from 40 to 200 (40, 50, 70, 100, 120, 150, 175, 200) base pairs long or more, in particular 40 to 300 (40, 50, 70, 100, 120, 150, 175, 200, 225, 250, 275 or 300) base pairs long or more. In some embodiments, the target sequence comprises less than 40 base pairs, in particular 30, 20 base pairs or less.

In some embodiments the recombinant cell further comprises a coding sequence that expresses a recombinant recombineering system. In some embodiments the recombinant cell further comprises the expression product of the mutagenized sequence.

In some embodiments the recombinant cell is a eukaryotic cell. In some embodiments the recombinant cell is a prokaryotic cell. In some embodiments the prokaryotic cell is a bacterial cell. In some embodiments the bacterial cell expresses mutL* (dominant negative mutL). In some embodiments the bacterial cell is an E. coli cell. In some embodiments the E. coli is deleted for the two exonucleases SbcB and RecJ to increase recombineering efficiency.

The invention further provides a kit for generating targeted nucleic acid diversity, comprising one or a plurality of recombinant expression plasmids together comprising coding sequences for the recombinant Cas protein, the recombinant error-prone reverse transcriptase (RT) and for the at least one recombinant spacer RNA comprising a target sequence, and coding sequence that expresses a recombinant recombineering system according to the present disclosure; in particular comprising coding sequences for the recombinant Cas protein, the recombinant DGR RT, recombinant DGR Avd, recombinant DGR spacer RNA(s) and recombinant SSAP mediating oligonucleotide recombineering according to the present disclosure; preferably further comprising coding sequence(s) for the recombinant CRISPR guide RNA(s); preferably comprising the plasmid pRL014 having the sequence SEQ ID NO: 17 and a plasmid comprising a coding sequence for the recombinant Cas protein, preferably comprising coding sequences for the recombinant Cas protein and recombinant CRISPR guide RNA(s).

### DETAILED DESCRIPTION

This disclosure reports the directed evolution of Cas proteins using the first targeted diversity generation system based on the use of a mutagenic reverse transcriptase from a natural Diversity Generating Retroelements (DGRs) system. An embodiment of the system is exemplified herein in the model laboratory organism E. coli, enabling various applications in directed evolution setups of Cas proteins. Based on this initial embodiment, several other embodiments are disclosed. The exemplified embodiment is in no way limiting.

In certain embodiments the system of the invention comprises any combination of one or more of the following features:
1) in vivo mutagenesis, so that the library of sequence variants does not need to be created in vitro, through expensive oligonucleotide library synthesis, for example, and it does not need to be transformed into the bacterium, a technical bottleneck for flexibility of the technique. In certain embodiments, in vivo mutagenesis may be coupled to a selection framework to enable continuous evolution, which may be a powerful combination for directed evolution.
2) mutagenesis of the target sequence in its native genomic context, which may enable transferability of the system to various targets of choice, and transferability of the system to different bacterial taxa.
3) tailored mutagenesis for exploring protein sequence diversity, by incorporating an error-prone reverse-transcriptase from a DGR system into the system, the ability to selectively mutate adenines (in the DGR spacer (TR)) into any nucleotides, allows dense mutagenesis over small protein domain-sized windows while maintaining a usefully low rate of nonsense mutations.

### Method

In a first aspect, the invention provides methods of generating targeted nucleic acid diversity in a Cas gene comprising expressing in a recombinant cell comprising a Cas gene, in particular a recombinant Cas gene, a recombinant error-prone reverse transcriptase (RT) and recombinant spacer RNA comprising a target sequence for mutagenesis of a DNA sequence in the Cas gene; making a mutagenized cDNA polynucleotide homologous to the DNA sequence in the recombinant cell; expressing a recombineering system in the recombinant cell; and recombining the mutagenized cDNA with the homologous DNA sequence of the Cas gene in the recombinant cell.

The methods of the invention may use any Cas gene. As used herein, a “Cas gene” refers to a gene encoding a Cas endonuclease. As used herein, a “Cas protein” refers to a Cas endonuclease. The Cas endonucleases (or Cas proteins) are site-specific DNA or RNA endonucleases that are directed by small CRISPR RNA guides (gRNA) to target and subsequently cleave complementary DNA or RNA sequences. CRISPR system involves two components, Cas protein and guide RNA (CRISPR guide RNA). Cas9 protein comprises two active cutting sites namely HNH nuclease domain and RuvC-like nuclease domain; Cas12a and Cas12f only have one RuvC domain; Cas13 has 2 HEPN domains. The Cas gene according to the invention is in particular a recombinant Cas gene encoding a recombinant Cas protein, i.e., comprising a coding sequence for a Cas protein. The Cas gene may have the sequence of a natural (wild-type) Cas gene or a variant thereof. A variant (protein or gene) includes at least one nucleotide or amino acid modification (insertion, substitution, deletion) as compared to wild-type. The Cas gene or Cas protein may be any Cas gene or Cas protein known in the art including homologs, orthologs thereof, or modified versions thereof. The Cas gene or Cas protein is advantageously derived from class 2 CRISPR/Cas systems which require only one effector protein to target recognition sequences and degrade nucleic acid. Class 2 systems include type II (Cas9), type V (Cas12) and type VI (Cas 13) and have been identified from several bacteria genera, including Streptococcus, Staphylococcus, Legionella, Neisseria, Francisella, Campylobacter, Prevotella and many others. All CRISPR/Cas type II systems (CRISPR/Cas9) require another sequence known as protospacer adjacent motif (PAM), present adjacent to the target site. In some embodiments, the Cas gene is Cas9 gene, Cas12 gene or Cas13 gene. Cas12 includes, Cas12a, Cas12b, Cas12f and others. Cas12a gene is in particular chosen from Acidaminococccus or Lachnospiracae Cas12a genes and homologs, orthologs, or modified versions thereof. In some particular embodiments, the Cas gene is Cas9 gene. Prototype Cas9 is from Streptococcuspyogenes (SpCas9). Exemplary Cas9 from Streptococcus pyogenes corresponds to the gene ID 69900934 having the 4107 bp sequence GenBank/NCBI accession number NZ_LS483338.1 (as accessed on 15 Jan. 2022) which codes for a 1368 amino acid Cas protein having the sequence GenBank/NCBI accession number WP_038431314.1 (as accessed on 28 Feb. 2022). Using SpCas9 as a reference sequence, the PAM interacting domain (PID) is predicted to correspond to positions 1096 to 1358. In some preferred embodiments, the Cas9 gene is chosen from Streptococcuspyogenes, Staphylococcus aureus, Streptococcus thermophilus or Streptococcus canis Cas9 genes, and homologs, orthologs thereof, or modified versions thereof.

In some embodiments, the Cas gene encodes an enzymatically active endonuclease, i.e., that binds to and cleaves the Cas-target sequence in DNA or RNA. The enzymatically active endonuclease may induce either a double-stranded break or a single-stranded break in DNA. In some embodiments, the Cas gene encodes an enzymatically inactive endonuclease (dead Cas or dCas), that binds to its target sequence in DNA but does not cleave the target sequence. In some embodiments, the homologous sequence of the Cas gene that is targeted for mutagenesis by the DGRec system (mutagenesis target) is in the PAM interacting domain (PID). In some embodiments, the Cas gene comprises at least one nonsense mutation (stop codon) in the mutagenesis target. The at least one nonsense mutation (stop codon) may be introduced in an initial Cas gene encoding an enzymatically active or inactive endonuclease. The presence of the nonsense mutation will generate a non-functional Cas protein in the recombinant cell. This allows the selection of functional Cas protein variants generated by targeted nucleic acid diversity using the DGRec system according to the method of the invention. In some particular embodiments, the nonsense mutation(s) are in the PAM interacting domain (PID), preferably at or in close proximity to one or more of positions L1111, R1122, K1123, D1135, Y1141, L1144, S1216, G1218, E1219, L1220, A1322, K1334, R1335, and T1337, said positions being indicated by alignment with SpCas9 reference sequence. In close proximity means that the stop codon and the disclosed position can be targeted by the same DGR. Both positions are preferably within the same DGR spacer and 10 nt from the edge of the DGR spacer. In some particular embodiments, the Cas gene encodes an enzymatically inactive endonuclease (dead Cas or dCas) and further comprises at least one nonsense mutation (stop codon) in the mutagenesis target, in particular the PAM interacting domain (PID), preferably at one or more of the above disclosed positions. In some embodiments, the recombinant cell further comprises at least one CRISPR guide RNA. The CRISPR guide RNA directs the Cas protein encoded by the Cas gene to target a complementary DNA sequence of interest (Cas-target sequence).

The diversity generation system according to the present invention has a modular arrangement as the different parts of both the diversity generating module and the recombineering module are independent, as shown in the examples. Therefore, they can a priori be arranged in several ways to function. The different parts of the diversity generating module can thus be placed all on the same recombinant vector(s) such as plasmids, split in different vectors, placed inside the host cell chromosome, or placed on vectors(s) such as plasmids and inside the host cell chromosome. Similarly, the recombineering module can be vector-borne such as plasmid-borne, inside the host genome, or mixed. Furthermore, the results obtained in the model laboratory organism E. coli presented in the examples show that the diversity generating module does not require the host cell environment to function and can thus be used in various host cells. In some embodiments, the Cas gene is inserted in a vector, in particular a plasmid. The Cas gene may be on the same vector as some components of the DGRec system or on a different vector. In some particular embodiments, the vector further comprises at least one CRISPR guide RNA. In some particular embodiments, the Cas gene is on a different vector as the components of the DGR system and preferably further comprises at least one CRISPR guide RNA.

The recombinant error-prone reverse transcriptase (RT) and recombinant spacer RNA form a functional enzymatic complex able to use the spacer RNA comprising the target sequence as a specific template for mutagenic reverse transcription. The target sequence called template region (TR) corresponds to the editable part of the reverse transcribed region of the spacer RNA. The recombinant error-prone reverse transcriptase (RT) uses the spacer RNA comprising the target sequence as RNA template to carry out the polymerization of the mutagenized cDNA polynucleotide homologous to a DNA sequence in the recombinant cell.

The method according to the invention may use any error-prone reverse transcriptase (RT) capable of forming a functional enzymatic complex with the spacer RNA that is able to use the spacer RNA comprising the target sequence as a specific template for mutagenic reverse transcription in the host cell. The recombinant error-prone reverse transcriptase (RT) may comprise the sequence of a natural error-prone reverse transcriptase (RT), or a variant or fragment thereof, that is functional in the host cell. Alternatively, the recombinant error-prone reverse transcriptase (RT) may be an engineered error-prone reverse transcriptase (RT), for example engineered from a non-mutagenic reverse-transcriptase. Most canonical RT have a conserved motif QGXXXSP (SEQ ID NO: 1) which directly interacts with the RT template. In all DGR RT, this motif is modified to I/LGXXXSQ (SEQ ID NO: 2), that has been linked to their selective infidelity at adenine positions (Handa et al., [25]). Non-limiting examples of error-prone reverse transcriptase (RT) that may be used to carry out the method of the invention include: reverse transcriptase from Diversity Generating retroelements and engineered error-prone reverse transcriptase. In some embodiments, the recombinant error-prone reverse transcriptase (RT) comprises the motif QGXXXSP or I/LGXXXSQ. In some particular embodiments, the recombinant error-prone reverse transcriptase (RT) is engineered from a non-mutagenic reverse-transcriptase by replacement of the QGXXXSP motif (canonical RT motif) with the I/LGXXXSQ motif (canonical DGR RT motif).

In some embodiments, the recombinant error-prone reverse transcriptase and spacer RNA are from Diversity-generating retroelement (DGR). Diversity-generating retroelements (DGRs) are a unique family of retroelements that generate sequence diversity of DNA to benefit their hosts by introducing sequence variations and accelerating the evolution of target proteins. They exist widely at least in bacteria, archae, phage and plasmid. The prototype DGR was found in Bordetella phage (BPP-1) and two other DGRs have been characterized in Legionella pneumophila and Treponema denticola (Wu et al., [3]). There are more than a thousand distinct DGR systems that have been predicted bioinformatically (Paul et al., [2]). The examples of the present application show that three components of the DGR are necessary and sufficient to assemble a functional diversity generation system, the reverse transcriptase major subunit RT, the accessory subunit such as Avd, and the spacer RNA (see FIG. 1). These three components have been identified in the putative DGR systems indicating that various known DGR systems can be used in the method according to the invention. Alternative DGR systems from these various native DGR systems could be screened for activity, using methods that are well-known in the art such as the mCherry fluorescence assay herein disclosed or similar screening systems that may be easily derived from this system. Known methods may be adapted to design a cell-free expression system (Garamella et al., [27]).

The two DGR proteins necessary to generate sequence diversity of DNA, the reverse transcriptase major subunit (RT) and accessory subunit such as Avd, together form the active mutagenic reverse transcriptase complex. The DGR spacer RNA is capable of recruiting the mutagenic reverse transcriptase complex and priming cDNA synthesis upstream of a modifiable part called TR (template region) (Handa et al., [6]). The spacer RNA (secondary and possibly tertiary) structure formation is important in this process in natural DGR systems (Handa et al., [6]). The spacer RNA sequence comprises a modifiable part called TR (template region) corresponding to the editable part of the reverse transcribed region, flanked by 5′ and 3′ conserved regions, as illustrated in FIG. 4 for BPP-1 DGR spacer RNA. The TR may correspond to all or part of the reverse transcribed region. The template region (TR) which can be modified within a flexible size range corresponds to the target sequence in recombinant DGR spacer RNAs according to the present invention. The 3′ region comprises a self-priming hairpin containing two self-annealing segments that are necessary to prime the mutagenic RT complex. The starting point of the cDNA polymerization corresponds to the A56 ribonucleotide in BPP-1 DGR spacer RNA and is about 4 nucleotides upstream of the TR region in BP-1 DGR spacer RNA. This ribonucleotide is covalently bound to the cDNA to form a DNA/RNA hybrid comprising a short RNA tail at the 5′ end of the cDNA (FIG. 4). Using BBP-1 DGR spacer RNA coding sequence (DNA sequence of SEQ ID NO: 3) as reference sequence, the 5′conserved region is from positions 1 to 20; the template region (TR) from positions 21 to 136; and the 3′conserved region is from position 137 to 158. The indicated positions are determined by alignment with BPP-1 DGR spacer RNA reference sequence. One skilled in the art can easily determine the sequence of another DGR spacer RNA and positions of the 5′, TR and 3′ regions in said DGR spacer RNA, by alignment with the reference sequence using appropriate software available in the art such as BLAST, CLUSTALW and others. In recombinant DGR spacer RNAs, the template region is replaced with a target sequence of interest. The target sequence thus corresponds to all or a subset of the reverse transcribed region of the DGR spacer RNA (the template region), where it is operably linked to the DGR spacer RNA, and in particular to its cDNA polymerization starting point. In recombinant DGR spacer RNAs, the template region sequence of the DGR spacer RNA is deleted and replaced with a target sequence of interest, usually the target sequence replaces all the template region sequence. The activity of a recombinant DGR RNA may be assessed using methods known by the skilled person such as the mCherry fluorescence assay herein disclosed.

DGR RTs are error-prone reverse transcriptases which range in size from about 300 to about 500 amino acids and contain RT motifs 1-7, which correspond to the palm and finger domain of other polymerases. DGR RT's contain motif 2a, located between motifs 2 and 3, which is found among group II introns, non-LTR retroelements and retrons, but not among other RTs such as retroviral or telomerase RTs (review in Wu et al., [3]). DGR RTs may be chosen from the RVT_1 pfam family (PF0078) that carry the I/LGXXXXSQ motif in place of the prototypical QGXXXSP motif (positions 133-140 of the pfam HMM logo).

The accessory gene avd encodes an essential 128 aa protein that has a barrel structure and forms a homopentamer. The avd genes are very poorly conserved but of similar length. Avd protein binds the reverse transcriptase (RT), and association between these two proteins is required for mutagenesis. Avd is highly basic and binds to both DNA and RNA in vitro, but without detectable sequence specificity. Consistent with a role in nucleic acid binding, Avd is highly basic with the average of calculated pI's being 9.5±0.7 (review in Wu et al., [3]).

In Bordetella bacteriophage BPP-1, the DGR reverse transcriptase is encoded by the brt gene (Gene ID: 2717203) which corresponds to the 987 bp sequence from the complement of positions 1756 to 2742 of BPP-1 complete genome sequence (GenBank/NCBI accession number NC_005357.1 as accessed on 20 Dec. 2020). BPP-1 DGR reverse transcriptase (bRT) has the 328 amino acid sequence GenBank/NCBI accession number NP_958675.1 as accessed on 20 Dec. 2020 or UniProtKB accession number Q775D8 as accessed on 2 Dec. 2020 (SEQ ID NO: 4). BPP-1 DGR accessory protein Avd is encoded by the avd gene (Gene ID: 2717200) which corresponds to the 387 bp sequence from the complement of positions 3021 to 3407 of BPP-1 complete genome sequence (GenBank/NCBI accession number NC_005357.1 as accessed on 20 Dec. 2020). BPP-1 Avd (bAvd) protein has the 128 amino acid sequence GenBank/NCBI accession number NP_958676.1 as accessed on 20 Dec. 2020 (SEQ ID NO: 5). One skilled in the art can easily determine the sequence of another DGR reverse transcriptase and accessory protein such as Avd, by alignment with the reference sequence using appropriate software available in the art such as BLAST, CLUSTALW and others.

The recombinant DGR RT, the recombinant DGR accessory protein such as Avd, and recombinant DGR spacer RNA according to the invention may be selected from the DGR of Bordetella bacteriophage BPP-1, Legionella pneumophila, Treponema denticola or their functional orthologs (Paul et al., [2]; Wu et al., [3]) and functional variants or fragments thereof.

By functional orthologs of Bordetella BPP-1, Legionella or Trepanoma DGR is intended ortholog RT, accessory protein(s) such as Avd or others, and spacer RNA encoded by ortholog genes and that form a functional enzymatic complex able to use the spacer RNA as a specific template for mutagenic reverse transcription.

Mutagenic reverse transcription on spacer RNA template may be assessed in assays that are well-known by the skilled person such as the mCherry fluorescence disclosed in the examples. Briefly, a reporter E. coli strain (sRL002) comprising a mCherry gene expression cassette integrated in its genome is co-transformed with a plasmid for expression of the tested DGR RT and Avd proteins derived from pRL014 and a plasmid for expression of the tested DGR spacer RNA engineered to target mCherry gene and oligonucleotide recombineering enzyme CspRecT derived from pAM011. The DGR RT to be assayed is cloned under the control of the PhlF promoter inducible by DAPG, replacing bRT in pRL014. The Avd protein to be assayed is cloned under the control of the J23119 promoter, replacing bAVd in pRL014. The DGR spacer RNA to be assayed is engineered to target mCherry gene by replacing its TR region with TR_AM011 (SEQ ID NO: 19; FIG. 3). The engineered DGR is then cloned under the control of the J23119 promoter, replacing the spacer RNA in pAM011. sRL002 co-transformed with control plasmid encoding inactivated RT are used as negative control. 48h post-induction of protein expression, the activity of the DGR system (RT, Avd, Spacer RNA) is measured by the percentage of non-fluorescent colonies. Non-fluorescent colonies are not detected in the negative control showing the specificity of the assay.

The use of functional orthologs of the previously characterized DGRs might improve the DGRec efficiency in E. coli, and the variety of DGRec variants will render the technology more amenable to transfer in other bacterial species or to be adapted in eukaryotic organisms.

In some particular embodiments, the recombinant DGR RT, the recombinant DGR Avd, and recombinant DGR spacer RNA are from bacteria, archae, phage or plasmid selected from the group consisting of: Legionella or Trepanoma chromosomal DGR, Bacteroides Hankyphage DGR or Bordetella bacteriophage BPP-1; preferably from the Bordetella bacteriophage BPP-1.

The recombinant DGR RT, the recombinant DGR accessory protein such as Avd, and recombinant DGR spacer RNA according to the invention may be from the same DGR (e.g, the same organism) or from different DGRs (e.g. from different organisms). In some embodiments, the recombinant DGR accessory protein such as Avd, and recombinant DGR spacer RNA according to the invention are from the same DGR; preferably from the Bordetella bacteriophage BPP-1.

In some particular embodiments, the recombinant DGR RT comprises the canonical motif I/LGXXXSQ.

In some particular embodiments, the recombinant DGR RT comprises a sequence having at least 70%, 75%, 80%, 85%, 90%, 95%, 96%, 97%, 98%, 99% identity, or 100% identity with SEQ ID NO: 4 preferably the sequence comprises the canonical motif I/LGXXXSQ.

In some particular embodiments, the recombinant DGR accessory subunit, in particular recombinant DGR Avd, comprises a sequence having at least 70%, 75%, 80%, 85%, 90%, 95%, 96%, 97%, 98%, 99% identity, or 100% identity with SEQ ID NO:5.

As used herein, the term “variant” refers to a polypeptide comprising an amino acid sequence having at least 70% sequence identity with the native sequence. The term “variant” refers to a functional variant having the activity of the native sequence. Functional fragments of the native sequence or variant thereof are also encompassed by the present disclosure. The activity of a variant or fragment may be assessed using methods well-known by the skilled person such as those disclosed herein. In particular, functional RT variant, accessory protein(s) variant and spacer RNA variant form a functional enzymatic complex able to use the spacer RNA as a specific template for mutagenic reverse transcription.

The percent amino acid sequence or nucleotide sequence identity is defined as the percent of amino acid residues or nucleotides in a Compared Sequence that are identical to the Reference Sequence after aligning the sequences and introducing gaps if necessary, to achieve the maximum sequence identity and not considering any conservative substitutions for amino acid sequences as part of the sequence identity. Alignment for purposes of determining percent amino acid sequence identity can be achieved in various ways known to a person of skill in the art, for instance using publicly available computer software such as the GCG (Genetics Computer Group, Program Manual for the GCG Package, Version 7, Madison, Wisconsin) pileup program, or any of sequence comparison algorithms such as BLAST (Altschul et al., J. Mol. Biol., 1990, 215, 403-), FASTA or CLUSTALW. When using such software, the default parameters, are preferably used.

In some embodiments, the term “variant” refers to a polypeptide having an amino acid sequence that differs from a native sequence by the substitution, insertion and/or deletion of less than 30, 25, 20, 15, 10 or 5 amino acids. In a preferred embodiment, the variant differs from the native sequence by one or more conservative substitutions, preferably by less than 15, 10 or 5 conservative substitutions. Examples of conservative substitutions are within the groups of basic amino acids (arginine, lysine and histidine), acidic amino acids (glutamic acid and aspartic acid), polar amino acids (glutamine and asparagine), hydrophobic amino acids (methionine, leucine, isoleucine and valine), aromatic amino acids (phenylalanine, tryptophan and tyrosine), and small amino acids (glycine, alanine, serine and threonine).

In some embodiments, the recombinant error-prone RT is an engineered recombinant error-prone RT derived from a non-mutagenic reverse-transcriptase such as the Ec86 retron reverse transcriptase. In some preferred embodiment, the recombinant error-prone RT is a mutant Ec86 retron reverse transcriptase substituted to carry the motif I/LGXXXSQ replacing the prototypical QGXXXSP motif. This conserved motif is present in DGR Reverse Transcriptase and has been linked to their selective infidelity at adenine positions (Handa et al., [25]).

In some embodiments, the recombinant error-prone RT, in particular recombinant DGR RT, has adenine mutagenesis activity. This means that the mutagenesis will happen randomly at adenine positions. An approximation of 25% chances of incorporation of any nucleotide at adenine (A) positions gives a convenient model to predict the variants and library size. However, the actual RT errors can deviate from this rule [25]: they can vary from one A position to another, and errors can also happen at much lower frequencies at non-A nucleotides.

In some particular embodiments, the recombinant error-prone RT, in particular recombinant DGR RT, comprises a mutation that modulates (increases or decreases) its error rate. In some preferred embodiments, the recombinant DGR RT comprises a mutation that decreases its error rate at adenine position selected from the group consisting of: R74A and 1181N, the positions being indicated by alignment with SEQ ID NO: 4. Such variants are disclosed in Handa et al. [25]. In some more preferred embodiments, the recombinant DGR RT comprising the R74A mutation is encoded by the sequence SEQ ID NO: 9; and/or the recombinant DGR RT comprising the 1181 mutation is encoded by the sequence SEQ ID NO: 10.

The method according to the invention uses a recombineering system which is different from the natural DGR recombination system (“retrohoming”). The recombineering system is a recombinant system comprising or consisting of a recombinant recombineering enzyme. The method according to the invention may use any single-stranded oligonucleotide-based recombineering methods that are well-known in the art (Wannier et al., 2021 [26]). Recombineering is in vivo homologous recombination-mediated genetic engineering. This process allows the incorporation of genetic DNA alterations to any DNA sequence, either in the chromosome or cloned onto a vector that replicates in E. coli or other recombineering-proficient cell. Recombineering with single-strand DNA can be used to create single or multiple clustered point mutations, small or large deletions and small insertions. Oligonucleotide recombineering rely on the annealing of synthetic single-stranded oligonucleotides to the lagging strands at open replication forks onto targeted DNA loci (Csörgö et al., [10]). Oligonucleotide recombineering requires specific single-stranded DNA annealing proteins (SSAP) such as those derived from the Red/ET recombination system, a powerful homologous recombination system based on the Red operon of lambda phage or RecE/RecT from Rec phage. Single-stranded DNA annealing proteins include in particular, the phage lambda's Red Beta protein for E. coli, the functional homolog RecT and variants thereof such as PapRecT and CspRecT, as well as similar systems (Wannier et al., PNAS, 2020, 117, 13689-13698 [40]). CspRecT protein has the 270 amino acid sequence GenBank/NCBI accession number WP_00672078.2 as accessed on 1 Jun. 2019 (SEQ ID NO: 6).

In some preferred embodiments, the cell, error-prone RT such as DGR RT, spacer RNA such as DGR spacer RNA and recombineering system are not from the same organism, which means that they are never found together in nature. The error-prone RT such as DGR RT, and spacer RNA such as DGR spacer RNA may be from the same organism or a different organism; preferably the DGR RT and DGR spacer RNA are from the same organism. In some preferred embodiments, the recombineering system is heterologous to the error-prone RT and spacer RNA, which means that the recombineering system originates from a different organism than the error-prone RT and spacer RNA. In some preferred embodiments, the cell is heterologous to the error-prone RT and spacer RNA, which means that the cell originates from a different organism than the error-prone RT and spacer RNA. In some preferred embodiments, the recombineering system is also heterologous to the cell and the error-prone RT and spacer, which means that the cell originates from a different organism than the error-prone RT and spacer RNA and also the recombineering system.

In some embodiments of the method, the recombineering system or enzyme is a recombinant single-stranded annealing protein (SSAP) mediating oligonucleotide recombineering selected from the group consisting of: the phage lambda's Red Beta protein, the functional homolog RecT or RecT and variants thereof such as PapRecT and CspRecT; preferably CspRecT.

In some embodiments, the recombinant single-stranded annealing protein (SSAP) mediating oligonucleotide recombineering comprises a sequence having at least 70%, 75%, 80%, 85%, 90%, 95%, 96%, 97%, 98%, 99% identity, or 100% identity with SEQ ID NO: 6.

The error-prone RT such as DGR RT uses the spacer RNA comprising the target sequence as template to generate a mutagenized target sequence in the form of a cDNA polynucleotide homologous to a DNA sequence of a Cas gene, in particular a recombinant Cas gene, in the recombinant cell. The recombineering system that is expressed in the recombinant cell will then recombine the mutagenized cDNA polynucleotide with the homologous DNA sequence of the (recombinant) Cas gene in the recombinant cell to generate a DNA sequence variant comprising the mutagenized target sequence (mutagenized DNA sequence). The homologous DNA sequence in the recombinant cell is named mutagenesis target, mutagenesis window, variable region, target gene region, targeted region or targeted sequence. The target sequence in the spacer RNA defines the mutagenesis window on the genome or recombinant vector in the recombinant cell. The target sequence does not have to be identical to the mutagenesis window but can have several mismatches compared to the targeted sequence. As explained just below, the target sequence may comprise a recoded version or mutated version of the mutagenesis window to allow more flexibility in the mutagenesis of the targeted sequence. The reverse transcribed region must contain homologies to the targeted region on the genome or recombinant vector that will enable recombination of the cDNA. Homology to the targeted region can occur throughout the cDNA, or only in part of the cDNA. Several discontiguous homology regions might exist in the cDNA. The non-homologous region present in between two homology regions will then replace the corresponding sequence in the targeted region after recombination.

The target sequence comprised in the recombinant spacer RNA may be any nucleic acid sequence of interest for mutagenesis or diversification of a Cas gene and derived Cas encoded protein using the method of the invention. The target sequence and mutagenized target sequence are usually from 20 to 500 bases/base pairs. In some embodiments of the methods the target sequence and/or mutagenized target sequence comprises 70 base pairs. In some embodiments of the method the target sequence and/or mutagenized target sequence is from 50 to 120 base pairs long. In some embodiments of the methods the target sequence and/or mutagenized target sequence is from 70 to 100 base pairs long. In some embodiments of the method the target sequence and/or mutagenized target sequence is from 40 to 200 (40, 50, 70, 100, 120, 150, 175, 200) base pairs or more, in particular 40 to 300 (40, 50, 70, 100, 120, 150, 175, 200, 225, 250, 275 or 300) base pairs long or more. In some embodiments of the method the target sequence and/or mutagenized target sequence comprises less than 40 base pairs, in particular 30, 20 base pairs or less. In some embodiments, the target sequence targets the PAM interacting domain (PID). This means that the homologous DNA sequence (mutagenesis target or targeted sequence) is in the PAM interacting domain (PID).

The mutagenized target sequence and mutagenesis target share a sufficient amount of sequence identity to allow homologous recombination to occur between them. Minimum length of sequence homology required for in vivo recombination are well-known in the art (see in particular Wannier et al., 2021 [26], Thomason, Curr. Protocol.Mol. Biol., 2014, 106:1.16.1-39).). Homology to the targeted region can occur throughout the cDNA, or only in part of the cDNA. Several discontiguous homology regions might exist in the cDNA. The non-homologous region present in between two homology regions will then replace the corresponding sequence in the targeted region on the genome or recombinant vector after recombination.

In some embodiments, the adenine content (percentage) and/or position(s) in the target sequence (TR region) and/or homologous DNA sequence (mutagenesis target or targeted sequence) in the recombinant cell is modified to modulate recombination frequency or control sequence diversity. In some preferred embodiments, the target sequence is modified to decrease the adenine content. In some preferred embodiments, the homologous DNA sequence (mutagenesis target or targeted sequence) is modified to decrease the adenine content. The adenine content may be decreased by lowering the adenine content on the top strand or the thymine content on the bottom strand of the homologous DNA sequence.

In some embodiments, the homologous DNA sequence (mutagenesis target or targeted sequence) comprises at least one nonsense mutation. The presence of the nonsense mutation will generate a non-functional Cas protein in the recombinant cell. This allows the selection of functional Cas protein variants generated by targeted nucleic acid diversity using the DGRec system according to the method of the invention.

In some preferred embodiments, the homologous DNA sequence (mutagenesis target or targeted sequence) is modified to decrease the adenine content and further comprises at least one nonsense mutation. The homologous sequence which is modified is in particular in the PAM interacting domain (PID).

Recombineering efficiency decreases with the number of mismatches between the ssDNA and the targeted sequence. As a consequence of these constraints, it may be desirable to maximize the identity between the cDNA produced by the RT and the targeted sequence. This can be done by minimizing the number of adenines in the target sequence (TR region). It is also possible to recode the target gene region in order to minimize the number of adenines in the targeted sequence, thereby enabling to also reduce the number of adenines in the TR region. As an example, a target sequence (TR region) containing 16% of adenines has been used with success. Importantly, recoding the target gene region also offers the benefit of giving more flexibility in the design of the TR to choose the positions that will be mutagenized by strategically selecting codons containing more adenines at those positions. Recoding can also be used to reduce the probability that the library contains variants with stop codons. Finally, the TR design provides another layer of flexibility and control in the mutagenesis profile, when adding mismatches between the TR sequence and its target sequence. A TR mismatch can ‘force’ the incorporation of a given nucleotide other than an adenine (thus forcing a given amino acid in a library of protein variants), or the mismatch can ‘force’ higher variability at this position by the addition of adenines.

In some embodiments, the target sequence orientation is designed to optimize recombination efficiency. Maximum recombineering efficiency is achieved when oligos anneal to the lagging strand during DNA replication, which can be identified for a given gene according to its position and orientation in the chromosome relative to its origin of replication and terminus (a process detailed in Wannier et al., [26]). Therefore, recombineering efficiency may be improved by designing target sequence orientation appropriately. If a doubt remains concerning the lagging strand of a genetic element (for example, phages or plasmids), it is always possible to design both TR orientations to ensure one will be annealing to the lagging strand of the targeted sequence.

In some embodiments of the method, the recombination frequency is at least 0.01%. In some embodiments of the methods the recombination frequency is 0.1%. In some embodiments of the method, the recombination frequency is at least 1%; preferably 3% or more; more preferably 10% or more.

In some embodiments of the method, the recombinant cell comprises at least two spacer RNAs comprising a target sequence; in particular at least two DGR spacer RNAs comprising a target sequence. In some preferred embodiments, the multiple spacer RNAs target the same gene in the recombinant cell.

As used herein, “expressing” a recombinant protein or RNA in a recombinant cell (host cell) refers to the process resulting from the introduction of the recombinant protein or RNA in the cell; the introduction of a nucleic acid molecule encoding said protein or RNA in expressible form or a combination thereof.

In some embodiments of the method, the recombinant cell comprises coding sequences for the recombinant Cas protein, the recombinant error-prone reverse transcriptase (RT), the recombinant spacer RNA(s) comprising a target sequence, and the recombineering system; in particular the recombinant cell comprises coding sequences for the recombinant Cas protein, the recombinant DGR reverse transcriptase major subunit (RT), the recombinant DGR accessory subunit (Avd), the recombinant DGR spacer RNA(s) comprising a target sequence and the recombineering system. In some preferred embodiments of the method, the recombinant cell further comprises coding sequence(s) for the CRISPR guide RNA(s).

In some particular embodiments, at least one of the coding sequences for the recombinant error-prone reverse transcriptase (RT), in particular the recombinant DGR reverse transcriptase major subunit (RT), the recombinant DGR accessory subunit (Avd) and the recombineering system, such as the recombinant SSAP, in particular CspRecT, are codon optimized for expression in the host cell. Codon optimization is used to improve protein expression level in living organism by increasing translational efficiency of target gene. Appropriate methods and softwares for codon optimization in the desired host are well-known in the art and publically available (see for example the GeneOptimizer software suite in Raab et al., Systems and Synthetic Biology, 2010, 4, (3), 215-225). Codon optimization of a nucleic acid construct sequence relates to the (protein) coding sequences but not to the other (non-coding) sequences of the nucleic acid construct.

In some preferred embodiments, the coding sequence according to the present disclosure is codon optimized for expression in E. coli.

In some particular embodiments, the coding sequence for the recombinant DGR reverse transcriptase major subunit (RT) has at least 80%, 85%, 90%, 95%, 96%, 97%, 98%, 99% identity, or 100% identity with any one of SEQ ID NO: 7, 9 or 10. In some particular embodiments, the coding sequence for the recombinant DGR accessory subunit (Avd) has at least 80%, 85%, 90%, 95%, 96%, 97%, 98%, 99% identity, or 100% identity with SEQ ID NO: 11. In some particular embodiments, the coding sequence for the recombinant CspRecT has at least 80%, 85%, 90%, 95%, 96%, 97%, 98%, 99% identity, or 100% identity with SEQ ID NO: 14.

The coding sequences according to the present disclosure are expressible in the recombinant cell (host cell or host). In some embodiments, the coding sequence is operably linked to appropriate regulatory sequence(s) for its expression in the recombinant cell (host cell). Such sequences which are well-known in the art include in particular a promoter, and further regulatory sequences capable of further controlling the expression of a transgene, such as without limitation, enhancer or activator, terminator, kozak sequence and intron (in eukaryote), ribosome-binding site (RBS) (in prokaryote).

In some particular embodiments, the coding sequence is operably linked to a promoter. The promoter may be a ubiquitous, constitutive or inducible promoter that is functional in the recombinant cell. Non-limiting examples of promoters suitable for expression in E. coli include: inducible promoters such as PhlF (inducible by DAPG), Pm (inducible by XylS), Ptet (inducible by Atc), Pbad (inducible by arabinose) and constitutive promoters such as J23119 (strong constitutive promoter), Pr (strong constitutive promoter from the Lambda phage). In some preferred embodiments, the coding sequence for the recombinant DGR RT is operatively linked to an inducible promoter, in particular PhlF promoter comprising the sequence SEQ ID NO: 13. In some preferred embodiments the coding sequences for the recombinant DGR Avd and recombinant DGR spacer RNA(s) are operatively linked to constitutive promoter(s). Polycistronic expression systems that are well-known in the art may be used to drive the expression of several DGR spacer RNAs from the same promoter. In some preferred embodiments, the coding sequence for the recombinant SSAP, in particular CspRecT is operably linked to an inducible promoter, in particular Pm promoter/XylS activator. In some preferred embodiments, the coding sequence is further operably linked to a ribosome binding site. In some particular embodiments, the coding sequence(s) for the recombinant Cas protein, and optional CRISPR guide RNA(s) are under the control of an inducible promoter.

The nucleic acid comprising the coding sequence according to the present disclosure may be recombinant, synthetic or semi-synthetic nucleic acid which is expressible in the recombinant cell. The nucleic acid may be DNA, RNA, or mixed molecule, which may further be modified and/or included in any suitable expression vector. As used herein, the terms “vector” and “expression vector” mean the vehicle by which a DNA or RNA sequence (e.g. a foreign gene) can be introduced and maintained into a host cell, so as to transform the host and promote expression (e.g. transcription and translation) of the introduced sequence. The recombinant vector can be a vector for eukaryotic or prokaryotic expression, such as a plasmid, a phage for bacterium introduction, a YAC able to transform yeast, a transposon, a mini-circle, a viral vector, or any other expression vector. The vector may be a replicating vector such as a replicating plasmid. The replicating vector such as replicating plasmid may be a low-copy or high-copy number vector or plasmid.

In some embodiments, the coding sequence is DNA that is integrated into the recombinant cell genome or inserted in an expression vector. In some particular embodiments, the expression vector is a prokaryote expression vector such as plasmid, phage, or transposon.

The diversity generation system has a modular arrangement as the different parts of both the diversity generating module and the recombineering module are independent, as shown in the examples. The different parts of the diversity generating and recombineering modules can thus be placed all on the same recombinant vector(s) such as plasmids, split in different vectors, placed inside the host cell chromosome, or placed on vectors(s) such as plasmids and inside the host cell chromosome. Similarly, the recombineering module can be vector-borne such as plasmid-borne, encoded within the host genome, or mixed.

In some embodiments, the recombinant DGR RT, recombinant DGR Avd, and recombinant DGR spacer RNA(s) are all expressed from one or a plurality of recombinant plasmids together comprising coding sequences for the recombinant DGR RT, recombinant DGR Avd, and recombinant DGR spacer RNA(s) (DGRec system plasmid(s)). In some embodiments, the coding sequence for the recombinant recombineering system, in particular recombinant single-stranded annealing protein (SSAP) mediating oligonucleotide recombineering, more particularly CspRecT is on a plasmid. In some particular embodiments, the recombinant DGR RT, recombinant DGR Avd, recombinant DGR spacer RNA(s), and recombinant recombineering system, in particular recombinant SSAP mediating oligonucleotide recombineering are all expressed from one or a plurality of recombinant plasmids together comprising coding sequences for the recombinant DGR RT, recombinant DGR Avd, recombinant DGR spacer RNA(s) and recombinant recombineering system, in particular recombinant SSAP mediating oligonucleotide recombineering (DGRec system plasmid(s)).

In some embodiments, the coding sequence for the recombinant Cas protein is inserted in a vector, in particular a plasmid. The vector comprising the coding sequence for the recombinant Cas protein may be on the same vector as components of the DGRec system or on a different vector. In some particular embodiments, the vector further comprises at least one CRISPR guide RNA. In some particular embodiments, the recombinant Cas gene is on a different vector as the components of the DGR system, in particular a plasmid, and preferably further comprises at least one CRISPR guide RNA.

In some embodiments, the Cas gRNA is targeted to a sequence with a non-canonical PAM sequence.

In some embodiments, the coding sequences for the recombinant DGR RT and recombinant DGR Avd are present on the same plasmid (DGRec helper plasmid). In some preferred embodiments, the plasmid is pRL014 (FIG. 2) or pRL038 (FIG. 5). pRL014 has the sequence SEQ ID NO: 17. In some embodiments, the coding sequences for the recombinant DGR RT, recombinant DGR Avd and recombinant DGR spacer RNA are present on the same plasmid (DGRec helper and targeting plasmid). In some preferred embodiments, the plasmid is derived from pRL038 (FIG. 5). pRL038 has the sequence SEQ ID NO: 20.

In some embodiments, the coding sequences for the recombinant recombineering system, in particular recombinant single-stranded annealing protein (SSAP) mediating oligonucleotide recombineering, more particularly CspRecT, and recombinant DGR spacer RNA are present on the same plasmid (DGRec targeting plasmid). In some preferred embodiments, the plasmid is derived from pRL021 (FIG. 5). pRL021 has the sequence SEQ ID NO: 18.

In some embodiments, the method comprises the step of cloning the target sequence into a plasmid comprising an engineered DGR spacer RNA comprising a cloning cassette in replacement of the template region (TR), preferably operably linked to a constitutive promoter. In some particular embodiments, the cloning cassette comprises a CcdB gene flanked by copies of the same type IIS restriction site in convergent orientation, forming non identical single stranded overhangs (sticky ends), and the target sequence is cloned into the plasmid using a synthetic double-stranded oligonucleotide comprising the target sequence flanked by copies of the same type IIS restriction site in divergent orientation, or double stranded nucleotides with 4 bases of single stranded overhangs (sticky ends) matching the recipient vector type IIS restriction sites overhangs. In some particular embodiments, a first type of plasmid further comprises the coding sequence for the recombinant recombineering system, in particular recombinant single-stranded annealing protein (SSAP) mediating oligonucleotide recombineering, more particularly CspRecT; preferably operably linked to an inducible promoter. In some preferred embodiments, the plasmid is pRL021 (FIG. 5). pRL021 has the sequence SEQ ID NO: 18. In some preferred embodiments, a second type of plasmid further comprises the coding sequence for the recombinant DGR RT and recombinant DGR Avd. In some more preferred embodiments, the plasmid is pRL038 (FIG. 5). pRL038 has the sequence SEQ ID NO: 20. In some particular embodiments, the plasmid comprises at least two cloning cassettes flanked by different type IIS restriction sites. This allows the cloning of different targets into the same plasmid. In some preferred embodiments, the method uses a first type and a second type of plasmid as defined above. This allows the mutagenesis of multiple targets simultaneously using only two plasmids for the cloning of the targets and expression of the DGRec.

There is complete freedom on the placement of the mutagenesis target, broadening the application possibilities of DGRec mutagenesis. Notably, the target can be anywhere in the host chromosome, it can be on a resident plasmid (for example, it can be added onto one of the DGRec system plasmids), the target can also be placed on a mobile genetic element to be transferred or received by the host, or it can be inside a phage genome that will serve to infect the host cell. Of note, if the target is in a high copy number within the host cell (for example, on a high-copy plasmid), not all targets will be mutagenized simultaneously. To observe the effect of a single variant of the target gene, cells will need to be grown until they segregate the plasmids carrying the distinct variants. On the other hand, a higher copy number of the target genes might favor more numerous DGR mutagenesis events, increasing the variant library size faster than with a single-copy target gene per cell. Multiple copies of a targeted sequence can also be placed in different locations inside the chromosome, or as repeated sequences inside a single gene to mutagenize in both positions in parallel. The target can be mutagenized during the lysogenic cycle or lytic cycle of a phage.

In some particular embodiments, the targeted sequence (mutagenesis target) is in the cell genome or on a mobile genetic element such as a plasmid, transposon or a phage. The mobile genetic element replicates in the recombinant cell. In some particular embodiments, the mutagenesis target is in the cell genome, on one of the DGRec plasmid or inside a phage genome of a recombinant phage that infects the recombinant cell.

In some embodiments of the methods the recombinant cell is a eukaryotic cell. In some embodiments of the methods the recombinant cell is a prokaryotic cell. Prokaryote cell is in particular bacteria. Eukaryote cell includes yeast, insect cell and mammalian cell. In some embodiments of the methods the prokaryotic cell is a bacterial cell. In some embodiments of the methods the bacterial cell is an E. coli cell. The recombinant error-prone RT, in particular recombinant DGR RT, and recombinant recombineering system may be chosen so as to achieve optimal efficiency in the recombinant cell. For example, PapRecT might be chosen to implement DGRec in Pseudomonas aeruginosa.

To increase recombineering efficiency, it may be advantageous to shut off some endogenous DNA repair genes in the host, in particular mutL/S, sbcB, and/or recJ in bacteria. In some embodiments of the method, at least one of the DNA repair genes is inactivated in the recombinant cell. In some particular embodiments, at least one of the mutL/S, sbcB, and recJ is inactivated. The DNA repair gene may be inactivated by standard methods that are known in the art such as deletion of the gene or expression of a dominant negative mutant of the gene. In some embodiments of the methods, the E. coli is deleted for the two exonucleases SbcB and RecJ to increase recombineering efficiency. In some embodiments of the methods, the bacterial cell expresses mutL* (dominant negative mutL), in particular mutL* is encoded by a nucleotide sequence comprising the sequence SEQ ID NO: 15. In some particular embodiments, mutL* is encoded by one of the DGRec system plasmids, in particular the DGRec targeting plasmid.

In some embodiments the methods further comprise expressing the mutagenized DNA sequence.

Because of its adenine randomization mechanism, this technique produces libraries of variants that vary by several orders of magnitude depending on the number of adenines and their placement in the coding sequence. For a TR sequence containing 7 adenines, the potential library size reaches 4⁷ (˜10⁴) DNA sequence variants. For a TR sequence containing 16 adenines, it reaches 4¹⁶ (˜10⁹) DNA sequence variants. In terms of protein sequence variants, library sizes vary even more broadly, depending on the strategic placement of adenines within codons. For example, the different TR designed against sacB disclosed in the examples are able to generate library sizes ranging from 10⁹ to 10¹⁵ potential protein sequence variants. However, there is still potential for improvement as the naturally occuring DGR system in Bordetella phage can potentially generate 10¹³ protein sequence variants, while another DGR system in Treponema can potentially generate 10²⁰ protein sequence variants.

### Library, Cell, Vector, System, Kit

Also provided are libraries of Cas gene mutagenized sequences (mutagenized sequences coding for Cas protein) made according to a method of this invention.

In some embodiments, a library of distinct TR sequences is made of sheared DNA fragments, for example using sonication. The fragments are repaired, tailed, and cloned into a custom vector for TR cloning such as pRL021 or pRL038. The creation of DGRec TR libraries—using, for example, a TR library made of sheared DNA fragments—allows a broader mutagenesis approach that can span entire biosynthetic gene clusters, as each individual DGRec system inside cells will be mutagenizing a different portion of the DNA region that was sheared in the first place. A similar approach was used for the Ec86 bacterial retroelement (Schubert et al., biorxiv 2020, [23]).

Also provided are libraries of recombinant cells comprising the library of Cas gene mutagenized sequences.

Also provided are recombinant cells comprising recombinant coding sequences for a recombinant Cas protein, a recombinant error-prone reverse transcriptase (RT) and at least one recombinant spacer RNA comprising a target sequence according to the present disclosure. In some embodiments the cell further comprises coding sequences for the CRISPR guide RNA(s) (recombinant CRISPR guide RNA(s)). In some embodiments the cell further comprises the recombinant error-prone reverse transcriptase (RT) and at least one recombinant spacer RNA comprising a target sequence.

In some embodiments, the recombinant cell comprises recombinant coding sequences for a recombinant Cas protein, a recombinant DGR RT, recombinant DGR Avd, and at least one recombinant DGR spacer RNA comprising a target sequence according to the present disclosure. In some embodiments the cell further comprises coding sequences for the CRISPR guide RNA(s) (recombinant CRISPR guide RNA(s)). In some particular embodiments, the cell comprises one or a plurality of recombinant plasmids that together comprise the coding sequences for the recombinant Cas protein, the recombinant DGR RT, recombinant DGR Avd, and at least one recombinant DGR spacer RNA comprising a target sequence; preferably together further comprise coding sequences for the CRISPR guide RNA(s) (recombinant CRISPR guide RNA(s)). In some particular embodiments, the cell further comprises the recombinant DGR RT, recombinant DGR Avd, and recombinant DGR spacer RNA comprising a target sequence. In some preferred embodiments, the coding sequence for the DGR RT is operatively linked to an inducible promoter. In some preferred embodiments, the coding sequences for the recombinant DGR Avd and recombinant DGR spacer RNA are operatively linked to constitutive promoters. In some preferred embodiments, the recombinant DGR RT, the recombinant DGR Avd, and recombinant DGR spacer RNA are from the Bordetella bacteriophage BPP-1. In some preferred embodiments, the coding sequences for the recombinant DGR RT and recombinant DGR Avd are present on the same plasmid, in particular pRL014. (DGRec helper plasmid). Optionally, the coding sequence for the recombinant Cas protein, preferably the coding sequences for the recombinant Cas protein and recombinant CRISPR guide RNA(s), are operatively linked to constitutive promoter(s). In some preferred embodiments, the coding sequence for the recombinant Cas protein is on a different plasmid, preferably together with the coding sequence for the recombinant CRISPR guide RNA(s).

In some preferred embodiments, the cell further comprises a coding sequence that expresses a recombinant recombineering system such as a recombinant single-stranded annealing protein (SSAP) mediating oligonucleotide recombineering, in particular recombinant CspRecT according to the present disclosure. In some particular embodiments, the coding sequences for the recombinant single-stranded annealing protein (SSAP) mediating oligonucleotide recombineering, in particular recombinant CspRecT, and DGR spacer RNA comprising a target sequence are present on the same plasmid, preferably derived from pRL021. In some preferred embodiments the cell further comprises the recombinant single-stranded annealing protein (SSAP) mediating oligonucleotide recombineering, in particular the recombinant CspRecT according to the present disclosure.

In some embodiments, the recombinant cell is a eukaryotic cell. In some embodiments, the recombinant cell is a prokaryotic cell. In some particular embodiments, the prokaryotic cell is a bacterial cell. In some particular embodiments, the bacterial cell is an E. coli cell. In some embodiments the bacterial cell expresses mutL* (dominant negative mutL), in particular mutL* comprising the sequence SEQ ID NO: 15. In some particular embodiments, mutL* is encoded by one of the DGRec system plasmids, in particular the DGRec targeting plasmid. In some embodiments the E. coli is deleted for the two exonucleases SbcB and RecJ to increase recombineering efficiency.

In some embodiments of the recombinant cell, the target sequence comprises 70 base pairs. In some embodiments of the recombinant cell, the target sequence is from 50 to 120 base pairs long. In some embodiments of the recombinant cell, the target sequence is from 70 to 100 base pairs long. In some embodiments of the recombinant cell, the target sequence is from 50 to 200 (50, 75, 100, 125, 150, 175, 200) base pairs long or more, for example 50 to 300 (50, 100, 125, 150, 175, 200, 225, 250, 275 or 300) base pairs long or more. In some embodiments of the recombinant cell, the target sequence comprises less than 50 base pairs, in particular 40, 30, 20 base pairs or less.

In some embodiments the recombinant cell further comprises the expression product of the mutagenized sequence.

Another aspect of the invention relates to a recombinant cell system for generating targeted nucleic acid diversity, comprising a recombinant cell according to the present disclosure.

Another aspect of the invention relates to a first kit for performing the method according to the present disclosure, comprising one or a plurality of recombinant expression vectors comprising coding sequences for the recombinant Cas protein, the recombinant error-prone reverse transcriptase (RT), the recombinant spacer RNA(s) comprising a target sequence, and the recombineering system; preferably further comprising coding sequence for the recombinant CRISPR guide RNA(s). In some particular embodiments, the kit comprises one or a plurality of recombinant expression plasmids together comprising coding sequences for the recombinant Cas protein, recombinant DGR RT, recombinant DGR Avd, recombinant DGR spacer RNA(s) and recombinant SSAP mediating oligonucleotide recombineering; preferably further comprising coding sequence for the recombinant CRISPR guide RNA(s). In some preferred embodiments, the system comprises the plasmid pRL014 and a plasmid comprising coding sequence for the recombinant Cas protein, preferably comprising coding sequences for the recombinant Cas protein and recombinant CRISPR guide RNA(s).

Another aspect of the invention relates to a second kit for performing the method according to the present disclosure, comprising:
a first recombinant expression plasmid comprising coding sequences for the recombinant DGR RT and recombinant DGR Avd according to the present disclosure;
a second recombinant expression plasmid comprising coding sequences for the recombinant single-stranded annealing protein (SSAP) mediating oligonucleotide recombineering;
a third recombinant expression plasmid comprising coding sequence for the recombinant Cas protein, preferably comprising coding sequences for the recombinant Cas protein and recombinant CRISPR guide RNA(s); and
an engineered DGR spacer RNA comprising a cloning cassette in replacement of the template region (TR) according to the present disclosure inserted on at least one, preferably both first and second recombinant plasmids.

In some embodiments of the second kit, the coding sequence for the DGR RT is operatively linked to an inducible promoter. In some preferred embodiments, the coding sequences for the recombinant DGR Avd and recombinant DGR spacer RNA are operatively linked to constitutive promoters. In some preferred embodiments, the recombinant DGR RT, the recombinant DGR Avd, and recombinant DGR spacer RNA are from the Bordetella bacteriophage BPP-1. In some preferred embodiments, the first plasmid is pRL014 or pRL038.

In some embodiments of the second kit, the recombinant single-stranded annealing protein (SSAP) mediating oligonucleotide recombineering is recombinant CspRecT. In some embodiments of the second kit, the recombinant single-stranded annealing protein (SSAP) mediating oligonucleotide recombineering is operably linked to an inducible promoter. In some embodiments, the cloning cassette comprises a CcdB gene flanked by copies of the same type IIS restriction site in convergent orientation. In some preferred embodiments, the second plasmid is pRL021. In some particular embodiments, the second plasmid comprises at least two cloning cassettes flanked by different type IIS restriction sites, thereby allowing cloning of different targets into the same plasmid. In some preferred embodiments, the first and second plasmids comprise a cloning cassette. This allows the mutagenesis of multiple targets simultaneously using only two plasmids for the cloning of the targets and expression of the DGR recombineering system.

In some embodiments, the second kit further comprises the target sequence; preferably a synthetic double-stranded oligonucleotide comprising the target sequence flanked by copies of the same type IIS restriction site in divergent orientation, forming non complementary sticky ends.

### Uses

Another aspect of the invention relates to the in vitro use of the recombinant cell system according to the present disclosure for the generation of targeted nucleic acid diversity in a Cas gene.

Another aspect of the invention relates to a method of generating a library of Cas protein variants, comprising:
expressing in a recombinant cell comprising a recombinant Cas gene, a recombinant error-prone reverse transcriptase (RT) and recombinant spacer RNA comprising a target sequence for mutagenesis of a DNA sequence in the recombinant Cas gene;
making a mutagenized cDNA polynucleotide homologous to the DNA sequence in the recombinant cell;
expressing a recombineering system in the recombinant cell;
recombining the mutagenized cDNA with the homologous DNA sequence of the recombinant Cas gene in the recombinant cell; and
expressing the recombinant Cas gene comprising the mutagenized DNA sequence in the recombinant cell to generate a library of expressed Cas protein variants.

The method generates a library of Cas protein variants. The method of generating a library of Cas proteins is performed as disclosed above for the method of generating nucleic acid diversity in a Cas gene. The various embodiments disclose above for the method of generating nucleic acid diversity in a Cas gene also apply to the method of generating a library of Cas protein variants.

In some particular embodiments of the above method of generating a library of Cas proteins, the target sequence for mutagenesis is first recoded to modulate the level of diversification as mentioned above for the method of generating nucleic acid diversity in a Cas gene.

In some particular embodiments of the above method of generating a library of Cas proteins, the cell comprises one or a plurality of recombinant plasmids that together comprise the coding sequences for the recombinant Cas protein, the recombinant DGR RT, recombinant DGR Avd, and at least one recombinant DGR spacer RNA comprising a target sequence; preferably together further comprising coding sequences for the CRISPR guide RNA(s) (recombinant CRISPR guide RNA(s)), according to the present disclosure. In some preferred embodiments of the above method of generating a library of Cas proteins, the recombinant cell comprises:
a first recombinant expression plasmid (DGRec helper plasmid) comprising coding sequences for the recombinant DGR RT and recombinant DGR Avd according to the present disclosure; preferably the coding sequence for the DGR RT is operatively linked to an inducible promoter and the coding sequence for the recombinant DGR Avd is operatively linked to a constitutive promoter according to the present disclosure. In some preferred embodiments, the first plasmid is pRL014 or derived from pRL038;
a second recombinant expression plasmid comprising a coding sequence for the recombinant single-stranded annealing protein (SSAP) mediating oligonucleotide recombineering, in particular recombinant CspRecT; preferably operatively linked to an inducible promoter according to the present disclosure; preferably the plasmid is derived from pRL021;
a third recombinant expression plasmid comprising a coding sequence for the recombinant Cas protein; preferably comprising coding sequences for a recombinant Cas protein and recombinant CRISPR guide RNA(s); optionally operatively linked to inducible promoter(s) according to the present disclosure; and
coding sequences for the at least one recombinant DGR spacer RNA according to the present disclosure inserted in the first and/or second expression plasmid; preferably operatively linked to constitutive promoter(s), preferably the plasmid is derived from pRL021 or pRL038.

Another aspect of the invention relates to a method of selection and/or screening of a library of Cas proteins, comprising:
a) generating a library of expressed Cas proteins in a recombinant cell according to the method of the present disclosure; and
b) selecting and/or screening the activity of the expressed Cas proteins.

The selecting and/or screening step is advantageously performed in the recombinant cell according to the present disclosure (i.e., in vivo). In some embodiments, the recombinant cell further comprises at least one marker for the selection and/or screening of the activity of the expressed Cas proteins. Such markers are well-known in the art and some are disclosed in the examples of the present application. The screening may use as marker, a reporter gene encoding a protein that produces a detectable signal in the recombinant cell. Such reporters are well-known in the art and include for example enzymes that produce visible or coloured reaction products and luminescent proteins such as fluorescent proteins and luciferases. In some embodiments, the recombinant cell comprises a fluorescent reporter gene, in particular the mCherry gene. The selection may be a positive selection (cells that have gained the specific gene survive), for example using antibiotic resistance marker or auxotrophy marker. Alternatively, the selection may be a negative selection or counterselection (cells that have lost the specific gene survive), for example using ccdB gene encoding the toxin CcdB or SacB gene encoding the enzyme levansucrase that converts sucrose into a toxic metabolite in gram-negative bacteria. In some embodiments, the recombinant cell comprises the SacB gene. In some embodiments, the recombinant cell comprises a selection marker and a screening marker, in particular the mCherry gene and the SacB gene. The at least one marker is expressible in the recombinant cell. In particular, the at least one marker is operatively linked to a promoter as disclosed herein. The at least one marker is advantageously included in an expression cassette comprising a promoter, in particular a constitutive promoter according to the present disclosure, a ribosome-binding site and at least one marker, in particular an operon coding for a selection marker and a screening marker, preferably the mCherry gene and the SacB gene. In some embodiments, the at least one marker is integrated into the genome of the recombinant cell.

For example, the recombinant cell may express a library of functional or non-functional dead Cas proteins and a CRISPR guide RNA targeting the dead Cas proteins to repress transcription of a screening marker, in particular mCherry and/or a selection marker, in particular SacB. Functional dead Cas proteins able to repress transcription of the screening and/or selection marker may be screened using mCherry fluorescence and/or selected using SacB-mediated toxicity on sucrose.

In some embodiments, the step a) (mutagenesis) and/or the step b) (selection and/or screening) are repeated at least one time. In particular embodiments, the selection of step b) is repeated at least one time. In some particular embodiments, the step a) and the selection of step b) are repeated at least one time. Examples of rounds of selection are shown in FIG. 12 and illustrated in the examples.

The method of selection and/or screening according to the invention allows the isolation of Cas proteins with modified amino acid sequences and novel properties, such as for example dCas variants with improved ability to repress transcription, or Cas variants that can recognize non-canonical PAM sequences.

Another aspect of the invention relates to a method of engineering a Cas protein having a desired function, comprising;
providing a sequence coding for a Cas protein;
generating a library of mutagenized sequences of the Cas protein according to a method of the present disclosure;
expressing the library; preferably in cell;
screening the activity of the expressed Cas proteins; and
identifying Cas protein(s) having the desired function.

The activity of the expressed proteins may be assessed by assays that are known in the art such as colorimetric enzymatic assays, or the binding of the expressed protein to a desired partner can be assessed by assays that are known in the art such as phage display, bacterial display or yeast display.

The DGRec in vivo targeted diversity system could be implemented in a vast number of applications in which one wants to improve, or change, a given Cas protein function. Because of the unique DGR mechanism of adenine mutagenesis, diversity can be targeted with precision and multiple amino acid changes can occur in a single recombination event within the mutagenesis window (FIG. 3C). The mutagenesis window being flexible in size, DGRec can be applied to mutagenize a specific Cas protein location, such as a Cas enzyme active site, or a Cas exposed domain mediating interaction. In addition, the predictability of adenine mutagenesis to drive the mutagenesis provides the option of recoding the target region to optimize the mutagenesis profile within the window, mutating more intensively some critical amino acids position of choice. The ability to multiplex the targeted mutagenesis window opens the possibility of driving intense mutagenesis on different genomic locations in parallel. Finally, the creation of DGRec libraries—using, for example, a library made of sheared DNA fragments—allows a broader mutagenesis approach that can span entire biosynthetic gene clusters.

The practice of the present invention will employ, unless otherwise indicated, conventional techniques, which are within the skill of the art. Such techniques are explained fully in the literature.

The invention will now be exemplified with the following examples, which are not limitative, with reference to the attached drawings in which:

### FIGURE LEGENDS

FIG. 1 shows a non-limiting general scheme for practicing certain embodiments of the invention.

FIG. 2 shows plasmid constructs successful for expression of a synthetic DGR system. CmR: chloramphenicol resistance gene; KanR: kanamycin resistance gene; CspRecT: single-stranded annealing protein mediating oligo recombineering; mutL*: a dominant negative mutL allele shutting down the DNA mismatch repair system, increasing recombineering efficiency.

FIG. 3—DGRec mutagenesis with varying TR targets. A) Serial dilution of two replicate cultures plated after 48h DGRec induction, showing the emergence of sucrose-resistant colonies with a functional DGRec system targeting the sacB gene (pRL014+pAM009), but not in a negative control containing an inactivated RT enzyme (pRL034+pAM009). B) Colonies after 48h DGRec induction of plasmids pRL014+pAM011 targeting the mCherry gene in the host chromosome. The picture is an overlay of mCherry fluorescence) with bright field. Colonies indicated with white arrows have lost their mCherry fluorescence due to DGRec mutagenesis. C) and D) The TR sequences used in the DGRec system are displayed in a box above its target region. For each TR tested, a selection of a few DGRec mutants obtained by Sanger sequencing of the target region are aligned to the reference. Mutations are highlighted by grey boxes on nucleotides, and adenine positions in the TR target are highlighted in grey. The mutations obtained predominantly follow the known DGR mutagenesis pattern of adenine mutagenesis.

FIG. 3C: TR_AM009 (SEQ ID NO: 24); TR_AM009 target wt/nt strand 1 (SEQ ID NO: 43); TR_AM009 target wt/nt strand 2 (SEQ ID NO: 44); TR_AM009 target wt/aa (SEQ ID NO: 45); Variant-TR_AM009 n° 1 to 4 (SEQ ID NO: 46 to 49). TR_AM010 (SEQ ID NO: 25); TR_AM010 target wt/nt strand 1 (SEQ ID NO: 50); TR_AM010 target wt/nt strand 2 (SEQ ID NO: 51); TR_AM010 target wt/aa (SEQ ID NO: 52); Variant-TR_AM010 n° 1 to 4 (SEQ ID NO: 53 to 56). TR_RL016 (SEQ ID NO: 42); TR_RL016 target wt/nt strand 1 (SEQ ID NO: 57); TR_RL016 target wt/nt strand 2 (SEQ ID NO: 58); TR_RL016 target wt/aa (SEQ ID NO: 59); Variant-TR_RL016 n° 1 to 4 (SEQ ID NO: 60 to 64). FIG. 3D: TR_AM004 (SEQ ID NO: 22); TR_AM004 target wt/nt strand 1 (SEQ ID NO: 64); TR_AM004 target wt/nt strand 2 (SEQ ID NO: 65); TR_AM004 target wt/aa (SEQ ID NO: 66); Variant-TR_AM004 (SEQ ID NO: 67). TR_AM007 (SEQ ID NO: 23); TR_AM007 target wt/nt strand 1 (SEQ ID NO: 68); TR_AM007 target wt/nt strand 2 (SEQ ID NO: 69); TR_AM007 target wt/aa (SEQ ID NO: 70); Variant-TR_AM007 n° 1 to 4 (SEQ ID NO: 71 to 74). TR_AM011 (SEQ ID NO: 19); TR_AM011 target wt/nt strand 1 (SEQ ID NO: 75); TR_AM011 target wt/nt strand 2 (SEQ ID NO: 76); Variant-TR_AM011 n° 1 to 4 (SEQ ID NO: 77 to 80).

FIG. 4—Spacer RNA structure in the DGRec system. A) Annotation of the Spacer RNA important features. Two grey boxes indicate the self-annealing segments necessary to prime the Reverse transcriptase complex. A triangle shows the A56 nucleotide which forms the starting point of the cDNA polymerization. B) Cartoon of the 3D conformation adopted by the spacer RNA allowing recruitment/priming of the Reverse Transcriptase complex.

FIG. 5—Plasmid map of pRL038 and pRL021. Detailed view section that enables fast cloning of new TR sequences inside the spacer RNA by Golden Gate assembly. T symbols indicate terminators. Brackets on each plasmid indicate ccdB cloning site.

FIG. 6—Multiplex DGRec mutagenesis. A) A selection of DGRec mutants sequenced after 48h DGRec induction of plasmids pAM030+pAM001. The results show that pAM030, derived from the pRL038 plasmid, is functional to drive DGRec mutagenesis through its encoded spacer RNA locus. B) Sequence of two clones obtained after 48h DGRec induction of plasmids pAM030+pAM011, which contain a TR driving mutagenesis in the sacB and mCherry genes, respectively. These clones, obtained by combining the sucrose and mCherry fluorescence assay, were simultaneously mutagenized in both target regions. FIG. 6A: TR_AM009 (SEQ ID NO: 24); TR_AM009 target wt/nt strand 1 (SEQ ID NO: 43); TR_AM009 target wt/nt strand 2 (SEQ ID NO: 44); TR_AM009 target wt/aa (SEQ ID NO: 45); Variant-TR_AM009 n° 5 to 8 (SEQ ID NO: 80 to 84). FIG. 6B: TR_AM011 (SEQ ID NO: 19); TR_AM011 target wt/nt strand 1 (SEQ ID NO: 85); TR_AM011 target wt/nt strand 2 (SEQ ID NO: 86); Variant-TR_AM011 n° 5 to 6 (SEQ ID NO: 87 to 88). TR_AM009 (SEQ ID NO: 24); TR_AM009 target wt/nt strand 1 (SEQ ID NO: 89); TR_AM009 target wt/nt strand 2 (SEQ ID NO: 90); TR_AM009 target wt/aa (SEQ ID NO: 45); Variant-TR_AM009 n° 9 to 10 (SEQ ID NO: 91 to 92).

FIG. 7—Amplicon sequencing of mutagenesis target regions. A) A selection of a few sucrose-resistant mutants of the sacB gene obtained after 48h DGRec mutagenesis inside the sacB gene and Sanger sequenced are aligned over the same mutagenesis target analyzed by Illumina amplicon sequencing after 48h DGRec induction (and no selection). The mutagenesis target sequence is highlighted in grey as well as adenine positions within this window. The mutations obtained predominantly follow the known DGR mutagenesis pattern of adenine mutagenesis and remain well-delineated within the target region. B) Same Illumina sequencing analysis plots for different targeted regions. FIG. 7A: mutagenesis target (SEQ ID NO: 24); wt/nt strand 1 (SEQ ID NO: 43); wt/nt strand 2 (SEQ ID NO: 44); wt/aa (SEQ ID NO: 45); Variant n° 1 to 4 (SEQ ID NO: 46 to 49). Sequence including mutagenesis target shown below plot (SEQ ID NO: 93).

FIG. 8—Theoretical protein library size obtained when diversifying adenine positions on one strand, or on the other strand (T), in a 90 nucleotide sliding window over the Cas9 PID sequence.

FIG. 9—Selection assay for functional Cas9, and testing of two Cas9 variants with recoded PAM-interacting domains (PIDs) which were optimised for low A (pWR55) or low T content (pWR56).

FIG. 10—Parts used for the dCas9 DGR library creation and selection or screening.

FIG. 11—First round of DGRec dCas9 screening (Further rounds can be carried out)

FIG. 12—DGRec screening rounds process. Further rounds increase selection strength and allow removal of SacB mutants.

FIG. 13—DGRec library 1 screening results.

DGR1 (SEQ ID NO: 108); DGR1 target *1141/nt strand 1 (SEQ ID NO: 130); DGR1 target *1141/nt strand 2 (SEQ ID NO: 131); DGR1 target *1141/aa (SEQ ID NO: 132); Variant-DGR1 v1 (SEQ ID NO: 133); Variant-DGR1 v3 (SEQ ID NO: 134); Variant-DGR1 v5 (SEQ ID NO: 135); Variant-DGR1 v6 (SEQ ID NO: 136); Variant-DGR1 v7 (SEQ ID NO: 137); Variant-DGR1 v9 (SEQ ID NO: 138).

FIG. 14—DGRec library 3 screening results.

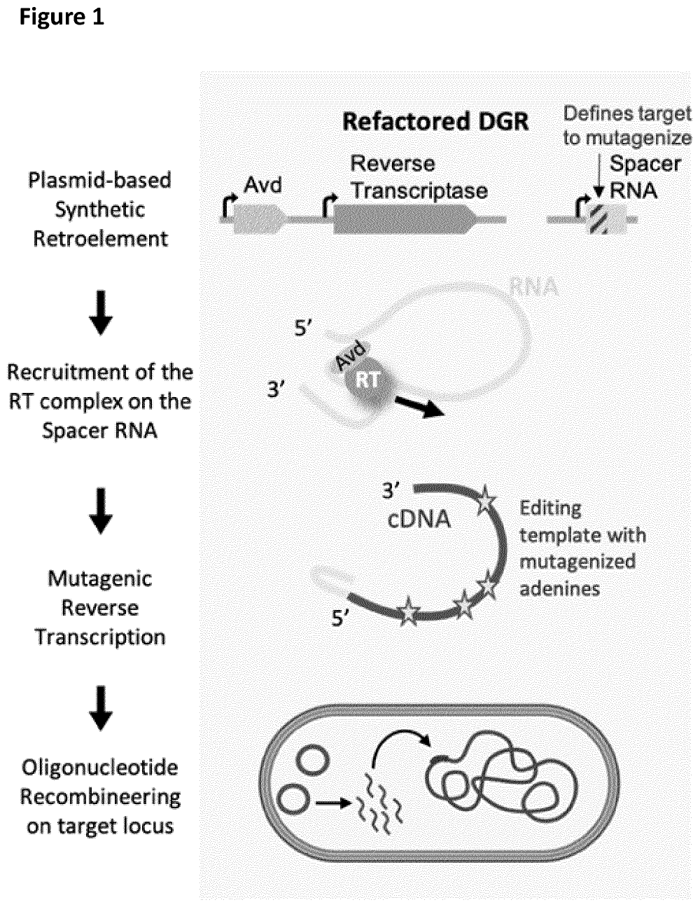

DGR3 (SEQ ID NO: 109); DGR3 target *1141/nt strand 1 (SEQ ID NO: 139); DGR3 target *1141/nt strand 2 (SEQ ID NO: 140); DGR3 target *1141/aa (SEQ ID NO: 141); Variant-DGR3 vsl (SEQ ID NO: 142); Variant-DGR3 vs2 (SEQ ID NO: 143); Variant-DGR1 vs3 (SEQ ID NO: 144); Variant-DGR3 vs4 (SEQ ID NO: 145).

### EXAMPLES

### Material and Methods

### Bacterial Strains, Plasmids, Media, and Growth Conditions

All bacterial strains and plasmids used in this work are listed in Table 6. For plasmid propagation and cloning the E. coli strain MG1655* was used. All the strains were grown in lysogeny broth (LB) at 37° C. and shaking at 180 RPM. For solid medium, 1.5% (w/v) agar was added to LB. The following antibiotics were added to the medium when needed: 50 μg ml⁻¹ kanamycin (Kan), 30 μg ml⁻¹ chloramphenicol (Cm). For counterselection with sacB, 5% of sucrose was added to the plating media before pouring.

### Cloning Procedures

Deletions were obtained by clonetegration [34], and combined by P1 transduction [35]. The sacB-mCherry cassette was inserted using OSIP plasmid pFD148.

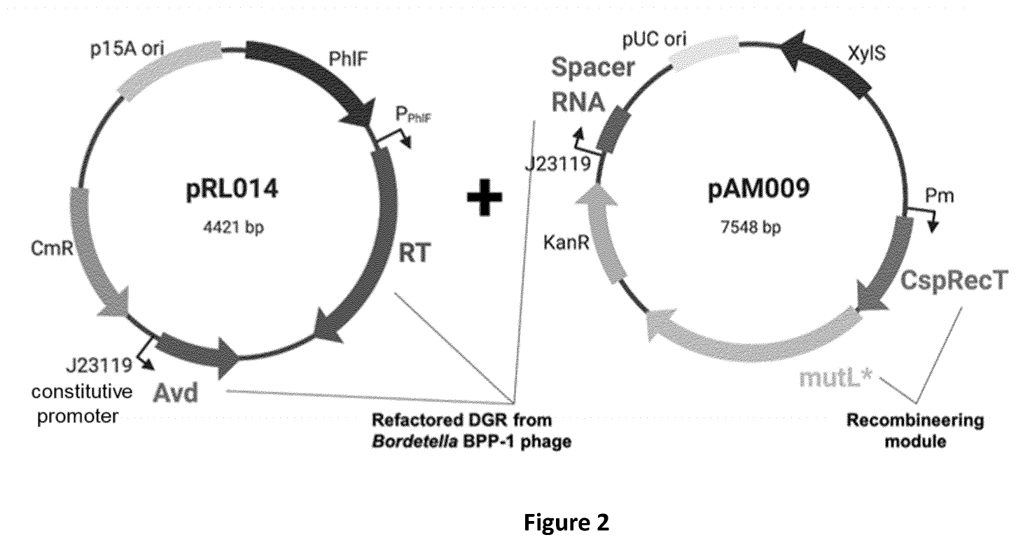

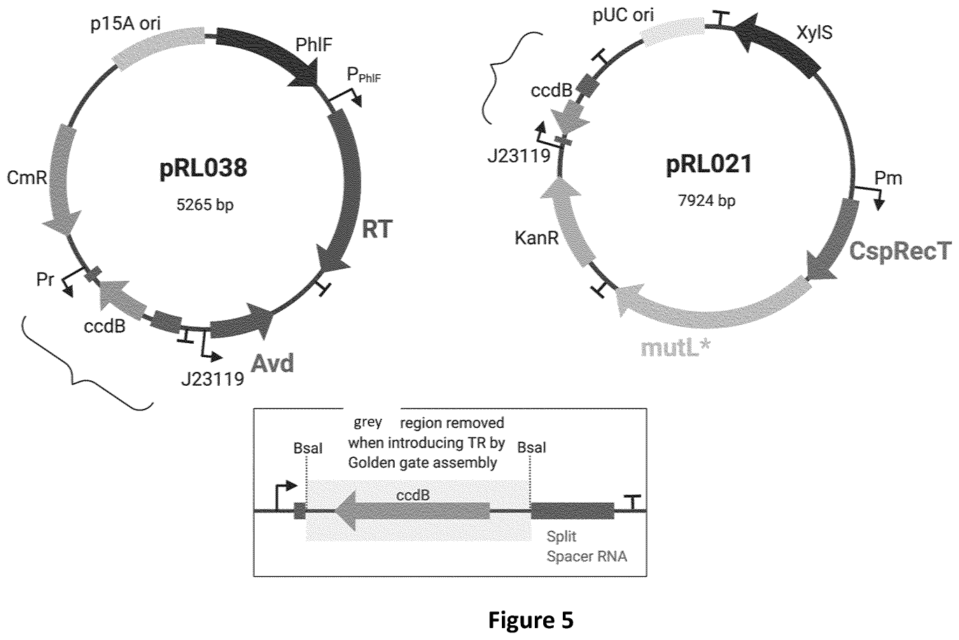

Plasmids were constructed by Gibson Assembly [36] unless specified. Plasmid sequences are presented in the sequence listing, plasmid maps are displayed in FIG. 2 and FIG. 5, and the relevant recoded gene sequences are listed in Table 7. Novel TR sequences can be cloned on pRL021 or pRL038 (FIG. 5) using Golden Gate assembly with BsaI restriction sites [37]. The plasmids contain a ccdB counter-selection cassette in between two BsaI restriction sites [38]. This ensures the selection of clones in which a TR was successfully added to the plasmid during cloning. All oligonucleotide sequences used for TR assembly are listed in Table 8.

### Induction of the DGRec System

To perform mutagenesis, the DGRec recipient strains listed in Table 6 were transformed with the two DGRec plasmids via electroporation and plated on Kan and Cm selective media. After overnight growth at 37° C., colonies were picked into 1 mL of LB Kan, Cm in a 96-well plate and allowed to grow 6-8 hours. These un-induced pre-cultures were diluted 500-fold into 1 mL of LB Kan, Cm, containing 1 mM m-toluic acid and 50 μM DAPG (inducing recombineering module and the RT, respectively) in a 96 deep-well plate, and allowed to grow for 24 hours at 34° C. with shaking at 700 rpm, reaching stationary phase. This 500-fold dilution and growth was repeated once more for all cultures to perform a 48h time point.

### Evaluation of Recombination Efficiency

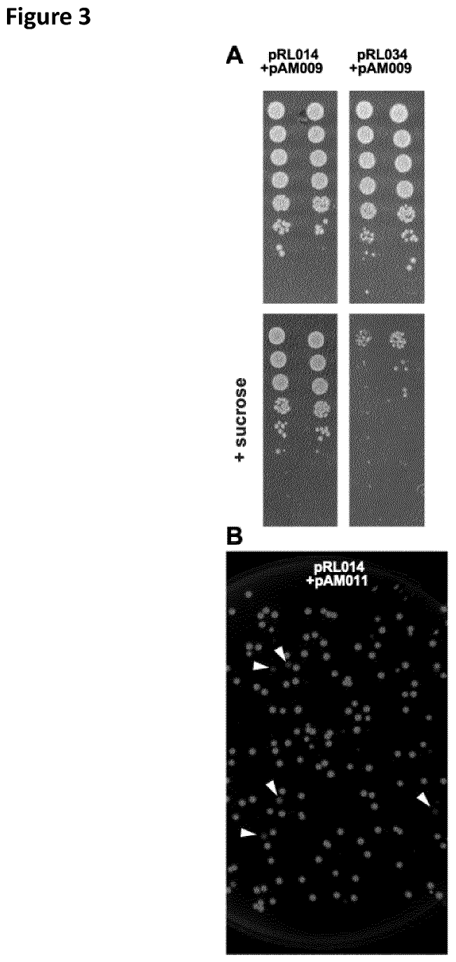

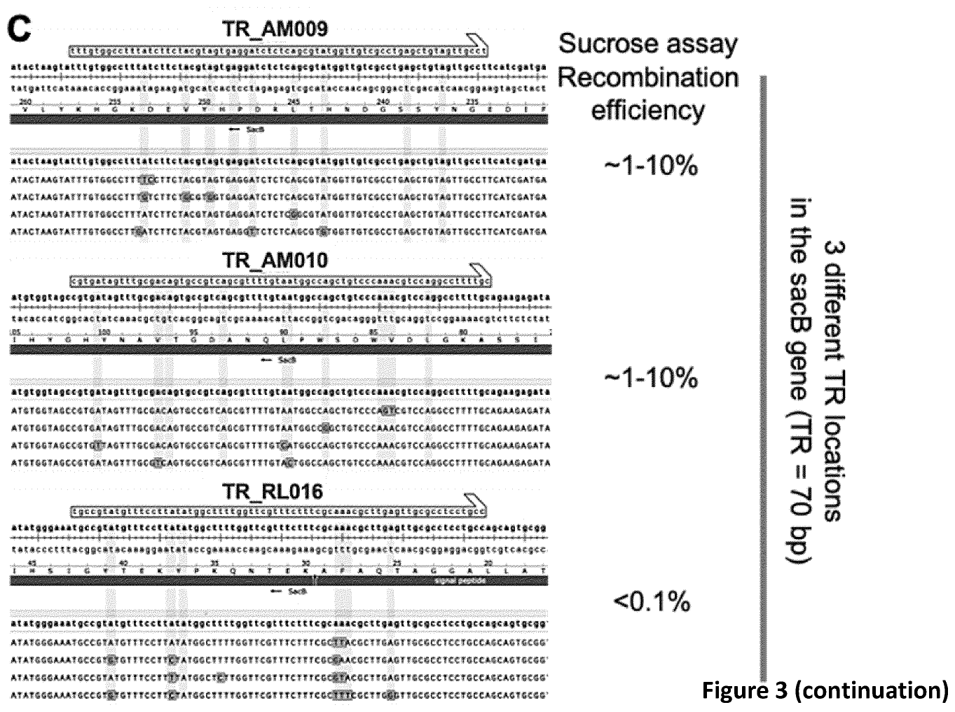

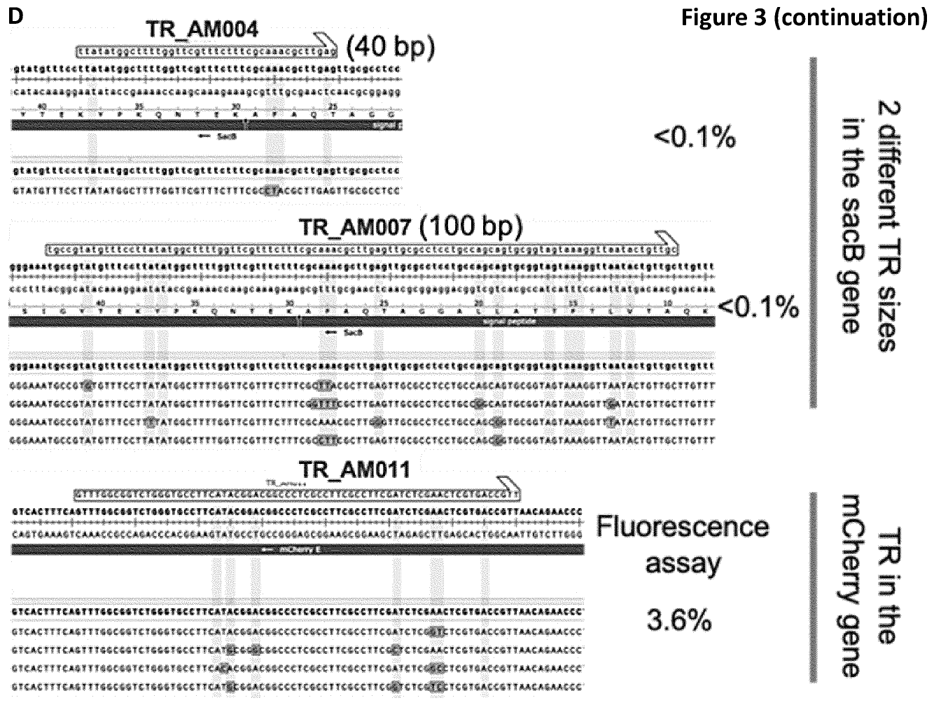

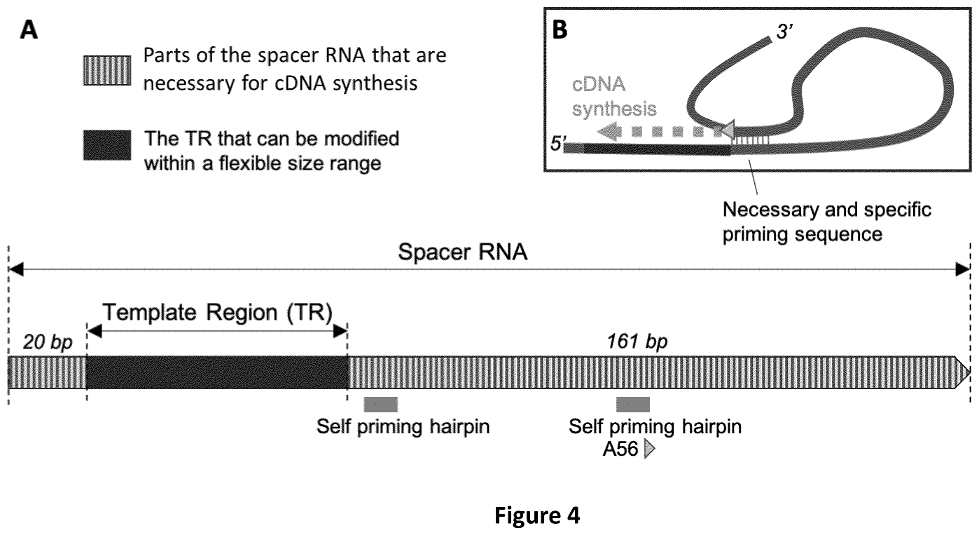

Sucrose assay: After 24 h and 48h DGRec mutagenesis targeted at sacB (plasmids pRL014 combined with pRL016, pAM004, pAM007, pAM009 or pAM010 in strain sRL002, compared with negative control reverse transcriptase plasmid pRL034 effect), the cells were serially diluted in LB and plated on selective media supplemented with and without 5% sucrose. The fraction of sucrose-resistant cells per sample were estimated for 4 biological replicates. 8 sucrose-resistant colonies were sent for Sanger sequencing and were confirmed to be DGRec mutants. Of note, the spontaneous rate of sacB mutations is elevated in this assay (reaching 10⁴ in the negative control samples), and some spontaneous sacB mutant could outcompete other cells during the 48h growth, resulting in a large uncertainty in the recombination efficiency evaluation (value ranges reported in FIG. 3C).

mCherry fluorescence assay: After 48h DGRec mutagenesis targeted at mCherry (plasmids pRL014+pAM011 in strain sRL002, compared negative control plasmids pRL034+pAM011), cultures were diluted and plated on LB plates to obtain ˜200 colonies per plate. Plates were then imaged using an Azure Biosystems Fluorescence Imager, and images were processed by ImageJ [39]. Colonies with and without fluorescence were counted for 4 biological replicates. 8 non-fluorescent colonies (only seen in pRL014+pAM011 replicates) were sent for Sanger sequencing and were confirmed to be DGRec mutants.

### Production of DGRec Mutated Samples

Induction of the DGRec system (see all DGRec constructs in Table 6) was performed as previously described: the DGRec recipient strains were transformed with the two DGRec plasmids via electroporation and plated on Kan and Cm selective media. After overnight growth at 37° C., colonies were picked into 1 mL of LB Kan, Cm in a 96-well plate and allowed to grow 6-8 hours. These un-induced pre-cultures were diluted 500-fold into 1 mL of LB Kan, Cm, containing 1 mM m-toluic acid and 50 μM DAPG (inducing recombineering module and the RT, respectively) in a 96 deep-well plate, and allowed to grow for 24 hours at 34° C. with shaking at 700 rpm, reaching stationary phase. This 500-fold dilution and growth was repeated once more for all cultures to reach 48h of induction.

### Genomic and Plasmid DNA Extraction

Genomic DNA was extracted from mutagenized strains using the NucleoSpin 96 Tissue, 96-well kit for DNA from cells and tissue (Macherey-Nagel), following manufacturer's protocols. When the DGRec targeted region was located on a plasmid, then plasmids were extracted using the QIAprep Spin Miniprep Kit (Qiagen).

### Example 1: Expression of a Functional Plasmid-Based DGR System in Escherichia coli

Heterologous expression of a protein is always a challenge, due to the possible problems in protein folding, toxicity, or lack of function in the new host. However, making a system work in E. coli multiplies its usability, as these bacteria have become by far the most widely used bacterial chassis for genetic applications. Indeed, the fact that DGRs are naturally absent from common laboratory bacterial and phage cloning strains[2] is probably the main reason why these attractive retroelements have not yielded any genetic tools so far.

Several approaches were employed by the inventors to express a functional reverse transcriptase complex in E. coli, and herein described is the one that was successful in the inventor's hands: a ‘refactored’ version of the native DGR system from the Bordetella phage BPP-1 was built, so that each of the DGR components are expressed independently from each other. There are three elements in the system that generate mutagenic cDNA: the reverse transcriptase major subunit (bRT), the reverse transcriptase accessory subunit (Avd), and the spacer RNA. These three elements are combined into an operon structure in the native DGR structure. In the method used in this example each of these elements was cloned under a separate promoter (FIG. 2).

This setup allowed for more flexibility in tuning the relative amount of each element: the bRT protein was expressed under a PhlF promoter (inducible by DAPG), while the Avd accessory protein and the spacer RNA were both expressed under a strong constitutive promoter (J23119) thus providing these components (required in higher copy numbers) in excess for the system. Furthermore, the bRT and avd coding sequences were codon-optimized for expression in E. coli.

Example 2 shows that this approach was successful to assemble a functional RT-avd enzymatic complex in E. coli, able to use the spacer RNA as a specific template for mutagenic reverse transcription.

### Example 2: Coupling DGR cDNA Production with Oligonucleotide Recombineering

Natural DGRs require a recognition sequence called IMH flanking their target sequence to enable the ‘retrohoming’ step (the introduction of mutations in the target region) [1], [9]. The inventors looked into oligonucleotide recombineering as a way to entirely bypass this poorly-understood ‘retrohoming’ step of natural DGRs.

Oligo-mediated recombineering uses incorporation of genomic modifications via oligonucleotide annealing at the replication fork onto target genomic loci [10]. A recombineering module was added onto one of the plasmids used for DGR expression (FIG. 2), and the inventors screened for activity in an E. coli strain deleted for SbcB and RecJ, two exonucleases shown to reduce recombineering efficiency [23].

For detecting the mutagenesis activity of the system, a sacB counter-selection assay in the recipient E. coli strain was used. SacB, encoded in the host genome, makes sucrose toxic to the cells, a way to negatively select them (see methods for detail). By engineering the DGR RNA to target the SacB gene, the appearance of mutants resistant to sucrose in the population could be detected. Those mutants were detected upon induction of the plasmid-borne DGR system, and Sanger sequencing in the area targeted by the synthetic DGR unmistakably showed that a majority of these mutants resulted from DGR mutagenesis activity (FIG. 3). Indeed, mutagenesis happened primarily at adenine positions, the hallmark pattern of DGR systems. Moreover, none of such mutants was ever obtained using an inactive RT variant (Table 1; FIG. 3A).

TABLE 1
Essentiality of DGR components. The DGR
components were inactivated as follows.

| | Obtention of confirmed DGR |
|---|---|
| DGRec component | mutants upon inactivation |
| Reverse Transcriptase | No |
| Avd | No |
| TR | No |
| CspRecT | No |
| mutL* | Yes |
| ΔsbcB + ΔrecJ in host genome | Yes |

Reverse Transcriptase: a SMAA substitution in the enzyme active site (plasmid pRL034); Avd: removal from plasmid (plasmid pRL035); TR: placing of a TR with no corresponding target inside host (plasmid pAM001); CspRecT: removal from plasmid (plasmid pAM014); mutL*: removal from plasmid (plasmid pAM015); ΔsbcB + ΔrecJ in host genome: strain without deletions (strain sRL003). To look for DGR mutants, the sacB target TR region from 4 sucrose resistant colonies were amplified by PCR and sent for Sanger sequencing. Any mutations in the target region was counted as a ‘confirmed DGR mutant’.

Recombination efficiency within the sacB gene can be estimated thanks to a sucrose counter-selection assay (see methods for details). Of note, TR_AM010 and TR_AM009 which target the active site position of SacB had much higher efficiencies (reaching 10% in some samples) than TR_RL016 targeting the C-terminal region of SacB, consistent with the fact that a larger number of DGRec variants will inactivate the enzyme within its active site (FIG. 3C).

The mCherry mutagenesis provides a different and more robust assay to estimate the DGRec recombination efficiency (no selection required), by counting the fraction of cells losing the mCherry fluorescence (see methods for details) (FIG. 3B). The average recombination efficiency obtained from 4 biological replicates after 48h of DGRec mutagenesis is 3.6% (standard deviation 1.6%) (FIG. 3C). Of note, like for the sucrose assay, this value is necessarily an underestimation of the actual mutagenesis frequency, since only the subset of mCherry variants that have lost fluorescence are counted in this process.

The essentiality of the various DGRec components was assessed, by removing or inactivating these components one by one and testing for the obtention of DGRec mutants. The drop in recombination efficiency when removing those components was further assessed by Amplicon sequencing (Example 4).

These results confirm the ability of the DGRec system to mutagenize multiple targets, in different genes, and using mutagenesis windows of varying sizes (FIG. 3).

### Example 3: Multiplex DGRec Mutagenesis

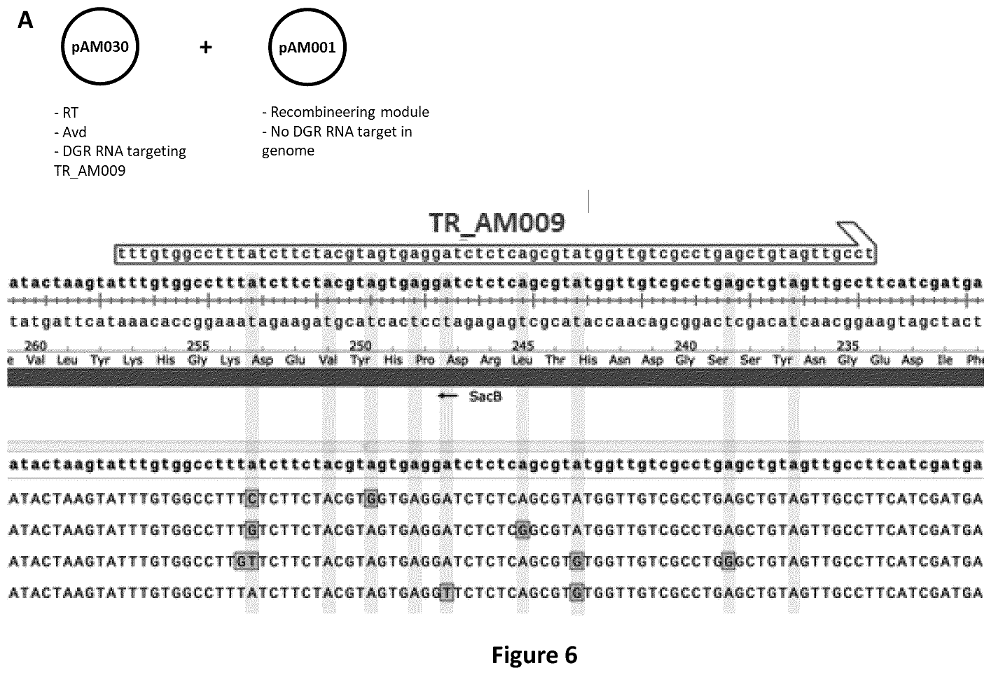

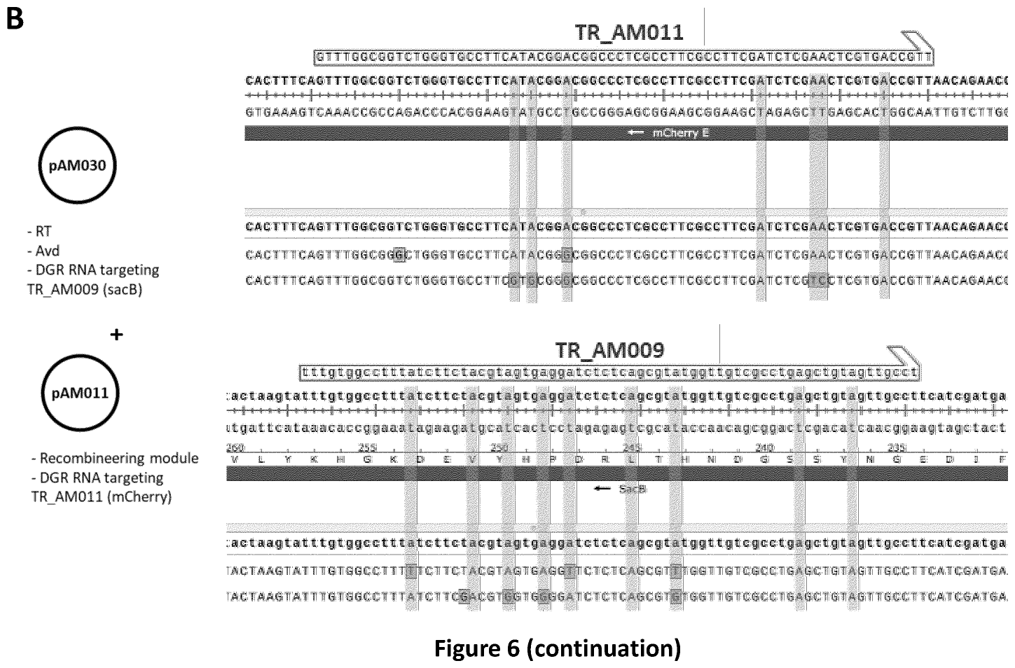

The sucrose and mCherry fluorescence assay were combined to mutagenize both target regions simultaneously. pAM030, derived from the pRL038 plasmid contains bRT, bAvd and DGR RNA targeting TR_AM009. pAM001 contains CspRecT recombineering module and no DGR RNA target in the genome. pAM011 contains CspRecT recombineering module and DGR RNA targeting TR_AM011 (mCherry). DGRec mutants were sequenced after 48h DGRec induction of plasmids pAM030+pAM00L. The results show that pAM030, derived from the pRL038 plasmid, is functional to drive DGRec mutagenesis through its encoded spacer RNA locus (FIG. 6A). DGRec mutants were sequenced after 48h DGRec induction of plasmids pAM030+pAM011 which contain a TR driving mutagenesis in the sacB and mCherry genes, respectively. These clones, obtained by combining the sucrose and mCherry fluorescence assay, were simultaneously mutagenized in both target regions (FIG. 6B).

These results confirm the ability of the DGRec system to mutagenize multiple targets simultaneously in different genes.

### Example 4: Amplicon Sequencing of Mutagenesis Target Regions

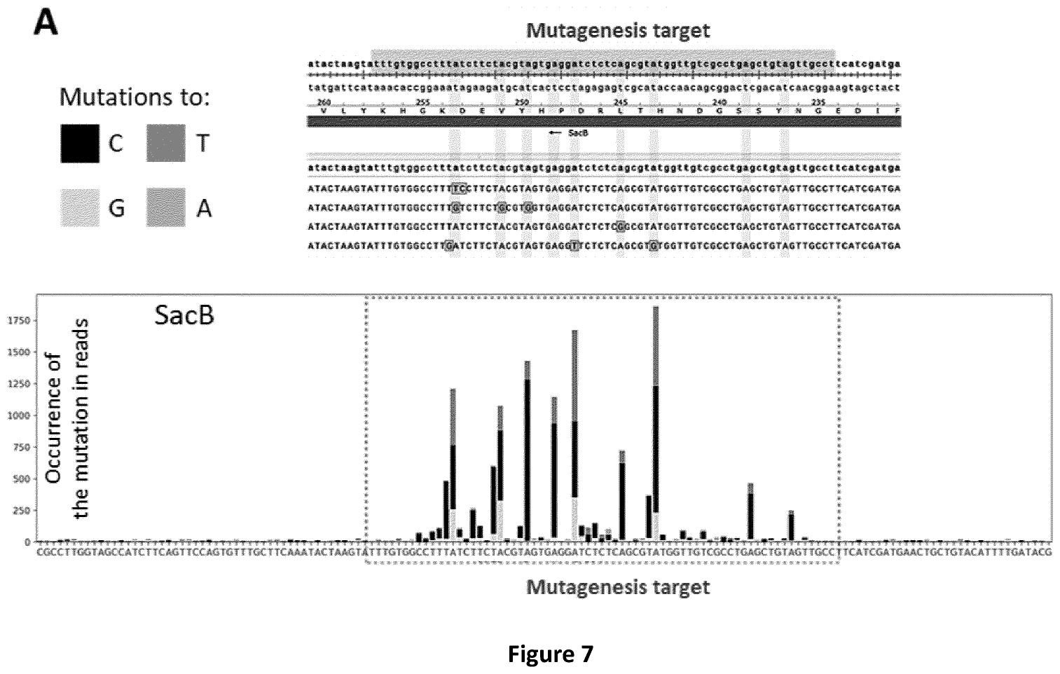

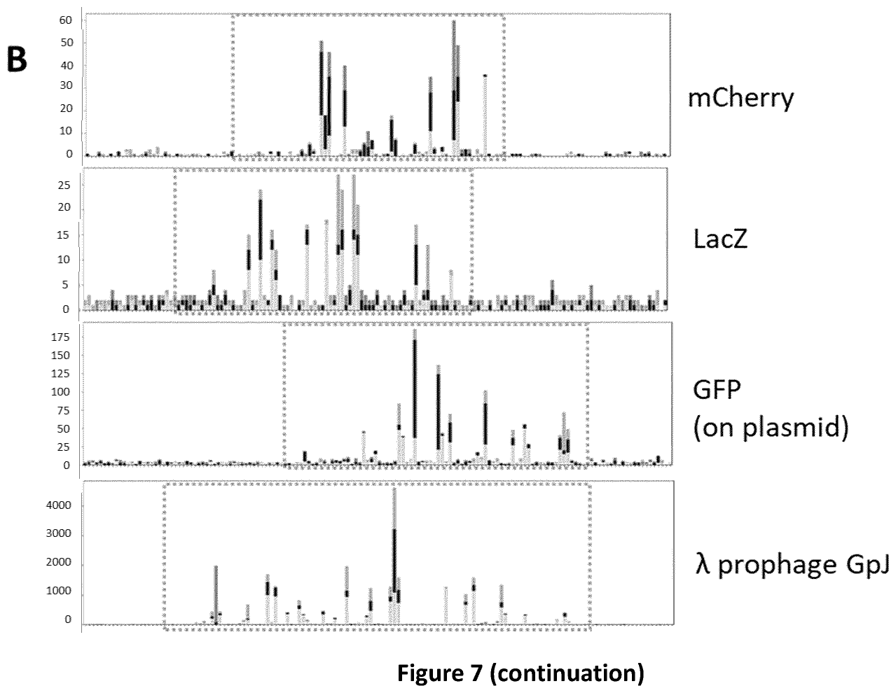

Sequencing results confirmed and strengthened the previous observations of DGrec mutagenesis using Sanger sequencing shown in Example 2 (FIG. 7A). A high mutagenesis well-constrained within the targeted region, and mainly concentrated on the RNA template adenine positions was observed. Moreover, deep sequencing allowed to detect mutagenesis on multiple gene targets without the need for selection of the mutants (FIG. 7B).

After 48h induction of the DGRec system, between 1,000 and up to 10,000 gene variants could be detected inside the targeted region (a large underestimate of the actual number of variants), with variant genotypes typically representing 20 to 100% of all genotypes sequenced within the cell population.

A measure of the DGRec mutagenesis in each sample can be obtained from a measure of the increase in mutation rate within the DGRec targeted region (mutation rate of adenines within the targeted region divided by the mutation rate of adenines outside of the targeted region). This value is named “Amut” in the following paragraphs. Note that mutations outside of the target region might be sequencing mistakes rather than actual mutation. This metric is thus a measure of signal over background rather than a measure of how much DGRec increase mutation rate over the spontaneous mutation rate of E. coli. Nonetheless this metric enables to compare the DGRec mutagenesis efficiency of different samples.

In the following, for each sample analyzed, the plasmids and E. coli strains are indicated under brackets.

### Essentiality of DGRec Components

Samples lacking a functional Reverse Transcriptase [pRL034+pRL016 in sRL002], lacking the AVD protein [pRL035+pRL016 in sRL002], or lacking CspRecT [pRL014+pAM014 in sRL002] show no detectable DGRec mutagenesis (Amut on average 1.56 for all these samples), confirming the essentiality of these components of the system.

### SbcB and RecJ DNA Repair Gene Shutdown Effect

On one targeted region, the deletions of sbcB and recJ exonucleases were assessed and show that their absence resulted in a reduction of DGRec efficiency of about 2-fold (Amut=97.0 with deletions [pRL014+pAM009 in sRL002] against 52.5 without deletions [pRL014+pAM009 in sRL003]).

Reverse Transcriptase Variants with Altered Adenine Infidelity

The Reverse Transcriptase variant I181N is functional and shows, as expected, a reduced level of DGRec mutagenesis ([pRL037+pRL031 in sRL002] Amut=9.0 compared to Amut=36.3 by the wild type Reverse Transcriptase [pRL014+pRL031 in sRL002]).

The Reverse Transcriptase variant R74N did not show detectable levels of DGRec mutagenesis [pRL036+pRL031 in sRL002](Amut=1.9), but would require additional controls to ensure that this variant is functional for the production of cDNA.

In conclusion, these results support previous results that these variants of the DGR reverse transcriptase have a reduced error rate at adenine positions in the RNA template.

pRL038 Backbone Compared to the pRL021 Backbone

These two plasmids have a cloning site allowing the addition of different TR sequences and their subsequent transcription as part of the DGR RNA. pRL038 is a medium copy plasmid, pRL021 is a high copy plasmid, and the DGR RNA surroundings are entirely different in those two plasmids, so that one could expect differences in the DGRec mutagenesis resulting from these two backbones. It was observed that SacB mutagenesis was 3 to 4 times higher when driven from the pRL021 backbone [pRL014+pAM009 in sRL002](Amut=97.0) than from the pRL038 backbone [pAM030+pAM001 in sRL002](Amut=37.3).

A caveat in this comparison, however, is that for the pRL038 DGR RNA expression, the partner plasmid was also producing a distinct DGR RNA with no targeted regions within the cell (pAM001 plasmid), which might have competed for the reverse transcriptase availability.

### Double Loci Targeting

Two DGR RNA were introduced in E. coli on two different backbones: pRL038 and pRL021. The first was programmed to target sacB and the second mCherry [pAM030+pAM011 in sRL002]. These DGR RNAs allowed to detect mutagenesis with good efficiency of both a sacB (Amut=33.14) and mCherry targeted regions (Amut=19.47), showing that two DGR RNA expressed simultaneously in the same cells can both be active.

### Template RNA Self-Targeting

Since the targeting in the DGRec system is solely driven by homology to the cDNA oligos, as opposed to the IMH requirement of the natural DGR systems, it was hypothesized that the DGRec system might be able to mutagenize the TR sequence carried on the DGRec plasmid, in addition to its target region within the E. coli chromosome. Indeed, it was detected self-targeting of the pRL021 backbone plasmid (Amut=93.5) and of the pRL038 backbone plasmid (Amut=113.8) within [pAM030+pAM011 in sRL002] cells.

Since the mutagenesis of the desired target could be obtained at high efficiency in some of those samples, the self-targeting of the DGR RNA is not an obstacle for the DGRec system. However, it should be taken into consideration in setups that would require longer mutagenesis induction times, as the TR sequence will likely mutate and degenerate over time, gradually losing its adenine nucleotides.

Note that it is also possible to take advantage of this phenomenon in a directed evolution setup where the TR and the target sequence will co-evolve to reach the desired phenotype. In such a setup, the sequence landscape explored by the DGRec system would initially be large, proportionally to the number of adenines in the TR. As adenines are progressively lost from the TR, the diversity of sequences that are explored in the target (VR) will reduce progressively. This phenomenon might help refine the desired activity without losing too many sequences to the exploration of invalid sequence space. Note that in this process, when an adenine in the TR is mutated to another base, this mutation will be transferred at a high rate to the target, thereby maintaining homology between TR and target during this evolutionary process. One can thus design TR sequences that contain A-rich segments, enabling a vast exploration of the sequence space and a progressive refinement over cycles of directed evolution.

### DGRec Mutagenesis on a Plasmid Target

It was possible to detect the mutagenesis of a target region located inside the GFP gene carried by a plasmid (pSC101 origin compatible with the DGRec plasmids, the pAM020 plasmid) (FIG. 7B). Interestingly, both orientations of the TR showed similar levels of mutagenesis (Amut=6.4 in forward direction [pRL014+pAM023+pAM020 in sRL001], Amut=14.9 in reverse direction [pRL014+pAM024+pAM020 in sRL001]), suggesting that the plasmid replication system produces single stranded DNA available for recombination on both strands. This is in contrast to the known preference of recombineering for the lagging strand when targeting the chromosome.

Note that the self-targeting of the DGR RNA described in the section above also occurs on a plasmid, demonstrating the ability of the DGRec system to mutagenize targeted regions on plasmids with different backbones (p15A ori and pUC on plasmids).

### Mutagenesis of an Integrated Prophage

Using a strain that was lysogenized with the k phage (strain sRL004), high mutagenesis levels inside the targeted region of that phage [pRL014+pRL029 in sRL004] were detected (Amut=65.3) (FIG. 7B).

### Example 5: TR and Targeted Region Design Rules

Next, the rules helping to properly design a TR sequence to tune the DGRec system towards producing the desired mutagenesis pattern were refined.

### Top and Bottom Strands Relation to the Lagging Strand

The Reverse Transcriptase can only randomize adenine nucleotides from the template RNA, but according to whether the TR sequence targets the coding or template strand of the target ORF, it can result in mutating either the A or T nucleotides of the coding sequence. This modifies the attainable amino acids, and which ones get mutated. If the target protein can be moved in forward or reverse orientation to be on the correct strand for mutagenesis, then even if limited to mutating the lagging strand, the DGRec system gives the option to target As or Ts.

### Attainable Amino Acids

“Attainable” amino acids were defined as the amino acids one can access using DGRec from a codon by mutating As (or Ts when targeting the reverse complement strand). For example, TTA can be mutated into 4 codons (TTA, TTG, TTC, TTT) and has 2 “attainable amino acids”: Leu (TTA/TTG) and Phe (TTC/TTT).

If randomizing Ts when targeting the reverse complement strand, attainable amino acids are very different. For instance, TTA has 13 “attainable amino acids reverse”.

The DGRec codon mutagenesis table (Table 2) shows, for each codon, the attainable amino acids, number of amino acids, and probability of attaining each amino acids (assuming random mutations), in forward and reverse orientation. There are large differences in the number of attainable amino acids between codons, even when they code for the same amino acids. For instance, AGA and CGC both code for Arginine, and have 6 and 1 attainable amino acids.

TABLE 2
DGRec codon mutagenesis table. For each codon, the table reports the number
of attainable amino acids (aas) with a TR in forward (fwd) direction
compared to its targeted ORF (randomizing adenines) and with a TR in
reverse (rvs) direction compared to its targeted ORF (randomizing thymines).
Codons that can be mutated by the DGRec towards stop codons are marked
with (*). These codons should be avoided in the TR design.

| | | Number of | Number of | | | Number of | Number of |
|---|---|---|---|---|---|---|---|
| | Amino | attainable | attainable | | Amino | attainable | attainable |
| Triplet | acid | aas fwd | aas rvs | Triplet | acid | aas fwd | aas rvs |
| TTT | F | 1 | 21 (*) | TCT | S | 1 | 4 |
| TTC | F | 1 | 15 | TCC | S | 1 | 4 |
| TTA | L | 2 | 13 (*) | TCA | S | 1 | 4 |
| TTG | L | 1 | 14 (*) | TCG | S | 1 | 4 |
| TAT | Y | 4 | 8 (*) | TGT | C | 1 | 6 (*) |
| TAC | Y | 4 | 4 | TGC | C | 1 | 4 |
| TAA | * | 7 (*) | 4 (*) | TGA | * | 3 (*) | 3 (*) |
| TAG | * | 4 (*) | 4 (*) | TGG | W | 1 | 3 |
| CTT | L | 1 | 5 | CCT | P | 1 | 1 |
| CTC | L | 1 | 4 | CCC | P | 1 | 1 |
| CTA | L | 1 | 4 | CCA | P | 1 | 1 |
| CTG | L | 1 | 4 | CCG | P | 1 | 1 |
| CAT | H | 4 | 2 | CGT | R | 1 | 1 |
| CAC | H | 4 | 1 | CGC | R | 1 | 1 |
| CAA | Q | 5 | 1 | CGA | R | 1 | 1 |
| CAG | Q | 4 | 1 | CGG | R | 1 | 1 |
| ATT | I | 4 | 7 | ACT | T | 4 | 1 |
| ATC | I | 4 | 4 | ACC | T | 4 | 1 |
| ATA | I | 5 | 4 | ACA | T | 4 | 1 |
| ATG | M | 3 | 4 | ACG | T | 4 | 1 |
| AAT | N | 15 | 2 | AGT | S | 4 | 2 |
| AAC | N | 15 | 1 | AGC | S | 4 | 1 |
| AAA | K | 21 (*) | 1 | AGA | R | 6 (*) | 1 |
| AAG | K | 14 (*) | 1 | AGG | R | 3 | 1 |
| GTT | V | 1 | 5 | GCT | A | 1 | 1 |
| GTC | V | 1 | 4 | GCC | A | 1 | 1 |
| GTA | V | 1 | 4 | GCA | A | 1 | 1 |
| GTG | V | 1 | 4 | GCG | A | 1 | 1 |
| GAT | D | 4 | 2 | GGT | G | 1 | 1 |
| GAC | D | 4 | 1 | GGC | G | 1 | 1 |
| GAA | E | 5 | 1 | GGA | G | 1 | 1 |
| GAG | E | 4 | 1 | GGG | G | 1 | 1 |

### Theoretical Library Size and ORF Recoding

The theoretical DNA library size for a given TR sequence can simply be approximated to 4\hat{ }(number of adenines), corresponding to the total number of DNA sequences that can be obtained by randomization of each adenine position within the TR sequence. For the theoretical peptide library size, the calculation depends on codons and their number of attainable amino acids. As a consequence, an ORF can be recoded to keep the same protein sequence but decrease or increase the size of the peptide library that can be attained.

### Recoding ORF for Low Diversity

While recoding to increase library size might seem like the obvious choice, there can be instances in which a portion of the targeted region of a protein must be conserved. There can also be instances in which the library size exceeds the selection capacity to screen it, making the recoding for low diversity useful when there is a need to comprehensively screen a (DNA) sequence space.

It was shown that it is also possible to recode a sequence in order to increase the peptide library size while keeping the DNA library size to a minimum, by removing “useless” codons such as CCA (Proline), which can mutate only to CCG, CCT or CCC, which all also code for Pro. These “useless” codons can decrease the recombineering efficiency of a cDNA oligo onto its targeted region, without adding any exploration of the protein sequence space.

### Internal Control

Of note, codons like CCA, which can mutate but only attain one amino acid, could also be used as a form of internal control to check for diversification without changing the amino acid sequence.

### Recoding for Adenines or for Thymines

There are significant differences between recoding for high/low diversity by changing adenines or thymines. This is due to two reasons:
After selecting the “best” codon (for high or low diversity), the average number of attainable codons is different for best adenines or best thymines codons (Table 3).
Not all amino acids have the same frequency inside proteins. For example, the high diversity generating amino acids when recoding adenines (asparagines (N) and lysines (K) having 15 and 14 attainable amino acids) tend to be frequent in proteins, while their counterpart when recoding thymines (Phenyalanine (F) with 15 attainable amino acids) is rarer.

TABLE 3
Mean number of attainable amino acids after
recoding for high or low diversity

| | A mutagenesis | T mutagenesis |
|---|---|---|
| Low diversity recoding | 3.5 aas | 2.7 aas |
| High diversity recoding | 4.5 aas | 4.3 aas |

Consequently, regardless of whether the targeted region is recoded for high or low diversity, mutating adenines generally leads to higher library sizes than mutating thymines.

### Enforcing Mismatch Between the TR and the Targeted ORF

In addition to recoding the ORF, the DGRec system offers the flexibility of adding mismatches between the TR sequence and the targeted region to “force” variability at any given amino acid whether its codon contains adenines or thymines.

### Saturation Mutagenesis

It is sometimes of interest to explore the largest possible number of amino acids at a few given positions. This might be achieved by optimizing for low diversity at positions that should stay constant and introducing adenines in the TR at positions to diversify. The design of the TR should avoid sequences that will lead to the introduction of stop codons in the targeted sequence. When the TR sequence matches that of the targeted coding strand this can be achieved using AAT or AAC codons. When the TR sequence matches that of the non-coding (template) strand, the TR should rather contain 5′-GAA-3′ at the desired position to diversify, which will lead to the generation of all 5′-NNC-3′ codons at the target position in the coding sequence. In this orientation the second codon with the highest diversity generation potential is obtained by using 5′-AAT-3′ in the TR which will lead to all 5′-ANN-3′ codons in the coding sequence, none of which are stop codons. Note that these codons reach amino-acids than cannot be encoded by the NNC or NNT codons (lysine and methionine). The use of multiple DGR RNAs in the same cell, targeting the same position but on different strands and with different codons can thus be advantageous to explore the full diversity of amino-acids while ensuring that no stop codons are introduced.

Using Stop Codons to Remove the WT Amino Acid Sequence from the Screen

It was shown that it is possible to introduce stop codons to “break” a targeted ORF, then fix it with DGRec mutagenesis, a strategy that might be useful to ensure the selection of variants only (removal of the wild type ORF sequence).

### Example 6: Generation of dCas9 Variants Using DGRec System

### Material and Methods

### 1. Screen Setup

### Recoding PIDs

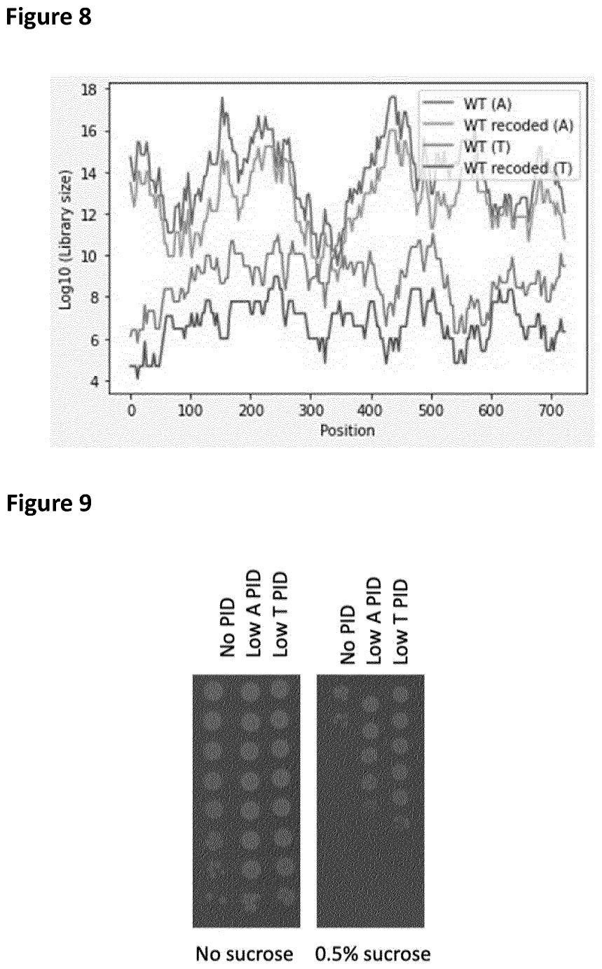

DGRec-based library generation uses diversity-generating reverse transcriptase which uses a programmable RNA template. The reverse transcriptase makes mutations at A positions. The generated mutagenic cDNA the recombines with the target sequence using the recombineering strategy which promotes the annealing of single stranded DNA to complementary sequences. The position of As in the DGRec RNA template can be designed to direct the diversification of codons of interest in the target gene. To maximize the control one have over which codon are diversified and which are not, it is desirable to eliminate the A positions on the target DNA strand. The inventors recoded the PAM-interacting domain (PID) of dCas9 to contain a low number of A bases on either the top strand of the ORF (Low A PID) or the bottom strand of the ORF (Low T PID). Recoding PIDs lowered the default library size complexity (FIG. 8).

### Positive Controls

The inventors first set up a system to select for functional dCas9 proteins and tested the functionality of the recoded PIDs. The system comprises a plasmid expressing dCas9 and a gRNA, targeted to an mCherry-SacB expression cassette. When functional dCas9 will silence the expression of the mCherry-SacB genes, making the E. coli less fluorescent while enabling growth on Sucrose which is toxic when SacB is expressed.

The mCherry-SacB expression cassette was derived from plasmid pFD148 and was integrated onto the genome of an E. coli MG1655:ΔrecJ,ΔsbcB strain, generating MG1655:ΔrecJ,ΔsbcB::SacB-Mcherry. Two dCas9 variants (optimised for low A or low T content) and a negative control (which contained a GFP protein instead of a PAM-interacting domain (PID) were grown then plated on LB with or without sucrose. It was found that a functional PAM caused a 1000× to 10 000× increase in colony forming units (FIG. 9) showing that the assay can be used to discriminate between plasmids containing a functional dCas9 and those which do not.

### 2. DGRec Plasmids and Strains

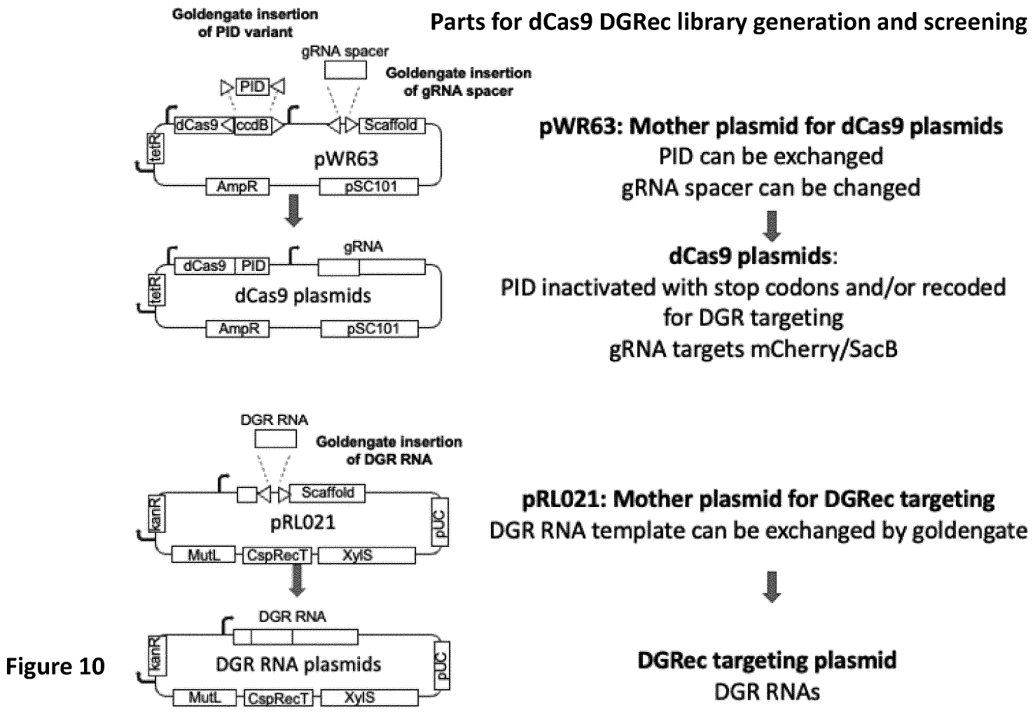

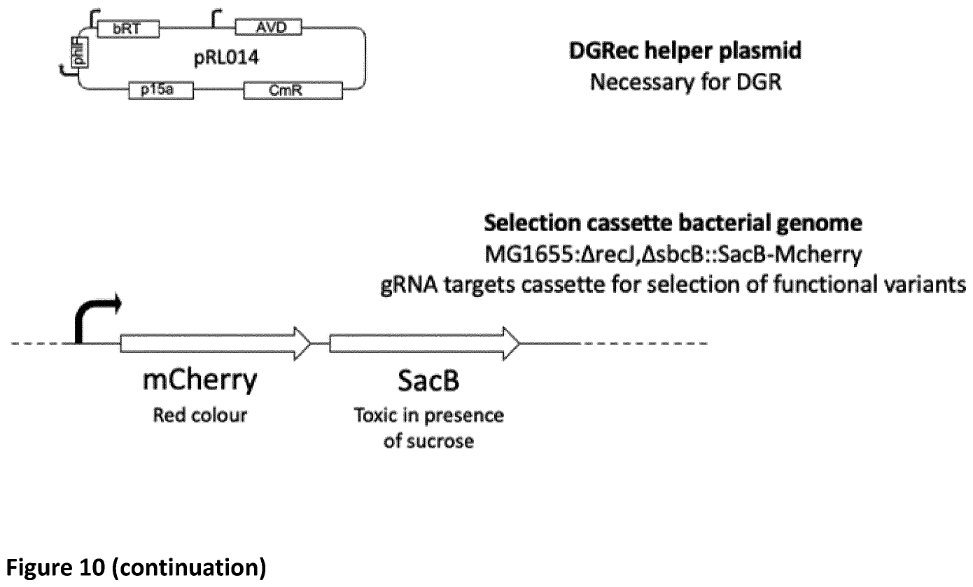

The screening setup consist in 4 parts: 3 compatible plasmids which contain the diversity-generating system and the dCas9 to be diversified, and a selection cassette which is integrated on the genome (FIG. 10). The selection cassette contains a constitutive promoter, a ribosome-binding site, and an operon coding for the fluorescent reporter mCherry and the counter-selection marker sacB, which is toxic in presence of sucrose. The cassette is inserted on the genome of an MG1655:ΔrecJ,ΔsbcB E. coli strain, as recJ and sbcB deletions increase the efficiency of DGRec.

The first plasmid contains a functional or a non-functional dCas9, and a gRNA targeting dCas9 to repress transcription of the selection cassette on the genome, allowing the discrimination between functional and non-functional dCas9 variants using either mCherry fluorescence or SacB-mediated toxicity on sucrose. Optionally, dCas9 is under the control of an inducible promoter.

The second plasmid, derived from pRL021, contains the DGR RNA, which targets the mutagenesis of the DGR system to the desired region of dCas9. The plasmid also expresses MutL and CspRecT, which increase recombineering efficiency and are part of the DGRec system, and XylS, which controls inducible expression of MutL and CspRecT by n-toluic acid.

The third plasmid expresses AVD and bRT, which form part of the DGR system, and PhlF which allows inducible expression of bRT.

To facilitate the construction of different versions of dCas9 variants targeted to different sites, a “mother” plasmid—pWR63—was constructed, which contains golden gate restriction sites instead of a PID domain of dCas9 as well as golden gate sites just upstream of a gRNA scaffold. This allows easy construction of new dCas9 plasmids. For construction of DGR RNAs, a “mother” plasmid (pRL021) is also used. If targeting DGR to a single site, pRL014 is used, but DGR can also be targeted to two sites simultaneously by cloning another DGR RNA into pRL038, which contains a DGR RNA cloning site as well as the parts contained on pRL014.

### 3. DGRec Setup

### Inserting Stop Codons in Cas9 to be Fixed by DGR

The DGR RNA is usually targeted to a region within dCas9 to create diversity at the chosen position. As any functional dCas9 should be selected for by the screening process, the system is typically started with a broken dCas9, where one or more stop codons have been inserted into the dCas9 position to be mutagenised. This way, only dCas9 variants where the stop codon was removed through DGR mutagenesis pass the selection process. The stop codon can be inserted at any position but choosing positions that can revert to the wild-type amino acid after mutagenesis of A bases, or positions that are known to be important for dCas9 binding, can be chosen to maximise chances of obtaining desired variants.

Using goldengate cloning into pWR63, the inventors constructed 6 dCas9 target plasmids containing PID variants and stop codons: pWR57 through 62. pWR57-59 (containing Cas9_T1, Cas9_T2 and Cas9_T3) are codon-optimised for low A content, and contain respectively: Y1141*, Y1141*+L1144*, and 51216*+L1220*. pWR60-63 (containing Cas9_T4, Cas9_T5 and Cas9_T6) are optimised for low T content, and contain respectively R1122*, R1122*+K1123*, and K1334+R1335*.

Choosing Regions with Low High Diversity, Choosing Regions with Known Interesting Amino Acids

The DGR RNA used as a template by DGRec consists of a Template Region (TR) inserted within constant regions of a DGR RNA. 21 different DGR RNAs (called DGR1 to DGR21) were constructed by inserting TRs into pRL021. The target regions of these TRs contain stop codons as described, which can be replaced by amino acid codons after the DGRec process. TRs were designed with lengths varying from 60 nt to 80 nt, and were either fully complementary to the target, or had extra A bases inserted at certain loci in order to increase library diversity. Some TRs contained an internal control, where an A nucleotide is added at the third position of codons where A mutagenesis will be silent. This internal control can be used to monitor the rate of diversification without the bias of selection.

### 4. DGRec+Screening

### Protocol

DGRec dCas9 library generation and screening starts with a single clone containing 3 plasmids: A dCas9 plasmid, a DGR RNA plasmid, and a DGRec helper. Alternatively, an in vitro generated library of DGR RNA plasmids, or a library of dCas9 plasmids, or both, can be used. The DGRec helper may contain a second DGR RNA, alternatively a library of DGR RNAs.

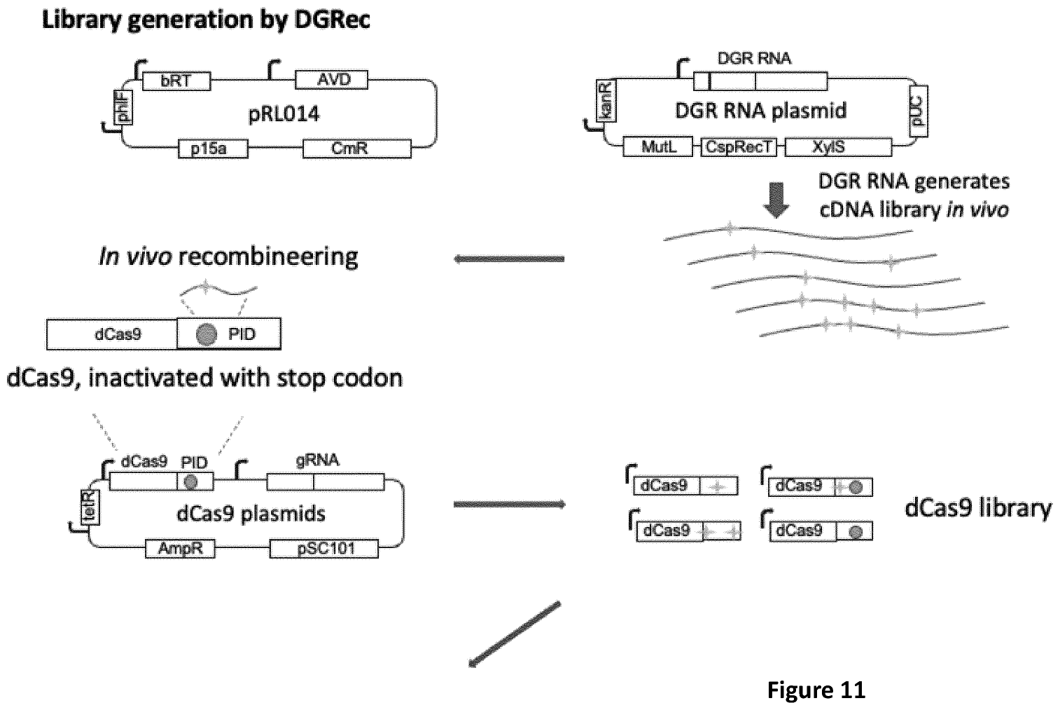

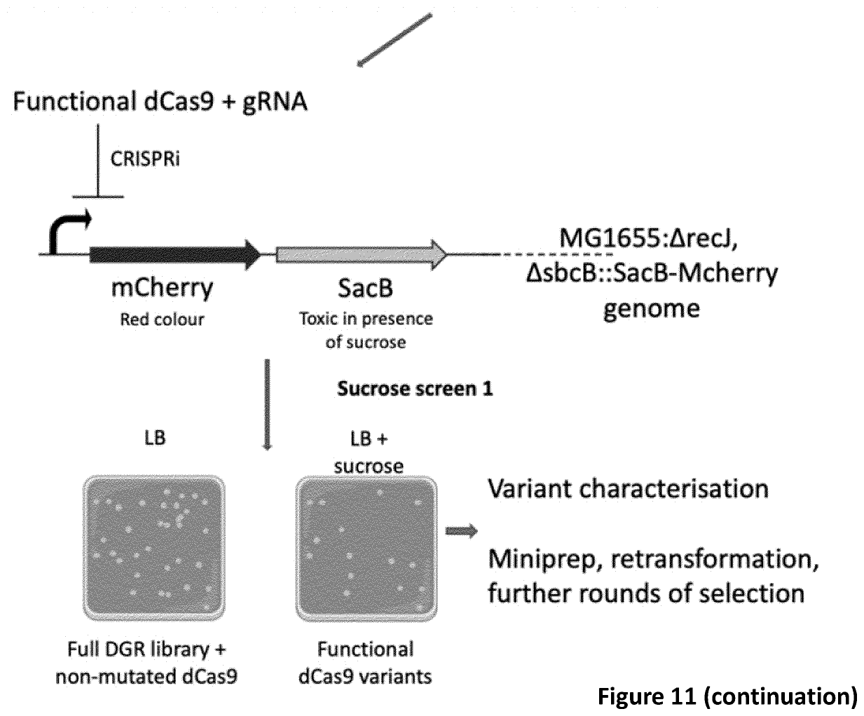

Clones or libraries are cultured overnight in LB at 37° C. with antibiotics (Carbenicillin 100 μg/mL, kanamycin 50 μg/mL, chloramphenicol 25 μg/mL). The next day, the culture is diluted 1:500 in 1 mL LB containing antibiotics supplemented with n-toluic acid and DAPG, grown overnight at 37° C., then rediluted again 1:500 in LB with inducers and antibiotics and grown overnight again, producing the DGRec library. For DGR1, 10 μL of culture was grown in 1 mL of LB overnight, then 100 and 10 μL of culture was plated on LB+carbenicillin (100 μg/mL) or LB+Carbenicillin+0.5% sucrose on 12×12 cm square plates. For DGRec3, 100 and 10 μL of the library was plated directly on LB+carbenicillin (100 μg/mL) or LB+Carbenicillin +0.5% sucrose. In parallel, spot drops of serially diluted cultures were plated in both conditions to count colony forming units (cfu). Single colonies of clones growing in either condition were then picked and sequenced by Sanger sequencing, while the rest of the plate was scraped off and diluted into LB, part of it was saved as DMSO stocks while plasmids were extracted from the rest (see FIG. 11).

### Further Rounds

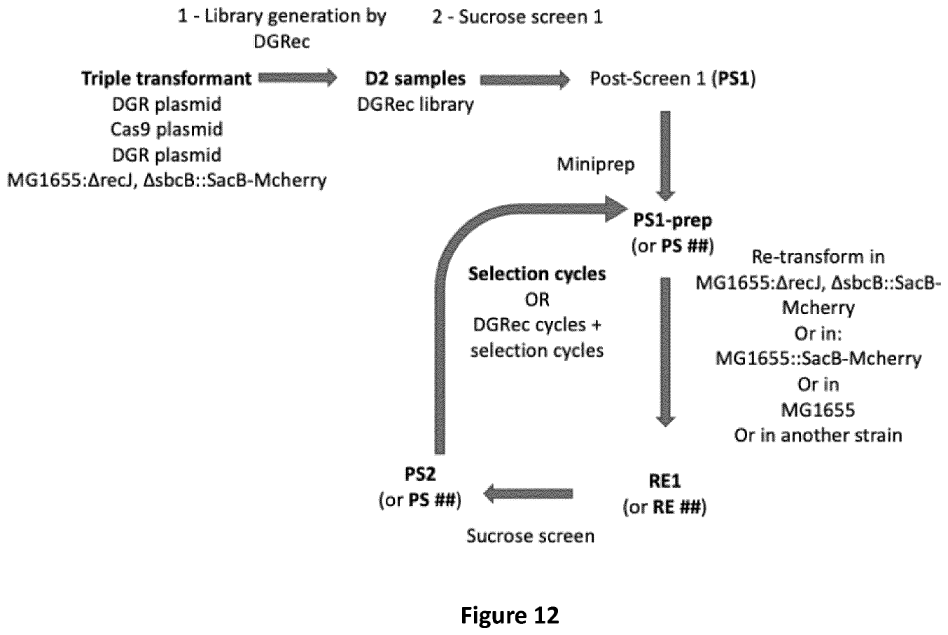

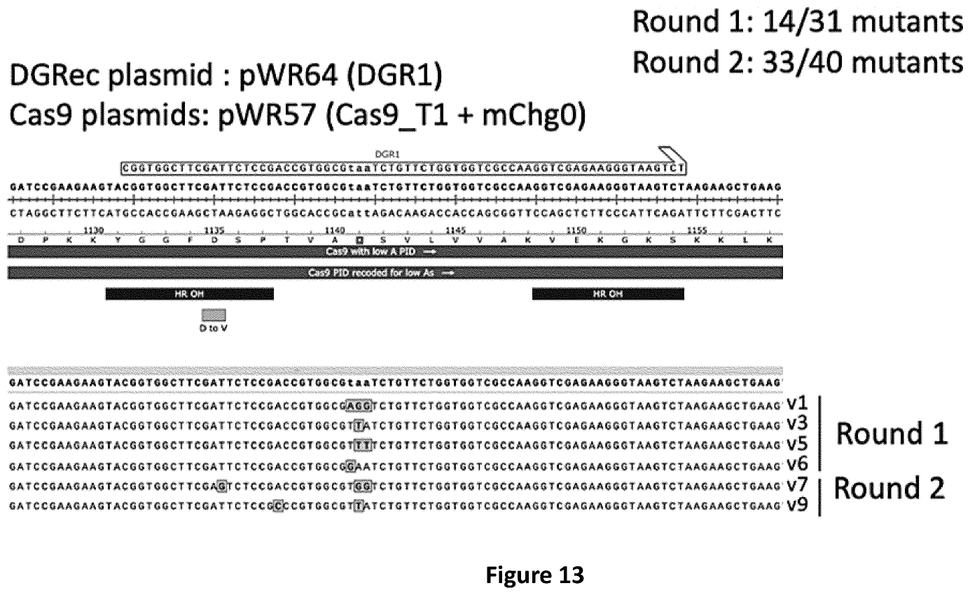

As SacB mutants can also arise spontaneously from the screen, but since these mutations are present on the genome, further rounds of selection can be carried out by extracting the Cas9 plasmid and re-transforming it into fresh MG1655:ΔrecJ,ΔsbcB::SacB-Mcherry cells. Further DGRec mutagenesis and selection on sucrose can then be carried out with the library obtained from the first round. Alternatively, if no further mutagenesis is required, the library can be transformed into MG1655: ::SacB-Mcherry or MG1655: ::SacB cells, or other strains containing a selection or screening marker targeted by dCas9, and further rounds of screening or selection without mutagenesis can be carried out (see FIG. 12).

Two rounds of selection were carried out with DGR1 after re-transformation into MG1655:ΔrecJ,ΔsbcB::SacB-Mcherry, while one round was carried out with DGR3.

### Results of First Screens: Variants Found

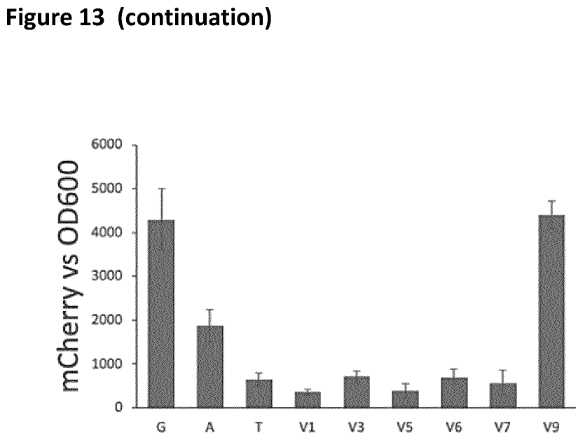

DGRec reaction 1 was carried out with pWR57 (Cas9_T1+mChgO), pWR64 (DGR1) and pRL014 as described above. DGR1 targets Cas9_T1, which contains one stop codon. After the first round, 31 clones were picked and sent for sequencing, and 14 were found to contain mutated amino acids in the target region, while none had mutations in any other region. A control DGRec library, which contained pWR57 and pWR64 but with pRL034 (which contains an inactive Reverse Transcriptase) was plated in parallel, but no mutants were found in 15 picked clones. This confirms that the generated mutants originate from DGRec mutagenesis and not from spontaneous mutation of the cas9 gene. Of the 14 clones with mutations, 4 variants were found, described in FIG. 13 and Table 4. A second round of selection was then carried out by re-transforming the purified library into fresh MG1655:ΔrecJ,ΔsbcB::SacB-Mcherry cells, growing overnight in LB, then plating again on LB+carbenicillin+0.5% sucrose. 40 clones were picked after this second screen, and 31 of these were found to contain mutations.

The variants were then characterised by growing a culture overnight in LB, in parallel to controls containing either an inactive dCas9 variant (G, containing NoPID_GFP) or active dCas9 variants (A or T, optimised for low A or T content respectively, containing Cas9PID_Recoded_low_A and Cas9PID_Recoded_low_T as PIDs), targeted with Cas9 gRNA mChgO. 5 of the variants were found to be active, repressing mCherry better than the original dCas9 protein from which they are derived.

TABLE 4
DGRec library 1 screening results

| Variant | | | STOP | Count | Count |
|---|---|---|---|---|---|
| (Round1) | D1135 | T1138 | (Y1141) | (/variants | (/total) |
| v1 | | | Arg | 5/14 | 5/31 |
| v3 | | | Leu | 7/14 | 7/31 |
| v5 | | | Phe | 1/14 | 1/31 |
| v6 | | | Glu | 1/14 | 1/31 |
| Variant | D1135 | T1138 | STOP | Count | Count |
| (Round2) | | | (Y1141) | (/variants) | (/total) |
| v1 | | | Arg | 1/33 | 1/40 |
| v7 | Glu | | Trp | 31/33 | 31/40 |
| v9 | | Pro | Leu | 1/33 | 1/40 |

These results show that DGRec can create a library of dCas9 variants in vivo starting from an inactivated dCas9, and that some of these variants may have a higher activity than the wild-type sequence.

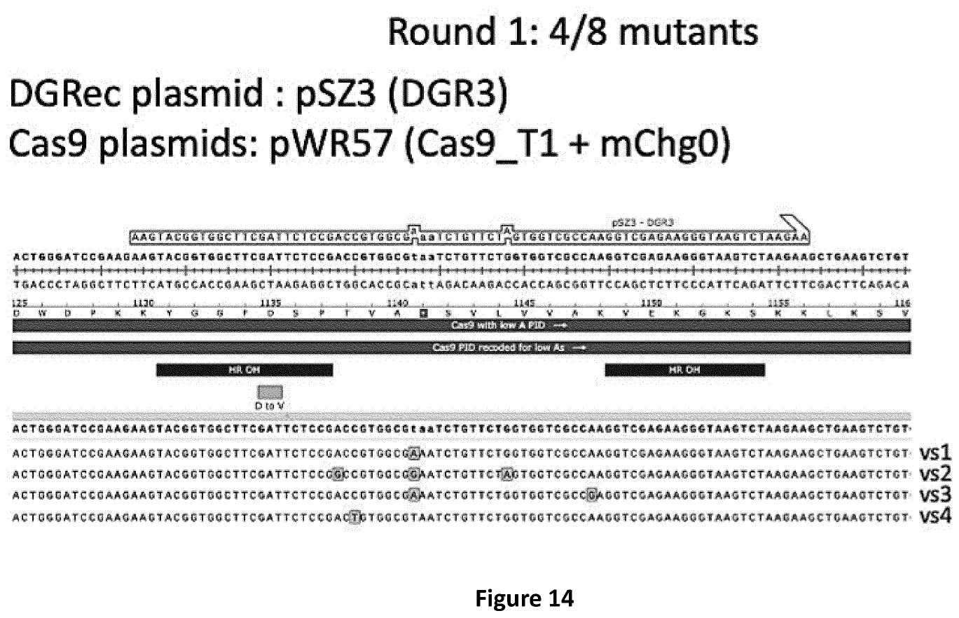

DGRec reaction 3 was carried out with pWR57 (Cas9_T1), pSZ3 (DGR3) and pRL014 as described above. DGR3 contains mismatches with the target, with one A base intended to create extra diversity at amino acid position 1141, and one internal control at position 1144. After one round of selection, 8 clones were picked and sent for sequencing, and 4 of which contained mutated amino acids in the target region, while none had mutations in any other region. Similarly, a control library targeted with a non-targeting DGR RNA (pAM001, containing DGR_control) was found to contain no mutants. All 4 variants had different sequences, with mutations described in FIG. 14 and Table 5.

TABLE 5
DGRec library 3 screening results

| | | STOP | | | Count |
|---|---|---|---|---|---|
| Variant | T1138 | (Y1141) | L1144 | K1144 | (/total) |
| vs1 | | Leu | | | ⅛ |
| vs2 | Ala | Glu | Leu (silent) | | ⅛ |
| vs3 | | Leu | | Glu | ⅛ |
| vs4 | Thr | Gly (tbc) | | | ⅛ |

TABLE 6
Strains and Plasmids.

| | |
|---|---|
| Description/relevant characteristics | Reference |

E. coli strains

| | | |
|---|---|---|
| MG1655 | F- lambda- ilvG- rfb-50 rph-1 derived | |
| | from E. coli K12 | |
| MG1655* | MG1655 ΔFhuA | |
| MG1655 ΔrecA | MG1655 recA::Tn10 | |
| MG1655:ΔrecJ, ΔsbcB | | This work |
| MG1655:ΔrecJ, | | This work |
| ΔsbcB::SacB-Mcherry | | |
| sRL001 | MG1655 ΔrecJ, ΔsbcB; recipient strain for DGRec | This work |
| | plasmids allowing targeted mutagenesis. | |
| sRL002 | MG1655 ΔrecJ, ΔsbcB, mCherry-sacB at λ site; Strain | This work |
| | for TR targeting sacB or mCherry. | |
| sRL003 | MG1655 mCherry-sacB at λ site; Strain for evaluation | This work |
| | of sbcB and recJ deletions. | |
| sRL004 | MG1655::λ, ΔrecJ, ΔsbcB ; Strain for mutagenesis of | This work |
| | the λ prophage | |

Plasmids

| | | |
|---|---|---|
| construction plasmids | | |
| pORTMAGE-Ec1 | Used as the source of recombineering module | [40] |
| | (CspRecT, mutL* under Pm promoter) | |
| pFD148 | derived from pOSIP-KL for mCherry-sacB | This work |
| | integration at λ site, Km^{R} | |
| pAM020 | sfGFP under Ptet inducible promoter, pSC101 | This work |
| | ori, Amp^{R}. | |

Reverse Transcriptase plasmids

| | | |
|---|---|---|
| pRL014 | RT under PhlF inducible promoter, Avd, p15A ori, Cm^{R}. | This work |
| pRL034 | pRL014, but RT with YMDD box in active site replaced | This work |
| | with residues SMAA | |
| pRL036 | pRL014, but RT with R74A mutation | This work |
| pRL037 | pRL014, but RT with I181N mutation | This work |
| pRL035 | pRL014, but Avd deleted | This work |

TR/Recombineering plasmids

| | | |
|---|---|---|
| pRL021-ccdB | DGR RNA with BsaI/ccdB cassette for Golden gate | This work |
| | assembly of TR, CspRecT-mutL* under Pm promoter, | |
| | pUC ori, Km^{R}. | |
| pRL016 | pRL021-ccdB with TR targeting sacB (residues | This work |
| | 20-43) (TR_RL016) | |
| pAM001 | pRL021-ccdB with the wild type B-PP1 phage TR | This work |
| | sequence (TR_AM001) | |
| pAM004 | pRL021-ccdB with a 40 bp TR targeting sacB | This work |
| | (residues 25-38) (TR_AM004) | |
| pAM007 | pRL021-ccdB with a 100 bp TR targeting sacB | This work |
| | (residues 10-43) (TR_AM007) | |
| pAM009 | pRL021-ccdB with TR targeting sacB active site | This work |
| | region (residues 235-237) (TR_AM009) | |
| pRL031 | pAM009 but with TR adding mismatch T > A at | This work |
| | nucleotide 4877 (TR_RL031) | |
| pAM010 | pRL021-ccdB with TR targeting sacB active site | This work |
| | region (residues 79-102) (TR_AM010) | |
| pAM011 | pRL021-ccdB with TR targeting mCherry (resdiues | This work |
| | 28-51) (TR_AM011) | |
| pAM014 | pRL016, but with CspRecT deleted (TR_RL016) | This work |
| pAM015 | pRL016, but with mutL* deleted (TR_RL016) | This work |
| pRL038-ccdB | pRL014, but with addition under a Pr promoter of | This work |
| | a DGR RNA with BsaI/ccdB cassette for Golden gate | |
| | assembly of TR. | |
| pAM030 | pRL038-ccdB with TR targeting sacB active site region | This work |
| | (residues 235-237) (TR_AM009) | |
| pAM021 | pRL021-ccdB with TR forward targeting lacZ (residues | This work |
| | 451-476) (TR_AM021) | |
| pAM022 | pRL021-ccdB with TR reverse targeting lacZ (residues | This work |
| | 451-476) (TR_AM022) | |
| pAM023 | pRL021-ccdB with TR forward targeting sfGFP (residues | This work |
| | 50-76) (TR_AM023) | |
| pAM024 | pRL021-ccdB with TR reverse targeting sfGFP (residues | This work |
| | 50-76) (TR_AM024) | |

Cm^{R}, chloramphenicol;
Km^{R}, kanamycin;
mutL*, mutL dominant negative allele;
RT, Bordetella phage B-PP1 DGR Reverse Transcriptase

TABLE 7
Description of the sequences

| Name | SEQ ID NO: | Sequence |
|---|---|---|
| RT canonical | 1 | QGXXXSP |
| motif | | |
| DGR RT | 2 | I/LGXXXSQ |
| canonical | | |
| motif | | |
| BPP-1 spacer | 3 | AAGGGCAGGCTGGGAAATAACGCTGCTGCGCTATTCGGCGGCAACTGGAA |
| RNA (DGR RNA) | | CAACACGTCGAACTCGGGTTCTCGCGCTGCGAACTGGAACAACGGGCCGT |
| sequence | | CGAACTCGAACGCGAACATCGGGGCGCGCGGCGTCTGTGCCCATCACCTT |
| | | CTTGCATGGCTCTGCCAACGCTACGGCTTGGCGGGCTGGCCTTTCCTCAA |
| | | TAGGTGGTCAGCCGGTTCTGTCCTGCTTCGGCGAACACGTTACACGGTTC |
| | | GGCAAAACGTCGATTACTGAAAATGGAAAGGCGGGGCCGACTTC |
| BPP-1 reverse | 4 | mgkrhrnlid qittwenlld ayrktshgkr rtwgylefke |
| transcriptase | | ydlanllalq aelkagnyer gpyreflvye pkprlisale |
| (bRT) protein | | fkdrlvqhal cnivapifea gllpytyacr pdkgthagvc |
| | | hvqaelrrtr athflksdfs kffpsidraa lyamidkkih |
| | | caatrillrv vlpdegvgip igsltsqlfa nvyggavdrl |
| | | lhdelkqrhw arymddivvl gddpeelrav fyrlrdfase |
| | | rlglkishwq vapvsrginf lgyriwpthk llrkssvkra |
| | | krkvanfikh gedeslqrfl aswsghaqwa dthnlftwme |
| | | eqygiach |
| BPP-1 Avd (bAvd) | 5 | mepieeatkc ydqmlivery ervisylypi aqsiprkhgv |
| protein | | aremflkcll gqvelfivag ksnqvsklya adaglamlrf |
| | | wlrflagiqk phamtphqve taqvliaevg rilgswiarv |
| | | nrkgqagk |
| CspRecT protein | 6 | mnqivkftdd sglavqvtpd dvrryicena tekevglflq |
| | | lcqtqrlnpf vkdaylvkyg gapasmitsy qvfnrracrd |
| | | anydgiksgv vvlrdgdvvh krgaacykka geeliggwae |
| | | vrfkdgreta yaevalddys tgksnwakmp gvmiekcaka |
| | | aawrlafpdt fqgmyaaeem dqaqqpeqvr aqaeqpvdlq |
| | | pirelfkpyc ehfgitpaeg mtavcgavga egmhsmteqq |
| | | arrarawmee emaapaveae yevvdegevf |
| Bordetella | 7 | ATGGGAAAGCGCCATCGTAACCTTATTGACCAAATCACCACCTGGGAAAA |
| phage B-PP1 | | TTTGTTAGATGCGTACCGTAAGACTAGCCACGGTAAGCGCCGCACTTGGG |
| Reverse | | GGTATCTTGAATTTAAGGAGTACGATTTGGCCAATTTGCTTGCACTTCAG |
| Transcriptase | | GCGGAGTTGAAGGCAGGCAATTACGAGCGCGGACCGTATCGCGAGTTTCT |
| gene* | | GGTTTACGAACCGAAACCCCGCTTGATTAGCGCACTGGAATTTAAAGATC |
| | | GTCTTGTTCAACACGCGCTGTGCAACATCGTTGCGCCAATCTTTGAAGCA |
| | | GGTTTGCTTCCATATACATACGCGTGTCGTCCGGATAAGGGGACCCATGC |
| | | AGGAGTGTGTCATGTTCAGGCAGAATTGCGTCGCACGCGCGCGACTCATT |
| | | TTTTGAAGAGTGACTTTAGCAAGTTCTTCCCAAGCATTGACCGTGCCGCT |
| | | CTTTATGCGATGATTGATAAAAAAATCCACTGCGCCGCTACACGTCGCCT |
| | | TTTGCGCGTTGTCCTGCCGGATGAGGGAGTTGGAATTCCCATTGGCAGCT |
| | | TAACCTCTCAGTTATTTGCCAACGTTTACGGGGGCGCGGTAGATCGTTTG |
| | | TTACACGACGAGTTAAAGCAGCGCCACTGGGCTCGTTACATGGATGACAT |
| | | TGTCGTACTTGGGGATGATCCAGAAGAACTGCGCGCGGTCTTCTATCGTT |
| | | TGCGTGATTTTGCGTCCGAACGCTTAGGTTTGAAGATTTCACATTGGCAG |
| | | GTAGCGCCTGTGTCTCGTGGAATCAATTTTCTTGGTTACCGCATCTGGCC |
| | | GACCCATAAATTGCTGCGTAAGAGTAGTGTGAAGCGCGCTAAGCGTAAGG |
| | | TGGCAAATTTCATCAAACACGGAGAAGACGAGTCACTGCAACGCTTCCTT |
| | | GCCTCGTGGTCGGGTCACGCCCAATGGGCGGACACTCACAATTTATTCAC |
| | | TTGGATGGAGGAGCAATATGGCATCGCGTGTCATTAA |
| Inactive RT | 8 | ATGGGAAAGCGCCATCGTAACCTTATTGACCAAATCACCACCTGGGAAAA |
| variant with | | TTTGTTAGATGCGTACCGTAAGACTAGCCACGGTAAGCGCCGCACTTGGG |
| SMAA residues | | GGTATCTTGAATTTAAGGAGTACGATTTGGCCAATTTGCTTGCACTTCAG |
| gene* | | GCGGAGTTGAAGGCAGGCAATTACGAGCGCGGACCGTATCGCGAGTTTCT |
| | | GGTTTACGAACCGAAACCCCGCTTGATTAGCGCACTGGAATTTAAAGATC |
| | | GTCTTGTTCAACACGCGCTGTGCAACATCGTTGCGCCAATCTTTGAAGCA |
| | | GGTTTGCTTCCATATACATACGCGTGTCGTCCGGATAAGGGGACCCATGC |
| | | AGGAGTGTGTCATGTTCAGGCAGAATTGCGTCGCACGCGCGCGACTCATT |
| | | TTTTGAAGAGTGACTTTAGCAAGTTCTTCCCAAGCATTGACCGTGCCGCT |
| | | CTTTATGCGATGATTGATAAAAAAATCCACTGCGCCGCTACACGTCGCCT |
| | | TTTGCGCGTTGTCCTGCCGGATGAGGGAGTTGGAATTCCCATTGGCAGCT |
| | | TAACCTCTCAGTTATTTGCCAACGTTTACGGGGGCGCGGTAGATCGTTTG |
| | | TTACACGACGAGTTAAAGCAGCGCCACTGGGCTCGTTCTATGGCGGCGAT |
| | | TGTCGTACTTGGGGATGATCCAGAAGAACTGCGCGCGGTCTTCTATCGTT |
| | | TGCGTGATTTTGCGTCCGAACGCTTAGGTTTGAAGATTTCACATTGGCAG |
| | | GTAGCGCCTGTGTCTCGTGGAATCAATTTTCTTGGTTACCGCATCTGGCC |
| | | GACCCATAAATTGCTGCGTAAGAGTAGTGTGAAGCGCGCTAAGCGTAAGG |
| | | TGGCAAATTTCATCAAACACGGAGAAGACGAGTCACTGCAACGCTTCCTT |
| | | GCCTCGTGGTCGGGTCACGCCCAATGGGCGGACACTCACAATTTATTCAC |
| | | TTGGATGGAGGAGCAATATGGCATCGCGTGTCATTAA |
| RT variant R74A | 9 | ATGGGAAAGCGCCATCGTAACCTTATTGACCAAATCACCACCTGGGAAAA |
| gene* | | TTTGTTAGATGCGTACCGTAAGACTAGCCACGGTAAGCGCCGCACTTGGG |
| | | GGTATCTTGAATTTAAGGAGTACGATTTGGCCAATTTGCTTGCACTTCAG |
| | | GCGGAGTTGAAGGCAGGCAATTACGAGCGCGGACCGTATCGCGAGTTTCT |
| | | GGTTTACGAACCGAAACCCGCATTGATTAGCGCACTGGAATTTAAAGATC |
| | | GTCTTGTTCAACACGCGCTGTGCAACATCGTTGCGCCAATCTTTGAAGCA |
| | | GGTTTGCTTCCATATACATACGCGTGTCGTCCGGATAAGGGGACCCATGC |
| | | AGGAGTGTGTCATGTTCAGGCAGAATTGCGTCGCACGCGCGCGACTCATT |
| | | TTTTGAAGAGTGACTTTAGCAAGTTCTTCCCAAGCATTGACCGTGCCGCT |
| | | CTTTATGCGATGATTGATAAAAAAATCCACTGCGCCGCTACACGTCGCCT |
| | | TTTGCGCGTTGTCCTGCCGGATGAGGGAGTTGGAATTCCCATTGGCAGCT |
| | | TAACCTCTCAGTTATTTGCCAACGTTTACGGGGGCGCGGTAGATCGTTTG |
| | | TTACACGACGAGTTAAAGCAGCGCCACTGGGCTCGTTACATGGATGACAT |
| | | TGTCGTACTTGGGGATGATCCAGAAGAACTGCGCGCGGTCTTCTATCGTT |
| | | TGCGTGATTTTGCGTCCGAACGCTTAGGTTTGAAGATTTCACATTGGCAG |
| | | GTAGCGCCTGTGTCTCGTGGAATCAATTTTCTTGGTTACCGCATCTGGCC |
| | | GACCCATAAATTGCTGCGTAAGAGTAGTGTGAAGCGCGCTAAGCGTAAGG |
| | | TGGCAAATTTCATCAAACACGGAGAAGACGAGTCACTGCAACGCTTCCTT |
| | | GCCTCGTGGTCGGGTCACGCCCAATGGGCGGACACTCACAATTTATTCAC |
| | | TTGGATGGAGGAGCAATATGGCATCGCGTGTCATTAA |
| RT variant | 10 | ATGGGAAAGCGCCATCGTAACCTTATTGACCAAATCACCACCTGGGAAAA |
| I181N gene* | | TTTGTTAGATGCGTACCGTAAGACTAGCCACGGTAAGCGCCGCACTTGGG |
| | | GGTATCTTGAATTTAAGGAGTACGATTTGGCCAATTTGCTTGCACTTCAG |
| | | GCGGAGTTGAAGGCAGGCAATTACGAGCGCGGACCGTATCGCGAGTTTCT |
| | | GGTTTACGAACCGAAACCCCGCTTGATTAGCGCACTGGAATTTAAAGATC |
| | | GTCTTGTTCAACACGCGCTGTGCAACATCGTTGCGCCAATCTTTGAAGCA |
| | | GGTTTGCTTCCATATACATACGCGTGTCGTCCGGATAAGGGGACCCATGC |
| | | AGGAGTGTGTCATGTTCAGGCAGAATTGCGTCGCACGCGCGCGACTCATT |
| | | TTTTGAAGAGTGACTTTAGCAAGTTCTTCCCAAGCATTGACCGTGCCGCT |
| | | CTTTATGCGATGATTGATAAAAAAATCCACTGCGCCGCTACACGTCGCCT |
| | | TTTGCGCGTTGTCCTGCCGGATGAGGGAGTTGGAATTCCCAACGGCAGCT |
| | | TAACCTCTCAGTTATTTGCCAACGTTTACGGGGGCGCGGTAGATCGTTTG |
| | | TTACACGACGAGTTAAAGCAGCGCCACTGGGCTCGTTACATGGATGACAT |
| | | TGTCGTACTTGGGGATGATCCAGAAGAACTGCGCGCGGTCTTCTATCGTT |
| | | TGCGTGATTTTGCGTCCGAACGCTTAGGTTTGAAGATTTCACATTGGCAG |
| | | GTAGCGCCTGTGTCTCGTGGAATCAATTTTCTTGGTTACCGCATCTGGCC |
| | | GACCCATAAATTGCTGCGTAAGAGTAGTGTGAAGCGCGCTAAGCGTAAGG |
| | | TGGCAAATTTCATCAAACACGGAGAAGACGAGTCACTGCAACGCTTCCTT |
| | | GCCTCGTGGTCGGGTCACGCCCAATGGGCGGACACTCACAATTTATTCAC |
| | | TTGGATGGAGGAGCAATATGGCATCGCGTGTCATTAA |
| Bordetella BPP- | 11 | TTATTTTCCGGCTTGTCCTTTACGGTTCACACGAGCAATCCAGGACCCTA |
| 1 phage Avd | | AGATACGGCCAACCTCCGCGATAAGCACCTGAGCAGTCTCGACCTGGTGC |
| gene* | | GGAGTCATGGCATGCGGCTTTTGAATGCCTGCCAAAAAGCGAAGCCAGAA |
| | | GCGTAACATCGCCAAACCCGCGTCAGCAGCATACAGTTTCGAGACCTGGT |
| | | TTGATTTACCTGCTACAATGAACAGTTCCACCTGTCCCAACAGACATTTC |
| | | AAAAACATTTCGCGTGCGACTCCATGTTTACGAGGGATTGACTGAGCAAT |
| | | GGGATACAAGTATGAGATGACGCGTTCATAGCGCTCTACAATCAACATCT |
| | | GATCGTAACACTTGGTAGCCTCTTCAATAGGTTCCAT |
| XylS (modified | 12 | TCAAGCCACTTCCTTTTTGCATTGACGCAGGGTGTCGGAAGGCAACTCGC |
| to remove BsaI | | CGAACGCGCTCCTATAGTTTTCAGCGAAGCGTCCCAAATGTAAGAAGCCG |
| gene | | TAGTCTAGGGCTATCTCAGTTATACTACGCACATTGGCACTGGGATCGTT |
| restriction | | CAAGCAGGCGCGGATGCTTTCGAGCTTGCGGTTGCGGATGTAGTTCTTCG |
| site) * | | GCGTGGTGCCGGCGTGCTTCTCGAACAAATTGTAGAGCGAGCGTGGACTC |
| | | ATCATCGCCAGCTCCGCTAACCGCTCAAGGCTGATATTCCGTTTGAGATT |
| | | CTCCTCAATGAATTGAACGACTCGCTCGAAAGACGGGTTACCTTTGCTGA |
| | | AAATTTCACGGCTGACATTGCTGCCCAGCATTTCGAGCAGCTTGGAAGCG |
| | | ATGATCCCCGCATAGTGCTCTTGGACCCGAGGCATCGACTTTGTATGTTC |
| | | CGCTTCGTCACAAACTAACCCGAGTAGATTGATAAAGCCATCGAGTTGCT |
| | | GGAGATTGTGTCGCGCGGCGAAACGGATACCCTCCCTCGGCTTGTGCCAA |
| | | TTGTTGTCACTGCACGCCCGATCAAGGACCACTGAGGGCAATTTAACGAT |
| | | AAATTTCTCGCAATCTTCTGAATAGGTCAGGTCGGCTTGGTCATCCGGAT |
| | | TGAGCAGCAATAGTTCGCCCGGCGCAAAATAGTGCTCCTGGCCATGGCCA |
| | | CGCCACAGGCAATGGCCTTTGAGTATTATTTGCAGATGATAACAGGTTTC |
| | | TAATCCAGGCGAGATTACCCTCACGCTACCGCCGTAGCTGATTCGACACA |
| | | GATCGAGGCATCCGAAGATTCTGTGGTGCAGCCTGCCTGCCGGGCGCCCG |
| | | CCCTTGGGCAGGCGAATAGAGTGCGTACCGACATACTGGTTAACATAATC |
| | | GGAGACTGCATAGGGCTCGGCGTGGACGAAGATCTGACTTTTCTCGTTCA |
| | | ATAAGCAAAAATCCAT |
| phlF promoter* | 13 | ATGGCACGTACCCCGTCACGTAGTAGCATTGGTAGCCTGCGTAGTCCGCA |
| | | TACCCATAAAGCAATTCTGACCAGTACCATCGAGATCCTGAAAGAATGTG |
| | | GTTATAGCGGACTGAGCATTGAAAGCGTTGCACGTCGTGCCGGAGCAAGC |
| | | AAACCGACCATTTATCGTTGGTGGACGAATAAAGCAGCACTGATTGCCGA |
| | | AGTGTATGAAAATGAAAGCGAACAGGTGCGTAAATTTCCGGATCTGGGTA |
| | | GCTTTAAAGCAGATCTGGATTTTTTACTGCGTAATTTATGGAAAGTTTGG |
| | | CGTGAAACTATTTGCGGTGAAGCATTTCGTTGTGTTATTGCAGAAGCTCA |
| | | GCTGGATCCTGCAACCCTGACCCAGTTAAAGGATCAATTTATGGAACGTC |
| | | GTCGTGAGATGCCGAAAAAACTGGTTGAAAATGCCATTAGCAATGGTGAA |
| | | CTGCCGAAAGATACCAATCGTGAACTTCTTCTGGATATGATTTTTGGTTT |
| | | TTGTTGGTATCGCCTGTTAACCGAACAGCTGACCGTTGAACAGGATATTG |
| | | AAGAATTTACCTTCCTTCTGATTAATGGTGTTTGTCCGGGTACTCAGCGT |
| cspRecT gene* | 14 | ATGAACCAAATCGTGAAGTTCACTGACGACTCTGGCCTGGCGGTTCAAGT |
| | | TACTCCAGACGATGTTCGCCGTTATATCTGTGAGAACGCTACTGAAAAAG |
| | | AGGTGGGCCTCTTTCTGCAACTCTGTCAGACTCAACGTCTGAATCCGTTT |
| | | GTGAAAGACGCTTACCTGGTGAAATACGGCGGTGCTCCAGCTTCTATGAT |
| | | TACTTCCTATCAAGTTTTTAACCGTCGCGCGTGTCGTGATGCTAACTATG |
| | | ATGGTATCAAATCTGGTGTGGTTGTTCTGCGTGACGGTGATGTTGTGCAT |
| | | AAACGTGGTGCTGCGTGCTACAAAAAGGCGGGTGAGGAGCTCATCGGTGG |
| | | TTGGGCGGAAGTTCGCTTTAAGGATGGCCGCGAGACTGCGTATGCTGAGG |
| | | TGGCGCTCGACGACTATTCCACCGGCAAATCTAATTGGGCGAAAATGCCG |
| | | GGTGTTATGATCGAAAAATGCGCGAAGGCTGCTGCTTGGCGCCTCGCGTT |
| | | CCCGGACACTTTTCAGGGCATGTACGCTGCGGAGGAAATGGATCAAGCGC |
| | | AACAGCCAGAACAGGTGCGCGCTCAGGCGGAGCAACCAGTGGATCTCCAG |
| | | CCAATCCGCGAACTCTTCAAGCCATATTGCGAACACTTCGGCATCACTCC |
| | | GGCTGAGGGTATGACTGCTGTTTGTGGTGCGGTGGGCGCTGAAGGCATGC |
| | | ACTCTATGACCGAGCAGCAAGCTCGCCGTGCTCGCGCTTGGATGGAGGAA |
| | | GAAATGGCTGCGCCAGCTGTGGAAGCGGAGTATGAGGTTGTTGACGAGGG |
| | | CGAGGTGTTTTAA |
| mutL gene* | 15 | ATGCCAATTCAGGTCTTACCGCCACAACTGGCGAACCAGATTGCCGCAGG |
| (E32K* | | TGAGGTGGTCGAGCGACCTGCGTCGGTAGTCAAAGAACTAGTGAAAAACA |
| | | GCCTCGATGCAGGTGCGACGCGTATCGATATTGATATCGAACGCGGTGGG |
| | | GCGAAACTTATCCGCATTCGTGATAACGGCTGCGGTATCAAAAAAGATGA |
| | | GCTGGCGCTGGCGCTGGCTCGTCATGCCACCAGTAAAATCGCCTCTCTGG |
| | | ACGATCTCGAAGCCATTATCAGCCTGGGCTTTCGCGGTGAGGCGCTGGCG |
| | | AGTATCAGTTCGGTTTCCCGCCTGACGCTCACTTCACGCACCGCAGAACA |
| | | GCAGGAAGCCTGGCAGGCCTATGCCGAAGGGCGCGATATGAACGTGACGG |
| | | TAAAACCGGCGGCGCATCCTGTGGGGACGACGCTGGAGGTGCTGGATCTG |
| | | TTCTACAACACCCCGGCGCGGCGCAAATTCCTGCGCACCGAGAAAACCGA |
| | | ATTTAACCACATTGATGAGATCATCCGCCGCATTGCGCTGGCGCGTTTCG |
| | | ACGTCACGATCAACCTGTCGCATAACGGTAAAATTGTGCGTCAGTACCGC |
| | | GCAGTGCCGGAAGGCGGGCAAAAAGAACGGCGCTTAGGCGCGATTTGCGG |
| | | CACCGCTTTTCTTGAACAAGCGCTGGCGATTGAATGGCAACACGGCGATC |
| | | TCACGCTACGCGGCTGGGTGGCCGATCCAAATCACACCACGCCCGCACTG |
| | | GCAGAAATTCAGTATTGCTACGTGAACGGTCGCATGATGCGCGATCGCCT |
| | | GATCAATCACGCGATCCGCCAGGCCTGCGAAGACAAACTGGGGGCCGATC |
| | | AGCAACCGGCATTTGTGTTGTATCTGGAGATCGACCCACATCAGGTGGAC |
| | | GTCAACGTGCACCCCGCCAAACACGAAGTGCGTTTCCATCAGTCGCGTCT |
| | | GGTGCATGATTTTATCTATCAGGGCGTGCTGAGCGTGCTACAACAGCAAC |
| | | TGGAAACGCCGCTACCGCTGGACGATGAACCCCAACCTGCACCGCGTTCC |
| | | ATTCCGGAAAACCGCGTGGCGGCGGGGCGCAATCACTTTGCAGAACCGGC |
| | | AGCTCGTGAGCCGGTAGCTCCGCGCTACACTCCTGCGCCAGCATCAGGCA |
| | | GTCGTCCGGCTGCCCCCTGGCCGAATGCGCAGCCAGGCTACCAGAAACAG |
| | | CAAGGTGAAGTGTATCGCCAGCTTTTGCAAACGCCCGCGCCGATGCAAAA |
| | | ATTAAAAGCGCCGGAACCGCAGGAACCTGCACTTGCGGCGAACAGTCAGA |
| | | GTTTTGGTCGGGTACTGACTATCGTCCATTCCGACTGTGCGTTGCTGGAG |
| | | CGCGACGGCAACATTTCACTTTTATCCTTGCCAGTGGCAGAACGTTGGCT |
| | | GCGTCAGGCACAATTGACGCCGGGTGAAGCGCCCGTTTGCGCCCAGCCGC |
| | | TGCTGATTCCGTTGCGGCTAAAAGTTTCTGCCGAAGAAAAATCGGCATTA |
| | | GAAAAAGCGCAGTCTGCCCTGGCGGAATTGGGTATTGATTTCCAGTCAGA |
| | | TGCACAGCATGTGACCATCAGGGCAGTGCCTTTACCCTTACGCCAACAAA |
| | | ATTTACAAATCTTGATTCCTGAACTGATAGGCTACCTGGCGAAGCAGTCC |
| | | GTATTCGAACCTGGCAATATTGCGCAGTGGATTGCACGAAATCTGATGAG |
| | | CGAACATGCGCAGTGGTCAATGGCACAGGCCATAACCCTGCTGGCGGACG |
| | | TGGAACGGTTATGTCCGCAACTTGTGAAAACGCCGCCGGGTGGTCTGTTA |
| | | CAATCTGTTGATTTACATCCGGCGATAAAAGCCCTGAAAGATGAGTGA |
| Engineered DGR | 16 | AAGGGCAGGCTGGGAAATAACGAGACCTGAATTGCGCGCAATTAACCCTC |
| Spacer RNA+ccdB | | ACTAAAGGGAACAAAAGCTGGAGCTCTTATATTCCCCAGAACATCAGGTT |
| | | AATGGCGTTTTTGATGTCATTTTCGCGGTGGCTGAGATCAGCCACTTCTT |
| | | CCCCGATAACGGACACCGGCACACTGGCCATATCGGTGGTCATCATGCGC |
| | | CAGCTTTCATCCCCGATATGCACCACCGGGTAAAGTTCACGGGAGACTTT |
| | | ATCTGACAGCAGACGTGCACTGGCCAGGGGGATCACCATCCGTCGCCCGG |
| | | GCGTGTCAATAATATCACTCTGTACATCCACAAACAGACGATAACGGCTC |
| | | TCTCTTTTATAGGTGTAAACCTTAAACTGCATCGTTTCACTCCATCCAAA |
| | | AAAACGGGTATGGAGAAACAGTAGAGAGTTGCGATAAAAAGCGTCAGGTA |
| | | GGATCCGCTGGTCTCATCTGTGCCCATCACCTTCTTGCATGGCTCTGCCA |
| | | ACGCTACGGCTTGGCGGGCTGGCCTTTCCTCAATAGGTGGTCAGCCGGTT |
| | | CTGTCCTGCTTCGGCGAACACGTTACACGGTTCGGCAAAACGTCGATTAC |
| | | TGAAAATGGAAAGGCGGGGCCGACTTC |
| pRL014 | 17 | GAAGATCATCTTATTAATCAGATAAAATATTTCTAGATTTCAGTGCAATT |
| (4421 bp) | | TATCTCTTCAAATGTAGCACgattttacggctagctcagtcctaggtaca |
| | | atgctagcgaatcattaaagaggagaaaggtactATGGCACGTACCCCGT |
| | | CACGTAGTAGCATTGGTAGCCTGCGTAGTCCGCATACCCATAAAGCAATT |
| | | CTGACCAGTACCATCGAGATCCTGAAAGAATGTGGTTATAGCGGACTGAG |
| | | CATTGAAAGCGTTGCACGTCGTGCCGGAGCAAGCAAACCGACCATTTATC |
| | | GTTGGTGGACGAATAAAGCAGCACTGATTGCCGAAGTGTATGAAAATGAA |
| | | AGCGAACAGGTGCGTAAATTTCCGGATCTGGGTAGCTTTAAAGCAGATCT |
| | | GGATTTTTTACTGCGTAATTTATGGAAAGTTTGGCGTGAAACTATTTGCG |
| | | GTGAAGCATTTCGTTGTGTTATTGCAGAAGCTCAGCTGGATCCTGCAACC |
| | | CTGACCCAGTTAAAGGATCAATTTATGGAACGTCGTCGTGAGATGCCGAA |
| | | AAAACTGGTTGAAAATGCCATTAGCAATGGTGAACTGCCGAAAGATACCA |
| | | ATCGTGAACTTCTTCTGGATATGATTTTTGGTTTTTGTTGGTATCGCCTG |
| | | TTAACCGAACAGCTGACCGTTGAACAGGATATTGAAGAATTTACCTTCCT |
| | | TCTGATTAATGGTGTTTGTCCGGGTACTCAGCGTTAACTAGGCCATAATC |
| | | GCTACCAAATTCCAGAAAACAGACGCTTTCGAGCGTCTTTTTTCGTTTTG |
| | | GTCACGACGTACTGAATCTGATTCGTTACCAATTGACATGATACGAAACG |
| | | TACCGTATCGTTAAGGTGGAGGCATATCAAAGGACGAGTGCAGGTGGCAA |
| | | AAATGGGAAAGCGCCATCGTAACCTTATTGACCAAATCACCACCTGGGAA |
| | | AATTTGTTAGATGCGTACCGTAAGACTAGCCACGGTAAGCGCCGCACTTG |
| | | GGGGTATCTTGAATTTAAGGAGTACGATTTGGCCAATTTGCTTGCACTTC |
| | | AGGCGGAGTTGAAGGCAGGCAATTACGAGCGCGGACCGTATCGCGAGTTT |
| | | CTGGTTTACGAACCGAAACCCCGCTTGATTAGCGCACTGGAATTTAAAGA |
| | | TCGTCTTGTTCAACACGCGCTGTGCAACATCGTTGCGCCAATCTTTGAAG |
| | | CAGGTTTGCTTCCATATACATACGCGTGTCGTCCGGATAAGGGGACCCAT |
| | | GCAGGAGTGTGTCATGTTCAGGCAGAATTGCGTCGCACGCGCGCGACTCA |
| | | TTTTTTGAAGAGTGACTTTAGCAAGTTCTTCCCAAGCATTGACCGTGCCG |
| | | CTCTTTATGCGATGATTGATAAAAAAATCCACTGCGCCGCTACACGTCGC |
| | | CTTTTGCGCGTTGTCCTGCCGGATGAGGGAGTTGGAATTCCCATTGGCAG |
| | | CTTAACCTCTCAGTTATTTGCCAACGTTTACGGGGGCGCGGTAGATCGTT |
| | | TGTTACACGACGAGTTAAAGCAGCGCCACTGGGCTCGTTACATGGATGAC |
| | | ATTGTCGTACTTGGGGATGATCCAGAAGAACTGCGCGCGGTCTTCTATCG |
| | | TTTGCGTGATTTTGCGTCCGAACGCTTAGGTTTGAAGATTTCACATTGGC |
| | | AGGTAGCGCCTGTGTCTCGTGGAATCAATTTTCTTGGTTACCGCATCTGG |
| | | CCGACCCATAAATTGCTGCGTAAGAGTAGTGTGAAGCGCGCTAAGCGTAA |
| | | GGTGGCAAATTTCATCAAACACGGAGAAGACGAGTCACTGCAACGCTTCC |
| | | TTGCCTCGTGGTCGGGTCACGCCCAATGGGCGGACACTCACAATTTATTC |
| | | ACTTGGATGGAGGAGCAATATGGCATCGCGTGTCATTAATAACGTTAAAG |
| | | TCAGTTTCACCTGTTTTACGTTAAAACCCGCTTCGGCGGGTTTTTACTTT |
| | | TGGtttAGCCGAACGCCCCAAAAAGCCTCGCTTTCAGCACCTGTCGTTTC |
| | | CTTTCTTTTCAGAGGGTATTTTAAATAAAAACATTAAGTTATGACGAAGA |
| | | AGAACGGAAACGCCTTAAACCGGAAAATTTTCATAAATAGCGAAAACCCG |
| | | CGAGGTCGCCGCCCCGTAACCTGTCGGATCACCGGAAAGGACCCGTAAAG |
| | | TGATAATGATTATCATCTACATATCACAACGTGCGTAAAGGGACTagtgg |
| | | atGtttGATCTCAAAAAAAGCACCTTATTTTCCGGCTTGTCCTTTACGGT |
| | | TCACACGAGCAATCCAGGACCCTAAGATACGGCCAACCTCCGCGATAAGC |
| | | ACCTGAGCAGTCTCGACCTGGTGCGGAGTCATGGCATGCGGCTTTTGAAT |
| | | GCCTGCCAAAAAGCGAAGCCAGAAGCGTAACATCGCCAAACCCGCGTCAG |
| | | CAGCATACAGTTTCGAGACCTGGTTTGATTTACCTGCTACAATGAACAGT |
| | | TCCACCTGTCCCAACAGACATTTCAAAAACATTTCGCGTGCGACTCCATG |
| | | TTTACGAGGGATTGACTGAGCAATGGGATACAAGTATGAGATGACGCGTT |
| | | CATAGCGCTCTACAATCAACATCTGATCGTAACACTTGGTAGCCTCTTCA |
| | | ATAGGTTCCATagaaactttctcctctttaataCTAGTattatacctagg |
| | | actgagctagctgtcagTCGGGTAGCACCAGAAGTCTATAGCATGtgcat |
| | | aCCTTTGGTCGAAAAAAAAAGCCCGCACTGTCAGGTGCGGGCTTTTTTCa |
| | | GTGTTTCCttgccggaTTACGCCCCGCCCTGCCACTCATCGCAGTATTGT |
| | | TGTAATTCATTAAGCATTCTGCCGACATGGAAGCCATCACAAACGGCATG |
| | | ATGAACTTGGATCGCCAGTGGCATTAACACCTTGTCGCCTTGCGTATAAT |
| | | ATTTTCCCATAGTGAAAACGGGGGCGAAGAAGTTGTCCATATTTGCTACG |
| | | TTTAAATCAAAACTGGTGAAACTCACCCACGGATTGGCACTGACGAAAAA |
| | | CATATTTTCGATAAACCCTTTAGGGAAATATGCTAAGTTTTCACCGTAAC |
| | | ACGCCACATCTTGACTATATATGTGTAGAAACTGCCGGAAATCGTCGTGG |
| | | TATTCTGACCAGAGCGATGAAAACGTTTCAGTTTGCTCATGGAAAACGGT |
| | | GTAACAAGGGTGAACACTATCCCATATCACCAGCTCACCGTCTTTCATTG |
| | | CCATACGAAACTCCGGATGTGCATTCATCAGGCGGGCAAGAATGTGAATA |
| | | AAGGCCGGATAAAACTTGTGCTTATTTTTCTTTACGGTTTTTAAAAAGGC |
| | | CGTAATATCCAGCTGAACGGTTTGGTTATAGGTGCACTGAGCAACTGACT |
| | | GGAATGCCTCAAAATGTTCTTTACGATGCCATTGACTTATATCAACTGTA |
| | | GTATATCCAGTGATTTTTTTCTCCATTTTAGCTTCCTTAGCTTGCGAAAT |
| | | CTCGATAACTCAAAAAATAGTAGTGATCTTATTTCATTATGGTGAAAGTT |
| | | GTCTTACGTGCAACATTTTCGCAAAAAGTTGGCGCTTTATCAACACTGTC |
| | | CCTCCTGTTCAGCTACCGGCCAGCCTCGCAGAGCAGGATTCCCGTTGAGC |
| | | ACCGCCAGGTGCGAATAAGGGACAGTGAAGAAGGAACACCCGCTCGCGGG |
| | | TGGGCCTACTTCACCTATCCTGCCCGGCTGACGCCGTTGGATACACCAAG |
| | | GAAAGTCTACACGAACCCTTTGGCAAAATCCTGTATATCGTGCGAAAAAG |
| | | GATGGATATACCGAAAAAATCGCTATAATGACCCCGAAGCAGGGTTATGC |
| | | AGCGGAAAAGCGCTGGTACCCAATTCGCCCTATAGTGAGTCTCCTGGAAG |
| | | TGAGAGGGCCGCGGCAAAGCCGTTTTTCCATAGGCTCCGCCCCCCTGACA |
| | | AGCATCACGAAATCTGACGCTCAAATCAGTGGTGGCGAAACCtGACAGGA |
| | | CTATAAAGATACCAGGCGTTTCCCCCTGGCGGCTCCCTCGTGCGCTCTCC |
| | | TGTTCCTGCCTTTCGGTTTACCGGTGTCATTCCGCTGTTATGGCCGCGTT |
| | | TGTCTCATTCCACGCCTGACACTCAGTTCCGGGTAGGCAGTTCGCTCCAA |
| | | GCTGGACTGTATGCACGAACCCCCCGTTCAGTCCGACCGCTGCGCCTTAT |
| | | CCGGTAACTATCGTCTTGAGTCCAACCCGGAAAGACATGCAAAAGCACCA |
| | | CTGGCAGCAGCCACTGGTAATTGATTTAGAGGAGTTAGTCTTGAAGTCAT |
| | | GCGCCGGTTAAGGCTAAACTGAAAGGACAAGTTTTGGTGACTGCGCTCCT |
| | | CCAAGCCAGTTACCTCGGTTCAAAGAGTTGGTAGCTCAGAGAACCTTCGA |
| | | AAAACCGCCCTGCAAGGCGGTTTTTTCGTTTTCAGAGCAAGAGATTACGC |
| | | GCAGACCAAAACGATCTCAA |
| pRL021-ccdB | 18 | GAAGATCCTTTGATCTTTTCTACGGGGTCTGACGCTCAGTGGAACGAAAA |
| (7924 bp) | | CTCACGTTAAGGGATTTTGGTCATGACTAGTGCTTGGAGAAGGCCATCCT |
| | | GACGGATGGCCTTTTatgcctttaattaaacgttcgtaatcaagccactt |
| | | cctttttgcattgacgcagggtgtcggaaggcaactcgccgaacgcgctc |
| | | ctatagttttcagcgaagcgtcccaaatgtaagaagccgtagtctagggc |
| | | tatctcagttatactacgcacattggcactgggatcgttcaagcaggcgc |
| | | ggatgctttcgagcttgcggttgcggatgtagttcttcggcgtggtgccg |
| | | gcgtgcttctcgaacaaattgtagagcgagcgtggactcatcatcgccag |
| | | ctccgctaaccgctcaaggctgatattccgtttgagattctcctcaatga |
| | | attgaacgactcgctcgaaagacgggttacctttgctgaaaatttcacgg |
| | | ctgacattgctgcccagcatttcgagcagcttggaagcgatgatccccgc |
| | | atagtgctcttggacccgaggcatcgactttgtatgttccgcttcgtcac |
| | | aaactaacccgagtagattgataaagccatcgagttgctggagattgtgt |
| | | cgcgcggcgaaacggataccctccctcggcttgtgccaattgttgtcact |
| | | gcacgcccgatcaaggaccactgagggcaatttaacgataaatttctcgc |
| | | aatcttctgaataggtcaggtcggcttggtcatccggattgagcagcaat |
| | | agttcgcccggcgcaaaatagtgctcctggccatggccacgccacaggca |
| | | atggcctttgagtattatttgcagatgataacaggtTtctaatccaggcg |
| | | agattaccctcacgctaccgccgtagctgattcgacacagatcgaggcat |
| | | ccgaagattctgtggtgcagcctgcctgccgggcgcccgcccttgggcag |
| | | gcgaatagagtgcgtaccgacatactggttaacataatcggagactgcat |
| | | agggctcggcgtggacgaagatctgacttttctcgttcaataagcaaaaa |
| | | tccatagttcacggttctcttattttaatgtgggctgcttggtgtgatgt |
| | | agaaaggcgccaagtcgatgaaaatgcatctcgacgtgatgcgtatacgg |
| | | gttacccccattgccacgttgcgccatcctttttgcaatcagtgaccact |
| | | tttccaagcaaaaataacgccaagcagaacgaagacgttctttttaagaa |
| | | gcgagaacaccagaagttcgtgctgtcggggcatggggcgacgaattggc |
| | | ggataaaggggatctgctggatattacggcctttttaaagaccgtaaaga |
| | | aaaataagcacaagttttatccggcctttattcacattcttgcccgcctg |
| | | atgaatgctcatccgtaattacgtatggcaatgaaagacggtgagctggt |
| | | gatatgggatagtgttcacccttgttacaccgttttccatgagcaaactg |
| | | aaacgttttcatcgctctggagtgaataccacgacgatttccggcagttt |
| | | ctacacatatattcgcaagatgtggcgtgttacggtgaaaacctggccta |
| | | tttccctaaagggtttattgagaatatgtttttcgtctcagccaatccct |
| | | gggtgagtttcaccagttttgatttaaacgtggccaatatggacaacttc |
| | | ttcgcccccgttttcaccatgcatgggaattagcttgatctgaccaacga |
| | | ccggtagcggagctatccaacggcggtataccaggaaaacacacagcagg |
| | | tacatcagaacagtaccatgactgaagaacaaatagttttttcctgatcc |
| | | ataaagcagaacggcctgctccatgacaaatctggctccccaactaatgc |
| | | cccatgcagccagcataaccagcataaagtgcagtgtccggtttgatagg |
| | | gataagtccagccttgcaagaagcggatacaggagtgcaaaaaatggcta |
| | | tctctagtaaggcctaccccttaggctttatgcaacagaaacaataataa |
| | | tggagtcatgaccatgcctaggccgcgggccgcgcgaattcagaaggaga |
| | | atataccatgaaccaaatcgtgaagttcactgacgactctggcctggcgg |
| | | ttcaagttactccagacgatgttcgccgttatatctgtgagaacgctact |
| | | gaaaaagaggtgggcctctttctgcaactctgtcagactcaacgtctgaa |
| | | tccgtttgtgaaagacgcttacctggtgaaatacggcggtgctccagctt |
| | | ctatgattacttcctatcaagtttttaaccgtcgcgcgtgtcgtgatgct |
| | | aactatgatggtatcaaatctggtgtggttgttctgcgtgacggtgatgt |
| | | tgtgcataaacgtggtgctgcgtgctacaaaaaggcgggtgaggagctca |
| | | tcggtggttgggcggaagttcgctttaaggatggccgcgagactgcgtat |
| | | gctgaggtggcgctcgacgactattccaccggcaaatctaattgggcgaa |
| | | aatgccgggtgttatgatcgaaaaatgcgcgaaggctgctgcttggcgcc |
| | | tcgcgttcccggacacttttcagggcatgtacgctgcggaggaaatggat |
| | | caagcgcaacagccagaacaggtgcgcgctcaggcggagcaaccagtgga |
| | | tctccagccaatccgcgaactcttcaagccatattgcgaacacttcggca |
| | | tcactccggctgagggtatgactgctgtttgtggtgcggtgggcgctgaa |
| | | ggcatgcactctatgaccgagcagcaagctcgccgtgctcgcgcttggat |
| | | ggaggaagaaatggctgcgccagctgtggaagcggagtatgaggttgttg |
| | | acgagggcgaggtgttttaaggatccaggagaatacacatgccaattcag |
| | | gtcttaccgccacaactggcgaaccagattgccgcaggtgaggtggtcga |
| | | gcgacctgcgtcggtagtcaaagaactagtgaaaaacagcctcgatgcag |
| | | gtgcgacgcgtatcgatattgatatcgaacgcggtggggcgaaacttatc |
| | | cgcattcgtgataacggctgcggtatcaaaaaagatgagctggcgctggc |
| | | gctggctcgtcatgccaccagtaaaatcgcctctctggacgatctcgaag |
| | | ccattatcagcctgggctttcgcggtgaggcgctggcgagtatcagttcg |
| | | gtttcccgcctgacgctcacttcacgcaccgcagaacagcaggaagcctg |
| | | gcaggcctatgccgaagggcgcgatatgaacgtgacggtaaaaccggcgg |
| | | cgcatcctgtggggacgacgctggaggtgctggatctgttctacaacacc |
| | | ccggcgcggcgcaaattcctgcgcaccgagaaaaccgaatttaaccacat |
| | | tgatgagatcatccgccgcattgcgctggcgcgtttcgacgtcacgatca |
| | | acctgtcgcataacggtaaaattgtgcgtcagtaccgcgcagtgccggaa |
| | | ggcgggcaaaaagaacggcgcttaggcgcgatttgcggcaccgcttttct |
| | | tgaacaagcgctggcgattgaatggcaacacggcgatctcacgctacgcg |
| | | gctgggtggccgatccaaatcacaccacgcccgcactggcagaaattcag |
| | | tattgctacgtgaacggtcgcatgatgcgcgatcgcctgatcaatcacgc |
| | | gatccgccaggcctgcgaagacaaactgggggccgatcagcaaccggcat |
| | | ttgtgttgtatctggagatcgacccacatcaggtggacgtcaacgtgcac |
| | | cccgccaaacacgaagtgcgtttccatcagtcgcgtctggtgcatgattt |
| | | tatctatcagggcgtgctgagcgtgctacaacagcaactggaaacgccgc |
| | | taccgctggacgatgaaccccaacctgcaccgcgttccattccggaaaac |
| | | cgcgtggcggcggggcgcaatcactttgcagaaccggcagctcgtgagcc |
| | | ggtagctccgcgctacactcctgcgccagcatcaggcagtcgtccggctg |
| | | ccccctggccgaatgcgcagccaggctaccagaaacagcaaggtgaagtg |
| | | tatcgccagcttttgcaaacgcccgcgccgatgcaaaaattaaaagcgcc |
| | | ggaaccgcaggaacctgcacttgcggcgaacagtcagagttttggtcggg |
| | | tactgactatcgtccattccgactgtgcgttgctggagcgcgacggcaac |
| | | atttcacttttatccttgccagtggcagaacgttggctgcgtcaggcaca |
| | | attgacgccgggtgaagcgcccgtttgcgcccagccgctgctgattccgt |
| | | tgcggctaaaagtttctgccgaagaaaaatcggcattagaaaaagcgcag |
| | | tctgccctggcggaattgggtattgatttccagtcagatgcacagcatgt |
| | | gaccatcagggcagtgcctttacccttacgccaacaaaatttacaaatct |
| | | tgattcctgaactgataggctacctggcgaagcagtccgtattcgaacct |
| | | ggcaatattgcgcagtggattgcacgaaatctgatgagcgaacatgcgca |
| | | gtggtcaatggcacaggccataaccctgctggcggacgtggaacggttat |
| | | gtccgcaacttgtgaaaacgccgccgggtggtctgttacaatctgttgat |
| | | ttacatccggcgataaaagccctgaaagatgagtgatctagagtcgacct |
| | | gcaggcatgcaagcttgcggccgcgtcgtgactgggaaaaccctggcgac |
| | | tagtcttggactcctgttgatagatccagtaatgacctcagaactccatc |
| | | tggatttgttcagaacgctcggttgccgccgggcgttttttattggtgag |
| | | aatccaggggtccccaataattacgatttaaatttgtgtctcaaaatctc |
| | | tgatgttacattgcacaagataaaaatatatcatcatgaacaataaaact |
| | | gtctgcttacataaacagtaatacaaggggtgttatgagccatattcagc |
| | | gtgaaacgagctgtagccgtccgcgtctgaacagcaacatggatgcggat |
| | | ctgtatggctataaatgggcgcgtgataacgtgggtcagagcggcgcgac |
| | | catttatcgtctgtatggcaaaccggatgcgccggaactgtttctgaaac |
| | | atggcaaaggcagcgtggcgaacgatgtgaccgatgaaatggtgcgtctg |
| | | aactggctgaccgaatttatgccgctgccgaccattaaacattttattcg |
| | | caccccggatgatgcgtggctgctgaccaccgcgattccgggcaaaaccg |
| | | cgtttcaggtgctggaagaatatccggatagcggcgaaaacattgtggat |
| | | gcgctggccgtgtttctgcgtcgtctgcatagcattccggtgtgcaactg |
| | | cccgtttaacagcgatcgtgtgtttcgtctggcccaggcgcagagccgta |
| | | tgaacaacggcctggtggatgcgagcgattttgatgatgaacgtaacggc |
| | | tggccggtggaacaggtgtggaaagaaatgcataaactgctgccgtttag |
| | | cccggatagcgtggtgacccacggcgattttagcctggataacctgattt |
| | | tcgatgaaggcaaactgattggctgcattgatgtgggccgtgtgggcatt |
| | | gcggatcgttatcaggatctggccattctgtggaactgcctgggcgaatt |
| | | tagcccgagcctgcaaaaacgtctgtttcagaaatatggcattgataatc |
| | | cggatatgaacaaactgcaatttcatctgatgctggatgaatttttctaa |
| | | taattaattggaccgcggtccgcgcgttgtccttttccgctgcataaccc |
| | | tgcttcggggtcattatagcgattttttcggtatatccatcctttttcgc |
| | | acgatatacaggattttgccaaagggttcgtgtagacCGGGCCCATTAAG |
| | | TTCTGTGCTAGGAGGTGACTGAAGTATATTTTAGGAATTCTAAAGATCTT |
| | | TGACAGCTAGCTCAGTCCTAGGTATAATACTAGTaagggcaggctgggaa |
| | | ataaCGAGACCTGAATTGCGCGCAATTAACCCTCACTAAAGGGAACAAAA |
| | | GCTGGAGCTCTTATATTCCCCAGAACATCAGGTTAATGGCGTTTTTGATG |
| | | TCATTTTCGCGGTGGCTGAGATCAGCCACTTCTTCCCCGATAACGGACAC |
| | | CGGCACACTGGCCATATCGGTGGTCATCATGCGCCAGCTTTCATCCCCGA |
| | | TATGCACCACCGGGTAAAGTTCACGGGAGACTTTATCTGACAGCAGACGT |
| | | GCACTGGCCAGGGGGATCACCATCCGTCGCCCGGGCGTGTCAATAATATC |
| | | ACTCTGTACATCCACAAACAGACGATAACGGCTCTCTCTTTTATAGGTGT |
| | | AAACCTTAAACTGCATCGTTTCACTCCATCCAAAAAAACGGGTATGGAGA |
| | | AACAGTAGAGAGTTGCGATAAAAAGCGTCAGGTAGGATCCGCTGGTCTCA |
| | | tctgtgcccatcaccttcttgcatggctctgccaacgctacggcttggcg |
| | | ggctggcctttcctcaataggtggtcagccggttctgtcctgcttcggcg |
| | | aacacgttacacggttcggcaaaacgtcgattactgaaaatggaaaggcg |
| | | gggccgacttcGGTAGTGCAGCGCGATCGTAATCAGGATCCCATGGTACG |
| | | CGTGCTAGAGGCATCAAATAAAACGAAAGGCTCAGTCGAAAGACTGGGCC |
| | | TTTCGTTTTATCTGTTGTTTGTCGGTGAACGCTCTCCTGAGTAGGACAAA |
| | | TCCGCCGCCCTAGACCCTCCACGCACGTTGTGATATGTAGATGATAATCA |
| | | TTATCACTTTACGGGTCCTTTCCGGTGATCCGACAGGTTACGGGGCGGCG |
| | | ACCTCGCGGGTTTTCGCTATTTATGAAAATTTTCCGGTTTAAGGCGTTTC |
| | | CGTTCTTCTTCGTCATAACTTAATGTTTTTATTTAAAATACCCTCTGAAA |
| | | AGAAAGGAAACGACAGGTGCTGAAAGCGAGGCTTTTTGGGGCGTTCGGCT |
| | | GCGGCGAGCGGTATCAGCTCACTCAAAGGCGGTAATACGGTTATCCACAG |
| | | AATCAGGGGATAACGCAGGAAAGAACATGTGAGCAAAAGGCCAGCAAAAG |
| | | GCCAGGAACCGTAAAAAGGCCGCGTTGCTGGCGTTTTTCCATAGGCTCCG |
| | | CCCCCCTGACGAGCATCACAAAAATCGACGCTCAAGTCAGAGGTGGCGAA |
| | | ACCCGACAGGACTATAAAGATACCAGGCGTTTCCCCCTGGAAGCTCCCTC |
| | | GTGCGCTCTCCTGTTCCGACCCTGCCGCTTACCGGATACCTGTCCGCCTT |
| | | TCTCCCTTCGGGAAGCGTGGCGCTTTCTCAATGCTCACGCTGTAGGTATC |
| | | TCAGTTCGGTGTAGGTCGTTCGCTCCAAGCTGGGCTGTGTGCACGAACCC |
| | | CCCGTTCAGCCCGACCGCTGCGCCTTATCCGGTAACTATCGTCTTGAGTC |
| | | CAACCCGGTAAGACACGACTTATCGCCACTGGCAGCAGCCACTGGTAACA |
| | | GGATTAGCAGAGCGAGGTATGTAGGCGGTGCTACAGAGTTCTTGAAGTGG |
| | | TGGCCTAACTACGGCTACACTAGAAGGACAGTATTTGGTATCTGCGCTCT |
| | | GCTGAAGCCAGTTACCTTCGGAAAAAGAGTTGGTAGCTCTTGATCCGGCA |
| | | AACAAACCACCGCTGGTAGCGGTGGTTTTTTTGTTTGCAAGCAGCAGATT |
| | | ACGCGCAGAAAAAAAGGATCTCAA |
| TR_AM011 | 19 | GTTTGGCGGTCTGGGTGCCTTCATACGGACGGCCCTCGCCTTCGCCTTCG |
| | | ATCTCGAACTCGTGACCGTT |
| pRL038-ccdB | 20 | GAAGATCATCTTATTAATCAGATAAAATATTTCTAGATTTCAGTGCAATT |
| (5265 bp) | | TATCTCTTCAAATGTAGCACgattttacggctagctcagtcctaggtaca |
| | | atgctagcgaatcattaaagaggagaaaggtactATGGCACGTACCCCGT |
| | | CACGTAGTAGCATTGGTAGCCTGCGTAGTCCGCATACCCATAAAGCAATT |
| | | CTGACCAGTACCATCGAGATCCTGAAAGAATGTGGTTATAGCGGACTGAG |
| | | CATTGAAAGCGTTGCACGTCGTGCCGGAGCAAGCAAACCGACCATTTATC |
| | | GTTGGTGGACGAATAAAGCAGCACTGATTGCCGAAGTGTATGAAAATGAA |
| | | AGCGAACAGGTGCGTAAATTTCCGGATCTGGGTAGCTTTAAAGCAGATCT |
| | | GGATTTTTTACTGCGTAATTTATGGAAAGTTTGGCGTGAAACTATTTGCG |
| | | GTGAAGCATTTCGTTGTGTTATTGCAGAAGCTCAGCTGGATCCTGCAACC |
| | | CTGACCCAGTTAAAGGATCAATTTATGGAACGTCGTCGTGAGATGCCGAA |
| | | AAAACTGGTTGAAAATGCCATTAGCAATGGTGAACTGCCGAAAGATACCA |
| | | ATCGTGAACTTCTTCTGGATATGATTTTTGGTTTTTGTTGGTATCGCCTG |
| | | TTAACCGAACAGCTGACCGTTGAACAGGATATTGAAGAATTTACCTTCCT |
| | | TCTGATTAATGGTGTTTGTCCGGGTACTCAGCGTTAACTAGGCCATAATC |
| | | GCTACCAAATTCCAGAAAACAGACGCTTTCGAGCGTCTTTTTTCGTTTTG |
| | | GTCACGACGTACTGAATCTGATTCGTTACCAATTGACATGATACGAAACG |
| | | TACCGTATCGTTAAGGTGGAGGCATATCAAAGGACGAGTGCAGGTGGCAA |
| | | AAATGGGAAAGCGCCATCGTAACCTTATTGACCAAATCACCACCTGGGAA |
| | | AATTTGTTAGATGCGTACCGTAAGACTAGCCACGGTAAGCGCCGCACTTG |
| | | GGGGTATCTTGAATTTAAGGAGTACGATTTGGCCAATTTGCTTGCACTTC |
| | | AGGCGGAGTTGAAGGCAGGCAATTACGAGCGCGGACCGTATCGCGAGTTT |
| | | CTGGTTTACGAACCGAAACCCCGCTTGATTAGCGCACTGGAATTTAAAGA |
| | | TCGTCTTGTTCAACACGCGCTGTGCAACATCGTTGCGCCAATCTTTGAAG |
| | | CAGGTTTGCTTCCATATACATACGCGTGTCGTCCGGATAAGGGGACCCAT |
| | | GCAGGAGTGTGTCATGTTCAGGCAGAATTGCGTCGCACGCGCGCGACTCA |
| | | TTTTTTGAAGAGTGACTTTAGCAAGTTCTTCCCAAGCATTGACCGTGCCG |
| | | CTCTTTATGCGATGATTGATAAAAAAATCCACTGCGCCGCTACACGTCGC |
| | | CTTTTGCGCGTTGTCCTGCCGGATGAGGGAGTTGGAATTCCCATTGGCAG |
| | | CTTAACCTCTCAGTTATTTGCCAACGTTTACGGGGGCGCGGTAGATCGTT |
| | | TGTTACACGACGAGTTAAAGCAGCGCCACTGGGCTCGTTACATGGATGAC |
| | | ATTGTCGTACTTGGGGATGATCCAGAAGAACTGCGCGCGGTCTTCTATCG |
| | | TTTGCGTGATTTTGCGTCCGAACGCTTAGGTTTGAAGATTTCACATTGGC |
| | | AGGTAGCGCCTGTGTCTCGTGGAATCAATTTTCTTGGTTACCGCATCTGG |
| | | CCGACCCATAAATTGCTGCGTAAGAGTAGTGTGAAGCGCGCTAAGCGTAA |
| | | GGTGGCAAATTTCATCAAACACGGAGAAGACGAGTCACTGCAACGCTTCC |
| | | TTGCCTCGTGGTCGGGTCACGCCCAATGGGCGGACACTCACAATTTATTC |
| | | ACTTGGATGGAGGAGCAATATGGCATCGCGTGTCATTAATAACGTTAAAG |
| | | TCAGTTTCACCTGTTTTACGTTAAAACCCGCTTCGGCGGGTTTTTACTTT |
| | | TGGGtttAGCCGAACGCCCCAAAAAGCCTCGCTTTCAGCACCTGTCGTTT |
| | | CCTTTCTTTTCAGAGGGTATTTTAAATAAAAACATTAAGTTATGACGAAG |
| | | AAGAACGGAAACGCCTTAAACCGGAAAATTTTCATAAATAGCGAAAACCC |
| | | GCGAGGTCGCCGCCCCGTAACCTGTCGGATCACCGGAAAGGACCCGTAAA |
| | | GTGATAATGATTATCATCTACATATCACAACGTGCGTAAAGGGACTagtg |
| | | gatGtttGATCTCAAAAAAAGCACCTTATTTTCCGGCTTGTCCTTTACGG |
| | | TTCACACGAGCAATCCAGGACCCTAAGATACGGCCAACCTCCGCGATAAG |
| | | CACCTGAGCAGTCTCGACCTGGTGCGGAGTCATGGCATGCGGCTTTTGAA |
| | | TGCCTGCCAAAAAGCGAAGCCAGAAGCGTAACATCGCCAAACCCGCGTCA |
| | | GCAGCATACAGTTTCGAGACCTGGTTTGATTTACCTGCTACAATGAACAG |
| | | TTCCACCTGTCCCAACAGACATTTCAAAAACATTTCGCGTGCGACTCCAT |
| | | GTTTACGAGGGATTGACTGAGCAATGGGATACAAGTATGAGATGACGCGT |
| | | TCATAGCGCTCTACAATCAACATCTGATCGTAACACTTGGTAGCCTCTTC |
| | | AATAGGTTCCATagaaactttctcctctttaataCTAGTattatacctag |
| | | gactgagctagctgtcagTCGGGTAGCACCAGAAGTCTATAGCATGtgca |
| | | taCCTTTGGTCGAAAAAAAAAGCCCGCACTGTCAGGTGCGGGCTTTTTTC |
| | | aGTGTTTCCttgccggagaagtcggccccgcctttccattttcagtaatc |
| | | gacgttttgccgaaccgtgtaacgtgttcgccgaagcaggacagaaccgg |
| | | ctgaccacctattgaggaaaggccagcccgccaagccgtagcgttggcag |
| | | agccatgcaagaaggtgatgggcacagaTGAGACCAGCGGATCCTACCTG |
| | | ACGCTTTTTATCGCAACTCTCTACTGTTTCTCCATACCCGTTTTTTTGGA |
| | | TGGAGTGAAACGATGCAGTTTAAGGTTTACACCTATAAAAGAGAGAGCCG |
| | | TTATCGTCTGTTTGTGGATGTACAGAGTGATATTATTGACACGCCCGGGC |
| | | GACGGATGGTGATCCCCCTGGCCAGTGCACGTCTGCTGTCAGATAAAGTC |
| | | TCCCGTGAACTTTACCCGGTGGTGCATATCGGGGATGAAAGCTGGCGCAT |
| | | GATGACCACCGATATGGCCAGTGTGCCGGTGTCCGTTATCGGGGAAGAAG |
| | | TGGCTGATCTCAGCCACCGCGAAAATGACATCAAAAACGCCATTAACCTG |
| | | ATGTTCTGGGGAATATAAGAGCTCCAGCTTTTGTTCCCTTTAGTGAGGGT |
| | | TAATTGCGCGCAATTCAGGTCTCGttatttcccagcctgcccttACTAtg |
| | | caaccattatcaccgccagaggtaaaattgtcaacacgcacggtgttagc |
| | | tcaaaaataaacaaaagagtttgtagaaacgcaaaaaggccatccgtcag |
| | | gatggccttctgcttaatttgatgcctggcagtttatggcgggcgtcctg |
| | | cccgccaccctccgggccgttgcttcgcaacgttcaaatccgctcccggc |
| | | ggatttgtcctTTACGCCCCGCCCTGCCACTCATCGCAGTATTGTTGTAA |
| | | TTCATTAAGCATTCTGCCGACATGGAAGCCATCACAAACGGCATGATGAA |
| | | CTTGGATCGCCAGTGGCATTAACACCTTGTCGCCTTGCGTATAATATTTT |
| | | CCCATAGTGAAAACGGGGGCGAAGAAGTTGTCCATATTTGCTACGTTTAA |
| | | ATCAAAACTGGTGAAACTCACCCACGGATTGGCACTGACGAAAAACATAT |
| | | TTTCGATAAACCCTTTAGGGAAATATGCTAAGTTTTCACCGTAACACGCC |
| | | ACATCTTGACTATATATGTGTAGAAACTGCCGGAAATCGTCGTGGTATTC |
| | | TGACCAGAGCGATGAAAACGTTTCAGTTTGCTCATGGAAAACGGTGTAAC |
| | | AAGGGTGAACACTATCCCATATCACCAGCTCACCGTCTTTCATTGCCATA |
| | | CGAAACTCCGGATGTGCATTCATCAGGCGGGCAAGAATGTGAATAAAGGC |
| | | CGGATAAAACTTGTGCTTATTTTTCTTTACGGTTTTTAAAAAGGCCGTAA |
| | | TATCCAGCTGAACGGTTTGGTTATAGGTGCACTGAGCAACTGACTGGAAT |
| | | GCCTCAAAATGTTCTTTACGATGCCATTGACTTATATCAACTGTAGTATA |
| | | TCCAGTGATTTTTTTCTCCATTTTAGCTTCCTTAGCTTGCGAAATCTCGA |
| | | TAACTCAAAAAATAGTAGTGATCTTATTTCATTATGGTGAAAGTTGTCTT |
| | | ACGTGCAACATTTTCGCAAAAAGTTGGCGCTTTATCAACACTGTCCCTCC |
| | | TGTTCAGCTACCGGCCAGCCTCGCAGAGCAGGATTCCCGTTGAGCACCGC |
| | | CAGGTGCGAATAAGGGACAGTGAAGAAGGAACACCCGCTCGCGGGTGGGC |
| | | CTACTTCACCTATCCTGCCCGGCTGACGCCGTTGGATACACCAAGGAAAG |
| | | TCTACACGAACCCTTTGGCAAAATCCTGTATATCGTGCGAAAAAGGATGG |
| | | ATATACCGAAAAAATCGCTATAATGACCCCGAAGCAGGGTTATGCAGCGG |
| | | AAAAGCGCTGGTACCCAATTCGCCCTATAGTGAGTCTCCTGGAAGTGAGA |
| | | GGGCCGCGGCAAAGCCGTTTTTCCATAGGCTCCGCCCCCCTGACAAGCAT |
| | | CACGAAATCTGACGCTCAAATCAGTGGTGGCGAAACCtGACAGGACTATA |
| | | AAGATACCAGGCGTTTCCCCCTGGCGGCTCCCTCGTGCGCTCTCCTGTTC |
| | | CTGCCTTTCGGTTTACCGGTGTCATTCCGCTGTTATGGCCGCGTTTGTCT |
| | | CATTCCACGCCTGACACTCAGTTCCGGGTAGGCAGTTCGCTCCAAGCTGG |
| | | ACTGTATGCACGAACCCCCCGTTCAGTCCGACCGCTGCGCCTTATCCGGT |
| | | AACTATCGTCTTGAGTCCAACCCGGAAAGACATGCAAAAGCACCACTGGC |
| | | AGCAGCCACTGGTAATTGATTTAGAGGAGTTAGTCTTGAAGTCATGCGCC |
| | | GGTTAAGGCTAAACTGAAAGGACAAGTTTTGGTGACTGCGCTCCTCCAAG |
| | | CCAGTTACCTCGGTTCAAAGAGTTGGTAGCTCAGAGAACCTTCGAAAAAC |
| | | CGCCCTGCAAGGCGGTTTTTTCGTTTTCAGAGCAAGAGATTACGCGCAGA |
| | | CCAAAACGATCTCAA |
| TR_AM001 | 21 | cgctgctgcgctattcggcggcaactggaacaacacgtcgaactcgggtt |
| | | ctcgcgctgcgaactggaacaacgggccgtcgaactcgaacgcgaacatc |
| | | ggggcgcgcggcg |
| TR_AM004 | 22 | ttatatggcttttggttcgtttctttcgcaaacgcttgag |
| TR_AM007 | 23 | tgccgtatgtttccttatatggcttttggttcgtttctttcgcaaacgct |
| | | tgagttgcgcctcctgccagcagtgcggtagtaaaggttaatactgttgc |
| TR_AM009 | 24 | tttgtggcctttatcttctacgtagtgaggatctctcagcgtatggttgt |
| | | cgcctgagctgtagttgcct |
| TR_AM010 | 25 | cgtgatagtttgcgacagtgccgtcagcgttttgtaatggccagctgtcc |
| | | caaacgtccaggccttttgc |
| AM001 | 26 | ATAACGCTGCTGCGCTATTCGGCGGCAACTGGAACAACACGTCGAACTCG |
| | | GGTTCTCGCGC |
| AM002 | 27 | CGCAGCGCGAGAACCCGAGTTCGACGTGTTGTTCCAGTTGCCGCCGAATA |
| | | GCGCAGCAGCG |
| AM030 | 28 | TGCGAACTGGAACAACGGGCCGTCGAACTCGAACGCGAACATCGGGGCGC |
| | | GCGGCG |
| AM031 | 29 | CAGACGCCGCGCGCCCCGATGTTCGCGTTCGAGTTCGACGGCCCGTTGTT |
| | | CCAGTT |
| AM007 | 30 | ATAATTATATGGCTTTTGGTTCGTTTCTTTCGCAAACGCTTGAG |
| AM008 | 31 | CAGACTCAAGCGTTTGCGAAAGAAACGAACCAAAAGCCATATAA |
| AM017 | 32 | ATAATGCCGTATGTTTCCTTATATGGCTTTTGGTTCGTTTCTTTCGCAAA |
| | | CGCT |
| AM018 | 33 | CTCAAGCGTTTGCGAAAGAAACGAACCAAAAGCCATATAAGGAAACATAC |
| | | GGCA |
| AM019 | 34 | TGAGTTGCGCCTCCTGCCAGCAGTGCGGTAGTAAAGGTTAATACTGTTGC |
| AM020 | 35 | CAGAGCAACAGTATTAACCTTTACTACCGCACTGCTGGCAGGAGGCGCAA |
| AM021 | 36 | ATAATTTGTGGCCTTTATCTTCTACGTAGTGAGGATCTCTCAGCGTATGG |
| | | TTGTCGCCTGAGCTGTAGTTGCCT |
| AM022 | 37 | CAGAAGGCAACTACAGCTCAGGCGACAACCATACGCTGAGAGATCCTCAC |
| | | TACGTAGAAGATAAAGGCCACAAA |
| AM024 | 38 | ATAACGTGATAGTTTGCGACAGTGCCGTCAGCGTTTTGTAATGGCCAGCT |
| | | GTCCCAAACGTCCAGGCCTTTTGC |
| AM025 | 39 | CAGAGCAAAAGGCCTGGACGTTTGGGACAGCTGGCCATTACAAAACGCTG |
| | | ACGGCACTGTCGCAAACTATCACG |
| AM027 | 40 | ATAAGTTTGGCGGTCTGGGTGCCTTCATACGGACGGCCCTCGCCTTCGCC |
| | | TTCGATCTCGAACTCGTGACCGTT |
| AM028 | 41 | CAGAAACGGTCACGAGTTCGAGATCGAAGGCGAAGGCGAGGGCCGTCCGT |
| | | ATGAAGGCACCCAGACCGCCAAAC |
| TR_RL016 | 42 | tgccgtatgtttccttatatggcttttggttcgtttctttcgcaaacgct |
| | | tgagttgcgcctcctgcc |
| TR_AM009 target | 43 | ATACTAAGTATTTGTGGCCTTTATCTTCTACGTAGTGAGGATCTCTCAGC |
| (wt/nt strand | | GTATGGTTGTCGCCTGAGCTGTAGTTGCCTTCATCGATGA |
| 1; FIG. 3C) | | |
| TR_AM009 target | 44 | TATGATTCATAAACACCGGAAATAGAAGATGCATCACTCCTAGAGAGTCG |
| (wt/nt strand | | CATACCAACAGCGGACTCGACATCAACGGAAGTAGCTACT |
| 2; FIG. 3C) | | |
| TR_AM009 target | 45 | VLYKHGKDEVYHPDRLTHNDGSSYNGEDIE |
| (wt/aa; FIG. 3C) | | |
| Variant- | 46 | ATACTAAGTATTTGTGGCCTTTTCCTTCTACGTAGTGAGGATCTCTCAGC |
| TR_AM009 | | GTATGGTTGTCGCCTGAGCTGTAGTTGCCTTCATCGATGA |
| (FIG.3C no 1) | | |
| Variant- | 47 | ATACTAAGTATTTGTGGCCTTTGTCTTCTGCGTGGTGAGGATCTCTCAGC |
| TR_AM009 | | GTATGGTTGTCGCCTGAGCTGTAGTTGCCTTCATCGATGA |
| (FIG.3C no 2) | | |
| Variant- | 48 | ATACTAAGTATTTGTGGCCTTTATCTTCTACGTAGTGAGGATCTCTCGGC |
| TR_AM009 | | GTATGGTTGTCGCCTGAGCTGTAGTTGCCTTCATCGATGA |
| (FIG.3C no 3) | | |
| Variant- | 49 | ATACTAAGTATTTGTGGCCTTGATCTTCTACGTAGTGAGGTTCTCTCAGC |
| TR_AM009 | | GTGTGGTTGTCGCCTGAGCTGTAGTTGCCTTCATCGATGA |
| (FIG.3C no 4) | | |
| TR_AM010 target | 50 | ATGTGGTAGCCGTGATAGTTTGCGACAGTGCCGTCAGCGTTTTGTAATGG |
| (wt/nt strand | | CCAGCTGTCCCAAACGTCCAGGCCTTTTGCAGAAGAGATA |
| 1; FIG. 3C) | | |
| TR_AM010 target | 51 | TACACCATCGGCACTATCAAACGCTGTCACGGCAGTCGCAAAACATTACC |
| (wt/nt strand | | GGTCGACAGGGTTTGCAGGTCCGGAAAACGTCTTCTCTAT |
| 2; FIG. 3C) | | |
| TR_AM010 target | 52 | IHYGHYNAVTGDANQLPWSDWVDLGKASSI |
| (wt/aa; FIG. 3C) | | |
| Variant- | 53 | ATGTGGTAGCCGTGATAGTTTGCGACAGTGCCGTCAGCGTTTTGTAATGG |
| TR_AM010 | | CCAGCTGTCCCAGTCGTCCAGGCCTTTTGCAGAAGAGATA |
| (FIG.3C no 1) | | |
| Variant- | 54 | ATGTGGTAGCCGTGATAGTTTGCGACAGTGCCGTCAGCGTTTTGTAATGG |
| TR_AM010 | | CCGGCTGTCCCAAACGTCCAGGCCTTTTGCAGAAGAGATA |
| (FIG.3C no 2) | | |
| Variant- | 55 | ATGTGGTAGCCGTGTTAGTTTGCGACAGTGCCGTCAGCGTTTTGTCATGG |
| TR_AM010 | | CCAGCTGTCCCAAACGTCCAGGCCTTTTGCAGAAGAGATA |
| (FIG.3C no 3) | | |
| Variant- | 56 | ATGTGGTAGCCGTGATAGTTTGCGTCAGTGCCGTCAGCGTTTTGTACTGG |
| TR_AM010 | | CCAGCTGTCCCAAACGTCCAGGCCTTTTGCAGAAGAGATA |
| (FIG.3C no 4) | | |
| TR_RL016 target | 57 | ATATGGGAAATGCCGTATGTTTCCTTATATGGCTTTTGGTTCGTTTCTTT |
| (wt/nt strand | | CGCAAACGCTTGAGTTGCGCCTCCTGCCAGCAGTGCGG |
| 1; FIG. 3C) | | |
| TR_RL016 target | 58 | TATACCCTTTACGGCATACAAAGGAATATACCGAAAACCAAGCAAAGAAA |
| (wt/nt strand | | GCGTTTGCGAACTCAACGCGGAGGACGGTCGTCACGCC |
| 2; FIG. 3C) | | |
| TR_AM009 target | 59 | IHSIGYTEKYPKONTEKAFAQTAGGALLAT |
| (wt/aa; FIG. 3C) | | |
| Variant- | 60 | ATATGGGAAATGCCGTATGTTTCCTTATATGGCTTTTGGTTCGTTTCTTT |
| TR_RL016 | | CGCTTACGCTTGAGTTGCGCCTCCTGCCAGCAGTGCGG |
| (FIG.3C no 1) | | |
| Variant- | 61 | ATATGGGAAATGCCGTGTGTTTCCTTCTATGGCTTTTGGTTCGTTTCTTT |
| TR_RL016 | | CGCGAACGCTTGAGTTGCGCCTCCTGCCAGCAGTGCGG |
| (FIG.3C no 2) | | |
| Variant- | 62 | ATATGGGAAATGCCGTATGTTTCCTTTTATGGCTCTTGGTTCGTTTCTTT |
| TR_RL016 | | CGCGTACGCTTGAGTTGCGCCTCCTGCCAGCAGTGCGG |
| (FIG.3C no 3) | | |
| Variant- | 63 | ATATGGGAAATGCCGTGTGTTTCCTTCTATGGCTTTTGGTTCGTTTCTTT |
| TR_RL016 | | CGCTTTCGCTTGGGTTGCGCCTCCTGCCAGCAGTGCGG |
| (FIG.3C no 4) | | |
| TR_AM004 target | 64 | GTATGTTTCCTTATATGGCTTTTGGTTCGTTTCTTTCGCAAACGCTTGAG |
| (wt/nt strand | | TTGCGCCTCC |
| 1; FIG. 3D) | | |
| TR_AM004 target | 65 | CATACAAAGGAATATACCGAAAACCAAGCAAAGAAAGCGTTTGCGAACTC |
| (wt/nt strand | | AACGCGGAGG |
| 2; FIG. 3D) | | |
| TR_AM004 target | 66 | YTEKYPKONTEKAFAQTAGG |
| (wt/nt aa; | | |
| FIG. 3D) | | |
| Variant- | 67 | GTATGTTTCCTTATATGGCTTTTGGTTCGTTTCTTTCGCCTACGCTTGAG |
| TR_AM004 | | TTGCGCCTCC |
| (FIG.3D) | | |
| TR_AM007 target | 68 | GGGAAATGCCGTATGTTTCCTTATATGGCTTTTGGTTCGTTTCTTTCGCA |
| (wt/nt strand | | AACGCTTGAGTTGCGCCTCCTGCCAGCAGTGCGGTAGTAAAGGTTAATAC |
| 1; FIG. 3D) | | TGTTGCTTGTTT |
| TR_AM007 target | 69 | CCCTTTACGGCATACAAAGGAATATACCGAAAACCAAGCAAAGAAAGCGT |
| (wt/nt strand | | TTGCGAACTCAACGCGGAGGACGGTCGTCACGCCATCATTTCCAATTATG |
| 2; FIG. 3D) | | ACAACGAACAAA |
| TR_AM007 target | 70 | SIGYTEKYPKQNTEKAFAQTAGGALLATTFTLVTAQK |
| (wt/aa; FIG. 3D) | | |
| Variant- | 71 | GGGAAATGCCGTGTGTTTCCTTATATGGCTTTTGGTTCGTTTCTTTCGCT |
| TR_AM007 | | TACGCTTGAGTTGCGCCTCCTGCCAGCAGTGCGGTAGTAAAGGTTAATAC |
| (FIG.3D no 1) | | TGTTGCTTGTTT |
| Variant- | 72 | GGGAAATGCCGTATGTTTCCTTATATGGCTTTTGGTTCGTTTCTTTCGGT |
| TR_AM007 | | TTCGCTTGAGTTGCGCCTCCTGCCGGCAGTGCGGTAGTAAAGGTTGATAC |
| (FIG.3D no 2) | | TGTTGCTTGTTT |
| Variant- | 73 | GGGAAATGCCGTATGTTTCCTTTTATGGCTTTTGGTTCGTTTCTTTCGCA |
| TR_AM007 | | AACGCTTGGGTTGCGCCTCCTGCCAGCGGTGCGGTAGTAAAGGTTTATAC |
| (FIG.3D no 3) | | TGTTGCTTGTTT |
| Variant- | 74 | GGGAAATGCCGTATGTTTCCTTATATGGCTTTTGGTTCGTTTCTTTCGCC |
| TR_AM007 | | TTCGCTTGAGTTGCGCCTCCTGCCAGCGGTGCGGTAGTAAAGGTTAATAC |
| (FIG.3D no 4) | | TGTTGCTTGTTT |
| TR_AM011 target | 75 | GTCACTTTCAGTTTGGCGGTCTGGGTGCCTTCATACGGACGGCCCTCGCC |
| (wt/nt strand | | TTCGCCTTCGATCTCGAACTCGTGACCGTTAACAGAACCC |
| 1; FIG. 3D) | | |
| TR_AM011 target | 76 | CAGTGAAAGTCAAACCGCCAGACCCACGGAAGTATGCCTGCCGGGAGCGG |
| (wt/nt strand | | AAGCGGAAGCTAGAGCTTGAGCACTGGCAATTGTCTTGGG |
| 2; FIG. 3D) | | |
| Variant- | 77 | GTCACTTTCAGTTTGGCGGTCTGGGTGCCTTCATACGGACGGCCCTCGCC |
| TR_AM011 | | TTCGCCTTCGATCTCGGTCTCGTGACCGTTAACAGAACCC |
| (FIG.3D no 1) | | |
| Variant- | 78 | GTCACTTTCAGTTTGGCGGTCTGGGTGCCTTCATGCGGGCGGCCCTCGCC |
| TR_AM011 | | TTCGCCTTCGCTCTCGAACTCGTGACCGTTAACAGAACCC |
| (FIG.3D no 2) | | |
| Variant- | 79 | GTCACTTTCAGTTTGGCGGTCTGGGTGCCTTCACACGGACGGCCCTCGCC |
| TR_AM011 | | TTCGCCTTCGATCTCGGCCTCGTGACCGTTAACAGAACCC |
| (FIG.3D no 3) | | |
| Variant- | 80 | GTCACTTTCAGTTTGGCGGTCTGGGTGCCTTCATGCGGACGGCCCTCGCC |
| TR_AM011 | | TTCGCCTTCGGTCTCGTCCTCGTGACCGTTAACAGAACCC |
| (FIG.3D no 4) | | |
| Variant- | 81 | ATACTAAGTATTTGTGGCCTTTCTCTTCTACGTGGTGAGGATCTCTCAGC |
| TR_AM009 | | GTATGGTTGTCGCCTGAGCTGTAGTTGCCTTCATCGATGA |
| (FIG.6A no 5) | | |
| Variant- | 82 | ATACTAAGTATTTGTGGCCTTTGTCTTCTACGTAGTGAGGATCTCTCGGC |
| TR_AM009 | | GTATGGTTGTCGCCTGAGCTGTAGTTGCCTTCATCGATGA |
| (FIG.6A no 6) | | |
| Variant- | 83 | ATACTAAGTATTTGTGGCCTTGTTCTTCTACGTAGTGAGGATCTCTCAGC |
| TR_AM009 | | GTGTGGTTGTCGCCTGGGCTGTAGTTGCCTTCATCGATGA |
| (FIG.6A no 7) | | |
| Variant- | 84 | ATACTAAGTATTTGTGGCCTTTATCTTCTACGTAGTGAGGTTCTCTCAGC |
| TR_AM009 | | GTGTGGTTGTCGCCTGAGCTGTAGTTGCCTTCATCGATGA |
| (FIG.6A no 8) | | |
| TR_AM011 target | 85 | CACTTTCAGTTTGGCGGTCTGGGTGCCTTCATACGGACGGCCCTCGCCTT |
| (wt/nt strand | | CGCCTTCGATCTCGAACTCGTGACCGTTAACAGAAC |
| 1; FIG. 6B) | | |
| TR_AM01 target 1 | 86 | GTGAAAGTCAAACCGCCAGACCCACGGAAGTATGCCTGCCGGGAGCGGAA |
| (wt/nt strand | | GCGGAAGCTAGAGCTTGAGCACTGGCAATTGTCTTG |
| 2; FIG. 6B) | | |
| Variant- | 87 | CACTTTCAGTTTGGCGGGCTGGGTGCCTTCATACGGGCGGCCCTCGCCTT |
| TR_AM011 | | CGCCTTCGATCTCGAACTCGTGACCGTTAACAGAAC |
| (FIG.6B no 5) | | |
| Variant- | 88 | CACTTTCAGTTTGGCGGTCTGGGTGCCTTCGTGCGGGCGGCCCTCGCCTT |
| TR_AM011 | | CGCCTTCGATCTCGTCCTCGTGACCGTTAACAGAAC |
| (FIG.6B no 6) | | |
| TR_AM009 target | 89 | ACTAAGTATTTGTGGCCTTTATCTTCTACGTAGTGAGGATCTCTCAGCGT |
| (wt/nt strand | | ATGGTTGTCGCCTGAGCTGTAGTTGCCTTCATCGATGA |
| 1; FIG. 6B) | | |
| TR_AM009 target | 90 | TGATTCATAAACACCGGAAATAGAAGATGCATCACTCCTAGAGAGTCGCA |
| (wt/nt strand | | TACCAACAGCGGACTCGACATCAACGGAAGTAGCTACT |
| 2; FIG. 6B) | | |
| Variant- | 91 | ACTAAGTATTTGTGGCCTTTTTCTTCTACGTAGTGAGGTTCTCTCAGCGT |
| TR_AM009 | | TTGGTTGTCGCCTGAGCTGTAGTTGCCTTCATCGATGA |
| (FIG.6B no 9) | | |
| Variant- | 92 | ACTAAGTATTTGTGGCCTTTATCTTCGACGTGGTGGGGATCTCTCAGCGT |
| TR_AM009 | | GTGGTTGTCGCCTGAGCTGTAGTTGCCTTCATCGATGA |
| (FIG.6B no 10) | | |
| Sequence below | 93 | CGCCTTGGTAGCCATCTTCAGTTCCAGTGTTTGCTTCAAATACTAAGTAT |
| plot in FIG. | | TTGTGGCCTTTATCTTCTACGTAGTGAGGATCTCTCAGCGTATGGTTGTC |
| 7A | | GCCTGAGCTGTAGTTGCCTTCATCGATGAACTGCTGTACATTTTGATACG |
| pAM020 | 94 | AGTCACACATAGACAGCCTGAAACAGGCGATGCTGCTTATCGAATCAAAG |
| | | CTGCCGACAACACGGGAGCCAGTGACGCCTCCCGTGGGGAAAAAATCATG |
| | | GCAATTCTGGAAGAAATAGCGCTTTCAGCCGGCAAACCTGAAGCCGGATC |
| | | TGCGATTCTGATAACAAACTAGCAACACCAGAACAGCCCGTTTGCGGGCA |
| | | GCAAAACCCGTACCCTAGGTCTAGGGCGGCGGATTTGTCCTACTCAGGAG |
| | | AGCGTTCACCGACAAACAACAGATAAAACGAAAGGCCCAGTCTTTCGACT |
| | | GAGCCTTTCGTTTTATTTGATGCCTGGTTTGTAGAGTTCATCCATGCCGT |
| | | GCGTGATACCTGCTGCAGTAACGAACTCCAGCAGCACCATGTGGTCGCGC |
| | | TTTTCGTTCGGGTCTTTGGACAGtttAGACTGGGTGGACAGGTAGTGGTT |
| | | ATCCGGCAGCAGAACCGGACCATCACCGATCGGAGTGTTCTGCTGGTAGT |
| | | GGTCCGCCAGCTGTACGCTACCGTCTTCAACGTTATGGCGAATTTTGAAG |
| | | TTAGCTTTGATACCGTTCTTCTGTTTGTCTGCGGTGATGTAAACGTTATG |
| | | GGAGTTGAAGTTATATTCCAGTTTGTGGCCCAGGATGTTGCCGTCCTCTT |
| | | TGAAATCAATGCCTTTCAGTTCAATACGGTTCACCAGAGTATCACCTTCA |
| | | AATTTAACCTCTGCACGGGTTTTGTAGGTGCCATCGTCTTTGAAAGAAAT |
| | | GGTGCGCTCCTGTACATAACCTTCCGGCATTGCAGATTTGAAGAAATCAT |
| | | GCTGCTTCATGTGATCCGGGTAACGAGAAAAACACTGAACACCATAGGTC |
| | | AGGGTAGTCACCAGAGTCGGCCATGGAACCGGCAGTTTACCGGTAGTGCA |
| | | GATGAATTTCAGGGTCAGTTTACCGTTGGTTGCATCACCTTCACCTTCAC |
| | | CACGAACAGAGAATTTGTGGCCGTTAACATCACCATCCAGTTCAACCAGG |
| | | ATCGGAACAACACCGGTGAACAGTTCTTCACCTTTACTCATTTTTGCCTC |
| | | CTAACTAGGTCATTTGATATGCCTCCGGATATCACTCTATCAATGATAGA |
| | | GAGCTTATTTTAATTATGCTCTATCAATGATAGAGTGTCAATATTTTTTT |
| | | TAGTTTTTCATGAACTCGAGGGGATCCAAATAAAAAACTAGTTTGACAAA |
| | | TAACTCTATCAATGATATAATGTCAACAAAAAGGAGGAATTAATGATGTC |
| | | TAGATTAGATAAAAGTAAAGTGATTAACAGCGCATTAGAGCTGCTTAATG |
| | | AGGTCGGAATCGAAGGTTTAACAACCCGTAAACTCGCCCAGAAGCTAGGT |
| | | GTAGAGCAGCCTACATTGTATTGGCATGTAAAAAATAAGCGGGCTTTGCT |
| | | CGACGCCTTAGCCATTGAGATGTTAGATAGGCACCATACTCACTTTTGCC |
| | | CTTTAGAAGGGGAAAGCTGGCAAGATTTTTTACGTAATAACGCTAAAAGT |
| | | TTTAGATGTGCTTTACTAAGTCATCGCGATGGAGCAAAAGTACATTTAGG |
| | | TACACGGCCTACAGAAAAACAGTATGAAACTCTCGAAAATCAATTAGCCT |
| | | TTTTATGCCAACAAGGTTTTTCACTAGAGAATGCATTATATGCACTCAGC |
| | | GCTGTGGGGCATTTTACTTTAGGTTGCGTATTGGAAGATCAAGAGCATCA |
| | | AGTCGCTAAAGAAGAAAGGGAAACACCTACTACTGATAGTATGCCGCCAT |
| | | TATTACGACAAGCTATCGAATTATTTGATCACCAAGGTGCAGAGCCAGCC |
| | | TTCTTATTCGGCCTTGAATTGATCATATGCGGATTAGAAAAACAACTTAA |
| | | ATGTGAAAGTGGGTCTTAAGACGTCGGAATTGCCAGCTGGGGCGCCCTCT |
| | | GGTAAGGTTGGGAAGCCCTGCAAAGTAAACTGGATGGCTTTCTTGCCGCC |
| | | AAGGATCTGATGGCGCAGGGGATCAAGATCTGATCAAGAGACAGGATGAG |
| | | GATCGTTTCGCTTTGTTTATTTTTCTAAATACATTCAAATATGTATCCGC |
| | | TCATGAGACAATAACCCTGATAAATGCTTCAATAATATTGAAAAAGGAAG |
| | | AGTATGAGTATTCAACATTTCCGTGTCGCCCTTATTCCCTTTTTTGCGGC |
| | | ATTTTGCCTTCCTGTTTTTGCTCACCCAGAAACGCTGGTGAAAGTAAAAG |
| | | ATGCTGAAGATCAGTTGGGTGCACGAGTGGGTTACATCGAACTGGATCTC |
| | | AACAGCGGTAAGATCCTTGAGAGTTTTCGCCCCGAAGAACGTTTTCCAAT |
| | | GATGAGCACTTTTAAAGTTCTGCTATGTGGCGCGGTATTATCCCGTGTTG |
| | | ACGCCGGGCAAGAGCAACTCGGTCGCCGCATACACTATTCTCAGAATGAC |
| | | TTGGTTGAGTACTCACCAGTCACAGAAAAGCATCTTACGGATGGCATGAC |
| | | AGTAAGAGAATTATGCAGTGCTGCCATAACCATGAGTGATAACACTGCGG |
| | | CCAACTTACTTCTGACAACGATCGGAGGACCGAAGGAGCTAACCGCTTTT |
| | | TTGCACAACATGGGGGATCATGTAACTCGCCTTGATCGTTGGGAACCGGA |
| | | GCTGAATGAAGCCATACCAAACGACGAGCGTGACACCACGATGCCTGCAG |
| | | CAATGGCAACAACGTTGCGCAAACTATTAACTGGCGAACTACTTACTCTA |
| | | GCTTCCCGGCAACAATTAATAGACTGGATGGAGGCGGATAAAGTTGCAGG |
| | | ACCACTTCTGCGCTCGGCCCTTCCGGCTGGCTGGTTTATTGCTGATAAAT |
| | | CTGGAGCCGGTGAGCGTGGGTCTCGCGGTATCATTGCAGCACTGGGGCCA |
| | | GATGGTAAGCCCTCCCGTATCGTAGTTATCTACACGACGGGGAGTCAGGC |
| | | AACTATGGATGAACGAAATAGACAGATCGCTGAGATAGGTGCCTCACTGA |
| | | TTAAGCATTGGTAAGCGGGACTCTGGGGTTCGAGAGCTCGCTTGGACTCC |
| | | TGTTGATAGATCCAGTAATGACCTCAGAACTCCATCTGGATTTGTTCAGA |
| | | ACGCTCGGTTGCCGCCGGGCGTTTTTTATTGGTGAGAATCCAAGCACTAG |
| | | GGACAGTAAGACGGGTAAGCCTGTTGATGATACCGCTGCCTTACTGGGTG |
| | | CATTAGCCAGTCTGAATGACCTGTCACGGGATAATCCGAAGTGGTCAGAC |
| | | TGGAAAATCAGAGGGCAGGAACTGCTGAACAGCAAAAAGTCAGATAGCAC |
| | | CACATAGCAGACCCGCCATAAAACGCCCTGAGAAGCCCGTGACGGGCTTT |
| | | TCTTGTATTATGGGTAGTTTCCTTGCATGAATCCATAAAAGGCGCCTGTA |
| | | GTGCCATTTACCCCCATTCACTGCCAGAGCCGTGAGCGCAGCGAACTGAA |
| | | TGTCACGAAAAAGACAGCGACTCAGGTGCCTGATGGTCGGAGACAAAAGG |
| | | AATATTCAGCGATTTGCCCGAGCTTGCGAGGGTGCTACTTAAGCCTTTAG |
| | | GGTTTTAAGGTCTGTTTTGTAGAGGAGCAAACAGCGTTTGCGACATCCTT |
| | | TTGTAATACTGCGGAACTGACTAAAGTAGTGAGTTATACACAGGGCTGGG |
| | | ATCTATTCTTTTTATCTTTTTTTATTCTTTCTTTATTCTATAAATTATAA |
| | | CCACTTGAATATAAACAAAAAAAACACACAAAGGTCTAGCGGAATTTACA |
| | | GAGGGTCTAGCAGAATTTACAAGTTTTCCAGCAAAGGTCTAGCAGAATTT |
| | | ACAGATACCCAGATCTCCCGGGAAAAGGACTAGTAATTATCATTGACTAG |
| | | CCCATCTCAATTGGTATAGTGATTAAAATCACCTAGACCAATTGAGATGT |
| | | ATGTCTGAATTAGTTGTTTTCAAAGCAAATGAACTAGCGATTAGTCGCTA |
| | | TGACTTAACGGAGCATGAAACCAAGCTAATTTTATGCTGTGTGGCACTAC |
| | | TCAACCCCACGATTGAAAACCCTACAAGGAAAGAACGGACGGTATCGTTC |
| | | ACTTATAACCAATACGCTCAGATGATGAACATCAGTAGGGAAAATGCTTA |
| | | TGGTGTATTAGCTAAAGCAACCAGAGAGCTGATGACGAGAACTGTGGAAA |
| | | TCAGGAATCCTTTGGTTAAAGGCTTTGAGATTTTCCAGTGGACAAACTAT |
| | | GCCAAGTTCTCAAGCGAAAAATTAGAATTAGTTTTTAGTGAAGAGATATT |
| | | GCCTTATCTTTTCCAGTTAAAAAAATTCATAAAATATAATCTGGAACATG |
| | | TTAAGTCTTTTGAAAACAAATACTCTATGAGGATTTATGAGTGGTTATTA |
| | | AAAGAACTAACACAAAAGAAAACTCACAAGGCAAATATAGAGATTAGCCT |
| | | TGATGAATTTAAGTTCATGTTAATGCTTGAAAATAACTACCATGAGTTTA |
| | | AAAGGCTTAACCAATGGGTTTTGAAACCAATAAGTAAAGATTTAAACACT |
| | | TACAGCAATATGAAATTGGTGGTTGATAAGCGAGGCCGCCCGACTGATAC |
| | | GTTGATTTTCCAAGTTGAACTAGATAGACAAATGGATCTCGTAACCGAAC |
| | | TTGAGAACAACCAGATAAAAATGAATGGTGACAAAATACCAACAACCATT |
| | | ACATCAGATTCCTACCTACGTAACGGACTAAGAAAAACACTACACGATGC |
| | | TTTAACTGCAAAAATTCAGCTCACCAGTTTTGAGGCAAAATTTTTGAGTG |
| | | ACATGCAAAGTAAGCATGATCTCAATGGTTCGTTCTCATGGCTCACGCAA |
| | | AAACAACGAACCACACTAGAGAACATACTGGCTAAATACGGAAGGATCTG |
| | | AGGTTCTTATGGCTCTTGTATCTATCAGTGAAGCATCAAGACTAACAAAC |
| | | AAAAGTAGAACAACTGTTCACCGTTAGATATCAAAGGGAAAACTGTCCAT |
| | | ATGCACAGATGAAAACGGTGTAAAAAAGATAGATACATCAGAGCTTTTAC |
| | | GAGTTTTTGGTGCATTTAAAGCTGTTCACCATGAACAGATCGACAATGTA |
| | | ACAGATGAACAGCATGTAACACCTAATAGAACAGGTGAAACCAGTAAAAC |
| | | AAAGCAACTAGAACATGAAATTGAACACCTGAGACAACTTGTTACAGCTC |
| | | AAC |
| TR_RL031 | 95 | TTTGTGGCCTTTATCTTCTACGTAGTGAGGATCTCACAGCGTATGGTTGT |
| | | CGCCTGAGCTGTAGTTGCCT |
| Cas9PID_Recoded_ | 96 | aAaAAGACTGAGGTGCAGACTGGCGGTTTCTCTAAGGAGTCCATTCTGCC |
| low_A | | GAAGCGCAACTCCGACAAGCTGATCGCGCGTAAGAAGGACTGGGATCCGA |
| | | AGAAGTACGGTGGCTTCGATTCTCCGACCGTGGCGTACTCTGTTCTGGTG |
| | | GTCGCCAAGGTCGAGAAGGGTAAGTCTAAGAAGCTGAAGTCTGTGAAGGA |
| | | GCTTCTGGGCATCACCATTATGGAGCGTTCTTCTTTCGAGAAGAACCCGA |
| | | TTGACTTCCTCGAGGCGAAGGGGTACAAGGAGGTGAAGAAGGATCTGATT |
| | | ATCAAGCTGCCGAAGTACTCCCTGTTCGAGCTGGAGAATGGTCGTAAGCG |
| | | TATGCTGGCGTCTGCGGGTGAGCTGCAGAAGGGGAACGAGTTGGCCCTTC |
| | | CGTCCAAGTACGTGAACTTCCTGTACCTGGCCTCGCACTACGAGAAGCTG |
| | | AAGGGTTCTCCGGAGGATAATGAGCAGAAGCAGCTGTTCGTGGAGCAGCA |
| | | CAAGCACTACCTGGACGAGATTATTGAGCAGATTTCTGAGTTTTCTAAGC |
| | | GCGTGATTCTGGCGGACGCGAATCTGGATAAGGTCCTGTCTGCCTACAAT |
| | | AAGCACCGTGATAAGCCGATCCGTGAGCAGGCGGAGAACATCATTCACCT |
| | | GTTCACGCTGACTAATCTTGGTGCTCCGGCGGCCTTCAAGTACTTCGACA |
| | | CCACGATCGATCGTAAGCGTTACACCTCCACTAAGGAGGTCCTGGATGCG |
| | | ACTCTTATTCACCAGTCTATCACTGGCCTGTACGAGACTCGTATTGATCT |
| | | GAGtCAGTTGGGCGGTGACTAATaa |
| Cas9PID_Recoded_ | 97 | aAAAAGACGGAGGTACAGACGGGCGGCTTCAGCAAGGAAAGCATCCTGCC |
| low_T | | GAAACGCAACAGCGACAAACTGATCGCGCGCAAGAAAGACTGGGACCCGA |
| | | AGAAATACGGCGGCTTCGACAGCCCAACCGTGGCATACAGCGTGCTGGTA |
| | | GTCGCCAAAGTCGAAAAGGGCAAAAGCAAAAAACTGAAAAGCGTGAAAGA |
| | | ACTGCTGGGCATCACCATCATGGAACGCAGCAGCTTCGAAAAAAACCCGA |
| | | TCGACTTCCTCGAAGCGAAGGGGTACAAGGAAGTAAAGAAAGACCTGATC |
| | | ATCAAACTGCCGAAGTACAGCCTGTTCGAACTGGAAAACGGCCGCAAACG |
| | | CATGCTGGCGAGCGCGGGCGAACTGCAAAAAGGGAACGAACTGGCCCTGC |
| | | CGAGCAAGTACGTGAACTTCCTGTACCTGGCCAGCCACTACGAGAAACTG |
| | | AAAGGCAGCCCGGAAGACAACGAGCAGAAACAGCTGTTCGTGGAACAGCA |
| | | CAAACACTACCTGGACGAGATCATCGAACAGATCAGCGAGTTCAGCAAAC |
| | | GCGTAATCCTGGCGGACGCGAACCTGGACAAAGTCCTGAGCGCCTACAAC |
| | | AAACACCGCGACAAACCGATCCGCGAACAGGCAGAAAACATCATCCACCT |
| | | GTTCACGCTGACGAACCTGGGCGCCCCGGCAGCCTTCAAATACTTCGACA |
| | | CCACGATCGACCGCAAACGCTACACCAGCACGAAAGAAGTCCTGGACGCA |
| | | ACGCTGATCCACCAGAGCATCACGGGCCTGTACGAAACGCGCATCGACCT |
| | | GAGtCAGCTGGGCGGCGACTAATaa |
| NOPID_GFP | 98 | AaaaagGGCGGTAGCGGTATGCGTAAAGGCGAAGAGCTGTTCACTGGTGT |
| | | CGTCCCTATTCTGGTGGAACTGGATGGTGATGTCAACGGTCATAAGTTTT |
| | | CCGTGCGTGGCGAGGGTGAAGGTGACGCAACTAATGGTAAACTGACGCTG |
| | | AAGTTCATCTGTACTACTGGTAAACTGCCGGTTCCTTGGCCGACTCTGGT |
| | | AACGACGCTGACTTATGGTGTTCAGTGCTTTGCTCGTTATCCGGACCATA |
| | | TGAAGCAGCATGACTTCTTCAAGTCCGCCATGCCGGAAGGCTATGTGCAG |
| | | GAACGCACGATTTCCTTTAAGGATGACGGCACGTACAAAACGCGTGCGGA |
| | | AGTGAAATTTGAAGGCGATACCCTGGTAAACCGCATTGAGCTGAAAGGCA |
| | | TTGACTTTAAAGAGGACGGCAATATCCTGGGCCATAAGCTGGAATACAAT |
| | | TTTAACAGCCACAATGTTTACATCACCGCCGATAAACAAAAAAATGGCAT |
| | | TAAAGCGAATTTTAAAATTCGCCACAACGTGGAGGATGGCAGCGTGCAGC |
| | | TGGCTGATCACTACCAGCAAAACACTCCAATCGGTGATGGTCCTGTTCTG |
| | | CTGCCAGACAATCACTATCTGAGCACGCAAAGCGTTCTGTCTAAAGATCC |
| | | GAACGAGAAACGCGATCATATGGTTCTGCTGGAGTTCGTAACCGCAGCGG |
| | | GCATCACGCATGGTATGGATGAACTGTACAAATAA |
| Cas9_T1 | 99 | aAaAAGACTGAGGTGCAGACTGGCGGTTTCTCTAAGGAGTCCATTCTGCC |
| | | GAAGCGCAACTCCGACAAGCTGATCGCGCGTAAGAAGGACTGGGATCCGA |
| | | AGAAGTACGGTGGCTTCGATTCTCCGACCGTGGCGtaaTCTGTTCTGGTG |
| | | GTCGCCAAGGTCGAGAAGGGTAAGTCTAAGAAGCTGAAGTCTGTGAAGGA |
| | | GCTTCTGGGCATCACCATTATGGAGCGTTCTTCTTTCGAGAAGAACCCGA |
| | | TTGACTTCCTCGAGGCGAAGGGGTACAAGGAGGTGAAGAAGGATCTGATT |
| | | ATCAAGCTGCCGAAGTACTCCCTGTTCGAGCTGGAGAATGGTCGTAAGCG |
| | | TATGCTGGCGTCTGCGGGTGAGCTGCAGAAGGGGAACGAGTTGGCCCTTC |
| | | CGTCCAAGTACGTGAACTTCCTGTACCTGGCCTCGCACTACGAGAAGCTG |
| | | AAGGGTTCTCCGGAGGATAATGAGCAGAAGCAGCTGTTCGTGGAGCAGCA |
| | | CAAGCACTACCTGGACGAGATTATTGAGCAGATTTCTGAGTTTTCTAAGC |
| | | GCGTGATTCTGGCGGACGCGAATCTGGATAAGGTCCTGTCTGCCTACAAT |
| | | AAGCACCGTGATAAGCCGATCCGTGAGCAGGCGGAGAACATCATTCACCT |
| | | GTTCACGCTGACTAATCTTGGTGCTCCGGCGGCCTTCAAGTACTTCGACA |
| | | CCACGATCGATCGTAAGCGTTACACCTCCACTAAGGAGGTCCTGGATGCG |
| | | ACTCTTATTCACCAGTCTATCACTGGCCTGTACGAGACTCGTATTGATCT |
| | | GAGtCAGTTGGGCGGTGACTAATaa |
| Cas9_T2 | 100 | aAaAAGACTGAGGTGCAGACTGGCGGTTTCTCTAAGGAGTCCATTCTGCC |
| | | GAAGCGCAACTCCGACAAGCTGATCGCGCGTAAGAAGGACTGGGATCCGA |
| | | AGAAGTACGGTGGCTTCGATTCTCCGACCGTGGCGtaaTCTGTTtagGTG |
| | | GTCGCCAAGGTCGAGAAGGGTAAGTCTAAGAAGCTGAAGTCTGTGAAGGA |
| | | GCTTCTGGGCATCACCATTATGGAGCGTTCTTCTTTCGAGAAGAACCCGA |
| | | TTGACTTCCTCGAGGCGAAGGGGTACAAGGAGGTGAAGAAGGATCTGATT |
| | | ATCAAGCTGCCGAAGTACTCCCTGTTCGAGCTGGAGAATGGTCGTAAGCG |
| | | TATGCTGGCGTCTGCGGGTGAGCTGCAGAAGGGGAACGAGTTGGCCCTTC |
| | | CGTCCAAGTACGTGAACTTCCTGTACCTGGCCTCGCACTACGAGAAGCTG |
| | | AAGGGTTCTCCGGAGGATAATGAGCAGAAGCAGCTGTTCGTGGAGCAGCA |
| | | CAAGCACTACCTGGACGAGATTATTGAGCAGATTTCTGAGTTTTCTAAGC |
| | | GCGTGATTCTGGCGGACGCGAATCTGGATAAGGTCCTGTCTGCCTACAAT |
| | | AAGCACCGTGATAAGCCGATCCGTGAGCAGGCGGAGAACATCATTCACCT |
| | | GTTCACGCTGACTAATCTTGGTGCTCCGGCGGCCTTCAAGTACTTCGACA |
| | | CCACGATCGATCGTAAGCGTTACACCTCCACTAAGGAGGTCCTGGATGCG |
| | | ACTCTTATTCACCAGTCTATCACTGGCCTGTACGAGACTCGTATTGATCT |
| | | GAGtCAGTTGGGCGGTGACTAATaa |
| Cas9_T3 | 101 | aAaAAGACTGAGGTGCAGACTGGCGGTTTCTCTAAGGAGTCCATTCTGCC |
| | | GAAGCGCAACTCCGACAAGCTGATCGCGCGTAAGAAGGACTGGGATCCGA |
| | | AGAAGTACGGTGGCTTCGATTCTCCGACCGTGGCGTACTCTGTTCTGGTG |
| | | GTCGCCAAGGTCGAGAAGGGTAAGTCTAAGAAGCTGAAGTCTGTGAAGGA |
| | | GCTTCTGGGCATCACCATTATGGAGCGTTCTTCTTTCGAGAAGAACCCGA |
| | | TTGACTTCCTCGAGGCGAAGGGGTACAAGGAGGTGAAGAAGGATCTGATT |
| | | ATCAAGCTGCCGAAGTACTCCCTGTTCGAGCTGGAGAATGGTCGTAAGCG |
| | | TATGCTGGCGTagGCGGGTGAGtagCAGAAGGGGAACGAGTTGGCCCTTC |
| | | CGTCCAAGTACGTGAACTTCCTGTACCTGGCCTCGCACTACGAGAAGCTG |
| | | AAGGGTTCTCCGGAGGATAATGAGCAGAAGCAGCTGTTCGTGGAGCAGCA |
| | | CAAGCACTACCTGGACGAGATTATTGAGCAGATTTCTGAGTTTTCTAAGC |
| | | GCGTGATTCTGGCGGACGCGAATCTGGATAAGGTCCTGTCTGCCTACAAT |
| | | AAGCACCGTGATAAGCCGATCCGTGAGCAGGCGGAGAACATCATTCACCT |
| | | GTTCACGCTGACTAATCTTGGTGCTCCGGCGGCCTTCAAGTACTTCGACA |
| | | CCACGATCGATCGTAAGCGTTACACCTCCACTAAGGAGGTCCTGGATGCG |
| | | ACTCTTATTCACCAGTCTATCACTGGCCTGTACGAGACTCGTATTGATCT |
| | | GAGtCAGTTGGGCGGTGACTAATaa |
| Cas9_T4 | 102 | aAAAAGACGGAGGTACAGACGGGCGGCTTCAGCAAGGAAAGCATCCTGCC |
| | | GAAACGCAACAGCGACAAACTGATCGCGCGCtagAAAGACTGGGACCCGA |
| | | AGAAATACGGCGGCTTCGACAGCCCAACCGTGGCATACAGCGTGCTGGTA |
| | | GTCGCCAAAGTCGAAAAGGGCAAAAGCAAAAAACTGAAAAGCGTGAAAGA |
| | | ACTGCTGGGCATCACCATCATGGAACGCAGCAGCTTCGAAAAAAACCCGA |
| | | TCGACTTCCTCGAAGCGAAGGGGTACAAGGAAGTAAAGAAAGACCTGATC |
| | | ATCAAACTGCCGAAGTACAGCCTGTTCGAACTGGAAAACGGCCGCAAACG |
| | | CATGCTGGCGAGCGCGGGCGAACTGCAAAAAGGGAACGAACTGGCCCTGC |
| | | CGAGCAAGTACGTGAACTTCCTGTACCTGGCCAGCCACTACGAGAAACTG |
| | | AAAGGCAGCCCGGAAGACAACGAGCAGAAACAGCTGTTCGTGGAACAGCA |
| | | CAAACACTACCTGGACGAGATCATCGAACAGATCAGCGAGTTCAGCAAAC |
| | | GCGTAATCCTGGCGGACGCGAACCTGGACAAAGTCCTGAGCGCCTACAAC |
| | | AAACACCGCGACAAACCGATCCGCGAACAGGCAGAAAACATCATCCACCT |
| | | GTTCACGCTGACGAACCTGGGCGCCCCGGCAGCCTTCAAATACTTCGACA |
| | | CCACGATCGACCGCAAACGCTACACCAGCACGAAAGAAGTCCTGGACGCA |
| | | ACGCTGATCCACCAGAGCATCACGGGCCTGTACGAAACGCGCATCGACCT |
| | | GAGtCAGCTGGGCGGCGACTAATaa |
| Cas9_T5 | 103 | aAAAAGACGGAGGTACAGACGGGCGGCTTCAGCAAGGAAAGCATCCTGCC |
| | | GAAACGCAACAGCGACAAACTGATCGCGtgatagAAAGACTGGGACCCGA |
| | | AGAAATACGGCGGCTTCGACAGCCCAACCGTGGCATACAGCGTGCTGGTA |
| | | GTCGCCAAAGTCGAAAAGGGCAAAAGCAAAAAACTGAAAAGCGTGAAAGA |
| | | ACTGCTGGGCATCACCATCATGGAACGCAGCAGCTTCGAAAAAAACCCGA |
| | | TCGACTTCCTCGAAGCGAAGGGGTACAAGGAAGTAAAGAAAGACCTGATC |
| | | ATCAAACTGCCGAAGTACAGCCTGTTCGAACTGGAAAACGGCCGCAAACG |
| | | CATGCTGGCGAGCGCGGGCGAACTGCAAAAAGGGAACGAACTGGCCCTGC |
| | | CGAGCAAGTACGTGAACTTCCTGTACCTGGCCAGCCACTACGAGAAACTG |
| | | AAAGGCAGCCCGGAAGACAACGAGCAGAAACAGCTGTTCGTGGAACAGCA |
| | | CAAACACTACCTGGACGAGATCATCGAACAGATCAGCGAGTTCAGCAAAC |
| | | GCGTAATCCTGGCGGACGCGAACCTGGACAAAGTCCTGAGCGCCTACAAC |
| | | AAACACCGCGACAAACCGATCCGCGAACAGGCAGAAAACATCATCCACCT |
| | | GTTCACGCTGACGAACCTGGGCGCCCCGGCAGCCTTCAAATACTTCGACA |
| | | CCACGATCGACCGCAAACGCTACACCAGCACGAAAGAAGTCCTGGACGCA |
| | | ACGCTGATCCACCAGAGCATCACGGGCCTGTACGAAACGCGCATCGACCT |
| | | GAGtCAGCTGGGCGGCGACTAATaa |
| Cas9_T6 | 104 | aAAAAGACGGAGGTACAGACGGGCGGCTTCAGCAAGGAAAGCATCCTGCC |
| | | GAAACGCAACAGCGACAAACTGATCGCGCGCAAGAAAGACTGGGACCCGA |
| | | AGAAATACGGCGGCTTCGACAGCCCAACCGTGGCATACAGCGTGCTGGTA |
| | | GTCGCCAAAGTCGAAAAGGGCAAAAGCAAAAAACTGAAAAGCGTGAAAGA |
| | | ACTGCTGGGCATCACCATCATGGAACGCAGCAGCTTCGAAAAAAACCCGA |
| | | TCGACTTCCTCGAAGCGAAGGGGTACAAGGAAGTAAAGAAAGACCTGATC |
| | | ATCAAACTGCCGAAGTACAGCCTGTTCGAACTGGAAAACGGCCGCAAACG |
| | | CATGCTGGCGAGCGCGGGCGAACTGCAAAAAGGGAACGAACTGGCCCTGC |
| | | CGAGCAAGTACGTGAACTTCCTGTACCTGGCCAGCCACTACGAGAAACTG |
| | | AAAGGCAGCCCGGAAGACAACGAGCAGAAACAGCTGTTCGTGGAACAGCA |
| | | CAAACACTACCTGGACGAGATCATCGAACAGATCAGCGAGTTCAGCAAAC |
| | | GCGTAATCCTGGCGGACGCGAACCTGGACAAAGTCCTGAGCGCCTACAAC |
| | | AAACACCGCGACAAACCGATCCGCGAACAGGCAGAAAACATCATCCACCT |
| | | GTTCACGCTGACGAACCTGGGCGCCCCGGCAGCCTTCAAATACTTCGACA |
| | | CCACGATCGACCGCtagtgaTACACCAGCACGAAAGAAGTCCTGGACGCA |
| | | ACGCTGATCCACCAGAGCATCACGGGCCTGTACGAAACGCGCATCGACCT |
| | | GAGtCAGCTGGGCGGCGACTAATaa |
| pFD148 | 105 | GGATCGGCCTATGAACTGTCGACTCGAGGCCTTGACAGGTACCTCATGGA |
| | | TACCTATAATGCCGTGGGTGATGGTTTGATACAAAGGAGAAATCTAGATG |
| | | GTTTCCAAGGGCGAGGAGGATAACATGGCTATCATTAAAGAGTTCATGCG |
| | | CTTCAAAGTTCACATGGAGGGTTCTGTTAACGGTCACGAGTTCGAGATCG |
| | | AAGGCGAAGGCGAGGGCCGTCCGTATGAAGGCACCCAGACCGCCAAACTG |
| | | AAAGTGACTAAAGGCGGCCCGCTGCCTTTTGCGTGGGACATCCTGAGCCC |
| | | GCAATTTATGTACGGTTCTAAAGCTTATGTTAAACACCCAGCGGATATCC |
| | | CGGACTATCTGAAGCTGTCTTTTCCGGAAGGTTTCAAGTGGGAACGCGTA |
| | | ATGAATTTTGAAGATGGTGGTGTCGTGACCGTCACTCAGGACTCCTCCCT |
| | | GCAGGATGGCGAGTTCATCTATAAAGTTAAACTGCGTGGTACTAATTTTC |
| | | CATCTGATGGCCCGGTGATGCAGAAGAAGACGATGGGTTGGGAGGCGTCT |
| | | AGCGAACGCATGTATCCGGAAGATGGTGCGCTGAAAGGCGAAATTAAACA |
| | | GCGCCTGAAACTGAAAGATGGCGGCCATTATGACGCTGAAGTGAAAACCA |
| | | CGTACAAAGCCAAGAAACCTGTGCAGCTGCCTGGCGCGTACAATGTGAAT |
| | | ATTAAACTGGACATCACCTCTCATAATGAAGATTATACGATCGTAGAGCA |
| | | ATATGAGCGCGCGGAGGGTCGTCATTCTACCGGTGGCATGGATGAGCTGT |
| | | ACAAATAATGCGTACATAAAAAAGGAGACATGAACGATGaacatcaaaaa |
| | | gtttgcaaaacaagcaacagtattaacctttactaccgcactgctggcag |
| | | gaggcgcaactcaagcgtttgcgaaagaaacgaaccaaaagccatataag |
| | | gaaacatacggcatttcccatattacacgccatgatatgctgcaaatccc |
| | | tgaacagcaaaaaaatgaaaaatatcaagttcctgaattcgattcgtcca |
| | | caattaaaaatatctcttctgcaaaaggcctggacgtttgggacagctgg |
| | | ccattacaaaacgctgacggcactgtcgcaaactatcacggctaccacat |
| | | cgtctttgcattagccggagatcctaaaaatgcggatgacacatcgattt |
| | | acatgttctatcaaaaagtcggcgaaacttctattgacagctggaaaaac |
| | | gctggccgcgtctttaaagacagcgacaaattcgatgcaaatgattctat |
| | | cctaaaagaccaaacacaagaatggtcaggttcagccacatttacatctg |
| | | acggaaaaatccgtttattctacactgatttctccggtaaacattacggc |
| | | aaacaaacactgacaactgcacaagttaacgtatcagcatcagacagctc |
| | | tttgaacatcaacggtgtagaggattataaatcaatctttgacggtgacg |
| | | gaaaaacgtatcaaaatgtacagcagttcatcgatgaaggcaactacagc |
| | | tcaggcgacaaccatacgctgagagatcctcactacgtagaagataaagg |
| | | ccacaaatacttagtatttgaagcaaacactggaactgaagatggctacc |
| | | aaggcgaagaatctttatttaacaaagcatactatggcaaaagcacatca |
| | | ttcttccgtcaagaaagtcaaaaacttctgcaaagcgataaaaaacgcac |
| | | ggctgagttagcaaacggcgctctcggtatgattgagctaaacgatgatt |
| | | acacactgaaaaaagtgatgaaaccgctgattgcatctaacacagtaaca |
| | | gatgaaattgaacgcgcgaacgtctttaaaatgaacggcaaatggtacct |
| | | gttcactgactcccgcggatcaaaaatgacgattgacggcattacgtcta |
| | | acgatatttacatgcttggttatgtttctaattctttaactggcccatac |
| | | aagccgctgaacaaaactggccttgtgttaaaaatggatcttgatcctaa |
| | | cgatgtaacctttacttactcacacttcgctgtacctcaagcgaaaggaa |
| | | acaatgtcgtgattacaagctatatgacaaacagaggattctacgcagac |
| | | aaacaatcaacgtttgcgccaagcttcctgctgaacatcaaaggcaagaa |
| | | aacatctgttgtcaaagacagcatccttGAACAAGGACAATTAACAGTTA |
| | | ACAAATAAAGGCATGCCTCGAGATGCATGGCGCCTAACCTAAACTGACAG |
| | | GCATCAAATTAAGCAGAAGGCCATCCTGACGGATGGCCTTTTTGCGTTTC |
| | | GAACAATTGAAAAAACCTCGCGCCTTACCTGTTGAGTAATAGTCAAAAGC |
| | | CTCCGGTCGGAGGCTTTTGACTTTCTGCTTACTGAATTTCGGTGGTGCCG |
| | | TTAATTAACCGGTGGGCCCTCATGATAATAATGGTTTCTTAGACGTCCGA |
| | | AGTTCCTATTCTCTAGAAAGTATAGGAACTTCcctaggTCAGCCAAACGT |
| | | CTCTTCAGGCCACTGACTAGCGATAACTTTCCCCACAACGGAACAACTCT |
| | | CATTGCATGGGATCATTGGGTACTGTGGGTTTAGTGGTTGTAAAAACACC |
| | | TGACCGCTATCCCTGATCAGTTTCTTGAAGGTAAACTCATCACCCCCAAG |
| | | TCTGGCTATGCAGAAATCACCTGGCTCAACAGCCTGCTCAGGGTCAACGA |
| | | GAATTAACATTCCGTCAGGAAAGCTTGGCTTGGAGCCTGTTGGTGCGGTC |
| | | ATGGAATTACCTTCAACCTCAAGCCAGAATGCAGAATCACTGGCTTTTTT |
| | | GGTTGTGCTTACCCATCTCTCCGCATCACCTTTGGTAAAGGTTCTAAGCT |
| | | TAGGTGAGAACATCCCTGCCTGAACATGAGAAAAAACAGGGTACTCATAC |
| | | TCACTTCTAAGTGACGGCTGCATACTAACCGCTTCATACATCTCGTAGAT |
| | | TTCTCTGGCGATTGAAGGGCTAAATTCTTCAACGCTAACTTTGAGAATTT |
| | | TTGTAAGCAATGCGGCGTTATAAGCATTTAATGCATTGATGCCATTAAAT |
| | | AAAGCACCAACGCCTGACTGCCCCATCCCCATCTTGTCTGCGACAGATTC |
| | | CTGGGATAAGCCAAGTTCATTTTTCTTTTTTTCATAAATTGCTTTAAGGC |
| | | GACGTGCGTCCTCAAGCTGCTCTTGTGTTAATGGTTTCTTTTTTGTGCTC |
| | | ATACGTTAAATCTATCACCGCAAGGGATAAATATCTAACACCGTGCGTGT |
| | | TGACTATTTTACCTCTGGCGGTGATAATGGTTGCATGTACTAAGGAGGTT |
| | | GTATacgcgtCGTCGATGGGTTCGGGAATGCAGGATATTCCCACCTCCGG |
| | | TTAAGGATGGAAGAGAGTATCTGTTCCACGAATCAGCGGTAAAGGTTGAC |
| | | TTAAATCGACCAGTAACAGGTGGCCTTTTGAAGAGGATCAGAAATGGGAA |
| | | GAAGGCGAAGTCATGAGCGCCGGGATTTACCCCCTAACCTTTATATAAGA |
| | | AACAATGGATATTACTGCTACAGGGACCCAAGGACGGGTAAAGAGTTTGG |
| | | ATTAGGCAGAGACAGGCGAATCGCAATCACTGAAGCTATACAGGCCAACA |
| | | TTGAGTTATTTTCAGGACACAAACACAAGCCTCTGACAGCGAGAATCAAC |
| | | AGTGATAATTCCGTTACGTTACATTCATGGCTTGATCGCTACGAAAAAAT |
| | | CCTGGCCAGCAGAGGAATCAAGCAGAAGACACTCATAAATTACATGAGCA |
| | | AAATTAAAGCAATAAGGAGGGGTCTGCCTGATGCTCCACTTGAAGACATC |
| | | ACCACAAAAGAAATTGCGGCAATGCTCAATGGATACATAGACGAGGGCAA |
| | | GGCGGCGTCAGCCAAGTTAATCAGATCAACACTGAGCGATGCATTCCGAG |
| | | AGGCAATAGCTGAAGGCCATATAACAACAAACCATGTCGCTGCCACTCGC |
| | | GCAGCAAAATCAGAGGTAAGGAGATCAAGACTTACGGCTGACGAATACCT |
| | | GAAAATTTATCAAGCAGCAGAATCATCACCATGTTGGCTCAGACTTGCAA |
| | | TGGAACTGGCTGTTGTTACCGGGCAACGAGTTGGTGATTTATGCGAAATG |
| | | AAGTGGTCTGATATCGTAGATGGATATCTTTATGTCGAGCAAAGCAAAAC |
| | | AGGCGTAAAAATTGCCATCCCAACAGCATTGCATATTGATGCTCTCGGAA |
| | | TATCAATGAAGGAAACACTTGATAAATGCAAAGAGATTCTTGGCGGAGAA |
| | | ACCATAATTGCATCTACTCGTCGCGAACCGCTTTCATCCGGCACAGTATC |
| | | AAGGTATTTTATGCGCGCACGAAAAGCATCAGGTCTTTCCTTCGAAGGGG |
| | | ATCCGCCTACCTTTCACGAGTTGCGCAGTTTGTCTGCAAGACTCTATGAG |
| | | AAGCAGATAAGCGATAAGTTTGCTCAACATCTTCTCGGGCATAAGTCGGA |
| | | CACCATGGCATCACAGTATCGTGATGACAGAGGCAGGGAGTGGGACAAAA |
| | | TTGAAATCAAATAATGATTTTATTTTGACTGATAGTGACCTGTTCGTTGC |
| | | AACAAATTGATAAGCAATGCCAAGCTTGGCACTGGCTGATCAGCTAGCCC |
| | | ATGGGTATGGACAGTTTTCCCTTTGATATGTAACGCACGTTGTGTCTCAA |
| | | AATCTCTGATGTTACATTGCACAAGATAAAAATATATCATCATGAACAAT |
| | | AAAACTGTCTGCTTACATAAACAGTAATACAAGGGGTGTTATGAGCCATA |
| | | TTCAACGGGAAACGTCTTGCTCCCGTCCGCGCTTAAACTCCAACATGGAC |
| | | GCTGATTTATATGGGTATAAATGGGCTCGCGATAATGTCGGGCAATCAGG |
| | | TGCGACAATCTATCGCTTGTATGGGAAGCCCGATGCGCCAGAGTTGTTTC |
| | | TGAAACATGGCAAAGGTAGCGTTGCCAATGATGTTACAGATGAGATGGTC |
| | | CGTCTCAACTGGCTGACGGAGTTTATGCCTCTCCCGACCATCAAGCATTT |
| | | TATCCGTACTCCTGATGATGCGTGGTTACTCACCACCGCGATTCCTGGGA |
| | | AAACAGCCTTCCAGGTATTAGAAGAATATCCTGATTCAGGTGAAAATATT |
| | | GTTGATGCGCTGGCCGTGTTCCTGCGCCGGTTACATTCGATTCCTGTTTG |
| | | TAATTGTCCTTTTAACAGCGATCGTGTATTTCGTCTTGCTCAGGCGCAAT |
| | | CACGCATGAATAACGGTTTGGTTGATGCGAGTGATTTTGATGACGAGCGT |
| | | AATGGCTGGCCTGTTGAACAAGTCTGGAAAGAAATGCACAAGCTCTTGCC |
| | | ATTCTCACCGGATTCAGTCGTCACTCATGGTGATTTCTCACTTGATAACC |
| | | TTATTTTTGACGAGGGGAAATTAATAGGTTGTATTGATGTTGGACGGGTC |
| | | GGAATCGCAGACCGTTACCAGGACCTTGCCATTCTTTGGAACTGCCTCGG |
| | | TGAGTTTTCTCCTTCATTACAGAAACGGCTTTTTCAAAAATATGGTATTG |
| | | ATAATCCTGATATGAATAAATTGCAGTTTCATTTGATGCTCGATGAGTTT |
| | | TTCTAATAATACTAGCAGAAATCATCCTTAGCGAAAGCTAAGGATTTTTT |
| | | TTATCTGATTACCGCCTTTGAGTGAGCGTCGACCTAGTGCGGCCGCAAGA |
| | | TCCGGCCACGATGCGTCCGGCGTAGAGGATCTGAAGATCAGCAGTTCAAC |
| | | CTGTTGATAGTACGTACTAAGCTCTCATGTTTCACGTACTAAGCTCTCAT |
| | | GTTTAACGTACTAAGCTCTCATGTTTAACGAACTAAACCCTCATGGCTAA |
| | | CGTACTAAGCTCTCATGGCTAACGTACTAAGCTCTCATGTTTCACGTACT |
| | | AAGCTCTCATGTTTGAACAATAAAATTAATATAAATCAGCAACTTAAATA |
| | | GCCTCTAAGGTTTTAAGTTTTATAAGAAAAAAAAGAATATATAAGGCTTT |
| | | TAAAGCTTTTAAGGTTTAACGGTTGTGGACAACAAGCCAGGGATGTAACG |
| | | CACTGAGAAGCCCTTAGAGCCTCTCAAAGCAATTTTGAGTGACACAGGAA |
| | | CACTTAACGGCTGACATGGGAATTAGGAAGTTCCTATTCCGAAGTTCCTA |
| | | TTCTCTAGAAAGTATAGGAACTTCCATATGCCATGGCATCACAGTATCGT |
| | | GATGACAGAGGCAGGGAGTGGGACAAAATTGAAATCAAATAATGATTTTA |
| | | TTTTGACTGATAGTGACCTGTTCGTTGCAACAAATTGATAAGCAATGCTT |
| | | TTTTATAATGCCAACTTAGTATAAAAAAGCTGAACGAGAAACGTAAAATG |
| | | ATATAAATATCAATATATTAAATTAGATTTTGCATAAAAAACAGACTACA |
| | | TAATACTGTAAAACACAACATATATGCAGTCACTATGAATCAACTACTTA |
| | | GATGGTATTAGTGACCTGTAACAGAGCATTAGCGCAAGGTGATTTTTGTC |
| | | TTCTTGCGCTAATTTTTTGTCATCAAACCTGTCGCACTCCAGAGAAGCAC |
| | | AAAGCCTCGCAATCCAGTGCAAAGCTAGCTTCTTCGTCTGTTTCTACTGG |
| | | TATTGGCACAAACCTGATTCCAATTTGAGCAAGGCTATGTGCCATCTCGA |
| | | TACTCGTTCTTAACTCAACAGAAGATGCTTTGTGCATACAGCCCCTCGTT |
| | | TATTATTTATCTCCTCAGCCAGCCGCTGTGCTTTCAGTGGATTTCGGATA |
| | | ACAGAAAGGCCGGGAAATACCCAGCCTCGCTTTGTAACGGAGTAGAGACG |
| | | AAAGTGATTGCGCCTACCCGGATATTATCGTGAGGATGCGTCATCGCCAT |
| | | TAATTCACTGATCAGTGATAGCTGTCAAACATGAGAATTGATCCGGCTGC |
| | | CTCGCGCGTTTCGGTGATGACGGTGAAAACCTCTGACACATGCAGCTCCC |
| | | GGAGACGGTCACAGCTTGTCTGTAAGCGGATGCCGGGAGCAGACAAGCCC |
| | | GTCAGGGCGCGTCAGCGGGTGTTGGCGGGTGTCGGGGCGCAGCCATGACC |
| | | CAGTCACGTAGCGATAGCGGAGTGTATGCTGCACATGACATTAACCTATA |
| | | AAAATAGGCGTATCACGAGGCCCTTTCGTCTTCAAGAATTAATTCCCAAT |
| | | TCCAGGCATCAAATAAAACGAAAGGCTCAGTCGAAAGACTGGGCCTTTCG |
| | | TTTTATCTGTTGTTTGTCGGTGAACGCTCTCCTGAGTAGGACAAATCCGC |
| | | CGGGAGCGGATTTGAACGTTGCGAAGCAACGGCCCGGAGGGTGGCGGGCA |
| | | GGACGCCCGCCATAAACTGCCAGGAATTAATTCCAGGCATCAAATAAAAC |
| | | GAAAGGCTCAGTCGAAAGACTGGGCCTTTCGTTTTATCTGTTGTTTGTCG |
| | | GTGAACGCTCTCCTGAGTAGGACAAATCCGCCGGGAGCGGATTTGAACGT |
| | | TGCGAAGCAACGGCCCGGAGGGTGGCGGGCAGGACGCCCGCCATAAACTG |
| | | CCAGGAATTAATTCCAGGCATCAAATAAAACGAAAGGCTCAGTCGAAAGA |
| | | CTGGGCCTTTCGTTTTATCTGTTGTTTGTCGGTGAACGCTCTCCTGAGTA |
| | | GGACAAATCCGCCGGGAGCGGATTTGAACGTTGCGAAGCAACGGCCCGGA |
| | | GGGTGGCGGGCAGGACGCCCGCCATAAACTGCCAGGAATTAATTCCAGGC |
| | | ATCAAATAAAACGAAAGGCTCAGTCGAAAGACTGGGCCTTTCGTTTTATC |
| | | TGTTGTTTGTCGGTGAACGCTCTCCTGAGTAGGACAAATCCGCCGGGAGC |
| | | GGATTTGAACGTTGCGAAGCAACGGCCCGGAGGGTGGCGGGCAGGACGCC |
| | | CGCCATAAACTGCCAGGAATTGG |
| pWR63_pIGO12 | 106 | gattagcagaaagtcaaaagcctccgaccggaggcttttgactaaaactt |
| (8493 bp) | | cccttggggttatcattggggctcactcaaaggcggtaatcagataaaaa |
| | | aaatccttagctttcgctaaggatgatttctgctagagctgtcagaccaa |
| | | gtttacgagctcgcttggactcctgttgatagatccagtaatgacctcag |
| | | aactccatctggatttgttcagaacgctcggttgccgccgggcgCttttt |
| | | attggtgagaatccaagcactagggacagtaagacgggtaagcctgttga |
| | | tgataccgctgccttactgggtgcattagccagtctgaatgacctgtcac |
| | | gggataatccgaagtggccagactggaaaatcagagggcaggaactgctg |
| | | aacagcaaaaagtcagatagcaccacatagcagacccgccataaaacgcc |
| | | ctgagaagcccgtgacgggcttttcttgtattatgggtagtttccttgca |
| | | tgaatccataaaaggcgcctgtagtgccatttacccccattcactgccag |
| | | agccgtgagcgcagcgaactgaatgtcacgaaaaagacagcgactcaggt |
| | | gcctgatggtcggagacaaaaggaatattcagcgatttgcccgagcttgc |
| | | gagggtgctacttaagcctttagggttttaaggtctgttttgtagaggag |
| | | caaacagcgtttgcgacatccttttgtaatactgcggaactgactaaagt |
| | | agtgagttatacacagggctgggatctattctttttatctttttttattc |
| | | tttctttattctataaattataaccacttgaatataaacaaaaaaaacac |
| | | acaaaggtctagcggaatttacagagggtctagcagaatttacaagtttt |
| | | ccagcaaaggtctagcagaatttacagatacccacaactcaaaggaaaag |
| | | gacatgtaattatcattgactagcccatctcaattggtatagtgattaaa |
| | | atcacctagaccaattgagatgtatgtctgaattagttgttttcaaagca |
| | | aatgaactagcgattagtcgctatgacttaacggagcatgaaaccaagct |
| | | aattttatgctgtgtggcactactcaaccccacgattgaaaaccctacaa |
| | | ggaaagaacggacggtatcgttcacttataaccaatacgctcagatgatg |
| | | aacatcagtagggaaaatgcttatggtgtattagctaaagcaaccagaga |
| | | gctgatgacgagaactgtggaaatcaggaatcctttggttaaaggctttg |
| | | agattttccagtggacaaactatgccaagttctcaagcgaaaaattagaa |
| | | ttagtttttagtgaagagatattgccttatcttttccagttaaaaaaatt |
| | | cataaaatataatctggaacatgttaagtcttttgaaaacaaatactcta |
| | | tgaggatttatgagtggttattaaaagaactaacacaaaagaaaactcac |
| | | aaggcaaatatagagattagccttgatgaatttaagttcatgttaatgct |
| | | tgaaaataactaccatgagtttaaaaggcttaaccaatgggttttgaaac |
| | | caataagtaaagatttaaacacttacagcaatatgaaattggtggttgat |
| | | aagcgaggccgcccgactgatacgttgattttccaagttgaactagatag |
| | | acaaatggatctcgtaaccgaacttgagaacaaccagataaaaatgaatg |
| | | gtgacaaaataccaacaaccattacatcagattcctacctacataacgga |
| | | ctaagaaaaacactacacgatgctttaactgcaaaaattcagctcaccag |
| | | ttttgaggcaaaatttttgagtgacatgcaaagtaagtatgatctcaatg |
| | | gttcgttctcatggctcacgcaaaaacaacgaaccacactagagaacata |
| | | ctggctaaatacggaaggatctgaggttcttatggctcttgtatctatca |
| | | gtgaagcatcaagactaacaaacaaaagtagaacaactgttcaccgttac |
| | | atatcaaagggaaaactgtccatatgcacagatgaaaacggtgtaaaaaa |
| | | gatagatacatcagagcttttacgagtttttggtgtattcaaagctgttc |
| | | accatgaacagatcgacaatgtaactactagagctaaatacattcaaata |
| | | tctatccgctcatgagacaataaccctgataaatgcttcaataatattga |
| | | aaaaggaagaatatgagtattcaacatttccgtgtcgcccttattccctt |
| | | ttttgcggcattttgccttcctgtttttgctcacccagaaacgctggtga |
| | | aagtaaaagatgccgaagatcagttgggtgcacgtgtgggttacatcgaa |
| | | ctggacctcaacagcggtaagattcttgagagttttcgccccgaagaacg |
| | | tttcccaatgatgagcacttttaaagttctgctctgtggcgcggtattat |
| | | cccgtattgacgccgggcaagagcaactcggtcgccgcatacactattct |
| | | cagaatgacttggttgagtactcaccagtcacagaaaagcatcttacgga |
| | | cggcatgacagtacgcgaattatgcagcgctgccataaccatgagtgata |
| | | acacggcggccaacttacttctgacaacgatcggaggaccgaaggagctt |
| | | accgcttttttgcacaacatgggtgatcatgtaactcgccttgatcgttg |
| | | ggaaccggagctgaatgaagccataccaaacgacgagcgtgacaccacga |
| | | tgcctgtagctatggcaacaacgttgcgcaaactcttaactggcgaactt |
| | | cttactctcgcttcccggcaacaattaatagactggatggaggcggataa |
| | | agttgcaggaccacttctgcgctcggcccttccggctggctggtttattg |
| | | ctgataaatctggagccggtgagcgtgggtcccgcggtattattgcagcc |
| | | ctggggccagatggtaagccctcccgtatcgtagttatctacacgacggg |
| | | gagccaggcaactatggacgaacgtaatcgccagatcgctgagataggtg |
| | | cctccctgattaagcattggtaataatactagctccggcaaaaaaacggg |
| | | caaggtgtcaccaccctgccctttttctttaaaaccgaaaagattacttc |
| | | gcgtttgccacctgacgtctaagaaaaggaatattcagcaatttgcccgt |
| | | gccgaagaaaggcccacccgtgaaggtgagccagtgagttgattgctacg |
| | | taattagtttttccataaaactaaagtaagtgtaaacctattcattgttt |
| | | taaaaatatctcttgccagtcacgttacgttattagttagttaccatcac |
| | | ggaaaaaggttatgctgcttttaagacccactttcacatttaagttgttt |
| | | ttctaatccgcatatgatcaattcaaggccgaataagaaggctggctctg |
| | | caccttggtgatcaaataattcgatagcttgtcgtaataatggcggcata |
| | | ctatcagtagtaggtgtttccctttcttctttagcgacttgatgctcttg |
| | | atcttccaatacgcaacctaaagtaaaatgccccacagcgctgagtgcat |
| | | ataatgcattctctagtgaaaaaccttgttggcataaaaaggctaattga |
| | | ttttcgagagtttcatactgtttttctgtaggccgtgtacctaaatgtac |
| | | ttttgctccatcgcgatgacttagtaaagcacatctaaaacttttagcgt |
| | | tattacgtaaaaaatcttgccagctttccccttctaaagggcaaaagtga |
| | | gtatggtgcctatctaacatctcaatggctaaggcgtcgagcaaagcccg |
| | | cttattttttacatgccaatacaatgtaggctgctctacacctagcttct |
| | | gggcgagtttacgggttgttaaaccttcgattccgacctcattaagcagc |
| | | tctaatgcgctgttaatcactttacttttatctaatctagacatcattaa |
| | | ttcctcctttttgttgacattatatcattgatagagttatttgtcaaact |
| | | agttttttatttggatcccctcgagttcatgaaaaactaaaaaaaatatt |
| | | gacactctatcattgatagagcataattaaaataagctccctatcagtga |
| | | tagagaggaggcatatcaaaggacgAgtgcaggtggcaaaaatggataag |
| | | aaatacagcataggcttagctatcggcacaaatagcgtcggatgggcggt |
| | | gatcactgatgaatataaagttccgtctaaaaagttcaaggtactgggta |
| | | atacagatcgccatagtatcaaaaagaacttaatcggtgcgcttctgttc |
| | | gattccggcgaaaccgcagaagcaacacgtctgaaacgcaccgctcgtcg |
| | | ccgttacacccgtcgtaaaaaccgcatctgctacctgcaagaaatcttct |
| | | ctaacgaaatggctaaagtagatgacagcttttttcaccgtctggaagaa |
| | | tcatttctggtggaagaagataaaaagcacgaacgtcatccaatcttcgg |
| | | caacattgtggacgaagtagcgtatcacgaaaaatacccgactatctatc |
| | | acctgcgcaaaaagctggtcgattcgacggataaggccgatctgcgtctg |
| | | atctatctggccttagcgcatatgattaagttccgtggtcatttcctgat |
| | | cgaaggcgacctgaatccagacaacagcgatgtagacaaactgttcatcc |
| | | agctggtgcaaacctataaccagctgtttgaagaaaacccaattaatgct |
| | | agcggtgttgacgcgaaagcgatcttgtccgcacgcctgtccaaatcccg |
| | | tcgtctggaaaacttaattgcgcaactgccgggtgagaagaaaaacggac |
| | | tgttcggcaatctgatcgctcttagcttgggactgaccccgaacttcaaa |
| | | agcaacttcgatctggcagaggacgcaaaacttcaacttagcaaagatac |
| | | gtatgacgatgacttggataacttactggcccagatcggagatcagtacg |
| | | ctgatctgtttctggcggcaaagaacttatcagacgctattctcctgtct |
| | | gatattcttcgtgtgaataccgaaatcaccaaagcaccgctttctgcatc |
| | | catgattaaacgctatgacgaacatcaccaagatctgactcttctgaaag |
| | | cgctggtacggcaacaactgccggagaagtacaaggagatcttctttgac |
| | | caatccaaaaacggctacgcgggttatattgacgggggtgcaagccaaga |
| | | ggagttctacaaattcatcaagccaatcttagaaaaaatggatggcacgg |
| | | aagaattacttgttaaactgaatcgtgaggatctgcttcgtaaacagcgt |
| | | accttcgacaacggtagcattccgcaccagatccacttaggtgaactgca |
| | | cgctatcctgcgtcgccaagaggatttttacccgttcctgaaagataatc |
| | | gtgaaaaaatcgaaaaaatcctgacctttcgtatcccgtattatgtcggc |
| | | ccgctggcgcgtggcaactcccgtttcgcgtggatgactcgcaaatccga |
| | | agaaactattaccccgtggaacttcgaggaagtggttgacaaaggcgcaa |
| | | gcgcccaatccttcatcgagcgcatgactaactttgataaaaacctgccg |
| | | aacgaaaaggtactgccgaaacactcccttctgtacgaatacttcaccgt |
| | | gtacaacgagctgactaaagtaaagtatgtgactgagggcatgcgtaaac |
| | | ctgcattcctgagcggtgaacagaaaaaagcaattgttgatttactgttt |
| | | aaaaccaaccgtaaagtaaccgttaaacagctgaaagaggactacttcaa |
| | | gaaaatcgaatgcttcgactccgtcgagattagtggagttgaagatcgtt |
| | | ttaatgcaagtttaggcacgtatcacgatttattaaagatcattaaagac |
| | | aaagatttcttggacaacgaagaaaatgaggacatcttagaggacatcgt |
| | | cctgaccctgactctgttcgaagatcgtgaaatgattgaagaacgcttga |
| | | agacgtatgctcacctgtttgacgataaagtaatgaaacaactgaaacgt |
| | | cgccgttatactggctggggccgtctgagccgtaaactgattaacggtat |
| | | ccgtgacaaacagtccggtaaaactattctggacttcctgaaatctgacg |
| | | gcttcgcaaaccgtaacttcatgcaactgattcacgacgattccctgacc |
| | | ttcaaagaggacatccagaaagctcaggtttctggtcaaggtgattctct |
| | | gcacgagcatatcgccaatttagcaggtagtccggcgatcaaaaaaggta |
| | | tcctgcaaaccgtgaaagtggtggatgagcttgtgaaagttatgggtcgt |
| | | cacaaaccggaaaacattgttatcgagatggctcgtgaaaaccaaacgac |
| | | ccagaagggacagaaaaactcccgcgaacgcatgaaacgtatcgaggagg |
| | | gtattaaagaacttggctctcagattctgaaagaacaccctgttgaaaat |
| | | acccaactgcaaaatgaaaaactgtacctgtactacctgcaaaatggtcg |
| | | tgacatgtatgtagatcaggagctggacatcaaccgcctctccgattacg |
| | | acgttgacgccattgttccgcagtcttttctgaaagatgattccattgat |
| | | aacaaagtactcacccgtagcgataaaaaccgtgggaagagtgacaacgt |
| | | tccatcggaagaagtagttaagaaaatgaagaactattggcgtcaactgc |
| | | ttaacgcgaaactgattactcaacgtaaatttgataacctgaccaaagct |
| | | gaacgtggcggtttgtctgagctggataaggcgggttttattaaacgtca |
| | | actggtagaaactcgccagattacaaaacatgttgctcagattctggact |
| | | ctcgtatgaacactaaatacgatgaaaatgacaaactgatccgcgaagtt |
| | | aaggttattaccctgaaatctaagctggtttccgacttccgtaaagattt |
| | | ccaattctataaagtgcgcgagattaacaactatcaccacgcgcacgacg |
| | | catatctgaatgcagttgttggcacggcactgatcaaaaaatatccgaaa |
| | | ctggaaagcgaatttgtgtacggcgattataaagtttacgacgtgcgcaa |
| | | aatgatcgccaaatctgaacaggaaattggcaaagcaaccgctaaatact |
| | | ttttctactcaaacattatgaatttcttcaaaaccgaaatcaccttagcg |
| | | aatggcgaaattcgtaaacgccctctgatcgaaaccaacggcgaaacggg |
| | | tgagatcgtgtgggacaaaggtcgtgatttcgctactgtccgcaaagttc |
| | | tgtccatgcctcaagtaaacatcgttaaaaacgagacctgaattgcgcgc |
| | | aattaaccctcactaaagggaacaaaagctggagctcttatattccccag |
| | | aacatcaggttaatggcgtttttgatgtcattttcgcggtggctgagatc |
| | | agccacttcttccccgataacggacaccggcacactggccatatcggtgg |
| | | tcatcatgcgccagctttcatccccgatatgcaccaccgggtaaagttca |
| | | cgggagactttatctgacagcagacgtgcactggccagggggatcaccat |
| | | ccgtcgcccgggcgtgtcaataatatcactctgtacatccacaaacagac |
| | | gataacggctctctcttttataggtgtaaaccttaaactgcatcgtttca |
| | | ctccatccaaaaaaacgggtatggagaaacagtagagagttgcgataaaa |
| | | agcgtcaggtaggatccgctggtctctaaggcgttaaaccaggcatcaaa |
| | | taaaacgaaaggctcagtcgaaagactgggcctttcgttttatctgttgt |
| | | ttgtcggtgaacgctctctactagagtcacactggctcaccttcgggtgg |
| | | gcctttctgcgtttataatatactcctaaattaacttttaaagcaatgaa |
| | | aatagtgaacattataactgttgtgtaacagaatgcaattagcatattac |
| | | tgttacacaaattagtacagtttctatgttttgacatacatttgatgaaa |
| | | attgtacataatttatgtgaaaaaaatcacaacaaacatgctacaattag |
| | | ttgagaccagtctaggtctcggttttagagctagaaatagcaagttaaaa |
| | | taaggctagtccgttatcaacttgaaaaagtggcaccgagtcggtgcttt |
| | | ttttggagatctgtccatacccatggattcttcgtctgtttctactccat |
| | | cagtctgacgaccaagagagccataaacaccaatagccttaac |
| mChg0 | 107 | CATGGATACCTATAATGCCG |
| DGR1 | 108 | CGGTGGCTTCGATTCTCCGACCGTGGCGtaaTCTGTTCTGGTGGTCGCCA |
| | | AGGTCGAGAAGGGTAAGTCT |
| DGR3 | 109 | AAGTACGGTGGCTTCGATTCTCCGACCGTGGCGaaaTCTGTTCTAGTGGT |
| | | CGCCAAGGTCGAGAAGGGTAAGTCTAAGAA |
| DGR_control | 110 | Cgctgctgcgctattcggcggcaactggaacaacacgtcgaactcgggtt |
| | | ctcgcgctgcgaactggaacaacgggccgtcgaactcgaacgcgaacatc |
| | | ggggcgcgcggcg |
| DGR2 | 111 | CGGTGGCTTCGATTCTCCGACCGTGGCGtaaTCTGTTtagGTGGTCGCCA |
| | | AGGTCGAGAAGGGTAAGTCT |
| DGR4 | 112 | AGCCGCCGTATTTCTTCGGGTCCCAGTCTTTctaGCGCGCGATCAGTTTG |
| | | TCGCTGTTGCGTTTCGGCAG |
| DGR5 | 113 | AGCCGCCGTATTTCTTCGGGTCCCAGTCTTTctatcaCGCGATCAGTTTG |
| | | TCGCTGTTGCGTTTCGGCAG |
| DGR6 | 114 | GGTGGATCAGCGTTGCGTCCAGGACTTCTTTCGTGCTGGTGTAtcactaG |
| | | CGGTCGATCGTGGTGTCGAAG |
| DGR7 | 115 | GCTTCGATTCACCGACCGTGGCGaaaTCTGTTCTGGTGGTCGCCAAGGTC |
| | | GAGAAGGGTA |
| DGR8 | 116 | GATTCTCCGACCGTGGCGaaaTCAGTTCTGGTGGTCGCCAAGGTCGAGAA |
| | | GGGTAAGTCTAAGAAGCTGA |
| DGR9 | 117 | TGGTCGTAAGCGTATGCTGGCGtagGCGGGTGAGtagCAGAAGGGGAACG |
| | | AGTTGGCCCTTCCGTCC |
| DGR10 | 118 | TACGGTGGCTTCAAATCTCCGACCGTGGCGaaaTCTGTTaaaGTAGTCGC |
| | | CAAGGTCGAG |
| DGR11 | 119 | TACGGTGGCTTCAAATCTCCGACCGTAGCGaaaTCTGTTaaaGTGGTCGC |
| | | CAAGGTCGAGAAGGGTAAGT |
| DGR12 | 120 | GAAGTACGGTGGCTTCAAATCTCCGACCGTGGCGTACTCTGTTCTGGTGG |
| | | TAGCCAAGGTCGAGAAGGGTAAGTCTAAGA |
| DGR13 | 121 | AAGCGTATGCTGGCGTCTGCAAAAAAACTGCAGAAGGGGAACGAGTTGGC |
| | | CCTTCCGTCC |
| DGR14 | 122 | AATGGTCGTAAGCGTATGCTGGCATCTGCGAAAAAACTGCAGAAGGGGAA |
| | | CGAGTTGGCCCTTCCGTCCA |
| DGR15 | 123 | CTGGAGAATGGTCGTAAGCGAATGCTGGCGTCTGCGAATGAACTGCAGAA |
| | | GGGGAACGAGTTGGCCCTTCCGTCCAAGTA |
| DGR16 | 124 | TCCCAGTCTTTAAAAAACGCGATCAGTTTGTCGCTGTTGCGTTTAGGAAA |
| | | GATGCTTTCC |
| DGR17 | 125 | CGGGTCCCAGTCTTTAAAAAAAGCGATCAGTTTGTCGCTGTTGCGTTTCG |
| | | GAAAGATGCTTTCCTTGCTG |
| DGR18 | 126 | GCCGCCGTATTTCTTCGGGTCCCAGTCTTTAAAAAACGCGATCAGTTTGT |
| | | CGCTGTTGCGTTTAGGAAAGATGCTTTCCT |
| DGR19 | 127 | GCGTTGCGTCCAGGACTTCTTTCGTGCTAAAGTAAAATTTACGATCGATC |
| | | GTGGTGTCG |
| DGR20 | 128 | GCTCTGGTGGATCAGCGTAGCGTCCAGGACTTCTTTCGTGCTAAAGTAAA |
| | | ATTTGCGATCGATCGTGGTGTCG |
| DGR21 | 129 | GCGTTGCGTCCAGGACTTCTTTCGTGCTAAAGTAAAATTTACGATCGATC |
| | | GTGGTGTCGAAGTATTTGAAGGCTGCCGGG |
| DGR1 target | 130 | GATCCGAAGAAGTACGGTGGCTTCGATTCTCCGACCGTGGCGtaaTCTGT |
| (*1141/nt | | TCTGGTGGTCGCCAAGGTCGAGAAGGGTAAGTCTAAGAAGCTGAAG |
| strand 1; | | |
| FIG. 13) | | |
| DGR1 target | 131 | CTAGGCTTCTTCATGCCACCGAAGCTAAGAGGCTGGCACCGCattAGACA |
| (*1141/nt | | AGACCACCAGCGGTTCCAGCTCTTCCCATTCAGATTCTTCGACTTC |
| strand 2; | | |
| FIG. 13 | | |
| DGR1 target | 132 | DPKKYGGFDSPTVA*SVLVVAKVEKGKSKKKLK |
| (*1141/aa; | | |
| FIG. 13) | | |
| Variant-DGR1 | 133 | GATCCGAAGAAGTACGGTGGCTTCGATTCTCCGACCGTGGCGAGGTCTGT |
| (v1 FIG. 13) | | TCTGGTGGTCGCCAAGGTCGAGAAGGGTAAGTCTAAGAAGCTGAAG |
| Variant-DGR1 | 134 | GATCCGAAGAAGTACGGTGGCTTCGATTCTCCGACCGTGGCGTTATCTGT |
| (v3 FIG. 13) | | TCTGGTGGTCGCCAAGGTCGAGAAGGGTAAGTCTAAGAAGCTGAAG |
| Variant-DGR1 | 135 | GATCCGAAGAAGTACGGTGGCTTCGATTCTCCGACCGTGGCGTTTTCTGT |
| (v5 FIG. 13) | | TCTGGTGGTCGCCAAGGTCGAGAAGGGTAAGTCTAAGAAGCTGAAG |
| Variant-DGR1 | 136 | GATCCGAAGAAGTACGGTGGCTTCGATTCTCCGACCGTGGCGGAATCTGT |
| (v6 FIG. 13) | | TCTGGTGGTCGCCAAGGTCGAGAAGGGTAAGTCTAAGAAGCTGAAG |
| Variant-DGR1 | 137 | GATCCGAAGAAGTACGGTGGCTTCGAGTCTCCGACCGTGGCGTGGTCTGT |
| (v7 FIG. 13) | | TCTGGTGGTCGCCAAGGTCGAGAAGGGTAAGTCTAAGAAGCTGAAG |
| Variant-DGR1 | 138 | GATCCGAAGAAGTACGGTGGCTTCGATTCTCCGCCCGTGGCGTTATCTGT |
| (v9 FIG. 13) | | TCTGGTGGTCGCCAAGGTCGAGAAGGGTAAGTCTAAGAAGCTGAAG |
| DGR3 target | 139 | ACTGGGATCCGAAGAAGTACGGTGGCTTCGATTCTCCGACCGTGGCGtaa |
| (*1141/nt | | TCTGTTCTGGTGGTCGCCAAGGTCGAGAAGGGTAAGTCTAAGAAGCTGAA |
| strand 1; | | GTCTGT |
| FIG. 14) | | |
| DGR3 target | 140 | TGACCCTAGGCTTCTTCATGCCACCGAAGCTAAGAGGCTGGCACCGCatt |
| (*1141/nt | | AGACAAGACCACCAGCGGTTCCAGCTCTTCCCATTCAGATTCTTCGACTT |
| strand 2; | | CAGACA |
| FIG. 14 | | |
| DGR3 target | 141 | DWDPKKYGGFDSPTVA* SVLVVAKVEKGKSKKLKSV |
| (*1141/aa; | | |
| FIG. 14) | | |
| Variant-DGR3 | 142 | ACTGGGATCCGAAGAAGTACGGTGGCTTCGATTCTCCGACCGTGGCGAAA |
| (vs1 FIG. 14) | | TCTGTTCTGGTGGTCGCCAAGGTCGAGAAGGGTAAGTCTAAGAAGCTGAA |
| | | GTCTGT |
| Variant-DGR3 | 143 | ACTGGGATCCGAAGAAGTACGGTGGCTTCGATTCTCCGGCCGTGGCGGAA |
| (vs2 FIG. 14) | | TCTGTTCTAGTGGTCGCCAAGGTCGAGAAGGGTAAGTCTAAGAAGCTGAA |
| | | GTCTGT |
| Variant-DGR3 | 144 | ACTGGGATCCGAAGAAGTACGGTGGCTTCGATTCTCCGACCGTGGCGAAA |
| (vs3 FIG. 14) | | TCTGTTCTGGTGGTCGCCGAGGTCGAGAAGGGTAAGTCTAAGAAGCTGAA |
| | | GTCTGT |
| Variant-DGR3 | 145 | ACTGGGATCCGAAGAAGTACGGTGGCTTCGATTCTCCGACTGTGGCGTAA |
| (vs4 FIG. 14 | | TCTGTTCTGGTGGTCGCCAAGGTCGAGAAGGGTAAGTCTAAGAAGCTGAA |
| | | GTCTGT |

*Recoded gene sequences

TABLE 8
TR cloning oligonucleotide sequences.

| Oligo | | |
|---|---|---|
| name | Sequence | TR |
| AM001 | ATAACGCTGCTGCGCTATTCGGCGGCAACTGGAACAACACGTCGAACTCG | Plasmid pAM001 |
| | GGTTCTCGCGC (SEQ ID NO: 26) | TR pair 1, fwd |
| AM002 | CGCAGCGCGAGAACCCGAGTTCGACGTGTTGTTCCAGTTGCCGCCGAATA | Plasmid pAM001 |
| | GCGCAGCAGCG (SEQ ID NO: 27) | TR pair 1, rvs |
| AM030 | TGCGAACTGGAACAACGGGCCGTCGAACTCGAACGCGAACATCGGGGCG | Plasmid pAM001 |
| | CGCGGCG (SEQ ID NO: 28) | TR pair 2, fwd |
| AM031 | CAGACGCCGCGCGCCCCGATGTTCGCGTTCGAGTTCGACGGCCCGTTGT | Plasmid pAM001 |
| | TCCAGTT (SEQ ID NO: 29) | TR pair 2, rvs |
| AM007 | ATAATTATATGGCTTTTGGTTCGTTTCTTTCGCAAACGCTTGAG | Plasmid pAM004 |
| | (SEQ ID NO: 30) | TR fwd |
| AM008 | CAGACTCAAGCGTTTGCGAAAGAAACGAACCAAAAGCCATATAA | Plasmid pAM004 |
| | (SEQ ID NO: 31) | TR rvs |
| AM017 | ATAATGCCGTATGTTTCCTTATATGGCTTTTGGTTCGTTTCTTTCGCAAA | Plasmid pAM007 |
| | CGCT (SEQ ID NO: 32) | TR pair 1, fwd |
| AM018 | CTCAAGCGTTTGCGAAAGAAACGAACCAAAAGCCATATAAGGAAACATAC | Plasmid pAM007 |
| | GGCA (SEQ ID NO: 33) | TR pair 1, rvs |
| AM019 | TGAGTTGCGCCTCCTGCCAGCAGTGCGGTAGTAAAGGTTAATACTGTTGC | Plasmid pAM007 |
| | ((SEQ ID NO: 34) | TR pair 2, fwd |
| AM020 | CAGAGCAACAGTATTAACCTTTACTACCGCACTGCTGGCAGGAGGCGCAA | Plasmid pAM007 |
| | (SEQ ID NO: 35) | TR pair 2, rvs |
| AM021 | ATAATTTGTGGCCTTTATCTTCTACGTAGTGAGGATCTCTCAGCGTATGG | Plasmid pAM009 |
| | TTGTCGCCTGAGCTGTAGTTGCCT (SEQ ID NO: 36) | TR fwd |
| AM022 | CAGAAGGCAACTACAGCTCAGGCGACAACCATACGCTGAGAGATCCTCAC | Plasmid pAM009 |
| | TACGTAGAAGATAAAGGCCACAAA (SEQ ID NO: 37) | TR rvs |
| AM024 | ATAACGTGATAGTTTGCGACAGTGCCGTCAGCGTTTTGTAATGGCCAGCT | Plasmid pAM010 |
| | GTCCCAAACGTCCAGGCCTTTTGC (SEQ ID NO: 38) | TR fwd |
| AM025 | CAGAGCAAAAGGCCTGGACGTTTGGGACAGCTGGCCATTACAAAACGCTG | Plasmid pAM010 |
| | ACGGCACTGTCGCAAACTATCACG (SEQ ID NO: 39) | TR rvs |
| AM027 | ATAAGTTTGGCGGTCTGGGTGCCTTCATACGGACGGCCCTCGCCTTCGCC | Plasmid pAM011 |
| | TTCGATCTCGAACTCGTGACCGTT (SEQ ID NO: 40) | TR fwd |
| AM028 | CAGAAACGGTCACGAGTTCGAGATCGAAGGCGAAGGCGAGGGCCGTCCG | Plasmid pAM011 |
| | TATGAAGGCACCCAGACCGCCAAAC (SEQ ID NO: 41) | TR rvs |

Oligonucleotide sequences used for TR cloning by Golden gate assembly.
Forward (fwd) and reverse (rvs) oligos are annealed, producing sticky ends compatible for Golden gate assembly into plasmid pRL021.
The longer TR sequences can be assembled by two or three pairs of oligos, annealed independently and further joined during the Golden Gate assembly reaction.

### REFERENCES

[1]S. Doulatov et al., “Tropism switching in Bordetella bacteriophage defines a family of diversity-generating retroelements,” Nature, vol. 431, no. 7007, pp. 476-481, September 2004.
[2]B. G. Paul et al., “Retroelement-guided protein diversification abounds in vast lineages of Bacteria and Archaea,” Nature Microbiology, vol. 2, no. 6, pp. 1-7, April 2017.
[3]L. Wu et al., “Diversity-generating retroelements: natural variation, classification and evolution inferred from a large-scale genomic survey,” Nucleic Acids Res., vol. 46, no. 1, pp. 11-24, January 2018.
[4]W. Dai, A. Hodes, W. H. Hui, M. Gingery, J. F. Miller, and Z. H. Zhou, “Three-dimensional structure of tropism-switching Bordetella bacteriophage,” Proc. Natl. Acad. Sci. U.S.A, vol. 107, no. 9, pp. 4347-4352, March 2010.
[5]H. Guo et al., “Target site recognition by a diversity-generating retroelement,” PLoS Genet., vol. 7, no. 12, p. e1002414, December 2011.
[6] In some embodiments, the step a) (mutagenesis) and/or the step b) (selection and/or screening) are repeated at least one time.
[7]S. A. McMahon et al., “The C-type lectin fold as an evolutionary solution for massive sequence variation,” Nat. Struct. Mol. Biol., vol. 12, no. 10, pp. 886-892, October 2005.
[8]S. Handa, B. G. Paul, J. F. Miller, D. L. Valentine, and P. Ghosh, “Conservation of the C-type lectin fold for accommodating massive sequence variation in archaeal diversity-generating retroelements,” BMC Struct. Biol., vol. 16, no. 1, p. 13, August 2016.
[9]S. S. Naorem et al., “DGR mutagenic transposition occurs via hypermutagenic reverse transcription primed by nicked template RNA,” Proc. Natl. Acad. Sci. U.S.A, vol. 114, no. 47, pp. E10187-E10195, November 2017.
[10]B.simon, A. Nyerges, and C. Pál, “Targeted mutagenesis of multiple chromosomal regions in microbes,” Curr. Opin. Microbiol., vol. 57, pp. 22-30, June 2020.
[11]A. J. Simon, S. d'Oelsnitz, and A. D. Ellington, “Synthetic evolution,” Nat. Biotechnol., vol. 37, no. 7, pp. 730-743, July 2019.
[12]K. M. Esvelt, J. C. Carlson, and D. R. Liu, “A system for the continuous directed evolution of biomolecules,” Nature, vol. 472, no. 7344, pp. 499-503, April 2011.
[13]S. O. Halperin, C. J. Tou, E. B. Wong, C. Modavi, D. V. Schaffer, and J. E. Dueber, “CRISPR-guided DNA polymerases enable diversification of all nucleotides in a tunable window,” Nature, vol. 560, no. 7717, pp. 248-252, August 2018.
[14]B. Álvarez, M. Mencía, V. de Lorenzo, and L. Á. Fernández, “In vivo diversification of target genomic sites using processive base deaminase fusions blocked by dCas9,” Nat. Commun., vol. 11, no. 1, p. 6436, December 2020.
[15]A. J. Simon, B. R. Morrow, and A. D. Ellington, “Retroelement-Based Genome Editing and Evolution,” ACS Synth. Biol., vol. 7, no. 11, pp. 2600-2611, November 2018.
[16]N. Crook, J. Abatemarco, J. Sun, J. M. Wagner, A. Schmitz, and H. S. Alper, “In vivo continuous evolution of genes and pathways in yeast,” Nat. Commun., vol. 7, p. 13051, October 2016.
[17]S. P. Finney-Manchester and N. Maheshri, “Harnessing mutagenic homologous recombination for targeted mutagenesis in vivo by TaGTEAM,” Nucleic Acids Res., vol. 41, no. 9, p. e99, May 2013.
[18]E. Sharon, S.-A. A. Chen, N. M. Khosla, J. D. Smith, J. K. Pritchard, and H. B. Fraser, “Functional Genetic Variants Revealed by Massively Parallel Precise Genome Editing,” Cell, vol. 175, no. 2, pp. 544-557.e16, October 2018.
[19]S. C. Lopez, K. D. Crawford, S. Bhattarai-Kline, and S. L. Shipman, “Improved architectures for flexible DNA production using retrons across kingdoms of life,” bioRxiv, p. 2021.03.26.437017, Mar. 26, 2021.
[20]B. Zhao, S.-A. A. Chen, J. Lee, and H. B. Fraser, “Bacterial retrons enable precise gene editing in human cells,” bioRxiv, p. 2021.03.29.437260, Mar. 29, 2021.
[21]E. M. Barbieri, P. Muir, B. O. Akhuetie-Oni, C. M. Yellman, and F. J. Isaacs, “Precise Editing at DNA Replication Forks Enables Multiplex Genome Engineering in Eukaryotes,” Cell, vol. 171, no. 6, pp. 1453-1467.e13, November 2017.
[22]F. Farzadfard and T. K. Lu, “Synthetic biology. Genomically encoded analog memory with precise in vivo DNA writing in living cell populations,” Science, vol. 346, no. 6211, p. 1256272, November 2014.
[23]M. G. Schubert et al., “High throughput functional variant screens via in-vivo production of single-stranded DNA,” bioRxiv, p. 2020.03.05.975441, Mar. 6, 2020.
[24]F. Farzadfard, N. Gharaei, R. J. Citorik, and T. K. Lu, “Efficient Retroelement-Mediated DNA Writing in Bacteria,” Cold Spring Harbor Laboratory, p. 2020.02.21.958983, Feb. 22, 2020.
[25]S. Handa, A. Reyna, T. Wiryaman, and P. Ghosh, “Determinants of Selective Fidelity in Diversity-Generating Retroelements,” Cold Spring Harbor Laboratory, p. 2020.04.29.068544, Apr. 30, 2020.
[26]T. M. Wannier et al., “Recombineering and MAGE,” Nature Reviews Methods Primers, vol. 1, no. 1, pp. 1-24, January 2021
[27]J. Garamella, R. Marshall, M. Rustad, and V. Noireaux, “The All E. coli TX-TL Toolbox 2.0: A System for Cell-Free Synthetic Biology,” ACS Synth. Biol., vol. 5, no. 4, pp. 344-355, April 2016.
[28]K. Yehl et al., “Engineering Phage Host-Range and Suppressing Bacterial Resistance through Phage Tail Fiber Mutagenesis,” Cell, vol. 179, no. 2, pp. 459-469.e9, October 2019.
[29]S. Lemire, K. M. Yehl, and T. K. Lu, “Phage-Based Applications in Synthetic Biology,” Annu Rev Virol, vol. 5, no. 1, pp. 453-476, September 2018.
[30]S. Chatterjee and E. Rothenberg, “Interaction of bacteriophage 1 with its E. coli receptor, LamB,” Viruses, vol. 4, no. 11, pp. 3162-3178, November 2012.
[31]E. Berkane, F. Orlik, J. F. Stegmeier, A. Charbit, M. Winterhalter, and R. Benz, “Interaction of bacteriophage lambda with its cell surface receptor: an in vitro study of binding of the viral tail protein gpJ to LamB (Maltoporin),” Biochemistry, vol. 45, no. 8, pp. 2708-2720, February 2006.
[32]J. R. Meyer, D. T. Dobias, J. S. Weitz, J. E. Barrick, R. T. Quick, and R. E. Lenski, “Repeatability and contingency in the evolution of a key innovation in phage lambda,” Science, vol. 335, no. 6067, pp. 428-432, January 2012.
[33]C. Anders, O. Niewoehner, A. Duerst, and M. Jinek, “Structural basis of PAM-dependent target DNA recognition by the Cas9 endonuclease,” Nature, vol. 513, no. 7519, pp. 569-573, September 2014.
[34]F. St-Pierre, L. Cui, D. G. Priest, D. Endy, I. B. Dodd, and K. E. Shearwin, “One-Step Cloning and Chromosomal Integration of DNA,” ACS Synth. Biol., vol. 2, no. 9, pp. 537-541, September 2013.
[35]L. C. Thomason, N. Costantino, and D. L. Court, “E. coli genome manipulation by P1 transduction,” Curr. Protoc. Mol. Biol., vol. Chapter 1, p. Unit 1.17, July 2007.
[36]D. G. Gibson, L. Young, R.-Y. Chuang, J. C. Venter, C. A. Hutchison 3rd, and H. O. Smith, “Enzymatic assembly of DNA molecules up to several hundred kilobases,” Nat. Methods, vol. 6, no. 5, pp. 343-345, May 2009.
[37]C. Engler, R. Gruetzner, R. Kandzia, and S. Marillonnet, “Golden gate shuffling: a one-pot DNA shuffling method based on type IIs restriction enzymes,” PLoS One, vol. 4, no. 5, p. e5553, May 2009.
[38]J. L. Hartley, G. F. Temple, and M. A. Brasch, “DNA cloning using in vitro site-specific recombination,” Genome Res., vol. 10, no. 11, pp. 1788-1795, November 2000.
[39]C. A. Schneider, W. S. Rasband, and K. W. Eliceiri, “NIH Image to ImageJ: 25 years of image analysis,” Nat. Methods, vol. 9, no. 7, pp. 671-675, July 2012.
[40]T. M. Wannier et al., “Improved bacterial recombineering by parallelized protein discovery,” Proc. Natl. Acad. Sci. U.S.A, vol. 117, no. 24, pp. 13689-13698, June 2020.

1. A method of generating targeted nucleic acid diversity in a CRISPR-associated (Cas) gene, comprising
a) expressing in a recombinant cell comprising a Cas gene, a recombinant error-prone reverse transcriptase (RT) and a recombinant spacer RNA comprising a target sequence for mutagenesis of a DNA sequence in the Cas gene;
b) generating a mutagenized cDNA polynucleotide homologous to the DNA sequence in the recombinant cell;
c) expressing a recombinant recombineering system in the recombinant cell; and
d) recombining the mutagenized cDNA with the homologous DNA sequence of the Cas gene in the recombinant cell.

2. The method according to claim 1, wherein the recombinant error-prone reverse transcriptase (RT) comprises a recombinant diversity-generating retroelement (DGR) reverse transcriptase (RT) major subunit and a recombinant DGR accessory subunit (Avd), and the recombinant spacer RNA comprises a recombinant DGR spacer RNA comprising the target sequence.

3. The method according to claim 1, wherein the recombinant error-prone reverse transcriptase (RT) comprises the motif I/LGXXXSQ (SEQ ID NO: 2).

4. The method according to claim 3, wherein the recombinant error-prone RT is a mutant Ec86 retron reverse transcriptase comprising the replacement of the motif QGXXXSP (SEQ ID NO: 1) with the motif I/LGXXXSQ (SEQ ID NO: 2).

5. The method according to claim 2, wherein the recombinant DGR RT, the recombinant DGR Avd, and the recombinant DGR spacer RNA are from the Bordetella bacteriophage BPP-1.

6. The method according to claim 5, wherein the recombinant DGR RT comprises a mutation that decreases its error rate at adenine position selected from the group consisting of: R74A and I181N, the positions being indicated by alignment with SEQ ID NO: 4.

7. (canceled)

8. The method according to claim 1, wherein the recombinant recombineering system is a recombinant single-stranded annealing protein mediating oligonucleotide recombineering selected from the group consisting of: the phage lambda's Red Beta protein, RecT, PapRecT and CspRecT.

9. The method according to claim 1, wherein the Cas gene is Cas9 gene Cas12 gene or Cas13 gene.

10. (canceled)

11. (canceled)

12. The method according to claim 1, wherein the homologous DNA sequence of the Cas gene is in the PAM interacting domain (PID).

13. The method according to claim 1, wherein, the Cas gene comprises at least one nonsense mutation in the homologous DNA sequence of the Cas gene in the PAM interacting domain (PID) at one or more of positions selected from L1111, R1122, K1123, D1135, Y1141, L1144, S1216, G1218, E1219, L1220, A1322, K1334, R1335, and T1337, said positions being indicated by alignment with SpCas9 amino acid sequence.

14. The method according to claim 1, wherein the recombinant cell further comprises at least on recombinant CRISPR guide RNA.

15. (canceled)

16. The method according to claim 3, wherein the recombinant Cas protein encoded by the Cas gene, recombinant DGR RT, recombinant DGR Avd, at least one recombinant DGR spacer RNA, recombinant recombineering system, and optionally at least one recombinant CRISPR guide RNA are all expressed from one or a plurality of recombinant plasmids together comprising coding sequences for the recombinant Cas protein, recombinant DGR RT, recombinant DGR Avd, at least one recombinant DGR spacer RNA, recombinant recombineering system and optionally at least one recombinant CRISPR guide RNA.

17. (canceled)

18. (canceled)

19. The method according to claim 1, wherein the recombination frequency is at least 10%.

20. The method according to claim 1, wherein the recombinant cell comprises at least two DGR spacer RNAs comprising the target sequence; wherein the multiple spacer RNAs target the same gene in the recombinant cell.

21. The method according to claim 1, wherein the recombinant cell is an E. coli cell.

22. The method according to claim 21, wherein the E. coli cell expresses dominant negative mutL; and/or is deleted for the two exonucleases SbcB and RecJ to increase recombineering efficiency.

23. A method of generating a library of Cas protein variants, comprising:
expressing in a recombinant cell population comprising a recombinant Cas gene, a recombinant error-prone reverse transcriptase (RT) and recombinant spacer RNA comprising a target sequence for mutagenesis of a DNA sequence in the recombinant Cas gene;
making a mutagenized cDNA polynucleotide homologous to the DNA sequence in the recombinant cell population;
expressing a recombineering system in the recombinant cell population;
recombining the mutagenized cDNA with the homologous DNA sequence of the recombinant Cas gene in the recombinant cell population; and
expressing the recombinant Cas gene comprising the mutagenized DNA sequence in the recombinant cell population to generate a library of expressed Cas protein variants.

24. A method of selection and/or screening of a library of Cas protein variants, comprising:
a) generating a library of expressed Cas protein variants in a recombinant cell population according to the method of claim 23; and
b) selecting and/or screening the activity of the expressed Cas protein variants.

25. (canceled)

26. (canceled)

27. (canceled)

28. (canceled)

29. (canceled)

30. (canceled)

31. A recombinant cell comprising:
a first recombinant expression plasmid comprising coding sequences for the recombinant DGR RT and recombinant DGR Avd;
a second recombinant expression plasmid comprising a coding sequence for the recombinant single-stranded annealing protein (SSAP) mediating oligonucleotide recombineering;
a third recombinant expression plasmid comprising a coding sequence for the recombinant Cas protein; and
coding sequences for the at least one recombinant DGR spacer RNA inserted in the first and/or second expression plasmid.

32. (canceled)

33. (canceled)

34. (canceled)

35. (canceled)

36. (canceled)

37. A library of Cas gene mutagenized sequences made according to the methods of claim 1.

38. (canceled)

39. (canceled)
